SEQUENCE LISTING (174 sequences)

SEQ ID NO: 1 - DNA, 1244 bp, Homo sapiens
  misc_feature 14, 15, 33, 34, 37, 39, 45, 47, 55, 60, 70, 78, 82, 120,142, 143, 145, 154, 206, 236, 272, 275, 276, 281, 290, 301,304, 313, 319, 325, 327, 335, 337, 340, 341, 343, 349, 356,358, 364, 372, 375, 377, 380, 382, 386, 389, 395, 401: n = A,T,C or G
  misc_feature 402, 405, 413, 415, 424, 426, 433, 437, 448, 449, 452,453, 455, 468, 476, 477, 485, 491, 496, 500, 503, 507, 511, 512,515, 517, 522, 525, 530, 532, 533, 538, 547, 551, 555, 558,562, 570, 574, 579, 580, 581, 587, 595, 597, 598, 605: n = A,T,C or G
  misc_feature 624, 625, 630, 642, 644, 648, 653, 654, 657, 664, 672,682, 684, 685, 693, 698, 708, 717, 718, 721, 722, 725, 728, 729,733, 734, 742, 753, 765, 773, 775, 782, 789, 790, 793, 794,798, 799, 800, 808, 815, 821, 830, 838, 839, 841, 842: n = A,T,C or G
  misc_feature 847, 852, 856, 865, 869, 879, 882, 884, 889, 891, 894,900, 906, 910, 911, 916, 917, 923, 924, 926, 928, 930, 931, 936,938, 939, 947, 951, 957, 958, 964, 967, 968, 982, 984, 985,988, 989, 994, 997, 998, 999, 1003, 1004, 1005, 1021: n = A,T,C or G
  misc_feature 1022, 1041, 1045, 1049, 1050, 1053, 1054, 1063, 1066,1067, 1069, 1073, 1075, 1079, 1085, 1092, 1094, 1095, 1096, 1105,1106, 1113, 1118, 1125, 1128, 1135, 1137, 1138, 1139, 1140,1141, 1162, 1164, 1171, 1184, 1187, 1191, 1193, 1196: n = A,T,C or G
  misc_feature 1197, 1204, 1207, 1212, 1217, 1220, 1221, 1222, 1224,1230, 1239, 1240, 1241: n = A,T,C or G
cttccttttaaaanncccttttgaaaaggcccnnacnanagtttncntcaagatntgccncagggcttgncaccgagncttngtatccactagtaacggtcgccagtgttttggaatttnccttaatcccatggaaattaanngnaaaaacagnatcctgctgggcactggaattataaacatgatcattaagttctgtcaattanccagacatcacaccatgaagcctactgganatccgactcaaaattacaaagaagcatatgctaacnggnntgtangaggaaaanggtcgtgtttnacntagatgggnagatangatgcncncgcccccncncanncncccccncctcccngnacccgnccgcaccncancnccncnccgntanccacgngcaaanngcnctttctcnanggtcccccngnccatccntgcnccctctaaacnnccnnanccctcggctcccnaaatcctnncgcctctnaaagcnccgtnacgngtnttanctcnncanancgcanaanctccncnnccacnctcaccaancctngccnctnctcncccccccnactncgccnnncatccnacacactngnntgcaccnctccacaccccccacccgnngcccncctcccccctcntngatngtacnnccncaccccncacgccancgcgcatctncnntccctcgnctacncccacccccncaccttccnnccnnctnatnnaccnnctatccgnaacaccccccncccctccgcacncgctcccncncgcggtncaccctnntcnnaacnnncccccacnaaacccncacctntacctaacnacccctanncnnccccncccantacnaccccccancacnacacttcccntcncnccacncntgnaacacntcacantcgnnctcannctaccnnancncnnctatncnncccctatnaganatcacnncctccnacnnacgacctacacctncnncannacacncannncagnnnctccctctaccatcanntgccactccacatacatgncctnaccnntanngcaccaccnatnncntcanantcancgcatntaatgcntnnnctcgcacannacgcatncacanatcacanctntacaagngnnnnncacctcatccgtcacgtcagncnatgcgcnacgacgccatcgnacntccncntcnncttcccngcnctctncctgnctnnntnctatgncacaacatnnnccc

SEQ ID NO: 2 - DNA, 1142 bp, Homo sapiens
  misc_feature 5, 6, 18, 19, 29, 33, 53, 55, 74, 82, 90, 91, 93, 100,102, 278, 398, 620, 646, 659, 690, 694, 722, 758, 761, 766, 767,774, 782, 795, 796, 805, 811, 813, 818, 829, 833, 844, 855,868, 870, 876, 878, 882, 893, 900, 930, 933, 934, 945: n = A,T,C or G
  misc_feature 948, 955, 958, 959, 963, 966, 971, 973, 977, 994, 1004,1010, 1014, 1027, 1028, 1030, 1031, 1043, 1046, 1049, 1053, 1056,1069, 1073, 1076, 1080, 1081, 1083, 1099, 1101, 1105, 1108,1114, 1115, 1121, 1123, 1128, 1129, 1132, 1133, 1138: n = A,T,C or G
  misc_feature 1139, 1142: n = A,T,C or G
ttaannccttttaagccnntgctcgggcnccgncagtgtgacggatatctgcngnaattcggcttactataggncacgcgtngtcgacgnntngggctgntncttgttaaaaatacgattctaagccccatcccaggctttccgactctgggattaaggcccagaaatctgtattttaaaagctgctctatgtgcagcaaactttgggagctcctattcccatgcttattattaagtacatagtaccttctggaccattatacttagccatttgtagntaattacctgtacatcttatctccccatctatattaaacacaaccattttcctcatacataccagttagcatatacttctttgtaattttgagtcggatatccagtaggcttcatggtgngatgtctggataattgacagaacttaatgatcatgtttataattccagtgcccagcaggatgctgtttttgaattaatttccatgggattaagccgaattccagcacactggcggccgttactagtggatccgagctcggtaccaagcttgatgcatagcttgagtattctatagtgtcacctaaatagcttggcgtaatcatggtcatagctgtttcctgngtgaaattgttatccgctcacaattncacacaacatacnagccggaagcataaagggcaaagcccggggngccnaatgagtgagctaactcacattaatgcnttgcgctcactgcccgcttccagtcgggaaacctgntcnctgcnnctgcatnaacggaancggccaacgcgcnncgagaggcngtttgngnattgngcgctctttcncttnctcgcatcacntaactcctggnccccggtcgctcngnctgccncnagcngcttcagctcntttaaangcggttactccgtatattcccagaaatcanggnntcaacgcatgnaangaacatngtnngcanaangtccncnatanggcccccgaacccctanaaaggccctngttggntgcntttttcccttgcnncnncccccttgcganctnccnaaanctnccccctcctcccntggnggntacnnanccgcaggatttatacngncccngcntttccnntcgcancntcctnntcnngctcnnccn

SEQ ID NO: 3 - DNA, 1210 bp, Homo sapiens
  misc_feature 13, 23, 30, 40, 46, 48, 51, 60, 88, 97, 100, 106, 110,119, 120, 155, 197, 296, 327, 353, 362, 382, 385, 406, 422, 433,434, 440, 446, 448, 449, 450, 454, 470, 474, 476, 495, 496,498, 499, 502, 504, 521, 523, 539, 552, 559, 561, 577: n = A,T,C or G
  misc_feature 578, 595, 599, 601, 604, 610, 613, 615, 638, 642, 646,648, 652, 653, 659, 660, 662, 678, 686, 687, 688, 689, 690, 694,695, 706, 717, 718, 719, 725, 728, 738, 739, 745, 751, 757,764, 767, 769, 770, 783, 784, 785, 805, 810, 814, 818: n = A,T,C or G
  misc_feature 822, 823, 828, 832, 836, 842, 843, 846, 854, 859, 860,867, 868, 872, 878, 880, 886, 888, 893, 898, 901, 909, 921, 927,934, 949, 955, 958, 966, 969, 980, 982, 987, 989, 991, 994,998, 1002, 1008, 1019, 1021, 1023, 1026, 1035, 1036: n = A,T,C or G
  misc_feature 1048, 1052, 1055, 1056, 1068, 1072, 1078, 1083, 1086,1090, 1103, 1106, 1123, 1128, 1133, 1135, 1144, 1145, 1148, 1153,1155, 1167, 1170, 1172, 1176, 1188, 1193, 1203, 1210: n = A,T,C or G
ttaaacccccttntaaaaccctnctgaaangtcccactanagtttncncanactatgccncaagcttggtaccgagcttggtatccantagcaacgnttngccagngttntggaattcnncttaatcccatggaaattaattcaaaaacagcatnctgctgggcactggaattataaacatgatcattaagttctgncaattatccagacatcacaccatgaagcctactggatatccgactcaaaattacaaagaagtatatgctaactggtatgtatgaggaaaatggttgtgnttaatatagatggggagataagatgtacagnctcttaactacaaatggctaagcctnatggcccanaaggcccacctccttaatantangcatgggaataggaccttccncaagttccctgcacanagagcagcctnnaaactntcagantnnntggncctcaatccccagagnctcnanagcctgggatggggtctcnnanntcntncttctaacaagcaccancnccgggcccgtcgcccncgcctgcccccanacacacncncgaaactcctgcactnnaaccatccacaccgccncccntncanagcacncancncaagggcccccttcccctcaacnccgncccntncccnnctctanntncccccccttcacaccntcgcagcnnnnncccnncccccgtcctnccacatttccnnncccctncancacctctccnntctctnactacncggccnccacagnccncnnccttcaccacccnnnccctcttccccccacttcgntcaanctcnactntctnnacccnctcncctncccctnnccncatctaanctctnnctccccnngccntctatncnccaccntnccccnctcanccnccccgccntcctaccacccntttccncaccttntctccctacccctantggccncgnccttcctnctncacaccctctncngtccnananccngctnaccncctctncgcgcccctcncncnaancccctcgcnncccgacaccccntccntcnnttaccacctgtnaccncctacngcccnctncgtncacacctgtcgtntcntcattctcatcccatcncacgnccctncntctatcacnntcnctcantnttaccatcactntcntncatnctctctctcctngcacngtctcatcanctccccn

SEQ ID NO: 4 - DNA, 1160 bp, Homo sapiens
  misc_feature 5, 6, 24, 25, 26, 28, 38, 39, 46, 52, 53, 92, 102, 524,549, 558, 669, 670, 692, 740, 748, 779, 801, 803, 812, 826, 831,841, 865, 869, 879, 885, 893, 904, 916, 926, 932, 936, 946,958, 960, 965, 966, 974, 981, 1000, 1007, 1009, 1014: n = A,T,C or G
  misc_feature 1017, 1018, 1037, 1051, 1064, 1066, 1076, 1077, 1082,1091, 1092, 1099, 1105, 1108, 1110, 1121, 1122, 1134, 1135, 1150,1155, 1156, 1160: n = A,T,C or G
ttaanncctttaaaaccttttaannngnccgctcgagnngccgccntgtganngatatctgcagtaattcggcttactatagggcacgcgtnttcgacggtncgggctggtacttgttaaaaatacgattctaagccccatcccaggctttccgactctgggattaaggcccagaaatctgtattttaaaagctgctctatgtgcagtaaactttgggagctcctattcccatgcttattattaagtacatagtaccttctggaccattatacttagccatttgtagttaattacctgtacatcttatctccccatctatattaaacacaaccattttcctcatacataccagttagcatatacttctttgtaattttgagtcggatatccagtaggcttcatggtgtgatgtctggataattgacagaacttaatgatcatgtttataattccagtgcccagcaggatgctgtttttgaattaatttccatgggattaagccgaattccagcacactggcggncgttactagtggatccgagctcggnaccaagctngatgcatagcttgagtattctatagtgtcacctaaatagcttggcgtaatcatggtcatagctgtttcctgtgtgaaattgttatccgctcacaattccacacaacatacnnagccggaagcctaaagtgcaanggctggggtgcctaatgagtgagcctaacttacattaattgcgttgcnctcacttncccgctttccagccgggaaaacctgtcgtgnccagctgcattaatgaatcggncnacgcgccgnggcagaggcggttntgcgnattgcgcgcnttttccgcttcctcgctcacctgnctcnctgcgccttngccgcnccggccgngggcgagccgncattcagctcanttaaaggcgntaatancggntatcccccancaatcctggggntnccccnngaaaacancatgtcnagccacaagggccagctanaagtccngnaaccntcnnaacgggcccctgttgttgnccgttttccatagngcccggccccccngnctagcttcannaaatncccttcaanntcatagntggcgnaancnccatcggcctnnccacatcccctnntgtcccccttggtangattnntccn

SEQ ID NO: 5 - DNA, 1155 bp, Homo sapiens
  misc_feature 6, 7, 21, 24, 25, 30, 34, 37, 44, 88, 91, 101, 102, 123,130, 131, 196, 241, 405, 431, 490, 535, 545, 555, 558, 559,568, 576, 586, 599, 612, 622, 626, 637, 648, 660, 665, 713,719, 729, 733, 747, 756, 758, 765, 766, 775, 777, 778: n = A,T,C or G
  misc_feature 782, 792, 798, 799, 809, 816, 828, 830, 850, 858, 867,868, 876, 878, 893, 905, 916, 927, 929, 932, 934, 938, 943, 950,956, 957, 959, 963, 964, 967, 980, 981, 986, 994, 996, 1006,1007, 1008, 1012, 1017, 1023, 1030, 1032, 1036, 1039: n = A,T,C or G
  misc_feature 1040, 1041, 1042, 1045, 1047, 1053, 1058, 1063, 1072,1073, 1083, 1087, 1090, 1094, 1101, 1108, 1110, 1125, 1126, 1130,1132, 1137, 1139, 1143, 1155: n = A,T,C or G
ttaaannccttttaaaacctntgnnaaagncccncancttgcanaaagcttgctccgagcttggatccacttgtaacggtcgccagtnttntggaattttnntactatagggcacgcgtgggncgacggnncgggctggtaaaagctgctctatgcgcagtaaactttgggagctcctatccccatgcttattatnaagcacatagtaccttctggaccattatacttagccatttgtagntaattacctgtacatcttatctccccatctatattaaacacaaccattttcctcatacataccagttagcatatacttctttgtaattttgagtcggatatccagtaggcttcatggtgtgatgtctggataattgacagaacttaatgatcatgtttataatnccagtgcccagcaggatgctgttttngaattaatttccatgggattaagccgaattctgcagatatccatcacactggcggccgntcgagcatgcatctagagggcccaattcgtcctatagtgagtcgnattacaattnactggccgtngtnnacaacgccntgactggnaaaaccctgncgttacccaactnaatcgccttgcancacatccccnttcnccagctggcgnaatagctgaanaggcccgcaccnatcgnccttcccaacagttgcgcagcctgaatggcgaatggacccgccctgtnccggcnccattaagcncggngggtgtggcggttncccgcagcngngacccgnncacttgccngnncccnagccccgctncttagnnttcttccctnccttttncggcccgttcgncngcttttccccccaaaggctntaaaatcngggcctccnntttagggntncccaaccagtgctcnacggcaacctcncccccaaaaanctctgatttgngngangnttcncgtancgcccantcgccnngntagnncgnttttcccccttcnnacccnggagcccncnctttttaaannnggtnctctnggtctnaaaactncncccnccnnnntcntntcccgnaccanttctnacctctctnnaggcattctnctcnctnctgngcgcttntcgatananacagccctcgcattnnccananctttncncgcnctatctaacccn

SEQ ID NO: 6 - DNA, 1157 bp, Homo sapiens
  misc_feature 7, 8, 17, 23, 50, 61, 101, 102, 110, 282, 290, 293, 298,302, 304, 315, 322, 333, 359, 360, 367, 400, 404, 412, 421,428, 430, 458, 469, 482, 486, 491, 505, 527, 537, 538, 549,563, 564, 570, 573, 581, 583, 589, 590, 594, 599, 600: n = A,T,C or G
  misc_feature 618, 620, 621, 628, 629, 641, 664, 670, 683, 686, 691,696, 711, 720, 723, 737, 738, 746, 747, 748, 760, 764, 773, 790,799, 823, 831, 838, 845, 859, 873, 879, 887, 900, 910, 915,921, 925, 928, 930, 931, 935, 940, 954, 960, 989, 992: n = A,T,C or G
  misc_feature 1000, 1005, 1007, 1015, 1016, 1024, 1032, 1037, 1039,1072, 1077, 1081, 1089, 1099, 1102, 1113, 1121, 1136, 1143, 1147,1149, 1157: n = A,T,C or G
tttaaanncccttttanagccgnctgctcgggcgcccgccagtgtgtatngcatatctgcngcaattcggcttaatcccatggtaaattaattcaaaaacnncatcctgntgggcactggaattataaacatgatcattaagttctgtcaattatccagacatcacaccatgaagcctactggatatccgactcaaaattacaaagaagtatatgctaactggtatgtatgaggaaaatggttgtgtttaatatagatggggagataagatgtacaggtcancaactacnaanggctnagtntnatggtccaganggtcctntgcacttaatnataagcatggggataggagcttccanngcttacngtccatagagcagcttttaccagcccgggccgncgancacgcctnccctatagnagccgcntnccaccacactggcggccgccactagtgnatccccgctcngcaccaagcttgntgcntagcncgcgtctctatagngccccctaaatagcttgccctntccccgctcnnttgctgtttcngtgtcaaattgttnncgttcnctnttcacacncntatacnntccnccccnncaagtcgcaagcctgatngnncctaacnncgtcacctctcncacattaacttgccgtttcgccnctcccnccccctcttccgngcnggaanccctncgcggccaccctctnttctgcctnccnccaccctccccccnngtcctttnnnttttcctgaacntccncccctttcntcctccaaccccttctntccctcctngcttatcgcctccggcctcgcccncccaccgngcctctntgcgttnattgcggcttttcnccttcccgtctccntccctncttccccnctcttcctcgctnccccccgccnccccngacccnctcnacncnntccncctgntccccccccaaccntgtccntccccccgctacgctccttccctcctcgnccnccttccgnccctnanccccctannggcctccnatctctcnctctntntcaattagtcctccccccctctccccggccttncctcntctnccccctcnatcttctcgngcnctttccccctntgcctccnttcgctcctgcgcgnttccccnccancncccccccn

SEQ ID NO: 7 - DNA, 1194 bp, Homo sapiens
  misc_feature 7, 13, 20, 36, 39, 43, 50, 57, 84, 96, 102, 107, 117,140, 151, 152, 189, 194, 205, 245, 293, 294, 295, 314, 315, 316,324, 333, 335, 345, 355, 358, 359, 361, 364, 365, 367, 371,376, 378, 391, 392, 393, 400, 403, 409, 411, 414, 416: n = A,T,C or G
  misc_feature 439, 440, 445, 450, 459, 460, 467, 468, 469, 471, 474,476, 481, 482, 484, 486, 489, 492, 493, 497, 499, 504, 506, 513,518, 520, 523, 534, 536, 540, 548, 549, 568, 573, 578, 592,595, 597, 600, 601, 607, 610, 614, 621, 622, 624, 638: n = A,T,C or G
  misc_feature 641, 647, 654, 664, 668, 676, 678, 682, 686, 691, 694,698, 712, 714, 726, 729, 731, 738, 743, 746, 751, 755, 769, 771,775, 777, 778, 781, 788, 790, 806, 809, 814, 820, 826, 834,846, 848, 849, 859, 871, 877, 882, 885, 888, 891, 892: n = A,T,C or G
  misc_feature 893, 900, 910, 920, 922, 925, 938, 940, 941, 946, 950,957, 968, 978, 987, 991, 993, 999, 1013, 1018, 1026, 1030, 1035,1037, 1055, 1056, 1061, 1069, 1071, 1072, 1075, 1083, 1090,1099, 1100, 1101, 1105, 1111, 1119, 1142, 1149, 1151: n = A,T,C or G
  misc_feature 1158, 1173, 1174, 1183, 1188, 1189, 1194: n = A,T,C or G
ttaaacnccttgnaaaaccncctgaaaggtcccacnaantttncccaagntatgccncaagctcggtaccgagcttggaaccantagccaacgttngccagngtttntggaattccncttaatcccatggaaattaattnaaaaacagcannctgctgggcactggaattataaacatgatcattaagntctgncaattatccanacatcacgccatgaagcctactggatatccgactcaaaantacaaagaagcatatgctaactggtatgtatgaggaaaacggctgcgnnnaatacagaaggggagacannncgccagcncatcaccgnanctaagccccntcccagggtntcnnangcnncnaccnccccnancaagctgcaccannnaagcccnctntacgtncnagnanctcgcgcagcctccaaccccccnncccancacanaccccccgnnccctccnnngncantntaccnngntntcnacnnctcncngcccngnccccccncatcnanggncaccatccccnanctcncccgcttnncccatcaccctcacacccnccccnactcncccccaccccaacnccngngcnngcaccnccnccancccctanncngaccccatctcgtntcncaccanccccccnaccccccccncgtnccaacacncncgcncccnccctngcngctncatccccacccccncncccccaccccancancnctccccngcccnctnccccncccncacccctccccccncntcancnnccnccacacncnccaatcccatcctccnccnacccnccctgntccccnctaccttncaccaccacccncnncctctcccanaaccccaccccncttccnccccnccnccnccnnncccgctncaccatcccnccctccattncnganacatctactatanannccccncccnttcaccntttcacacccncgaccctccnactcatctncccncncctctnaccccaccacactnacccncccccccngccnccccngncttacccctcactaaccnnccaantctccacntnncancacccaancaccccntcgcatccnnnatcntcttcncacccacnccacccacccatactcccacctntccatcnancatctcnacgctcccctcacannctcattgcncgccnnccccn

SEQ ID NO: 8 - DNA, 1158 bp, Homo sapiens
  misc_feature 6, 7, 22, 26, 29, 30, 42, 43, 57, 58, 68, 107, 109, 516,529, 552, 663, 714, 718, 725, 728, 737, 750, 785, 789, 797,810, 818, 820, 838, 845, 849, 850, 853, 855, 859, 861, 870,871, 872, 882, 894, 901, 908, 910, 914, 916, 922, 927: n = A,T,C or G
  misc_feature 936, 943, 951, 952, 969, 976, 982, 992, 998, 1000, 1004,1009, 1012, 1019, 1025, 1041, 1042, 1047, 1055, 1058, 1060,1062, 1066, 1067, 1071, 1078, 1081, 1083, 1092, 1099, 1104,1105, 1108, 1113, 1114, 1118, 1119, 1122, 1123, 1136: n = A,T,C or G
  misc_feature 1142, 1144, 1147, 1158: n = A,T,C or G
ttaaannccttttaaaaccctnttanagnngcccgctcgggnngccgccagtgtganngatatctgcngtaattcggcttactatagggcacgcgtgttcgacggtncnggctggtaaaagctgctctatgtgcagtaaactttgggagctcctattcccatgcctattattaagtacatagtaccttctggaccattatacttagccatttgtagttaattacctgtacatcttatctccccatctatattaaacacaaccattttcctcatacataccagttagcatatacttctttgtaattttgagtcggatatccagtaggcttcatggcgtgatgtctggataattgacagaacttaatgatcatgtttataattccagtgcccagcaggatgctgtttttgaattaatttccatgggattaagccgaattccagcacactggcggccgttactagtggatccgagctcggtaccaagcttgatgcatagcttgagtattctatagtgtnacctaaatagctnggcgtaatcatggtcatagctgnttcctgtgtgaaattgttatccgctcacaattccacacaacatacgagccggaagcataaagtgtaaagcctggggcgcctaatgagtgagctaactcacattaattgcgntgcgctcactgcccgcttccagacgggaaactgtcgtgccagctgcattantganatccccnacncgccggggngagggcggtttgncgtattgggcgctccttcgcttcctcgctcactgnctcngctgcgcntcggtcgctcggnttgcgctnancggttatcagcttcactnaaaaggncggnnatncngctntnccacagcannncgggcgattncccacggaaagnaccctgngagccananggcncncaaaangcccngggaacccnaaaaaangccccccnnttgccggtttttccatnggctccncccccngccaggcatnccaaananttcnccctncangtcaganggggcnctacccctcaggcctnncaagnttccctcnctntntncccnnggangctcctntgnanctctttctntttcgcncctcnncgnttacnnggannccnntcgacttctttcncttggngnagncgcgccctccn

SEQ ID NO: 9 - DNA, 1155 bp, Homo sapiens
  misc_feature 6, 7, 10, 57, 67, 76, 92, 94, 95, 105, 127, 199, 273,275, 278, 351, 377, 505, 538, 553, 595, 612, 636, 644, 655, 657,680, 711, 712, 733, 762, 775, 776, 782, 785, 786, 793, 819,824, 845, 866, 882, 889, 893, 898, 900, 908, 911, 914: n = A,T,C or G
  misc_feature 924, 933, 950, 963, 972, 978, 980, 984, 986, 987, 990,993, 999, 1000, 1003, 1009, 1015, 1017, 1024, 1027, 1039, 1040,1045, 1046, 1085, 1099, 1102, 1114, 1117, 1121, 1133, 1146,1155: n = A,T,C or G
ttaaannccntttaaaaacctctgcaaaagtcccctcaaactttgccaaagcttgcnaccgagcttngatccactngtaacggtcgccagtntnntggaattttncttactatagggcacgcgtggncgacggtccgggctggtctgggatgtccttttgcttcccttctagtcatctttccagggacagttcagattncatatataccattaaaccttcttaattattctagcctgaaacaatctctcctctgaatttgatttttctaaaangntgnataagagccacacctttgagaatcgactgagtacttgttaaaaatacgattctaagccccatcccaggctttncgactctgggattaaggcccagaaanctgtattttaaaagctgctctatgtgcagtaaactttgggagctcctattcccatgcttattattaagtacatagtaccttctggaccattatacttagccatttgtagttaattacctgtacatctnatctccccatctatattaaacacaaccattttnctcatacataccagntagcatatacttctttgtaatttcgagtcggatatccagtangcttcatggtgtgatgnctggataattgacagaaccttaangatcatgntcataattccngngcccagcacggatgctgttttgnaattaattcccatgggcatcaagccgaaatnntgcagatatccatcacactgncggcccgcttgagcatgcatctagagggnccccaattctccnntagcgngtnntattacnattcactggcccgtcggttcacaacnctcgngactgggaaaaccctgcggtncccaactttaatcggcctgcnagccattccccttttntcagctngcgnaatangnaaagaggnccngtnccttcgctcnttccaacanctggcgccagactgaangccgaaaggcacncccctcttnccggcncnatcnannccnggngggtgnnggnggtccncccacngngaccggntcnccttctcagcgnncctannccggctcctctccgcatcttccctctctttctcgccggntacgcgctctctcnccnctcagctctaanctntggncccccattaggnttccctctacacnctcttaccn

SEQ ID NO: 10 - DNA, 1170 bp, Homo sapiens
  misc_feature 1, 8, 21, 34, 40, 94, 97, 105, 357, 499, 503, 504, 510,530, 535, 546, 552, 567, 568, 575, 596, 601, 614, 616, 623, 631,635, 637, 640, 643, 650, 654, 656, 662, 663, 665, 671, 679,683, 690, 695, 705, 712, 716, 735, 737, 741, 743: n = A,T,C or G
  misc_feature 744, 750, 753, 765, 769, 771, 782, 789, 791, 802, 806,807, 815, 816, 819, 820, 822, 823, 830, 837, 841, 852, 855, 858,860, 868, 869, 870, 874, 878, 884, 885, 886, 887, 896, 897,902, 905, 906, 907, 908, 909, 915, 917, 925, 941, 954: n = A,T,C or G
  misc_feature 957, 958, 960, 962, 963, 966, 969, 970, 972, 982, 988,993, 998, 1000, 1003, 1004, 1010, 1011, 1012, 1015, 1016, 1018,1024, 1027, 1032, 1034, 1035, 1036, 1037, 1041, 1059, 1061,1063, 1064, 1066, 1071, 1077, 1078, 1079, 1081, 1083: n = A,T,C or G
  misc_feature 1089, 1091, 1093, 1097, 1099, 1100, 1101, 1113, 1114,1118, 1121, 1126, 1127, 1128, 1131, 1138, 1141, 1144, 1148, 1153,1162, 1165, 1166: n = A,T,C or G
ntttaaancccttttatagcncttgctcgggcgnccgccntgtgatggatatctgcagaattcggcttaatcccatggtaaattaattcaaaancancatcctgntgggcactggaattataaacatgatcattaagttctgtcaattatccagacatcacaccatgaagcctactggatatccgactcaaaattacaaagaagtatatgctaactggtatgtatgaggaaaatggttgtgtttaatatagatggggagataagatgtacaggtaattaactacaaatggctaagtataatggtccagaaggtactatgtacttaataataagcatgggaataggagctcccaaagntcactgcacatagagcagcttttaaaatacagatttctgggccttaatcccagagccggaaagcctgggatggggcttagaatcgcattttaacaagtactcagtcgattctcaaaggcgtggctcttatacaacattttanaaanntcaaantcagaggagagattgcttcnggctngaataattaanaaggtntaactggtatatatnnaactctnaacctgcccctggaaagatgnctcgnaacggaagcaaangnccttccnagacccancccngnccntcntccactncctncncctatnncncagccnaattccancacnctggctncccgntacttgtggntcctcancccngctccaatcttgacgcacncntcgngnntctatnggngtcccccacttnctcncncttattctttnctctctncntcttccctgcntganntcgggtcnnccnncnncttcccnccttctncccnccccttaagcnctncantnccgctccnnncccncctnaatggnnnnccttcactnncattnatnnnnnggcctncncttcctgnccctttccccgttctncaactccggctcnccnncntnntantanncntctccccctncccccntcttntcggncnccnnagtatnnnccnncncttccnttncttgntnnnnctcntcccttcccttttcgctncncnncncttcnctctcnnncncntactcncntnctcngnnnctttcccgtttnncccnctnctccnnnctntccctcnctnctntctnccttngctccgtcntcnncccc

SEQ ID NO: 11 - DNA, 1176 bp, Homo sapiens
  misc_feature 2, 3, 4, 9, 10, 18, 25, 26, 30, 35, 37, 82, 215, 422,453, 569, 583, 606, 619, 624, 633, 649, 650, 653, 658, 676, 681,682, 687, 705, 718, 733, 743, 755, 760, 761, 762, 767, 768,772, 773, 786, 789, 792, 795, 797, 804, 807, 818, 832: n = A,T,C or G
  misc_feature 833, 843, 846, 849, 850, 851, 854, 858, 861, 863, 867,874, 881, 887, 890, 906, 908, 913, 927, 932, 934, 951, 953, 955,959, 965, 966, 984, 985, 986, 990, 1001, 1016, 1028, 1040,1042, 1043, 1045, 1047, 1055, 1058, 1066, 1068, 1069: n = A,T,C or G
  misc_feature 1070, 1079, 1080, 1082, 1084, 1085, 1091, 1094, 1101,1102, 1103, 1107, 1110, 1124, 1126, 1128, 1140, 1149, 1151, 1157,1158, 1161, 1166, 1169, 1170: n = A,T,C or G
cnnnctctnnttttaaanccctttnnaaanccccngnaattatatacatgatcattaagttctgtcaattatccaggacatnacaccatgtaagcctactggatatccgtctcaaaattacaaagaagtatatgctaactggtatgtatgaggaaaatggttgtgtttaatatagatggggagataagatgtacaggtaattaactacaaatggnctaagtataatggtccagaaggtactatgtacttaataataagcatgggaataggagctcccaaagtttactgcacatagagcagcttttaaaatacagatttctgggccttaatcccagagtcggaaagcctgggatggggcttagaatcgtatttttaacaagtactcagtcgattctcaaaggtgtggctcttatacaacattntagaaaaatcaaattcagaggagagattgtntcaggctagaataattaagaaggtttaatggtatatataaaatctgaactgtccctggaaagatgactagaagggaagcaaaaggacatcccagaccagcccggccgttgaccacncgtgccctatagtngccgaattccagcacactggcgnccgttactagtgnatccnagcctcggnccaagcttgatgcatnnctngagtntttatagtgccacctcantagcnngccgnaaccatggccttagcttnctcctgcgcgacnttgctatccccttcncaattccacnccccataccganctcgnnngcctnngtgnnacgcctggcgtgnctnatnagngngctaacnccnattaatctgcngttgcgtctccctnnccgcttttcncanctnnnganaccngtncngccncttcccnttaatgncttcgnccntccccccggtgagagntntctcncgtaatgggtccgntcttncncctttctgcctcacttncncnctcngcccgnncctctctcggcttcgtgnnngcgnccttcacctcncctcacaggccggtntttcctttcatntccccgcatccntnntntncccttccnacncacctctncnnntaacgccanncntnngcctcnacncctcctnnngccntcntcctcctccttttncntntagcttcccccncctcgtccntnttctcnnaantcccnccnncaaccc

SEQ ID NO: 12 - DNA, 1164 bp, Homo sapiens
  misc_feature 7, 8, 14, 15, 26, 27, 64, 85, 92, 101, 104, 105, 114,123, 124, 145, 211, 506, 631, 673, 695, 713, 714, 734, 750, 756,784, 795, 815, 828, 830, 831, 848, 849, 870, 877, 880, 883,890, 894, 896, 899, 900, 916, 919, 934, 937, 940, 946: n = A,T,C or G
  misc_feature 954, 962, 972, 986, 988, 989, 991, 1008, 1014, 1018,1029, 1034, 1036, 1042, 1055, 1057, 1058, 1061, 1063, 1093, 1099,1104, 1105, 1109, 1113, 1121, 1126, 1127, 1138, 1149, 1158,1161: n = A,T,C or G
tttaaanncccttnntaaaaaccctnntgaaaaggtccccactatagtttactcaagctatgcntcaagcttggtaccgagcttngatccantagcaacgnttnnccagtgttntggaattcnncttactatagggcacgcgtgntcgacggcccgggctggtctgggacgcccttttgcttcccttctagtcatctttccagggacagtncagattttatatataccattaaaccttcttaattattctagcctgaaacaatctctcctctgaatttgatttttctaaaatgttgtataagagccacacctttgagaatcgactgagtacttgttaaaaatacgattctaagccccatcccaggctttccgaccctgggattaaggcccagaaatctgtattttaaaagctgctctatgtgcagtaaactttgggagctcctattcccatgcttattattaagtacatagtaccttctggaccattatacttagccatttgtagntaattacctgtacatcttatctccccatctatattaaacacaaccattttcctcatacataccagttagcatatacttctttgtaattctgagtcggatatccagtaggcttcatggtgtgatgnctggataattgacagaacttaatgatcatgtttataattccngcgcccagcaggatgctgcttngaattaattccatgggannaagccgaattctgcagatanccatcacactggcggncgctcnagcatgcatttagagggcccaactcgcnctatagtgagntgtattacaattactggccnacgttctacaccntnngtgactggcaaaacccnngcgtctaccccactttaatcnccttgcnccncanccccctnttancncanntggctgtaaatacggnaangcccgcacctcttcnccnttntccacnagtcgccnaaccctgnatggccgacngacccgcccctgtngnngnggcattaaagccgcggntgggtncgcntggcctctccncactngnacccgntccaccttctcancnnccncnccccgttctttcgccttccttccctctcantctctngcccnnctcnccantctcctcnacagnncctacatcggncgccctccctntggtgtccnccnccc

SEQ ID NO: 13 - DNA, 1160 bp, Homo sapiens
  misc_feature 3, 5, 6, 7, 24, 28, 49, 106, 107, 281, 287, 289, 293,309, 314, 320, 329, 330, 331, 338, 348, 351, 374, 383, 395, 403,406, 407, 431, 435, 444, 445, 447, 455, 458, 463, 477, 485,491, 492, 500, 506, 512, 517, 518, 521, 526, 532, 534: n = A,T,C or G
  misc_feature 544, 550, 561, 562, 564, 572, 573, 574, 578, 580, 583,591, 594, 599, 605, 606, 607, 612, 615, 616, 619, 632, 633, 640,641, 646, 650, 655, 657, 658, 662, 668, 672, 682, 685, 698,700, 706, 709, 715, 716, 719, 737, 739, 741, 744, 748: n = A,T,C or G
  misc_feature 749, 758, 767, 769, 773, 774, 775, 787, 789, 793, 800,812, 813, 816, 833, 840, 845, 847, 855, 872, 875, 880, 882, 899,905, 909, 910, 913, 914, 917, 918, 926, 935, 941, 948, 966,967, 969, 971, 972, 976, 986, 987, 997, 999, 1005: n = A,T,C or G
  misc_feature 1016, 1017, 1032, 1035, 1042, 1043, 1049, 1050, 1052,1061, 1063, 1065, 1067, 1073, 1086, 1088, 1093, 1094, 1096, 1098,1103, 1104, 1109, 1111, 1113, 1125, 1137, 1142, 1148, 1152: n = A,T,C or G
ttnannncctttaaaacccttttnaagntgcccgctcgggcggccgccntgtgacggatatctgcagtaattcggcttaatcccatgggaaattaattcaaaaacnncatcctgctgggcactggaattataaacatgatcattaagttctgtcaattatccagacatcacaccatgaagcctactggatatccgactcaaaattacaaagaagtatatgctaactggtatgtatgaggaaaatggttgtgtttaatatagatggggagataagatgtacnggtaantnactncaaatggctaagtatnacggnccaganggtactatnnnctcaatnataagcatgngantaggagctccaacgcttactgcncatagagcnccttttaaaatncagatttntgnnccctaatcccagggtcggaacagnctgngatggggcnnanaatcgcanttntaacnagccctcagtccgncctttaanggtgcnncccttatncaaccnttttanacaannccncctcngaggancngaccgtttcnggctcnactacttaacnngntctaacgnnntctntncanctttaacncgnctctnccacgnnncactngcnntgnacgccaacgctcnnccacccnngtccngtcncgtcncnncacnctcccnctcnccccagcctnttnttagcaccctccncncgcccnctnagcgcnntcnccctcgtccccatcctcncncnctnctcnngcctccccncctttctcncntccnnncccttccctccncncccntcccctntctcccctcctnnccnctttctcccttcttccnttccccnccccntntctccccnctccctcctcccctctnccncctcncnccccctccacccccccnccctcntccnntcnnctnncccccttntcctcttanccaccnctccccnccctcctccctctctctnncntnnttcntctcttcccnncctccttccntntctccntctcctctctnntcttccctttctccnccnccctctnnccctcnntntcctccccntncncnctcccncctcctcttcccntnctctnncntncttcnncctcncntnccccctccctcnccctcttcttcntctcntccacnctcncttccccc

SEQ ID NO: 14 - DNA, 1148 bp, Homo sapiens
  misc_feature 7, 8, 23, 25, 32, 33, 34, 36, 40, 56, 84, 103, 104,112, 169, 180, 192, 198, 232, 237, 271, 274, 315, 324, 391, 487,523, 528, 534, 535, 536, 565, 577, 613, 644, 657, 666, 678,679, 680, 773, 783, 784, 789, 838, 843, 857, 860, 864: n = A,T,C or G
  misc_feature 869, 881, 898, 902, 904, 909, 910, 927, 932, 938, 943,948, 954, 957, 959, 961, 967, 971, 982, 987, 989, 991, 997, 1003,1005, 1009, 1021, 1023, 1027, 1037, 1038, 1041, 1042, 1048,1059, 1061, 1073, 1086, 1088, 1090, 1095, 1099, 1108: n = A,T,C or G
  misc_feature 1123, 1128, 1135, 1138, 1145: n = A,T,C or G
tttaaanncccttttaaaaaccntnttcaaannncncctnaaatattcatgcatcnttaagttctgtcaattatccagcacatnacaccatgcaagcctactnnatatccgnctcaaaattacaaagtaagtatatgctaactggtatgtatgaggaaaatggttgtgncctaatatagntgcagcttaatngccttncagcacatccccccttcgccagctggcgtaatancgaanaggccctcaccgatcgcccttcccaacagttgcncancctgaatggcgaatggacgcgccctgtagcggcgcattaancgcggcggntgtggtggttacgcgcagcgtgaccgctacacttgccagcgccctagcgcccgctcctttcgctttnttcccttcctttctcgccacgttcgccggctttccccgtcaagctctaaatcgggggctccctttagggttccgatttagtgctttacggcacctngaccccaaaaaacttgattagggtgatggttcacgnagtgngccatnnncctgatagacggtttttcgccctttgacnttggagtccacnttctttaatagtggactcttgttccaaactggaacnaccctcaaccctatctcggtctattcttttnattcataagggantttgccganttccccctattnnntaaaaaatgagcctgacttaacaaaaatttaacgccgaattttaacaaaatattaacgcttacaatttcctgatgcggtatttcctccttacnccatcctgtnncggtntttcacaccctatatggtgcactctcagtacaatctgctcttgatgccncatanttaagccagtcccnacncccncccancacccgcctgtntcccccttactgctttncctncncccgnnatccgctccagacaaanctttnaccctntccgnacccncctgcnttncnanatttcncccncttcccctaanccctncnanccaacngctccntntaanccccccttctanancctncgtcctacannctnngcctcntaaaacccatngntcccctttcccngccaatgtccccncncnctttncccnattttcttnacccatatcaaactnactcncgtcttntcncacattnccc

SEQ ID NO: 15 - DNA, 1051 bp, Homo sapiens
  misc_feature 13, 15, 28, 83, 154, 208, 231, 238, 249, 270, 272, 273,278, 288, 290, 292, 304, 315, 319, 329, 330, 334, 336, 337, 338,344, 358, 363, 370, 378, 383, 384, 390, 400, 403, 408, 411,412, 414, 420, 423, 426, 427, 431, 434, 436, 444: n = A,T,C or G
  misc_feature 446, 450, 451, 453, 455, 468, 471, 477, 480, 483, 501,508, 513, 515, 518, 533, 545, 546, 547, 548, 552, 556, 560, 566,568, 573, 588, 594, 598, 599, 600, 603, 608, 616, 619, 620,626, 628, 629, 636, 639, 643, 648, 650, 652, 654, 657: n = A,T,C or G
  misc_feature 666, 667, 689, 693, 702, 704, 705, 708, 711, 713, 718,721, 724, 726, 727, 732, 739, 750, 755, 759, 763, 766, 767, 775,779, 795, 799, 814, 816, 817, 828, 831, 833, 834, 838, 842,848, 851, 852, 866, 868, 876, 884, 885, 887, 888, 903: n = A,T,C or G
  misc_feature 906, 907, 910, 922, 928, 930, 936, 941, 942, 960, 962,969, 973, 974, 976, 982, 991, 995, 1003, 1004, 1010, 1025, 1031,1038, 1042: n = A,T,C or G
tttgaaacccttntnaaaggccttctanatttcactcaactttccatcaagcttgctaccgagctcggatccactagtaacgnccgccagtgtgctggaattcggcttggtgagactttattggctagtgataaatgcggtgaagcaatcaggnctctccaagaagcagaaaaattgtatgcaaaggcagaagcactgtgtaaagaanatggagaaaccaaaggacctggnccaacanccaaaccttcnggacatctgttttctaggaancnnggaanccccgtgaanancnccctaaaaaaanaaccccataancccnccctcgcctnntggnannnataacncaaccctcctcccnccccntcaattnatccaacncccgnncctacnctcacctcgnttnctcgntanntncccacnccnccnncttngcntnttccaccncnttcnngntnttttcccaattantanttattnacnctnctttcctctcccgctccnctcaccntcccntnctncacccttctctcccnctaacactcttnnnnccgnttcncccnctttcnanccacnatccctcctcctctnctgccnttannnccntctcntcaccccnccnnaatcancnnacctccntcntccncctcncncngnccncccctcaannccccctcctcctctcatccccntctncttctatcntnncanacntnaccgncantcncnntcatntcctctntccaacctctncattncttncttnctnncccccacncccncttcctcatcacatancccnccccacctccctacncnntctacctctcnccncnncccntctnttctcnacnnccttacatcacccncnctccctcnatctcacnncnncaatcaccacgccanctnnacncacaacccctantgtacntnctttanctctnnaatcttgtcccccccctncncctctcnctcnncnaatacncccccctcncccnccctcaanncactcnactttaatcacctcnatctcncatcccnctcnccctcacct

SEQ ID NO: 16 - DNA, 1030 bp, Homo sapiens
  misc_feature 6, 7, 13, 17, 18, 20, 29, 31, 45, 46, 57, 123, 132,134, 136, 137, 138, 143, 144, 146, 148, 151, 155, 165, 166, 167,171, 180, 183, 185, 186, 187, 203, 209, 210, 211, 214, 216,218, 225, 226, 227, 228, 231, 232, 235, 239, 241, 249: n = A,T,C or G
  misc_feature 251, 252, 253, 254, 255, 256, 257, 262, 263, 264, 265,266, 273, 274, 275, 283, 284, 287, 288, 290, 298, 300, 303, 305,307, 308, 314, 317, 319, 336, 338, 343, 345, 365, 366, 367,388, 396, 398, 404, 406, 413, 416, 431, 433, 437, 446: n = A,T,C or G
  misc_feature 448, 455, 459, 466, 469, 470, 480, 483, 489, 499, 507,519, 524, 549, 552, 568, 571, 572, 573, 574, 582, 586, 592, 600,606, 610, 615, 616, 625, 632, 633, 639, 641, 642, 668, 673,679, 682, 685, 687, 693, 698, 708, 710, 728, 740, 744: n = A,T,C or G
  misc_feature 753, 754, 771, 774, 775, 776, 786, 792, 793, 796, 816,821, 829, 839, 844, 845, 854, 860, 862, 864, 868, 873, 877, 884,885, 888, 894, 897, 900, 907, 920, 927, 932, 942, 954, 971,972, 973, 979, 984, 995, 1008, 1015, 1016, 1025: n = A,T,C or G
ttaaannccttcntctnnancctgctcgngnggccgccagtgtgnnggatatctgcngcaattcggcttaagcagtggtaacaacgcagagtacttttttttttttttttttttttttttttngggggggtntncnnnggggnncnangtntccnaaaaaacaannntccngagggtttnccngnnngccccggcctttgggnttcccnnnttntntncccccgnnnnttnnccnttcnanccaatttntnnnnnnntgggnnnnncccctcnnntccccccnnttnntnccaccccnanaangncnncccccnccncnctccaccccccgggggngnggccncngggcccccccccccctcccnnncctctccccccctctcgtctntcttcttncngccccnanacttccnggnccccacctaggcccntncccncccctctcncnccgcaancccnttccctnccnncccccccccnccncccctnctcccccccncccttctnctccccctctcnccctnccccccctccactccccccccctcncanccccttccgccccccntcnnnncctcccancctntctccnttctgctnccttcncccnctccnngtatcccanccctctnncgtccncnnccctcaccttttcaccccccctctcnctctncccctntcntcntncttttnccctncctcttctantnccctctccctccctcccntcctccctcccncgcncgtcccccnnccccctctcttgccccnccnnncctcctccanctcacnncantcccctcatcctccctcacnctccnccctccantctactccanctccnntcctccccncccctncntncccnacctncttnctgcctnnccnttcccntcnccncctctcncccccctcacccncctaccnttccncccctcctcnttccccctcctncactctctgccccctcnnncccccnctccnccccttccctnttccccactttcntcctccnnccctcccgncccct

SEQ ID NO: 17 - DNA, 1100 bp, Homo sapiens
  misc_feature 3, 19, 24, 31, 53, 97, 102, 108, 118, 123, 127, 129,139, 142, 149, 157, 163, 167, 180, 209, 215, 217, 224, 236, 241,247, 263, 264, 268, 277, 281, 292, 313, 318, 332, 334, 342,343, 345, 347, 361, 365, 366, 369, 370, 372, 373, 375: n = A,T,C or G
  misc_feature 377, 380, 383, 388, 392, 397, 399, 400, 403, 404, 406,408, 409, 410, 414, 417, 418, 421, 424, 428, 430, 433, 436, 437,438, 443, 445, 448, 449, 451, 454, 468, 469, 470, 472, 473,480, 481, 482, 484, 487, 492, 494, 496, 497, 500, 505: n = A,T,C or G
  misc_feature 506, 508, 511, 512, 517, 521, 522, 524, 529, 532, 536,537, 538, 541, 543, 544, 546, 549, 550, 551, 555, 556, 566, 570,579, 585, 587, 588, 590, 595, 597, 599, 602, 606, 611, 613,615, 624, 627, 628, 634, 638, 645, 646, 648, 650, 652: n = A,T,C or G
  misc_feature 654, 656, 660, 672, 673, 675, 676, 679, 681, 683, 684,692, 695, 700, 705, 707, 710, 715, 722, 729, 736, 739, 740, 750,751, 752, 756, 758, 759, 761, 763, 766, 768, 772, 775, 778,780, 787, 790, 793, 795, 797, 799, 802, 805, 807, 809: n = A,T,C or G
  misc_feature 811, 822, 826, 830, 831, 834, 839, 842, 848, 854, 863,864, 867, 871, 872, 875, 877, 879, 881, 885, 889, 890, 892, 894,895, 900, 902, 912, 914, 915, 919, 920, 921, 923, 925, 930,931, 935, 943, 946, 948, 951, 955, 967, 976, 977, 978: n = A,T,C or G
  misc_feature 979, 980, 999, 1001, 1008, 1009, 1010, 1011, 1016,1019, 1024, 1027, 1036, 1037, 1042, 1046, 1051, 1052, 1053, 1059,1060, 1061, 1066, 1067, 1069, 1082, 1083, 1084, 1086, 1087,1088, 1089, 1094, 1096, 1097: n = A,T,C or G
ganccccccttttgaaaanccctntgaaaantccacacaaaatttccctaaancttcccatcaagcttggtaccgagctcgggattcactagtaacntccgncagatntgctggaatncggcntggngnggactttatnggntaatganaaatgccntgaggnaatnaagtctttccaanaagcagaaaaacgtatgcaaaggcagaancaccgngnaaagaanatggagaaaccnaaggncctggnccaacagccaaacccnncagncacatccgnaccnttaggaaatcnggaaaacctctcgcaaccaanacccntgaaaaacgccccnanaagaaaanngncncccataactctccncaanncanncnnantnccnccnacccntctncaccntnnaanntntnnncganccnngantgngctncngtnggnnntcacncnctnnancancagccgagccctcnnncnncttcacnnnanctnataantncnnccnataanntnctnnccggnaaanncnacacnacncccnnncancnncnctnnnccanncccccacacncccncccctccancgcttnannantaacnantnccnatcnaccangnanctcctcccntcnnaccttncgancgacccnncntncncntntctngcccctcctacnncnnccnancnncttgcatnacnacacnccacnancanccaancaccatnacccccngcacacntanntcgcaaatcnnncccngnntngnaancncatnacncgntncaagccnctnacnancngngcnccncncnancgccctcatcncccnaccnncangcctnccnctcccncccagncgccctaannctncccnnacncncnancccncctnncngnnccaancnccgtacagcncnncccnnncncngccgnnctancacccacnccncncanctanagctgactcagnagacacccnnnnncaccgcacctccactctcncnccccccnnnncaccngcnccccncantcaaaccanncctcnacanccccnnncgaaannnccttnncnagcctcccacaannntnnnnaccgnanncct

SEQ ID NO: 18 - DNA, 1063 bp, Homo sapiens
  misc_feature 15, 21, 28, 29, 38, 41, 52, 82, 90, 128, 129, 135, 140,142, 144, 155, 156, 158, 159, 162, 177, 178, 180, 181, 182, 188,194, 200, 203, 206, 207, 217, 219, 220, 222, 227, 229, 235,236, 242, 243, 245, 246, 248, 250, 254, 261, 267: n = A,T,C or G
  misc_feature 269, 275, 280, 283, 287, 288, 295, 297, 306, 309, 310,313, 314, 324, 332, 339, 352, 372, 375, 377, 379, 381, 383, 384,385, 390, 398, 399, 402, 405, 406, 412, 426, 444, 445, 448,454, 462, 474, 478, 484, 490, 495, 498, 500, 503, 515: n = A,T,C or G
  misc_feature 525, 529, 531, 541, 543, 551, 555, 561, 566, 572, 587,589, 598, 608, 611, 616, 621, 623, 626, 629, 631, 636, 641, 644,655, 660, 668, 679, 696, 699, 702, 708, 711, 721, 728, 731,738, 747, 748, 749, 755, 764, 767, 768, 769, 771, 777: n = A,T,C or G
  misc_feature 788, 794, 808, 809, 815, 831, 833, 844, 847, 853, 863,873, 880, 891, 899, 901, 902, 920, 923, 924, 937, 955, 959, 963,964, 989, 997, 998, 1007, 1013, 1019, 1021, 1024, 1027,1028, 1037, 1044, 1056: n = A,T,C or G
accccctttttaaancccttnctctatnncatgctcgntgnccgccatgtgntggatatctgcagaattcggcttaagcagnggtaacancgcagagtactttttttttttttttttttttttttttnngggggnaaaanangncctttttgggnncnnctntacccagggggctcnncnnnagggtnccccanttcccnaanaannctccccctangnntncaccncncccccnnttcccnntnntncncctntttctcnaccacncngccccnccccnccncacnnaatcctncnctttacggnaannccnntttcctttancgcccttnttctttncccaccacccccncccccccccccgttactccntcncncntncnnnctcanccccctcnnctnccnncttttncaccaaccgcccgncatcttctcccttcatcnncantacaangcaccctnatccactccttnaacnattcantaaccnaatcnccnanccnccttcacccccnccttcccttncccntntcatctttcntnccccatcnctcncacatntttgncctacntctccactcctcccncnctctccccnctactctaanccnctccnctccncnacncancnccccnagctnccncaccatccccntcccncccctccncccccccctcnccctctaccccctcttnctnctnctaccngcntccccccctnatcacanacncactcgnctcccctcnnnctcccntgtattccntcnnncnccctcnctcccctttcnccctcncactcccctcatcnntccccntccccctccactttcnancctacagcccnttncccccnccatcccacnatcccccccntcccgtnatctatcctcngctcctcncnntctacctcctctaccctnccnnccacctccacacnaaccctcccctctctctnctcnctcnnctccccccctccttcacactccccntcacccgnnccgcccacnctcccntcctcncntantcnnccatcccanacacccncaatccatcctncacacct

SEQ ID NO: 19 - DNA, 1044 bp, Homo sapiens
  misc_feature 21, 28, 32, 38, 41, 52, 62, 71, 88, 99, 130, 138, 160,272, 294, 328, 337, 347, 348, 352, 358, 363, 366, 369, 379, 381,387, 395, 399, 405, 408, 428, 429, 430, 432, 435, 439, 440,441, 447, 452, 453, 457, 459, 461, 462, 468, 470, 475: n = A,T,C or G
  misc_feature 478, 483, 484, 485, 486, 490, 492, 494, 496, 497, 501,504, 510, 515, 522, 534, 536, 538, 543, 544, 546, 560, 565, 571,582, 585, 591, 599, 608, 613, 620, 624, 633, 642, 643, 646,650, 653, 655, 659, 662, 665, 668, 669, 675, 681, 690: n = A,T,C or G
  misc_feature 697, 701, 707, 710, 713, 714, 719, 721, 722, 727, 728,731, 733, 737, 750, 751, 752, 755, 758, 759, 767, 769, 781, 787,792, 803, 807, 815, 819, 820, 822, 827, 829, 835, 837, 840,848, 852, 855, 859, 868, 869, 872, 879, 882, 884, 887: n = A,T,C or G
  misc_feature 898, 904, 906, 911, 915, 919, 920, 924, 927, 931, 936,951, 963, 965, 968, 969, 972, 975, 1004, 1008, 1020, 1022, 1034: n = A,T,C or G
tttgaacccccttttaaaaangcccttnctanatttcnctnaactttccatnaagcttgccnaccgagctnggatccactagtaacgnccgccagtgtnctggaattcggcttggtgagactttattggntagtgatnaatgcggtgaagcaatcagggntcttcaagaagcagaaaaattgtatgcaaaggcagaagcactgtgtaaagaatatggagaaaccaaaggacctggaccaacagtcaaaccttcaggacatcctgttttttangaaacttggaaaccttgtgaanaacaccctagaaaaatgccagagagaaaatgganttatttacnttgaaaaaannccancagacnccccncanccngaacttaaancnccctcncgctctcncagngcctanacncttcgaatttctcctacaannngntanctgnnncaccanacccnnccgnangnntctgantnctctnaangaccnnnngatngncncnncccnacnccctcnaattngtatccncacttaccccancncnctctnntnttttcactatgatncccgncctacntttttaaaccnccncctacnccaccttncccacacanatccnatcaccncctntacccttcntcacctacnntantccnccntnctcnacntcnccnntccctnctctanatcaacccncccgctnaccnctatcnccnccnnttctncnnctccnnatncnttcnctgcccccccccnnnacnccnntccttccntncccctgtccccnccctcncctcncctactttccntccntacccccntccnncnccccncntccatncnttncaactatntcantanttcnaccaccctnnctncctcccnctncnacncccctccctcntcaccnancccanccanttcnncgantcntctntctcntcccctcaccccttntccttctccttntnctnnctntcntccagtcatctccccaccccacccagacncccncatccccatccncncctctctccccnacccacccct

SEQ ID NO: 20 - DNA, 987 bp, Homo sapiens
  misc_feature 1, 5, 7, 12, 13, 14, 15, 26, 39, 52, 87, 120, 121, 122,123, 124, 125, 128, 130, 132, 133, 137, 141, 151, 152, 154, 160,167, 174, 182, 184, 187, 189, 190, 192, 198, 200, 205, 219,220, 221, 222, 225, 231, 233, 237, 238, 242, 246: n = A,T,C or G
  misc_feature 250, 251, 252, 253, 259, 264, 266, 269, 271, 277, 278,281, 298, 300, 301, 304, 305, 308, 309, 315, 322, 324, 329, 334,346, 347, 349, 351, 356, 358, 359, 364, 365, 375, 376, 377,384, 392, 395, 405, 428, 432, 442, 446, 449, 454, 461: n = A,T,C or G
  misc_feature 462, 464, 473, 479, 484, 485, 488, 491, 503, 512, 518,539, 540, 543, 544, 555, 556, 561, 565, 569, 573, 575, 577, 578,579, 580, 582, 584, 589, 591, 600, 602, 614, 616, 621, 636,649, 650, 663, 665, 670, 678, 684, 694, 705, 709, 711: n = A,T,C or G
  misc_feature 712, 717, 718, 720, 723, 734, 737, 755, 756, 760, 762,764, 765, 773, 785, 789, 797, 798, 799, 800, 804, 807, 822, 823,825, 826, 830, 831, 837, 848, 852, 868, 875, 887, 893, 904,907, 908, 911, 932, 939, 946, 954, 962, 971, 974, 976: n = A,T,C or G
  misc_feature 978, 980, 981: n = A,T,C or G
ncccntnctctnnnncctgctcgagnggccgccatgtgncgggatatctgcngcaattcggcttaagcagtggtaacaacgcagagnactttttttttttttttttttttttttgggggnnnnnnccngncnncctngggnttttgggggnnanaaaagncccccanggttttnggggcccncngancnntngggggngnaaatncaaagggtttgggnnnnccnaacccntntttnntttngggncccnnnngggggnttttngnggnantttttnnaangaaaaaaaccccttttncnntcnnccnnttttcnggggggntntcaanagggnttccaagctccnnananaccanannccctnncggttccccnnnggccccncctccttnggnttttccatcnttctttacccccgtttccccccncccncacacctctnttgntcntggcncaaaacnntntttgcttcncccctnttttnnccnccnccctattccctnccccccccntttccncccccttgcggcccctctttnnctnnctccctcctcnnccccncgcntccnctcncntnnnncncnccccncnccccccccncnacctcccccccncnccccncccccccccctcccntcccccccccccnnccccccccccccncntcccnccccaccncccccnccccctctcncctcatccccncccncnnccctnncnccncccctcccctnctnctactcccccccctcccnncccncncnncctccctntcctcccctccncccncctcctcnnnnctcnccncccccccctcccccnncnntccnncccccntcccactcctncctncccccccccttccccnctcctcncgcccccacccnctcctnctccccccctnccnnccnccccctccccctctctatctntctctcnccccccnccctcccnactcccanccccctctntcncncncnnctccct

SEQ ID NO: 21 - DNA, 1038 bp, Homo sapiens
  misc_feature 2, 3, 5, 18, 29, 44, 53, 76, 92, 94, 95, 113, 124, 183,210, 230, 232, 253, 311, 316, 325, 327, 336, 357, 363, 387, 399,405, 409, 413, 417, 421, 430, 436, 441, 444, 460, 466, 477,492, 495, 496, 501, 504, 508, 509, 517, 523, 525: n = A,T,C or G
  misc_feature 533, 534, 537, 544, 549, 554, 558, 568, 570, 572, 573,582, 586, 600, 603, 606, 611, 613, 627, 631, 636, 640, 648, 653,655, 659, 672, 678, 682, 689, 692, 693, 695, 698, 699, 705,706, 711, 712, 713, 714, 724, 733, 742, 751, 753, 754: n = A,T,C or G
  misc_feature 765, 766, 772, 780, 782, 787, 792, 797, 814, 815, 816,821, 827, 831, 837, 838, 840, 841, 856, 858, 879, 880, 889, 892,899, 903, 910, 914, 925, 926, 927, 933, 940, 943, 947, 949,964, 974, 975, 978, 986, 987, 1001, 1012, 1017, 1027: n = A,T,C or G
  misc_feature 1029, 1032: n = A,T,C or G
anncnccctttttaaacnccttttaaaantccacacaattttcnctaaactanccatcaagcttggaaccgagctnggatttcactagtaantnncgccagtgtgctggaatncggctccttgnagatgtgttggtacacagcttatgcttactggtaccatggtgagactttattggctagngataaatgcggtgaagcaatcaggtcnctccaagaagcagaaaaatngnatgcaaaggcagaagcactgngtaaagaatatggagaaaccaaaggacctggaccaacagccaaaccttcaggacatcngttcnttaggaaantnggaaacctngtgaagaacaccctagaaaancgtcanagagaaaatggatttatttacttncaaaaactccancagaancctnacanctgnaacncaaagaacnccatgntctcntancgccctatacgtctcnaacttntttttacaagngttcagcggacactnccnncattnccncctnntgaccctnacccanangacctccnnccngacagtnctaancccanatcnatgacctaanangnncacatcccncgtncagcacccctgacntcntanacccncntagggacactagcngctnctccncccncctaacancaccntnttcnctcctcacacttnacctcnagcnacgcccncanncntannattccnnccccnnnncctcttaccntctctcctngcccctccncccccccantnntccctcccccnntctcancttcaccncncttcnttccncttcnctattccccacatcttnnnccccncacccncccnctcatnncnncctctaccttcagancnacctccccatccctcccacannctccccccntanccctccncttnccccccntcanccccccccccnnncttctncggcttntcnacancnctacttccccccttnttcatccccnnccnctcctaanntcccccaccccccntcccccccccnccttnccctcctctncnttntcccct

SEQ ID NO: 22 - DNA, 1048 bp, Homo sapiens
  misc_feature 11, 15, 16, 23, 25, 38, 40, 41, 45, 73, 91, 102, 144,145, 155, 157, 158, 162, 165, 167, 169, 176, 177, 180, 184, 190,193, 197, 200, 206, 207, 209, 210, 214, 215, 216, 218, 219,220, 231, 232, 233, 237, 239, 240, 249, 251, 253, 258: n = A,T,C or G
  misc_feature 259, 266, 267, 271, 272, 276, 278, 280, 281, 283, 291,292, 302, 303, 304, 310, 311, 314, 315, 318, 323, 328, 330, 337,344, 346, 347, 351, 353, 356, 359, 367, 376, 378, 380, 395,397, 406, 413, 417, 419, 420, 421, 432, 439, 442, 444: n = A,T,C or G
  misc_feature 453, 454, 457, 463, 464, 465, 471, 472, 474, 475, 476,477, 487, 490, 497, 500, 501, 505, 513, 514, 520, 524, 526, 529,531, 534, 536, 538, 540, 546, 552, 556, 572, 573, 579, 580,581, 583, 586, 590, 591, 594, 605, 611, 612, 615, 623: n = A,T,C or G
  misc_feature 624, 626, 648, 655, 677, 678, 679, 684, 688, 692, 695,697, 700, 705, 707, 710, 711, 714, 716, 723, 725, 726, 733, 740,742, 744, 750, 757, 763, 767, 771, 772, 781, 787, 792, 799,805, 807, 808, 811, 812, 818, 820, 824, 833, 835, 839: n = A,T,C or G
  misc_feature 846, 852, 853, 855, 858, 860, 863, 864, 869, 871, 872,877, 884, 887, 888, 890, 892, 893, 894, 905, 907, 930, 937, 947,948, 951, 954, 958, 961, 972, 974, 975, 978, 983, 991, 992,997, 999, 1001, 1020, 1027, 1039: n = A,T,C or G
ccccctttttnaaannccttctntnttccatgctcgtntnnccgncatgtgccggatatctgcagaattcggnttctaatacgactcactntagggcaagcngtggtaacaacgcagagtactttttttttttttttttttttnngggggggggngnntccnttngngncccaggnnggngggnaaaaanccntttnaanaaaaanntnntttnnnannnttaaaaaaaannntttngnnaaaaaaaancngnaattnnggggttnntttnnttancngnnanaaaaattnnaaggccaggnnntttttnnggnnatntagcncccangnccttttnggggttngnngccngntcnttncaaggttncactttttntncnggttggggctaacancngcaattttngggctcntccngnnnaaaaaaaaccnccccccnaangnctttggggnnccnttacannncttggnngnnnnacactcccangcntttcacnccnncccnacccccannccttcncttncnctncnctncntngnggctcnttgtcnggancttttccacaatctcnnaaaccnnntnttntgtnntancctcttcatcncagccnntcnctcccccnntnctccccacccctccaccaaaanccctccnctccttcaccttctcccttctnnncaatntccncccncangntcnctccnanccnntcncnccacctntnntcccccnctcaacncncncttttntcctccntcacgnacancccnncttcttccncccctntcccncctctcncccccncnntcnntccttncntacnccccctctncncccncctctcntcttcnnancancnccnntccantnnccctnctctctnccnncntnnncgccatctccntnccttcacccctcctccttccgcnccctcancttacacctnnctntcncccntcncacctcccacncnnctnccacncgccccannactcncnanttcatcccactactcagcncctctcncccacactctcnttcttccct

SEQ ID NO: 23 - DNA, 1033 bp, Homo sapiens
  misc_feature 3, 9, 21, 25, 28, 35, 37, 47, 57, 63, 66, 76, 93, 95,103, 115, 145, 183, 347, 447, 602, 604, 630, 634, 639, 648, 680,681, 685, 732, 744, 745, 766, 780, 786, 804, 815, 818, 821,823, 825, 826, 839, 846, 850, 852, 860, 869, 875, 882: n = A,T,C or G
  misc_feature 894, 898, 902, 905, 906, 924, 927, 943, 952, 955, 957,959, 964, 986, 989, 1029: n = A,T,C or G
ttntaaaancccttttaaaancctnttnaaaacancngcacctggtntcaacagggnaaaccnttnaggacatttnttctttaggtaaacttntnaaaccttntttaagaacacnctagaaaaatgtcagagagggggatggttngatttactttaaaaaaattccaacagaagccccacagntggaacctcaaaacaaattatggtctcgtagagcctatacctttcgaatttcctcctacaagtgttcagtggacaccagaaacattggctgcatttgatctcaccaaaagacccaaggatgacagtactaaacccaaaaaaaaaaaaaaaaaaaaaaaaaaggncctctgcgttgttaccactgcttaagccgaattctgcagatatccatcacactggcggccgctcgagcatgcatctagagggcccaattcgccctatagngagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcncngcctgaatggcgaatggacgcccccngtancggcncattaagcncggcgggtgtggtggttaccccagcgtgaccnntacncttgccagcgccctagcgccccgctccttttcggttttcttcccttnctttcccgcccnngttcgccgggttttccccgtnaaggcttttaaaancggggncccccttttaggggttcncatttaatggnttnacngngnnccttcgacccccnaaaaaantttnanttaggggnggatggctncaccgnaagcggncccatcgccccngganagancgnntttttcgccctttggacnctnggagtcccccgttttnttaataggnggncntntgttnccaactgggaccaacactttanacncttttttgggcctattttttttggatttaaaaagggattntccc

SEQ ID NO: 24 - DNA, 1023 bp, Homo sapiens
  misc_feature 3, 8, 9, 15, 25, 26, 32, 35, 37, 51, 52, 64, 88, 92, 99,101, 102, 110, 120, 130, 139, 143, 144, 146, 166, 171, 190,227, 269, 286, 321, 340, 369, 382, 407, 533, 579, 591, 635,662, 668, 684, 692, 709, 713, 728, 731, 766, 773, 774: n = A,T,C or G
  misc_feature 776, 778, 793, 794, 800, 809, 818, 828, 829, 830, 832,834, 844, 860, 894, 915, 917, 922, 932, 935, 952, 955, 971, 981,995, 999, 1001, 1002, 1007, 1015: n = A,T,C or G
ttntaaanncccttntaaaaccctnntgaaanacncnacaaaaagttagcnnggaaaaggcttnatttactttcaaaaaattccaacngtangccccantnntggaactnaaaacaaatnatggtctcgnggagccttngccnntngaatttcctcctacaagcgntcagnggacaccagaaacattggntgcatttgatctcaccaaaagacccaaggatgacagnactaaacccaaaaaaaaaaaaaaaaaaaaaaaaaaggtcctntgcgttgttaccactgnttaagccgaattctgcagatatccatcacactggnggccgctcgagcatgcatntagagggcccaattcgccctatagtgagnggtattacaattnactggccgccgttttacaacgtcgngactgggaaaaccctggcgttacccaacttaatcgccttgcagcacatccccctttcgccagctggcgtaatagcgaagaggcccgcaccgatcgcccttcccaacagttgcgcagcctgaatggngaatggacgcgccctgtagcggcccattaagcgcggcgggtgtggnggttaccccagngtgaccgttacacttgccagcgccctagcgcccgctcctttcgntttcttcccttcctttctcgcccgttngccggntttccccgtcaagctnctaaatcnggggttccctttagggntccnatttagggctttccngcnccctgcacccccaaaaaacttgattagggtgaatngttcacnnangnggcccattgcccctnntagacngttttttcncccctttgncccttgggcnnncncntttctttaanagctggacctctttcnttccccaaacttggaaccacacccttaacccttntttggggtctattttttttgnanttatnaagggggacnttnccccgctttcggccctnttngggttaaaaaaccgcncctgccttcnacccaaaaatcttncacncnnttttnaacaaaanttccgccc

SEQ ID NO: 25 - DNA, 1031 bp, Homo sapiens
  misc_feature 4, 10, 18, 26, 38, 48, 52, 55, 56, 66, 72, 74, 102, 112,132, 133, 134, 139, 140, 477, 540, 569, 585, 589, 593, 605,624, 664, 666, 682, 688, 697, 708, 722, 733, 740, 741, 744,745, 763, 793, 817, 844, 846, 858, 865, 870, 873, 877: n = A,T,C or G
  misc_feature 878, 880, 881, 883, 884, 904, 909, 913, 920, 922, 927,928, 949, 952, 966, 969, 981, 998, 1001, 1003, 1023, 1028: n = A,T,C or G
tccntaaaancccttttnaaaacccnttacccaggttncaacagacantccntgnnggacatctgntctttngntaaacttggtaaaccttgtgcaagaacntcctagaaanttgtcagagagaaaatggannnatttnntttcaaaaaattccaacagaagccccacagctggaactcaaagcacattatggtctcgtagagcctatacctttcgaatttcctcctacaagtgttcagtggacaccagaaacattggctgcatttgatctcaccaaaagacccaaggatgacagtactaaacccaaaccagaagaagaagtgaaacctgtgaaagaaccagacatcaaacctcaaaaggacactgggtgctacatctcctaaaatacaacttgcacttagaatttctctagcagtaaataagataaaccacagaatttcagttcttatttctcaaaatgatttctctgaagctcgnagaataactattatattcagagggttatctgcactccagcctgggcaacagagggagactccncctcagaaaaaaaaaaaaaaaaaaaaaangtcctctgcgttgttncccnctgnttgccctatagngagtcgtattagaagcccnatttctgcagatatccatcacactggcggccgctcgagcntncatctagaggggcccnaatttncccttatangtgagtcggtntaccaattccctgnccgtccctttnccaccgnncgnngactggggaaaacccttngccttccccaacttaatcggcttcgcagcncatccccctttccccaggtggccntaatagccgaagaggctccgccccgancngcctttcccaanagtttcnccacnctncacnncnnannggaccccccccctgttacgnccccnttanccccggngnggtgnnggtgggtttcctcgccaacgnganccgcttaaccttgnccngcggccctagcngcccccctctcttcccnttntnttcccctcctttcttcggcncacgnccc

SEQ ID NO: 26 - DNA, 1032 bp, Homo sapiens
  misc_feature 3, 4, 7, 12, 21, 35, 37, 41, 48, 52, 53, 56, 57, 64, 71,75, 78, 84, 95, 96, 97, 102, 115, 123, 137, 138, 141, 142,151, 178, 203, 231, 232, 233, 237, 238, 245, 248, 249, 295,296, 304, 323, 327, 333, 339, 358, 376, 382, 384, 394: n = A,T,C or G
  misc_feature 397, 431, 432, 462, 472, 476, 478, 479, 482, 490, 498,518, 523, 524, 527, 544, 548, 560, 563, 585, 590, 592, 604, 605,612, 615, 619, 620, 642, 643, 653, 657, 666, 678, 681, 686,687, 691, 692, 694, 699, 702, 709, 711, 727, 735, 736: n = A,T,C or G
  misc_feature 738, 739, 741, 744, 750, 751, 765, 770, 780, 783, 797,804, 805, 814, 828, 835, 837, 843, 845, 852, 855, 858, 865, 869,870, 872, 873, 881, 885, 887, 890, 891, 906, 908, 923, 930,936, 942, 943, 944, 945, 946, 948, 950, 955, 956, 963: n = A,T,C or G
  misc_feature 968, 969, 971, 973, 977, 979, 982, 997, 998, 1003, 1005,1008, 1011, 1015, 1023, 1028: n = A,T,C or G
ttnnaanccttntaaaacccnctcaaaagtccggngnaaantggcccnattnnccnngaaaaantttcaanagtnttncccatnccgggcaactnnnagaantttatggccccgnagagcctngggctttgggattnnttnntacaagtgntcaacggacaccagaaacattggtccncatttgatctcaccaaaagacccanggatgacagtactaaacccaaaccagannnaganntgaaacntgnnaaagaaccagacatcaaacctcaaaaggacactgggctgctacatnncctaaaanacaacttgcacttagaatntctntagcantaaacnagataaaccacagaatttnagccttatttctcaaaangatttntntgaagcttgnccnaataactattatatttagagggttatctgcactnnagcctgggcaacagagggagacttcacctnagaacaaaanaaanannaangcacaagncccctgcnctgttaccactggttgcccnatagnnagncgaattagaagcctcanttcngcagatatccanccncctggccggccgcttgagcctncctcncntaggccccaaannccccctntangganncgcatttccaattaactgggcnncgctttacanaccngcgacactngggaaaaacctntgnccgcnncccnncnctaanacngccttgncncccattcccccttccnccaggctnncnnanatncacctnnaaagcccccccacnctctngcttttcccncanttctcccccccctnaaccctnnctcgcatcnccctttctttatcnccccttnanccccgngnccccaangtngtntaacccnggcnncnnttcgttcnattntnccnnagcccctaacgcccncnaccctatccaatttnttcacantccttnttcgannnnncngnccctnntcccccntctannancnaacnanccnggcccttcctcacgnnccccncnctnaangccnttaccccnccccncctt

SEQ ID NO: 27 - DNA, 993 bp, Homo sapiens
  misc_feature 4, 34, 55, 56, 64, 77, 95, 100, 102, 117, 124, 125, 137,158, 164, 166, 167, 203, 205, 215, 236, 240, 341, 372, 418,426, 482, 500, 558, 572, 630, 644, 709, 714, 747, 749, 765,771, 778, 787, 788, 794, 804, 809, 813, 831, 835, 849: n = A,T,C or G
  misc_feature 850, 866, 877, 878, 880, 894, 899, 900, 906, 907, 908,911, 915, 919, 921, 927, 933, 939, 940, 958, 963, 967, 968, 970,971, 976, 977, 983, 987, 989, 991: n = A,T,C or G
tttngaaaacctcctttggaaacctcctccaaanatcctacactattttaatccnnaagctccnccatcaagccttngtaccgatctcggatccnctagnanttgttcgccagtgtnctggaanntcggcttaactngctttcttggggagatccatncaaagnanntatgaaacctatagaaaagtacaagacattttagcntntagaagaatnagaagaaatattgagtgttcngttnggaaaaattttcagattacttttccaggtgtctgaatgaaacttcttgctctcttccaaagaatacagtctggaagagtaaactaatcaaatttgaaaagncaaagaattaaatgtaaacttttagacagtnttttaaaaaacctcaagttcatataaataactaaccttgtaaaagngagaaatntgaatctgctgcattcttcatcatttctggattacagctcaaatcagtgaacagtncaaggagtcgtgccctgnatgacctcaagtcacttataacagatacaaaaagacagtaaagttagctgcactgcantcaatctacataanagtggcttccaggtttcaatgtcaaagctttaaaacacaccacatacacacacgcacnaggtttcaatgtanaagctctaaaccacacacacccatgcacacgcctaccttcctctaagggattaacaacatggaanttaancagggactaaaatctagcgaacaaaacttctantnacctgtttcaaaagcnttctanctaaacnaaccctcannccaacnaaaaggggcnaatancggnattccaattccataattnaaantatctcgtccatgnntttactctaaaaacantcaaccaacanncntgcccctcaaacantgtgnnggcaannnaangaancaananccctancccctncccccnntttcggctcctcgaaaanaagcnaaanntnncctgnncccccntctnananct

SEQ ID NO: 28 - DNA, 955 bp, Homo sapiens
  misc_feature 7, 15, 28, 31, 34, 41, 43, 55, 61, 69, 70, 78, 87, 89,95, 97, 106, 108, 129, 161, 175, 373, 432, 433, 566, 638, 641,723, 740, 744, 759, 764, 794, 795, 799, 801, 809, 821, 827,835, 841, 843, 857, 876, 877, 887, 892, 896, 900, 906: n = A,T,C or G
  misc_feature 914, 917, 923, 924, 927, 930, 948, 951, 953: n = A,T,C or G
ttgaccnccttttanaacctttcctaangcnctntgctcgntnggccgccagtgncatggntatctgtnncaattcgncttgtaatncnactcantntagggcacncntggtcgacggcccgggctggnctgtttgtaattggcaatacatacatactttnttggaataagcctncttagagttagtactaatcaataagactcaagggatataccattcaggattaagtctgtgctctcttctaaacatcactgctaccttatagtctaaatctcaaaagaaaatatttgaaaagttattctttatccactcaacaagtaattttgcatatgtgctttatcctaggcacctaccaaattctggatatacagnctctactctcttggtgcttaccatttaaatttagtctttctttaatggtttacacatanntcaagggtctttggccatacccatgtattgtaatttcggaagcctcactaagctaattgccttctggattcgatttggaactggaatagaaccaagtgaaagctaatgatggagatctaggaattgatatagntgaagctatactaataggtgagatctcaaaagagagaaaaccagaaagccaaagactgaacatttttggagntgngaatgaaaaggagacacagaaaaaaatataaaaggaacttaaaagagacaaggggagcacctggaaaatttagtggtattgnaatcaaaggagaaaatncttncaagaaggaaaaggntgganctataacatttatcgctatagagaagtaanngganantgtagatnagggagcagttncaaaancaactgcnggcccncntttcacacattctnggatggatatggccgaatnncctgtcaagnatatntaangcanattcantttctttnaantattcnnatnccncctatttttcaacccttntcngnct

SEQ ID NO: 29 - DNA, 1064 bp, Homo sapiens
  misc_feature 5, 6, 16, 21, 24, 25, 33, 39, 72, 110, 209, 214, 231,232, 235, 237, 238, 244, 245, 246, 256, 282, 292, 297, 306, 319,321, 323, 330, 334, 340, 349, 354, 355, 363, 372, 376, 378,397, 405, 432, 437, 454, 455, 457, 458, 459, 468, 470: n = A,T,C or G
  misc_feature 485, 487, 488, 494, 496, 499, 511, 524, 527, 552, 557,562, 583, 600, 611, 613, 623, 624, 652, 654, 674, 681, 687, 691,694, 701, 713, 716, 720, 721, 725, 731, 734, 735, 739, 743,744, 781, 782, 785, 789, 799, 803, 821, 823, 847, 852: n = A,T,C or G
  misc_feature 858, 878, 884, 886, 896, 897, 901, 917, 926, 932, 939,948, 957, 961, 965, 981, 991, 993, 1001, 1002, 1005, 1011, 1018,1043, 1047, 1049, 1051, 1054, 1056: n = A,T,C or G
ttttnnccccctttanaaacncannttcaaganccaaanggtggtatctataacatttattgctataggagnaagtaaaggagaatgtagattagggagcagttttattnacaactgatggccacgttttacatattcttgatgcatatgttgaattacatgtcaaggatatgtaatgtaatttcattttttaaatatttttacgcagnatatnttcacattttcgcgttnnatntnnggcgannnaccccccagnggctttagaggtgatgacgcaccctncttccccacntcccnctcacttcnttcccgctcccgntncnccccctnatcncccctnccccctccntcccnncctccaantcctccccnctcntnactcccccccccctccccncccctccnacccccctcccccccccccctcccctnccccncccccccccctcccccnncnnncccccctcncntcccccccccccccncnnccctcncnctnctctcccccccncccctcccccccnccnccccccccccctcctaccccctccnccccnccccncccaaccccccccccccctcntctcccctccctccccntccccccctcncncccccccccnntccccctccctccccccccccccccccncncccccctccctccccccccnccctccncccccncctntcnccccccnccccctcacctnccncccnnctcnccctcnccnncccncccnncttcccccccctaccccccccttcccctccccccccnnccncccntccccccccncccnccccccccctcccccccncncccccttctccctccccccttccnccccntccccnctcctcctccacctcccccnctcctncnccctcccccnncctntccctccccctctctntcccccccncccccncctcccncctcccccntctttcccncccntccnccttctcccccccccncccctccccncnctccctcnnccncctccnctccccnctcctccccccccccccacaccccnctcncncnacncnctcccccc

SEQ ID NO: 30 - DNA, 1058 bp, Homo sapiens
  misc_feature 7, 9, 88, 462, 505, 531, 582, 586, 607, 624, 658, 679,680, 687, 700, 702, 707, 719, 724, 730, 736, 737, 742, 747, 748,756, 761, 764, 765, 767, 768, 769, 775, 776, 779, 781, 782,785, 787, 788, 792, 793, 794, 796, 799, 800, 807, 817: n = A,T,C or G
  misc_feature 818, 819, 821, 822, 823, 828, 829, 830, 832, 837, 838,849, 850, 851, 853, 855, 856, 860, 861, 862, 863, 864, 871, 872,873, 879, 881, 883, 886, 888, 891, 892, 893, 896, 900, 903,904, 905, 906, 909, 913, 914, 916, 919, 921, 925, 929: n = A,T,C or G
  misc_feature 930, 931, 950, 951, 954, 957, 963, 965, 966, 967, 968,969, 973, 980, 981, 984, 988, 990, 991, 994, 996, 998, 999, 1001,1006, 1008, 1009, 1010, 1017, 1020, 1021, 1023, 1024, 1025,1027, 1031, 1034, 1035, 1039, 1042, 1043, 1044, 1045: n = A,T,C or G
  misc_feature 1046, 1058: n = A,T,C or G
ttttaancntttttcagtccttatagcgctacaaaaggcagtaaagttagctgtactgcaatcaatctacataaaagtggcttccagntttcaatgtaaaagctttaaaacacacacacatacacacacgcaccaggtttcaatgtaaaagctttaaaacacacacacgcatgcacatgcctacctcctctaagggattaacaaatgaatttaatcaggactaaatctagagaaaaaatttctaataactgttttaaaagttgtagtaaaataaactcagaccaattaaaggggcaaataatgaattcaattacataataaaaatatatgtcaagttttaattaaaaacaatcaaacaaatgcatgcacttaagatgtggggaaagaagaataaaaaactaagcagttcacaataagtttcttgaaaaatgaaaatttatctgtgtatcattttaatatctncttgaagtttctaaacctgacataaaacacaaaaatgtgaaantatactgcataaaaatatttaaaaantgaaattacattacatatccttgacatgtaattcaacatatacatcaagantatntaaaacgtggccatcagttgntttaaaactgctcctantctacattcttcttttctttctctatagcaatanatgttattggattcccctttnncttcttngaaaatattttcncntttgntttccaataacnctaanattttncacggnncctcnccttnncctttttnaagtntcnntnnntttttnntcngnnccncnnttcnnntnccnnccccccnatttttcctnnncnnnccttnnncnttctnncctcttccccnnntncnncctnnnnnttcttcnnncccttntncnctncntcnnntcncctntcnnnntcnttcnncntcntntttncctnnnttccctccccttctcctcnntcnttnatttcntnnnnnttcnctccccnnttntctntnnttncncnntnccccntnnncccttantcnntnnntnttcntcnncctntcnnnnnctctcccccccn

SEQ ID NO: 31 - DNA, 971 bp, Homo sapiens
  misc_feature 4, 7, 25, 48, 50, 70, 86, 87, 88, 95, 98, 125, 132, 134,219, 253, 523, 619, 668, 717, 750, 752, 771, 782, 804, 806,811, 815, 840, 848, 849, 851, 854, 896, 917, 920, 941, 944,950, 957, 962, 963, 968, 969: n = A,T,C or G
ttgnacnccctttttataccattcnacactatcttaatactcattctncncatcaaccttggtaccgatntcggatccactagtannngccgccngtntgctggaattcggcttaacttgctttnttgggtngnttcatccaaagaatttatgaaacctatagaaaagtacaagacattttagttttagaagaattagaagaaatattgagtgctctgnttggaaaaattttcagattacttttccaggtgtntgaatggaacttcttgctcttttccaaagaatacagtctggaagagtaaactaatcaaatttgaaaagtcaaagaattaaatgtaaacttttagacagttttttaaaaaacctcaagttcatataaataactaaccttgtaaaagtgagaaatatgaatctgctgcattcttcatcatttctggattacagctcaaatcagtgaacagttcaaggagtcgtgccctggatgacctcaagtcacttataagagatacaaaaagacagtaangttagctgtactgcaatcaatctacataaaagtggcttccaggtttcaatgtaaaagctttaaaacacacacacatacacacacgcaccaggtttnaacgtaaaagctttaaaacacacacacgcatgcacatgcctacctcctntaagggattaaccaaatgaatttaatcaggactaaatctagagaaaaantttctaataactggttttaaaaggttgcgtaanantaaacccagccccaattanaagggggccanataactgaattcactccataantnaaaantttnatgtcaaggttttaattaaaaccantcaaaacnnangcntgccccttaagaatggggggaaaggaagaataacaaaacctnagcccgttcccaataagcttnttngaaaaatggaaaatttatccntgnggttcnatttttnacaannccctnnct

SEQ ID NO: 32 - DNA, 950 bp, Homo sapiens
  misc_feature 3, 12, 13, 14, 29, 41, 44, 50, 63, 64, 80, 91, 98, 99,122, 154, 160, 189, 191, 197, 204, 205, 207, 216, 219, 310, 324,342, 345, 394, 409, 433, 444, 654, 670, 685, 739, 741, 745,774, 793, 800, 813, 814, 825, 843, 844, 849, 850, 862: n = A,T,C or G
  misc_feature 882, 891, 892, 906, 909, 937, 939, 940, 944, 946: n = A,T,C or G
acncctttttcnnncttccatgctcgctnggccgacttgtncangggatntctgcagaaccgnnttgtaatacgactcantatagggctcncgtggtnnacggcccgggctggtctgtttgnaattggtaatacatacatactttcttggaatnagcctncttagagttagtactaatcaataagactnangggatntaccatnnangatcaagtntgngctctcttataaacatcactgctaccttatagtctaaatctcaaaagaaaatatttgaaaagttattctttttccactcaacaagtaattntgaatatgtgcttnatcctaggcacctaccanatnctggatatacagcctctactctcttggtgcttaccatttaaatttagtntttctttaatggttnacacatattccaagggtctttggncatacccatgnattgtaatttcggaagcctcactaagctaattgccttctggattcgatttggaactggaatagaaccaagtgaaagctaatgatggagatctaggaattgatatagttgaagctatactaataggtgagatctcaaaggagagaaaaccagaaagccaaagactgaacatttctggagttgggaatgaaaaggagacacagaaaaaaatntaaatggaacttaaangagacaaggggagcncctggaaaattagtgttattggaatcaaaggagaaaataatttcaagaaggaanangggnacctataacatttattgctctcgagaagnaaaggagactcccgattanggagcanttttaaaacaacnngatggccacgntccacatattcttgaggnnatggnngcaattaccatngtccaagggttacgcccccntaactttcnntttttttaaattanttntaatgcagttatattcctcacattttcncnntttnanggct

SEQ ID NO: 33 - DNA, 1065 bp, Homo sapiens
  misc_feature 14, 21, 27, 33, 36, 42, 72, 101, 103, 207, 208, 221,223, 227, 235, 240, 242, 243, 247, 248, 259, 263, 269, 273, 278,296, 321, 322, 324, 330, 332, 335, 336, 340, 367, 371, 385,390, 393, 399, 401, 407, 415, 421, 447, 454, 471, 475: n = A,T,C or G
  misc_feature 479, 494, 501, 508, 511, 513, 525, 539, 540, 579, 596,605, 623, 624, 633, 653, 654, 656, 658, 664, 670, 679, 711, 713,721, 729, 735, 743, 747, 748, 754, 776, 778, 779, 780, 783,798, 802, 808, 810, 813, 814, 820, 822, 824, 825, 838: n = A,T,C or G
  misc_feature 842, 847, 888, 900, 909, 910, 916, 926, 927, 943, 948,962, 973, 1002, 1005, 1028, 1029, 1034, 1057, 1065: n = A,T,C or G
ccccccttttaaanccctttnaaatancttcangangtaaangtggatctataacatttattgctatagagnaagtaaaggagcaatgtagattagggagntnttttaaaacaactgatggccacgttttacatattcttgatgtatatgttgaattacatgtcaaggatatgtaatgtaatttcattttttaaatatttttatgcnntatattctcacantnttgngtcccatnccacntnnaccnnactccaccgcncctntcccancccncacancctaaatttccactttcncacgcaactcactcccccccccccnntncccccntnccnncccnccccccccacctcctcccccccccccnctcncccccctctcttcnccccnccncctctncnctcctnccctcctnttccanccccccccccccctcccccccccctntctctcncccccccccctcccctncccnctcncaccccccttccccntcccccntccctcntcncncccccccccccnccccccctcctctnnctccctcccccccccccctcctctcccccccctcccccnccccccttccccctccnccccccccnccccccccccccccctcnnccccccccnaccccccccccccccccttnncncnccctcncccccnccttcccanccctcctccctctccccccccctccccctccncnattccccnccttcccncccccncccccccncctnnccctcncccttctcccccctcctccccncnnnccncccccctcccccccncccncccttntnacnncccccncncnnccccccctcactncccnccccncccccccccctcccccttcccttcccccctccatcttctancccccccccctnctcccctcnncccccncccccctccnntcccccccccccctcnctccnccctccccccccanccccttccccntcccccccccctctccctccccctctctnctntccttccccttcatcccctccanncttcncccctccccctccctctcccccncccccccn

SEQ ID NO: 34 - DNA, 1079 bp, Homo sapiens
  misc_feature 3, 11, 21, 28, 38, 40, 41, 91, 99, 531, 566, 582, 589,590, 615, 616, 620, 624, 629, 637, 643, 653, 655, 659, 663, 667,700, 702, 710, 720, 727, 734, 735, 743, 747, 748, 749, 759,761, 768, 769, 773, 779, 781, 784, 787, 792, 793, 794: n = A,T,C or G
  misc_feature 796, 808, 812, 818, 820, 821, 824, 830, 838, 839, 847,850, 854, 863, 865, 866, 870, 891, 898, 900, 901, 902, 910, 913,914, 930, 939, 944, 948, 953, 957, 960, 961, 963, 965, 966,967, 971, 973, 978, 982, 988, 990, 998, 1000, 1002: n = A,T,C or G
  misc_feature 1004, 1007, 1008, 1013, 1020, 1021, 1035, 1039, 1050,1055, 1057, 1058, 1060, 1068, 1079: n = A,T,C or G
tcnctaaaccntttaaacctnccctcantccttatagngnnacaaaaggcagtaaagttagctgtactgcaatcaatctacataaaagtgntttccagntttcaatgtaaaagctttaaaacacacacacatacacacacgcaccaggtttcaacgtaaaagctttaaaacacacacacgcatgcacatgcctacctcctctaagggattaacaaatgaatttaatcaggactaaatctagagaaaaaatttctaataactgttttaaaagttgtagtaaaataaactcagaccaattaaaggggcaaataatgaattcaattacataataaaaatatatgtcaagttttaattaaaaacaatcaaacaaatgcatgcacttaagatgtggggaaagaagaataaaaaactaagcagttcacaataagtttcttgaaaaatgaaaatttatctgtgtatcattttaatatctccttgaagtttctaaatctgacataaaacacaaaaatgtgaaaatatactgcataaaantatttacaaaaatgaaattcattacatatccttgncatgtaattcaacatntacatcnngcaatatgtaaaacgtggccatcannttgntttncactnctccctantctacnttctcccttncntctntatngcantacatgtattgatcccaccttttcctttcttgnanattatttntttccttctntctccantcacctnnatctcctncagnnncttccctttntntttttcnngctncccccntntcntcntcccnnncntcccttctcctncccnccttcncnnctnttctcnttttcccnncttctttnctnctcncctcctatncnncccncccttctccctccttcctccnccttctncnnntctctctnctnntcttcttcctcttttntctccctcnccccnctcncttcnctcntcnncntnnnttcncnctccnttcncctccncncctccccncncntnttnnttttnttcctcnntcctcctctccctntttncctttccttcntctcncnntncctcctcnccctcctcccn

SEQ ID NO: 35 - DNA, 1009 bp, Homo sapiens
  misc_feature 3, 8, 29, 31, 33, 59, 78, 79, 87, 95, 96, 98, 105, 106,127, 134, 135, 475, 734, 738, 739, 740, 773, 797, 806, 813, 822,847, 848, 854, 857, 864, 878, 879, 880, 883, 898, 905, 918,919, 927, 944, 947, 951, 956, 960, 963, 979, 984: n = A,T,C or G
  misc_feature 987, 988, 990, 996, 1006: n = A,T,C or G
ttntaaancccttttgaaaccctttctanananccgacactattttacactcaagctcngcatcaagcttggtaccgnnctcggatncactagtnncngccgccnntgtgctggaattcggcttttnaggtatnncaaatatgcttcaagactggctggaaaagaaaatataacagaacatgaagcaaaagaagttcatcgaagcctaaagattgcagctgggatttttaaacatttaaaggaaagtcatctcccaaaactcattacacctgcggaaaaaggaagagatttagagtcacgactcatagaagcatacgttattcaatgtcaggctgaagctcaagaagtaacaattgctcgagcaattgaactaaaacatgctcctggactaattgctgcactggcgtatgaaacagccaatttctatcaaaaagctgtttggagcctgcatattctgccaaatggagaaaatatnttcacttgaagatgtgtttctacacagcttatgcttactgttaccatggtgagactttattggctagtgataaatgcggtgaagcaatcaggtctctccaagaagcagaaaaattgtatgcaaaggcagaagcactgtgtaaagaatatggagaaaccaaaggacctggaccaacagtcaagccgaattctgcagatatccatcacactggcggccgctcgagcatgcatctagagggcccaactcgcccctatagtgngtcnnnttacaattcactggccctcgttttacaccgtcntgacttggggaaaacccctggcgntaccccacntaaatcngccttgcangcacatcccccttttgccagctggnncaacanccnaagaggncccgcacccggatnnnccnttccaaacagttggnccagccnggaatgggcgaannggcccccncccctggaatcggcgcnttnagcnccggngggngtnggtgggtttccccccnaacgnggnncnctaccntttggccagnccc

SEQ ID NO: 36 - DNA, 987 bp, Homo sapiens
  misc_feature 6, 21, 22, 26, 29, 41, 57, 75, 94, 103, 124, 126, 131,132, 139, 698, 728, 749, 764, 774, 803, 806, 808, 822, 838, 849,861, 870, 873, 877, 888, 891, 895, 896, 936, 940, 942, 943,944, 946, 970, 972, 979: n = A,T,C or G
ttaaanccctacgaaaacccnntccnaangcccagctcgtncggccgccagtgccangggatatctgcagaattntgcttgactgttggtcctngtcctttgntttctccatattctttacacngngcttnngcctttncatacaatttttctgcttcttggagagacctgattgcttcaccgcatttatcactagccaataaagtctcaccatggtaacagtaagcataagctgtgtagaaacacatcttcaagtgaagatattttctccatttggcagaatatgcaggctccaaacagctttttgatagaaattggctgtttcatacgccagtgcagcaattagtccaggagcatgttttagttcaattgctcgagcaattgttacttcttgagcttcagcctgacattgaataacgtatgcttctatgagtcgtgactctaaatctcttcctttttccgcaggtgtaatgagttttgggagatgactttcctttaaatgtttaaaaatcccagctgcaatctttaggcttcgatgaacttcttttgcttcatcttctgttatattttcttttccagccagtcttgaagcatatttggtatacctaaaaagccgaattccagcacactggcggccgttactagtggatccgagctcggtaccaagcttgatgcatagcttgagtattctatagtgncacctaaatagcttggcgtaatcatggtcntagctggttcctgggtgaaantggtatccctcacanttccacacancataccagccggaagcataaagtgtaaanccngnggtgcctaatgagngagccaacctcaccantaattggcgtngggctcacctgncccgctttncangccnggaaaccctgncgngccnncctgcatttaatggaatccgccccccccccggggagaggncggntnnncnttattggccgctctttcccctttncncccttanttgacccc

SEQ ID NO: 37 - DNA, 1010 bp, Homo sapiens
  misc_feature 8, 13, 15, 23, 30, 31, 38, 43, 48, 54, 70, 81, 83, 88,90, 99, 108, 109, 128, 136, 143, 144, 149, 150, 151, 579, 630,640, 684, 702, 788, 825, 833, 838, 841, 846, 847, 866, 872,876, 891, 899, 907, 923, 927, 940, 952, 965, 972, 975: n = A,T,C or G
  misc_feature 985, 998, 999, 1001: n = A,T,C or G
tcctaaancctcntnaaaccccntccgacnngtccggncgacnttgcncagggnatatccgcgggaattntgctttttatngnatacnanatatgcttnaagcactgnntggaaaagaaaatataacnggggatgnggcaaanntaagnnnatcgaagcctaaagattgcagctgggatttctaaacatttaaaggaaagtcatctcccaaaactcattacacctgcggaaaaaggaagagatttagagtcacgactcatagaagcatacgttattcaatgtcaggctgaagctcaagaagtaacaattgctcgagcaattgaactaaaacatgctcctggactaattgctgcactggcgtatgaaacagccaatttctatcaaaaagctgatcatactttatccagtttggagcctgcatattctgccaaatggagaaaatatcttcacgtgaagatgtgtttctacacagcttatgcttactgttaccatggtgagactttattggctagtgataaatgcggtgaagcaatcaggtctctccaagaagcagaaaaattgtatgcaaaggcagaagcnctgtgtaaagaatatggagaaaccaaaggacctggaccaacagtcaagccnaattccagcncactggcggccgttactagtgggatccgagctcggtaccaagcntggatgcatagcttgagnattctatagtgtcacctaaatagcttggcgtaatcatgggcatagctgtttcctgtgtgaaaatgttctccccctcaccaattccncacaacatacgagcccggcaagcataaagtgtaaacncccggggntgccntantgagnngagcctacctcccattaanttgcgntgcncctcacctgccccgntttccagnccgggaanaccctgtccgggcccncttncattaaatggaantcgccccccccncggggaggaggcngcttgcntantggccgcttnttccgcttcctcnncnctgactccc

SEQ ID NO: 38 - DNA, 1014 bp, Homo sapiens
  misc_feature 9, 47, 66, 76, 83, 91, 94, 114, 124, 125, 129, 135, 136,143, 148, 385, 388, 531, 552, 565, 627, 628, 645, 697, 698,706, 715, 718, 723, 730, 739, 751, 769, 788, 803, 806, 809,810, 811, 822, 827, 834, 841, 850, 855, 863, 869, 875: n = A,T,C or G
  misc_feature 876, 881, 903, 908, 923, 928, 931, 936, 937, 942, 952,953, 963, 979, 982, 984, 985, 990, 993, 1005, 1006: n = A,T,C or G
tcgcaaaancccccgaaaccctcttacaaaattcaaactaattctcngcatcaagcttggtaccgntctcggatcnactagtntcggccgnctntgtgctgtaattcggctcgngcttctgggnntgcntgcaanntttctgnttctnggagagacctgattgcttcaccgcatcttatcactagccaataaagtctcaccatggtaacagtaagcataagctgtgtagaaacacatcttcaagtgaagatattttctccatttggcagaatatgcaggctccaaactggataaagtatgatcagctttttgatagaaattggctgtttcatacgccagtgcagcaattagtccaggagcatgttttagttcaattgctcgagcnatngttacttcttgagcttcagcctgacattgaataacgtatgcttctatgagtcgtgactctaaatctcttcctttttccgcaggtgtaatgagttttgggagatgactttcctttaaatgtttaaaaatcccagctgcaatctntaggctttgacgaacctcttntgcttcatcttcngttatattcccttttccagccagccttgaagcatatttcggtataccataaagctacattannatcccatggaaattaanttcaaaaacagcatccctgctgggcccttgggaaccctgtcctttgcaatgnntcaccccncttgaaaanttngaatnaagcccnattttgcanatatccttcacnctggccggccgttcagcntgccatctagagggccccnatttcgccctatagngangcnnnttaccaattcncctgncccctcncttttancaacccccngcacntgggaaancccctngccctnnccccncctcaaatcgcccttgcaccancatcncccctttcgcccacnttggncgntaaanncccanaaaggccccnncccctatcgncccttttcccaacaanttncnncatcnctngaatggccaaanngccccccc

SEQ ID NO: 39 - DNA, 1024 bp, Homo sapiens
  misc_feature 2, 4, 7, 12, 23, 27, 44, 54, 57, 63, 71, 98, 107, 115,116, 125, 144, 150, 171, 296, 340, 351, 358, 378, 388, 404, 407,408, 434, 435, 448, 464, 504, 506, 538, 546, 547, 561, 572,584, 585, 609, 611, 612, 613, 618, 627, 629, 638, 642: n = A,T,C or G
  misc_feature 644, 649, 653, 656, 663, 668, 671, 673, 675, 677, 704,705, 707, 713, 714, 715, 716, 721, 729, 734, 735, 736, 747, 750,752, 754, 760, 761, 764, 766, 767, 768, 772, 775, 784, 785,794, 799, 807, 808, 809, 813, 815, 820, 822, 823, 827: n = A,T,C or G
  misc_feature 830, 832, 840, 846, 859, 862, 863, 873, 878, 888, 896,900, 904, 912, 923, 929, 930, 934, 935, 938, 941, 945, 966, 974,992, 994, 999, 1020: n = A,T,C or G
tngnaanccttntaaacccttcngcangtccggccgaaattgcnagggatatcngcngaattnctttattnaaaatttttgtggacttttacattgtnggacaggtnccaagtgnngggcaggtngctgattttgaattaattnccatgngatttaatgtagctttatcgnataccaaatatgcttcaagactggctggaaaagaaaatataacagaagatgaagcaaaagaagttcatcgaagcctaaagattgcagctgggatttttaaacatttaaaggaaagtcatctcccnaaactcattacacctgcggaaaaaggaagagatttagagtcacnactcatagaancatacgntattcaatgtcaggctgaanctcaagaagnaacaattgctcgagcnatnnaactaaaacatgctcctggactaatnnctgcactggccgnatgaaacacccaattnctatcaaaaagctgatcatactttatccagtttggagccngnatattctgccaaaaggagaaaacatcttcacntgaagatnngcttctacccaccntatgccttccngtcaccatggcnngactttattggctagtgacaaaancnnngacancaatcacgncncttccaagncgcncnaacanttgnctncccaaangccgnaancncnanccccaccccacgacactcgccggcccnntnctaggnnnntcccnagcctcgnacccnnnctctcgatccntancntngaggannttnannnctgnccncctcaccannccttggccncaatnatgggccnnncttncntcccncnncgancantncctatccnctcacnaattccccccccncannaccagcccgnaagcnctacacgtgnccacctcntggnctgnccctacgnagggcaccccnccctcnncttnnccnggngccnggcccccccccccgcttttcncctccgcnacaacactttctctcccncnttgcntttaactaatccgcccccccnccct

SEQ ID NO: 40 - DNA, 691 bp, Homo sapiens
atctagtactttgctattcaaaagtgtctggggaccagcagcatcagcatctcaggtcctgccccaggctattgaattagagcctgcattggacaaaaccacccacccccagaaaacatctggagaaaatgacccattggtttcataggaacccattaaaagccacagctcctgtgtcttttaattactatggtgtagtcactggcccttctgcttcaaaaatatgcaatgacttgaggtcatccagggcacgactccttgaactgttcactgatttgagctgtaatccagaaatgatgaagaatgcagcagattcatatttctcacttttacaaggtttcataaattctttggatgaatctacccaagaaagcaagttacgatatattcaaaatttcaagtggactgatacattgcaaggacaggttccaagtataacagaagatgaagcaaaagaagttcatcgaagcctaaagattgcagctgggatttttaaacatttaaaggaaagtcatctcccaaaactcattacacctgcggaaaaaggaagagatttagagtcacgactcatagaagcatacgttattcaatgtcaggctgaagctcaagaagtaacaattgctcgagcaattgaactaaaacatgctcctggactaattgctgcactggcgtatgaaacagcccaatttct

SEQ ID NO: 41 - DNA, 681 bp, Homo sapiens
  misc_feature 14, 31, 35, 54, 70, 74, 77, 79, 118, 173, 198, 350, 435,556, 593, 602, 603, 634, 666, 673: n = A,T,C or G
tgggtgggcttttnttggctagggataaatncggngaagcaatcagggtctctncaaggagcagagaatngtangcnanggcagaagcactgtgtaaagaatatggaggaaccaaagnccggggcaacagtcaaaccttcaggacatctgttctttaggaaacttgggaaccntgtgaagaacaccctagaaaaatgncagagagaaaatggatttatttactttcaaaaaattccaacagaagccccacagctggaactcaaagcaaattatggtctcgtagagcctatacctttcgaatttcctcctacaagtgttcagtggacaccagaaacattggctgcatttgntctcaccaaaagacccaaggatgacagtactaaacccaaaccagaagaagaagtgaaacctgtgaaagaaccagacatcaaaccncaaaaggacactgggtgctacatctcctaaaatacaacttgcacttagaatttctctagcagtaaataagataaaccacagaatttcagttcttatttctcaaaatgatttctctgaagcntgtagaataactattattcagagggttatctgccttnaacttacannttcttaatttttaatacagcggagatgtttnttgaaaaaaaaaaaaaaaaaaattgcggccgnaagcttnaaagggtt

SEQ ID NO: 42 - DNA, 759 bp, Homo sapiens
  misc_feature 18, 23, 24, 83, 88, 92: n = A,T,C or G
aacccttttgccctcgcngccannaattcggcacgaggccgatatattgcccttcttcccttagaaggaactgctgaaccgtntctgngttnatttgttgtccaaccaaaatctagtactttgctattcaaaagtgtctggggaccagcagcatcagcatctcaggtcctgccccaggctattgaattagagcctgcattggacaaaaccacccacccccagaaaacatctggagaaaatgacccattggtttcataggaacccattaaaagccacagctcctgtgtcttttaattactatggtgtagtcactggcccttctgcttcaaaaatatgcaatgacttgaggtcatccagggcacgactccttgaactgttcactgatttgagctgtaatccagaaatgatgaagaatgcagcagattcatatttctcacttttacaaggtttcataaattctttggatgaatctacccaagaaagcaagttacgatatattcaaaatttcaagtggactgatacattgcaaggacaggttccaagtataacagaagatgaagcaaaagaagttcatcgaagcctaaagattgcagctgggatttttaaacatttaaaggaaagtcatcttccaaaactcattacacctgcggaaaaaggaagagatttagagtcacgactcatagaagcatacgttattcaatgtcaggctgaagctcaagaagtaacaattgctcgagcaattgaactaaaacatgctcc

SEQ ID NO: 43 - DNA, 351 bp, Homo sapiens
  misc_feature 8, 26, 54, 57, 62, 66, 73, 77, 81, 105, 129, 134, 212,284, 292, 294: n = A,T,C or G
ttcctccnacaagtgttcagtggacnccagaaacattggctgcatttgatctcnccnaaagncccnaggatgncagnactnaacccaaaccagaagaagaagtgnaacctgtgaaagaaccagacatcnaaccncaaaaggacactgggtgctacatctcctaaaatacaacttgcacttagaatttctctagcagtaaataagataaaccncagaatttcagttcttatttctcaaaatgatttctctgaagcttgtagaataactattattcagagggttanaagccttnancttacttgttcttaatttttaatacagcggagatgtttcttgaaaaaaaaaaaaaaa

SEQ ID NO: 44 - DNA, 763 bp, Homo sapiens
  misc_feature 67, 93, 104, 105, 120, 125, 128, 200, 235, 248, 271, 276: n = A,T,C or G
gcacgaggccgatatattgcccttcttcccttagaagaactgctgaaccgactctgtttttttgttntccaaccaaaatctagtactttgctnttcaaaagtgnntggggaccagcagcntcagnatntcaggtcctgccccaggctattgaattagagcctgcattggacaaaaccacccacccccagaaaacatctgnagaaaatgacccattggtttcataggaacccattnaaagccacagctnctgtgtcttttaattactatggngtagncactggcccttctgcttcaaaaatatgcaatgacttgaggtcatccagggcacgactccttgaactgttcactgatttgagctgtaatccagaaatgatgaagaatgcagcagattcatatttctcacttttacaaggtttcataaattctttggatgaatctacccaagaaagcaagttacgatatattcaaaatttcaagtggactgatacattgcaaggacaggttccaagtataacagaagatgaagcaaaagaagttcatcgaagcctaaagattgcagctgggatttttaaacatttaaaggaaagtcatctcccaaaactcattacacctgcggaaaaaggaagagatttagagtcacgactcatagaagcatacgttattcaatgtcaggctgaagctcaagaagtaacaattgctcgagcaattgaactaaaacatgctcctgggctaattgctgcactggcgtatgaaacagccaaa

SEQ ID NO: 45 - DNA, 522 bp, Homo sapiens
  misc_feature 14, 25, 32, 44, 47, 106, 166, 191, 354, 434, 441, 443,444, 475, 507, 514: n = A,T,C or G
aaacttggaaaccntgtgaagagcnccctagnaaaatgtcaganagnaaatggatttatttactttcaaaaaattccaacagaagccccacagctggaactcaaancaaattatggtctcgtagagcctatacctttcgaatttcctcctacaagtgttcagtggncaccagaaacattggctgcatttgntctcaccaaaagacccaaggatgacagtactaaacccaaaccagaagaagaagtgaaacctgtgaaagaaccagacatcaaacctcaaaaggacactgggtgctacatctcctaaaatacaacttgcacttagaatttctctagcagtaaataagataaaccncagaatttcagttcttatttctcaaaatgatttctctgaagcttgtagaataactattattcagagggttatctgccttnaacttanannttcttaatttttaatacagcggagatgtttnttgaaaaaaaaaaaaaaaaaaattgcggccgnaagcttnaaagggtt

SEQ ID NO: 46 - DNA, 746 bp, Homo sapiens
  misc_feature 332, 337: n = A,T,C or G
aggtttcataaattctttggatgaatctacccaagaaagcaagttacgatatattcaaaatttcaagtggactgatacattgcaaggacaggttccaagtgcccagcaggatgctgtttttgaattaatttccatgggatttaatgtggctttatggtataccaaatatgcttcaagactggctggagaagaaaatataacagaagatgaaggaaaagaagttcatcgaagcctaaagattgcagctgggatttttaaacatttaaaggaaagtcatctcccaaaactcattacacctgacggaaaaaggaagagatttagagtcacgactncatanaagcatacgttattccaatgtcaggctgaagctcaagaagtaacaattgctcgagcaattgaactaaaacatgctcctggactaattgctgcactggcgtatgaaacagccaatttctatcaaaaagctgatcatactttatccagtttggagcctgcatattctgccaaatggagaaaatatcttcacttgaagatgtgtttctacacagcttatgcttactgttaccatggtgagactttattggctagtgataaatgcggtgaagcaatcaggtctctccaagaagcagaaaaattgtatgcaaaggcagaagcactgtgtaaagaatatggagaaaccaaaggacctggaccaacagtcaaaccttcaggacatctgttctttaggaaacttggaaaccttgtga

SEQ ID NO: 47 - DNA, 852 bp, Homo sapiens
  misc_feature 39, 71, 90, 94, 136, 813, 816, 832, 843: n = A,T,C or G
acggatatctgcagaattcggcttatgacccattggttncataggaacccattaaaagccacagcttctgngtcttttaattactatggngtantcactggcccttctgcttcaaaaatatgcaatgacttgaggncatccagggcacgactccttgaactgttcactgatttgagctgtaatccagaaatgatgaagaatccagcagattcatatttctcacttttacaaggtttcataaattctttggatgaatctacccaagaaagcaagttacgatatattcaaaatttcaagtggactgatacattgcaaggacaggttccaagtgcccagcaggatgctgtttttgaattaatttccatgggatttaatgtagctttatggtataccaaatatgcttcaagactggctggaaaagaaaatataacagaagatgaagcaaaagaagttcatcgaagcctaaagattgcagctgggatttttaaacatttaaaggaaagtcatctcccaaaactcattacacctgcggaaaaaggaagagatttagagtcacgactcatagaagcatacgttattcaatgtcaggctgaagctcaagaagtaacaattgctcgagcaattgaactaaaacatgctcctggactaattgctgcactggcgtatgaaacagccaatttctatcaaaaagctgatcatactttatccagtttggagcctgcatattctgccaaatggagaaaatatcttcacttgaagatgtgttctacacagcttatgcttactgtaccatgggtgagactttattggctnggngataaatgcggtgaancaatcaggctnttcaagaag

SEQ ID NO: 48 - DNA, 881 bp, Homo sapiens
  misc_feature 1, 11, 85, 94, 863, 874: n = A,T,C or G
ntcttttaacnctatggtgtagtcactggcccttctgcttcaaaaatatgcaatgacttgaggtcatccagggcacgactccttnaactgttcnctgatttgagctgtaatccagaaatgatgaagaatccagcagattcatatttctcacttttacaaggtttcataaattctttggatgaatctacccaagaaagcaagttacgatatattcaaaatttcaagtggactgatacattgcaaggacaggttccaagtgcccagcaggatgctgtttttgaattaatttccatgggatttaatgtagctttatggtataccaaatatgcttcaagactggctggaaaagaaaatataacagaagatgaagcaaaagaagttcatcgaagcctaaagattgcagctgggatttttaaacatttaaaggaaagtcatctcccaaaactcattacacctgcggaaaaaggaagagatttagagtcacgactcatagaagcatacgttattcaatgtcaggctgaagctcaagaagtaacaattgctcgagcaattgaactaaaacatgctcctggactaattgctgcactggcgtatgaaacagccaatttctatcaaaaagctgatcatactttatccagtttggagcctgcatattctgccaaatggagaaaatatcttcacttgaagatgtgtttctacacagcttatgcttactgttaccatggtgagactttattggctagtgataaatgcggtgaagcaatcaggtctctccaagaagcagaaaaattgtatgcaaaggcaagaagcactggtaaagagtatggagaagccaaaggacctggaccaacagtcaaaccttnggacatctgtntttagga

SEQ ID NO: 49 - DNA, 747 bp, Homo sapiens
  misc_feature 86, 87, 88, 602, 669, 719, 734, 736, 737: n = A,T,C or G
tttctggttccttcacaggtttcacttcttcttctggtttgggtttagtactgtcatccttgggtcttttggtgagatcaaatgcnnncaatgtttttggtgtccactgagcacttgtaggaggaaattcgaaaggtataggctctacgagaccataatttgctttgagttccagctgtggggcttctgttggaattttttgaaagtaaataaatccattttctctctgacatttttctagggtgttcttcacaaggtctccaagtttcctaaagaacagatgtcctgaaggtttgactgttggtccaggtcctttggcttctccatactctttacacagtgcttctgcctttgcatacaatttttctgcttcttggagagacctgattgcttcaccgcatttatcactagccaataaagtctcaccatggtaacagtaagcataagctgtgtagaaacacatcttcaagtgaagatattttctccatttggcagaatatgcaggctccaaactggataaagtatgatcagctttttgatagaaattggctgtttcatacgccagtgcagcaattagtccaggagcatgttttagttcaatngctcgagcaattgttacttcttgagccttcagcctgacattgaataacgtatgcttctatgagtcgngactctaaatctcttcctttttccgcaggtgtaatgagtttgggagatgncttttctttaaatgntnnaaaatcccag

SEQ ID NO: 50 - DNA, 755 bp, Homo sapiens
  misc_feature 24, 25, 32, 66, 70, 80, 144, 191, 434, 462, 601, 603,673, 719, 745: n = A,T,C or G
gtcaggcttgaagctcaagaagtnnaattgcncgagcaattgaactaaaacatgctcctggactanattnctgcactggnggtatgaaacagccaatttctatcaaaaagctgatcatactttatccagtttggagcctgcatnttctgccaaatggagaaaatatcttcacttgaagatgtgtttctacncagcttatgcttactgttaccatggtagactttattggctagtgataaatgcggtgaagcaatcaggtctctccaagaagcagaaaaattgtatgcaaaggcagaagcactgtgtaaagagtatggagaagccaaaggacctggaccaacagtcaaaccttcaggacatctgttctttaggaaacttggagaccttgtgaagaacaccctagaaaaatgtcagagagaaaatggatttatttnctttcaaaaaattccaacagaagccccncagctggaactcaaagcaaattatggtctcgtagagcctatacctttcgaatttcctcctacaagtgctcagtggacaccagaaacattggctgcatttgatctcaccaaaagacccaaggatgacagtactaaacccnanccagaagaagaagtgaaacctgtgaaggaaccagacatcaaacctcaaaagggctctggaaaatacatanacaagccgaattccagcacactgacggccgttactagtggatccgnggggggtttttagccaaatgcatagngaagtttttt

SEQ ID NO: 51 - DNA, 499 bp, Homo sapiens
  misc_feature 18, 19, 37, 56, 67, 82, 95, 98, 105, 110, 132, 153, 163,186, 196, 205, 206, 214, 253, 255, 263, 275, 280, 292, 325,326, 343, 355, 357, 359, 389, 398, 400, 425, 430, 432, 440,446, 450, 453, 458, 469, 478, 492: n = A,T,C or G
aaaggccccataggaacnnagtaaaagggacagcttntgtgacttttaattactanggcgcagtcantggccgttatgcttnaaaaatatgcaanganttgaggncatcnagggcacgactccttgaactgntcactgatttgagctgtaatncagaaatgangaagaatgcagcagattcatatntctcactttnacaaggttnnataaattntttggatgaatctacccaagaaagcaagttacgatatananaaaatttnaagtggactganacatngcaaggacaggntccaagtataacagaagatgaagcaaaagaagnncatcgaagcctaaagantgcagatgggantntnaaacatctaaaggaaagcaactaccaaaantcattacancngcggaaaaaggaagagattagaganacgantnatagaagnatacgntatnaantgatnggctgaagctnaagaagtanaatcggccgagcanctgaaca

SEQ ID NO: 52 - DNA, 850 bp, Homo sapiens
  misc_feature 83, 792, 822, 834, 848: n = A,T,C or G
ttttaattactatggtgtagtcactggcccttctgcttcaaaaatatgcaatgacttgaggtcatccagggcacgactccttntactgttcactgatttgagctgtaatccagaaatgatgaagaatgcagcagattcatatttctcacttttacaaggtttcataaattctttggatgaatctacccaagaaagcaagttacgatatattcaaaatttcaagtggactgatacattgcaaggacaggttccaagtataacagaagatgaagcaaaagaagttcatcgaagcctaaagattgcagctgggatttttaaacatttaaaggaaagtcatctcccaaaactcattacacctgcggaaaaaggaagagatttagagtcacgactcatagaagcatacgttattcaatgtcaggctgaagctcaagaagtaacaattgctcgagcaattgaactaaaacatgctcctggactaattgctgcactggcgtatgaaacagccaatttctatcaaaaagctgatcatactttatccagtttggagcctgcatattctgccaaatggagaaaatatcttcacttgaagatgagtttctacacagcttatgcttactgttaccatggtgagactttattggctagtgataagtgcggtgaagcaatcaggtctctccaagaagcagaaaaattgtatgcaaaggcagaagcctgtgtaaagaatatgggagaagccaaaggacctggaccaacagtcaaaccttcaggacatctgttctttaggaaactggnaaccttgtgaagaacaccctagaaaaatgntagagagaaaangatttattactttnaa

SEQ ID NO: 53 - DNA, 868 bp, Homo sapiens
  misc_feature 7, 17, 41, 51, 68, 142, 167, 770, 771: n = A,T,C or G
ggatgantctcccaagnaagcaagttcgatatattcaaatnttcaagtggnctgatacattgcaaggncaggttccaagataacagaagatgaagcaaaagaagttcatcgaagcctaaagattgcagctgggatttttaancatttaaaggaaagtcatctcccanaactcattacacctgcggaaaaaggaagagatttagagtcacgactcatagaagcatacgttattcaatgtcaggctgaagctcaagaagtaacaattgctcgagcaattgaactaaaacatgctcctggactaattgctgcactggcgtatgaaacagccaatttctatcaaaaagctgatcatactttatccagtttggagcctgcatattctgccaaatggagaaaatatcttcacttgaagatgagtttctacacagcttatgcttactgttaccatggtgagactttattggctagtgataagtgcggtgaagcaatcaggtctctccaagaagcagaaaaattgtatgcaaaggcagaagcactgtgtaaagaatatggagaagccaaaggacctggaccaacagtcaaaccttcaggacatctgttctttaggaaacttggaaaccttgtgaagaacaccctagaaaaatgttagagagaaaatggatttatttactttcaaaaaattccaacagaagccccacagctggaactcaaagcaaattatggtctcgtagagcctatacctttcgaatttcctcctacaagtgtccagnngacaccaaaaacattggctgcatttgatctcaccaaaagacccaaggatgacagtactaaacccaaaccagaagaagaagtgaaacctgtgaaagag

SEQ ID NO: 54 - DNA, 862 bp, Homo sapiens
  misc_feature 5, 37, 40, 41, 51, 75, 87, 104, 113, 160, 360, 782, 812,827, 856, 857: n = A,T,C or G
gcaanagaagttcgtcgaaggcctaaagattgcagcnggnntttttagacntttaaaggagagtcatctcccaanactcattacacntgggaaaaagaagaganttagagtcncgactcatagaagcatacgttattcaatgtcaggctgaagctcaagnagtaacaattgctcgagcaattgaactaaaacatgctcctggactaattgctgcactggcgtatgaaacagccaatttctatcaaaaagctgatcatactttatccagtttggagcctgcatattctgccaaatggagaaaatatcttcacttgaagatgagtttctacacagcttatgcttactgttaccatggtgagnctttattggctagtgataagtgcggtgaagcaatcaggtctctccaagaagcagaaaaattgtatgcaaaggcagaagcactgtgtaaagaatatggagaagccaaaggacctggaccaacagtcaaaccttcaggacatctgttctttaggaaacttggaaaccttgtgaagaacaccctagaaaaatgttagagagaaaatggatttatttactttcaaaaaattccaacagaagccccacagctggaactcaaagcaaattatggtctcgtagagcctatacctttcgaatttcctcctacaagtgtccagtggacaccagaaacattggctgcatttgatctcaccaaaagacccaaggatgacagtactaaacccaaaccagaagaagaagtgaaacctgtgaaagaaccagacatnaaacctcaagaggacactgggtgctacatnctccaagccgaattnctgcagatatccatcacactggcggccgnncgagc

SEQ ID NO: 55 - DNA, 876 bp, Homo sapiens
  misc_feature 4, 786, 857: n = A,T,C or G
ggcncgccagtgtggatgggatatctgcaggaattcggcttatgacccattggtttcataggaacccattaaaagccacagcttctgtgtcttttaattactatggtgtagtcactggcccttctgcttcaaaaatatgcaatgacttgaggtcatccagggcacgactccttgaactgttcactgatttgagctgtaatccagaaatgatgaagaatgcagcagattcatatttctcacttttacaaggtttcataaattctttggatgaatctacccaagaaagcaagttacgatatattcaaaatttcaagtggacggatacattgcaaggacaggttccaagaagatgaagcaaaagaagttcatcgaagcctaaagattgcagctgggatttttaaacatttaaaggaaagtcatctcccaaaactcattacacctgcggaaaaaggaagagatttagagtcacgactcatagaagcatacgttattcaatgtcaggctgaagctcaagaagtaacaattgctcgagcaattgaactaaaacatgctcctggactaattgctgcactggcgtatgaaacagccaatttctatcaaaaagctgtttggggcctgcatattctgccaaatggagaaaatatcttcacttgaagatgtgtttctacacagcttatgcttactgttaccatggtgagactttattggctagtgataaatgcggtgaagcaatcaggtctctccaagaagcagaaaaattgtatgcaaaggcagaagcactgtgtnaagaatatggagaaaccaaaggacctggccaacagacaaaccttcaggacattgttctttaggaacttggnaaccttgggaagaacaccc

SEQ ID NO: 56 - DNA, 868 bp, Homo sapiens
  misc_feature 84, 805: n = A,T,C or G
cttttaattactatggtgtagtcactggcccttctgcttcaaaaatatgcaatgacttgaggtcatccagggcacgactccttnaactgttcactgatttgagctgtaatccagaaatgatgaagaatgcagcagattcatatttctcacttttacaaggtttcataaattctttggatgaatctacccaagaaagcaagttacgatatattcaaaatttcaagtggacggatacattgcaaggacaggttccaagaagatgaagcaaaagaagttcatcgaagcctaaagattgcagctgggatttttaaacatttaaaggaaagtcatctcccaaaactcattacacctgcggaaaaaggaagagatttagagtcacgactcatagaagcatacgttattcaatgtcaggctgaagctcaagaagtaacaattgctcgagcaattgaactaaaacatgctcctggactaattgctgcactggcgtatgaaacagccaatttctatcaaaaagctgtttggggcctgcatattctgccaaatggagaaaatatcttcacttgaagatgtgtttctacacagcttatgcttactgttaccatggtgagactttattggctagtgataaatgcggtgaagcaatcaggtctctccaagaagcagaaaaattgtatgcaaaggcagaagcactgtgtaaagaatatggagaaaccaaaggacctggaccaacagacaaaccttcaggacatctgttctttaggaaacttggaaaccttgtgaagaacaccctagaaaaatgtcaagngagaaaatggatttatttactttcaaaaaattccaacagaagcccacagctggaactcaagca

SEQ ID NO: 57 - DNA, 588 bp, Homo sapiens
  misc_feature 18, 23, 28, 33, 36, 40, 170, 583: n = A,T,C or G
catgctcctgactaattnctgcnctggngtatnaancagncaatttctatcaaaaagctgtttggggcctgcatattctgccaaatggagaaaatatcttcacttgaagatgtgtttctacacagcttatgcttactgttaccatggtgagactttattggctagtgatnaatgcggtgaagcaatcaggtctctccaagaagcagaaaaattgtatgcaaaggcagaagcactgtgtaaagaatatggagaaaccaaaggacctggaccaacagacaaaccttcaggacatctgttctttaggaaacttggaaaccttgtgaagaacaccctagaaaaatgtcagagagaaaatggatttatttactttcaaaaaattccaacagaagccccacagctggaactcaaagcaaattatggtctcgtagagcctatacctttcgaatttcctcctacaagtgttcagtggacaccagaaacattggctgcatttgatctcaccaaaagacccaaggatgacagtactaaacccaaaccagaagaagaagtgaaacctgtgaaagaaccagacatcaaacctcanaagga

SEQ ID NO: 58 - DNA, 551 bp, Homo sapiens
  misc_feature 14, 60, 467, 476: n = A,T,C or G
aactaattgctgcnctggcgtatgaaacagccaatttctatcaaaaagctgtttggggcntgcatattctgccaaatggagaaaatatcttcacttgaagatgtgtttctacacagcttatgcttactgttaccatggtgagactttattggctagtgataaatgcggtgaagcaatcaggtctctccaagaagcagaaaaattgtatgcaaaggcagaagcactgtgtaaagaatatggagaaaccaaaggacctggaccaacagacaaaccttcaggacatctgttctttaggaaacttggaaaccttgtgaagaacaccctagaaaaatgtcagagagaaaatggatttatttactttcaaaaaattccaacagaagccccacagctggaactcaaagcaaattatggtctcgtagagcctatacctttcgaatttcctcctacaagtgttcagtggacaccanaaacattgnaagcatttgatctcaccaaaagacccaaggatgacagtactaaacccaaaccagaagaagaagtgaaacctgtga

SEQ ID NO: 59 - DNA, 485 bp, Homo sapiens
  misc_feature 108, 181, 477, 485: n = A,T,C or G
tttgcgagggccagggataggcttaccttcaagctcgcccttatgacccattggtttcataggaacccattaaaagccacagctcctgtgtcttttaattactatggngtagtcactggcccttctgcttcaaaaatatgcaatgacttgaggtcatccagggcacgactccttgaactgntcactgatttgagctgtaatccagaaatgatgaagaatgcagcagattcatatttctcacttttacaaggtttcataaattctttggatgaatctacccaagaaagcaagttacgatatattcaaaatttcaagtggactgatacattgcaaggacaggttccaagtgcccagcaggatgctgtttttgaattaatttccatgggatttaatgtagctttatggtataccaaatatgcttcaagactggctggaaaagaaaatataacagaagatgaagcaaaagaagttcatcgnagcctaan

SEQ ID NO: 60 - DNA, 472 bp, Homo sapiens
  misc_feature 3, 5, 12, 42, 84, 109, 161, 198, 289, 412, 414: n = A,T,C or G
ccncngatttgngctgtaatccagaaatgatgaagaatgcancagattcatatttctcacttttacaaggtttcataaattctntggatgaatctacccaagaaagcangttacgatatattcaaaatttcaagtggactgatacattgcaaggacaggtnccaagtgcccagcaggatgctgtttttgaattaattnccatgggatttaatgtagctttatggtataccaaatatgcttcaagactggctggaaaagaaaatataacagaagatgaagcaaaagaagntcatcgaagcctaaagattgcagctgggatttttaaacatttaaaggaaagtcatctcccaaaactcattacacctgcggaaaaaggaagagatttagagtcacgactcatagaagcatacgntnttcaatgtcaggctgaagctcaagaagtaacaattgctcgagcaattgaactaaaaca

SEQ ID NO: 61 - DNA, 472 bp, Homo sapiens
  misc_feature 4, 18, 61, 94, 118, 137, 153, 171, 180, 238, 275, 315,335, 368, 369, 380, 443, 456: n = A,T,C or G
atgnaacagccaatttcnatcaaaaagctgatcatactttatccagtttggagcctgcatnttctgccaaatggagtaaatatcttcacttgangatgtgtttctacgcagcttatgnttactgttaccatggtgangctttattggctagtnataaatgcggtgaagcantcaggtctntccaagaagcagaaaaattgtatgcaaaggcagaagcactgtgtaaagaatatggagntaccaaaggacctggaccaacagtcaaaccttcaggncatctgttctttaggaaacttgggagccttgtgaagaacnccctagagaaatgtcagagngaaaatggatttatttactttcaaaaaattccnncagaagcctcncagctggaactcaaagcaaattatggtctcgtagagcctatacctttcgaatttcctcctacnagtgttcagtggnggtcctgggaaaatgg

SEQ ID NO: 62 - DNA, 488 bp, Homo sapiens
  misc_feature 26, 35, 36, 38, 113, 118, 120, 195, 301, 329, 394, 406: n = A,T,C or G
gtggagcaatcgaggtctcttccaanaaatcagannanattgtttgcagatggcataagctctgtgtaggggatatggagaagccaaaggacctggaccaacagtcaagcctncaggncntctgttctttaggaaacttggaaaccttgtgaagaacaccctagaaaaatgtcagagagaaaatggatttatttnctttcaaaaaattccaacagaagcctcacagctggaactcaaagcaaattatggtctcgtagagcctatacctttcgaatttcctcctacaagtgttcagtggacnccagaaacattggctgcatttgatctcnccgaaagacccaaggatgacagtactaaacccaagccagaagaagaagtgaaacctgtgaaaganccagacatcaancctcaaaaggacactgggtgctacatctccaagggcgagcttgtcatcgtcatcaccggatccagagcccatgggtatgtat

SEQ ID NO: 63 - DNA, 577 bp, Homo sapiens
  misc_feature 2, 207, 387, 522, 540, 562: n = A,T,C or G
cngaactgttcactgatttgagctgtaatccagaaatgatgaagaatgcagcagattcatatttctcacttttacaaggtttcataaattctttggatgaatctacccaagaaagcaagttacgatatattcaaaatttcaagtggactgatacattgcaaggacaggttccaagtgcccagcaggatgctgtttttgaattaattnccatgggatttaatgtagctttatggtataccaaatatgcttcaagactggctggaaaagaaaatataacagaaggtgaagcaaaagaagttcatcgaagcctaaagattgcagctgggatttttaaacatttaaaggaaagtcatctcccaaaactcattacacctgcggaaaaaggangagatttagagtcacgactcatagaagcatacgttattcaatgtcaggctgaagctcaagaagtaacaattgctcgagcaattgaactaaaacatgctcctggactaattgctgcactggcgtatgaaacagccnatttctatcaaaaagccnatcatacttttatccagtttgnagccctgcatattct

SEQ ID NO: 64 - DNA, 693 bp, Homo sapiens
  misc_feature 4, 30, 35, 43, 44, 46, 65, 80, 111, 149, 152, 435, 502,685: n = A,T,C or G
agangttcatcgaagcctaaagattgcagntgggntttttaanncntttaaaggaaagtcatctnccaaaactcatttcncctgcgggaaaaagggggagatttagagtcncgactcatagaaagcatacgttattcaatgtcaggctnaangctcaggaagtaacaattgctcgagcaattgaactaaaacatgctcctggactaattgctgcactggcgtatgaaacagccgatttctatcaaaaagctgatcatactttatccagtttggagcctgcatattctgccaaatggagaaaatatcttcacttgaagatgtgtttctacacagcttatgcttactgttaccatggtgagactttattggctagtgataaatgcggtgaagcaatcaggtctctccaagaagcagaaaaattgtatgcaaaggcanaagcactgtgtaaagaatatggagaaaccaaaggacctggaccaacagtcaaaccttcaggacatcngttctttaggaaacttggaaaccttgtgaagaacaccctagaaaaatgtcagagagaaaatggatttatttactttcaaaaaattccaacagaagccccacagctggaactcaaagcaaattatggtctcgtagagcctatacctttcgaatttcctcctacaagtgttcagtggacaccagnggcattgg

SEQ ID NO: 65 - DNA, 437 bp, Homo sapiens
  misc_feature 198, 274, 290, 312, 365, 379, 380, 398, 405, 434: n = A,T,C or G
ttgagagggctagggataggcttaccttcaagctcgcccttatggacccattggtttcataggaacccattaaaagccacagctcctgtgtcttttaattactatggtgtagtcactggcccttctgcttcaaaaatatgcaatgacttgaggtcatccagggcacgactccttgaactgttcactgatttgagctgnaatccagaaatgatgaagaatgcagcagattcatatttctcacttttacaaggtttcataaattctttggatgaanctacccaagaaagcangttacgatatattcaaaatttnaagtggactgatacattgcaaggacaggttccaagtgcccagcaggatgctgnttttgaattaattnncatgggatttaatgtagntttatgntataccaaatatgcttcaagactggttgnaaa

SEQ ID NO: 66 - DNA, 426 bp, Homo sapiens
  misc_feature 31, 55, 57, 58, 61, 89, 107, 134, 143, 153, 155, 158,160, 166, 168, 180, 185, 193, 203, 205, 235, 247, 254, 266, 268,290, 293, 311, 320, 361, 365, 373, 374, 388, 400: n = A,T,C or G
catcacttttacaaggtttcataaattcttnggatgaatctacccaagaaagcangnnacnacatattcaaaattccaagaggactganacattgcaaggacaggcnccaagtgcccagcaggatgctgttttngaattaatntccatgggancnaangnagctcnanggtataccaaanatgcntcaagacnggctggaaangnaaatataacagaagatgaagcaaaagaagntcatcgaagccnaaagatngcagctgggatntntaaacatttaaaggaaagacangtnccaaaacccactacaccngcggaaaanggaagagatttagagcacgactcatagaagcatacgttatncaangtcaggcnnaagctcaagaagtnacaaattgctcnagcaactgaactaaaacatgctcctg

SEQ ID NO: 67 - DNA, 453 bp, Homo sapiens
  misc_feature 12, 32, 33, 57, 144, 156, 188, 245, 308, 317, 366, 409,417, 418, 445: n = A,T,C or G
aaactcattacncctgcggaaaaaggaagagnnttagagtcacgactcatagaagcntacgttattcaatgtcaggctgaagctcaagaagtagcaattgctcgagcaattgaactaaaacatgctcctggactaattgctgcnctggcgtataanacatgccaatttctatcaaaaagctggtcatnctttatccagtttggagcctgcatattctgtcaaatggagaaaatatcttcacttgnagatgtgtttctacacagcttatgcttactgttaccatggtgaggctttattggctagtgatnaatgcggtnaagcaatcaggtctctccaagaagcagaaaaattgtatgcaaaggcagnagcactgtgtaaagaatatggagaaaccaaaggacctggaccnacagtcannccttcagggcatctgttctttaggaancgtggttg

SEQ ID NO: 68 - DNA, 560 bp, Homo sapiens
  misc_feature 7, 28, 29, 46, 69, 175, 199, 329, 370, 384, 434, 446,457, 469, 476, 482, 502, 522, 540, 545: n = A,T,C or G
tatcccnactctctactgtttctccatnnccgtttttttgggctanttttaattttttttaactttaanaaggagatatacatacccatgggctctggatccggtgatgacgatgacaagctcgcccttattggtttcataggaacccattaaaagccacagctcctgtgtcttntaattactatggtgtagccactgncccttctgcttcaaaaatatgcaatgacttgaggtcatccagggcacgactccttgaactgttcactgatttgagctgtaatccagaaatgatgaagaatgcagcagattcatatttctcacttttacanggtttcataaattctttggatgaatctacccaagaaagcangttacgatatattnaaaatttcaagtggactgatacattgcaaggacaggttccaagtgcccancaggatgctgtntttgaattaanttccatgggatntaatgtngctttntggtataccaaatatgcttnaagactggctggaaaagaanatataacagaagatgaancaatngaagtctattgtacc

SEQ ID NO: 69 - DNA, 516 bp, Homo sapiens
  misc_feature 7, 8, 32, 57, 130, 159, 188, 199, 234, 249, 254, 380,445, 457, 462, 476, 492: n = A,T,C or G
gactccnngaactgttcactgatttgagctgnaatccagaaatgatgaagaatgcancagattcatatttctcacttttacaaggtttcataaattctttggatgaatctacccaagaaagcaagttacnatatattcaaaatttcaagtggactgatncattgcaaggacaggttccaagtgcccancaggatgctgnttttgaattaattcccatgggatttaatgtagctntatggtataccaaanatgcntcaagactggctggaaaagaaaatataacagaagatgaagcaaaagaagttcatcgaagcctaaagattgcagctgggatttttaaacatttaaaggaaagtcatctcccaaaactcattacaccngcggaaaaaggaagagatttagagtcacgactcatagaagcatacgttattcaatgtcaggctgnagctcaagaagnaacanttgctcgagcaatntgaactaaaacatgcncctggactaactgctgcactggcg

SEQ ID NO: 70 - DNA, 529 bp, Homo sapiens
  misc_feature 4, 18, 21, 26, 29, 40, 55, 108, 210, 230, 404, 436, 458,485, 511, 522, 524, 529: n = A,T,C or G
aggntcgtcgaagcctanagnttgcngcngggatttttanaccttttaaaggaangtcatctcccaaaactcattacacctgcggaaaaaggaagagatttagagtcncaactcatagaagcatacgttattcaatgtcaggctgaagctcaagaagtagcaattgctcgagcaattgaactaaaacatgctcctggactaattgctgcnctggcgtatgaaacaggccnatttctaccaaaaagctgatcatactttatccagtttggagcctgcatattctgccgaatggagagaatatcttcgcttgaagatgtgtttctacacagcttatgcttactgttgccatggtgagactttattggctagtgataaatgcggtgaagcaatcaggtctctccaanaagcagaaaaattgtatgcaaaggcagaagcnctgtgtaaagaatatggagaanccaaaggacctggaccagcagtcaganccttcaggacatctgttctttagganacttggaaacngngtgan

SEQ ID NO: 71 - DNA, 514 bp, Homo sapiens
  misc_feature 18, 53, 58, 199, 247, 252, 291, 327, 347, 425: n = A,T,C or G
aacatgctcctgggctanttgcttgcactggcgtatgaaacagccaatttctnccaanaagctgatcatactttatccagtttggagcctgcatattctgccaaatggagaaaatatcttcacttgaagatgtgtttctacacagcttatgcttactgttaccatggtgagactttattggctagtgataaatgcggtnaagcaatcaggtctctccaagaagcagaaaaattgtatgcaaaggcanaagcnctgtgtaaagaatatggagaaaccaaaggacctggaccnacagtcaaaccttcaggacatctgttctttaggaancttggaaaccttgtgaagancaccctagaaaaatgtcagagagaaaatggatttatactaaacccaaaccagaagaagaagtgaaacctgtgaaaganccagacatcaaacctcaaaaggacactgggtgctacatctccaagggcgagcttgaaggtaagcctatccctaaccctctcctcggtcg

SEQ ID NO: 72 - protein, 379 aa, Homo sapiens
MTHWFHRNPLKATAPVSFNYYGVVTGPSASKICNDLRSSRARLLELFTDLSCNPEMMKNAADSYFSLLQGFINSLDESTQESKLRYIQNFKWTDTLQGQVPSITEDEAKEVHRSLKIAAGIFKHLKESHLPKLITPAEKGRDLESRLIEAYVIQCQAEAQEVTIARAIELKHAPGLIAALAYETANFYQKADHTLSSLEPAYSAKWRKYLHLKMCFYTAYAYCYHGETLLASDKCGEAIRSLQEAEKLYAKAEALCKEYGETKGPGPTVKPSGHLFFRKLGNLVKNTLEKCQRENGFIYFQKIPTEAPQLELKANYGLVEPIPFEFPPTSVQWTPETLAAFDLTKRPKDDSTKPKPEEEVKPVKEPDIKPQKDTGCYIS

SEQ ID NO: 73 - protein, 379 aa, Homo sapiens; identical to SEQ ID NO: 72

SEQ ID NO: 74 - protein, 411 aa, Homo sapiens
  VARIANT 346: Xaa = Any Amino Acid
MTHWFHRNPLKATAPVSFNYYGVVTGPSASKICNDLRSSRARLLELFTDLSCNPEMMKNAADSYFSLLQGFINSLDESTQESKLRYIQNFKWTDTLQGQVPSAQQDAVFELISMGFNVALWYTKYASRLAGKENITEDEAKEVHRSLKIAAGIFKHLKESHLPKLITPAEKGRDLESRLIEAYVIQCQAEAQEVTIARAIELKHAPGLIAALAYETANFYQKADHTLSSLEPAYSAKWRKYLHLKMCFYTAYAYCYHGETLLASDKCGEAIRSLQEAEKLYAKAEALCKEYGETKGPGPTVKPSGHLFFRKLGNLVKNTLEKCQRENGFIYFQKIPTEAPQLELKXNYGLVEPIPFEFPPTSVQWTPETLAAFDLTKRPKDDSTKPKPEEEVKPVKEPDIKPQKDTGCYIS

SEQ ID NO: 75 - DNA, 1748 bp, Homo sapiens
  misc_feature 24, 29, 30, 95, 103, 1605, 1614, 1615, 1646, 1678, 1685,1697, 1716, 1745, 1746, 1747, 1748: n = A,T,C or G
ttttaaaacccttttgccctcgcngccannacttcggcacgaggccgatatattgcccttcttcccttagaagaactgctgaaccgactctgttnatttgttntccaaccaaaatctagtactttgctattcaaaagtgtctggggaccagcagcatcagcatctcaggtcctgccccaggctattgaattagagcctgcattggacaaaaccacccacccccagaaaacatctggagaaaatgacccattggtttcataggaacccattaaaagccacagctcctgtgtcttttaattactatggtgtagtcactggcccttctgcttcaaaaatatgcaatgacttgaggtcatccagggcacgactccttgaactgttcactgatttgagctgtaatccagaaatgatgaagaatgcagcagattcatatttctcacttttacaaggtttcataaattctttggatgaatctacccaagaaagcaagttacgatatattcaaaatttcaagtggactgatacattgcaaggacaggttccaagtgcccagcaggatgctgtttttgaattaatttccatgggatttaatgtagctttatggtataccaaatatgcttcaagactggctggaaaagaaaatataacagaagatgaagcaaaagaagttcatcgaagcctaaagattgcagctgggatttttaaacatttaaaggaaagtcatctcccaaaactcattacacctgcggaaaaaggaagagatttagagtcacgactcatagaagcatacgttattcaatgtcaggctgaagctcaagaagtaacaattgctcgagcaattgaactaaaacatgctcctggactaattgctgcactggcgtatgaaacagccaatttctatcaaaaagctgatcatactttatccagtttggagcctgcatattctgccaaatggagaaaatatcttcacttgaagatgtgtttctacacagcttatgcttactgttaccatggtgagactttattggctagtgataaatgcggtgaagcaatcaggtctctccaagaagcagaaaaattgtatgcaaaggcagaagcactgtgtaaagaatatggagaaaccaaaggacctggaccaacagtcaaaccttcaggacatctgttctttaggaaacttggaaaccttgtgaagaacaccctagaaaaatgtcagagagaaaatggatttatttactttcaaaaaattccaacagaagccycacagctggaactcaaagcaaattatggtctcgtagagcctatacctttcgaatttcctcctacaagtgttcagtggacaccagarrcattggctgcatttgatctcaccraaagacccaaggatgacagtactaaacccaarccagaagaagaagtgaaacctgtgaaagaaccagacatcaaacctcaaaaggacactgggtgctacatctcctaaaatacaacttgcacttagaatttctctagcagtaaataagataaaccacagaatttcagttcttatttctcaaaatgatttctctgaagcttgtagaataactattattcagagggttatctgccttnaacttacdnnttcttaatttttaatacagcggagatgtttnttgaaaaaaaaaaaaaaaaaaattgcggccgnavgcttnaaagggttttangagtcgtattagaagcccnatttctgcagatatccatcacactggcgnnnn

SEQ ID NO: 76 - DNA, 1648 bp, Homo sapiens
  misc_feature 24, 29, 30, 95, 103, 1181, 1509, 1518, 1519, 1550, 1582,1589, 1601, 1620: n = A,T,C or G
ttttaaaacccttttgccctcgcngccannacttcggcacgaggccgatatattgcccttcttcccttagaagaactgctgaaccgactctgttnatttgttntccaaccaaaatctagtactttgctattcaaaagtgtctggggaccagcagcatcagcatctcaggtcctgccccaggctattgaattagagcctgcattggacaaaaccacccacccccagaaaacatctggagaaaatgacccattggtttcataggaacccattaaaagccacagctcctgtgtcttttaattactatggtgtagtcactggcccttctgcttcaaaaatatgcaatgacttgaggtcatccagggcacgactccttgaactgttcactgatttgagctgtaatccagaaatgatgaagaatgcagcagattcatatttctcacttttacaaggtttcataaattctttggatgaatctacccaagaaagcaagttacgatatattcaaaatttcaagtggactgatacattgcaaggacaggttccaagtataacagaagatgaagcaaaagaagttcatcgaagcctaaagattgcagctgggatttttaaacatttaaaggaaagtcatctcccaaaactcattacacctgcggaaaaaggaagagatttagagtcacgactcatagaagcatacgttattcaatgtcaggctgaagctcaagaagtaacaattgctcgagcaattgaactaaaacatgctcctggactaattgctgcactggcgtatgaaacagccaatttctatcaaaaagctgatcatactttatccagtttggagcctgcatattctgccaaatggagaaaatatcttcacttgaagatgtgtttctacacagcttatgcttactgttaccatggtgagactttattggctagtgataaatgcggtgaagcaatcaggtctctccaagaagcagaaaaattgtatgcaaaggcagaagcactgtgtaaagaatatggagaaaccaaaggacctggaccaacagtcaaaccttcaggacatctgttctttaggaaacttggaaaccttgtgaagaacaccctagaaaaatgtcagagagaaaatggatttatttactttcaaaaaattccaacagaagccccacagctggaactcaaancaaattatggtctcgtagagcctatacctttcgaatttcctcctacaagtgttcagtggacaccagaaacattggctgcatttgatctcaccaaaagacccaaggatgacagtactaaacccaaaccagaagaagaagtgaaacctgtgaaagaaccagacatcaaacctcaaaaggacactgggtgctacatctcctaaaatacaacttgcacttagaatttctctagcagtaaataagataaaccacagaatttcagttcttatttctcaaaatgatttctctgaagcttgtagaataactattattcagagggttatctgccttnaacttacdnnttcttaatttttaatacagcggagatgtttnttgaaaaaaaaaaaaaaaaaaattgcggccgnavgcttnaaagggttttangagtcgtattagaagcccnatttctgcagatatccatcacactggcg

SEQ ID NO: 77 - DNA, 750 bp, Homo sapiens
  misc_feature 19, 29, 44, 65, 136, 137, 145, 153, 163, 164, 185, 192,193, 496, 497, 498, 499, 500, 501, 502, 503, 504, 505, 506, 507,508, 509, 510, 511, 683, 734: n = A,T,C or G
atgcttccggctggtatgntgtgtggaantgtgagggataccantttcacccaggaaccagctangaccatgattacgccaagctatttagsygacactatwkwayactcaagctmtgcatcaagcttggtaccgnnctcggatncactagtnacggccgccnntgtgctggaattcggcttttnaggtatnncaaatatgcttcaagactggctggaaaagaaaatataacagaavatgaagcaaaagaagttcatcgaagcctaaagattgcagctgggatttttaaacatttaaaggaaagtcatctcccaaaactcattacacctgcggaaaaaggaagagatttagagtcacgactcatagaagcatacgttattcaatgtcaggctgaagctcaagaagtaacaattgctcgagcaattgaactaaaacatgctcctggactaattgctgcactggcgtatgaaacagccaatttctatcaaaaagctgnnnnnnnnnnnnnnnntttggagcctgcatattctgccaaatggagaaaatatcttcacttgaagatgtgtttctacacagcttatgcttactgttaccatggtgagactttattggctagtgataaatgcggtgaagcaatcaggtctctccaagaagcagaaaaattgtatgcaaaggcagaagcnctgtgtaaagaatatggagaaaccaaaggacctggaccaacagtcaagccnaattctgcagatatcc

SEQ ID NO: 78 - DNA, 1744 bp, Homo sapiens
  misc_feature 24, 29, 30, 95, 103, 1605, 1614, 1615, 1646, 1678, 1685,1697, 1716: n = A,T,C or G
ttttaaaacccttttgccctcgcngccannacttcggcacgaggccgatatattgcccttcttcccttagaagaactgctgaaccgactctgttnatttgttntccaaccaawatcymgwmyytykmywktywmwmswktcysgkkwyywksrgcwwcwkywwmtywkktyywrcyyyargmwrkwgawwtasakmcysmttgggacdgvahcvgvtavbchcavhdacaagctcgcccttatgacccattggtttcataggaacccattaaaagccacagctcctgtgtcttttaattactatggtgtagtcactggcccttctgcttcaaaaatatgcaatgacttgaggtcatccagggcacgactccttgaactgttcactgatttgagctgtaatccagaaatgatgaagaatgcagcagattcatatttctcacttttacaaggtttcataaattctttggatgaatctacccaagaaagcaagttacgatatattcaaaatttcaagtggactgatacattgcaaggacaggttccaagtgcccagcaggatgctgtttttgaattaatttccatgggatttaatgtagctttatggtataccaaatatgcttcaagactggctggaaaagaaaatataacagaagatgaagcaaaagaagttcatcgaagcctaaagattgcagctgggatttttaaacatttaaaggaaagtcatctcccaaaactcattacacctgcggaaaaaggaagagatttagagtcacgactcatagaagcatacgttattcaatgtcaggctgaagctcaagaagtaacaattgctcgagcaattgaactaaaacatgctcctggactaattgctgcactggcgtatgaaacagccaatttctaycaaaaagctgatcatactttatccagtttggagcctgcatattctgccaaatggagaaaatatcttcacttgaagatgtgtttctacacagcttatgcttactgttaccatggtgagactttattggctagtgataaatgcggtgaagcaatcaggtctctccaagaagcagaaaaattgtatgcaaaggcagaagcactgtgtaaagaatatggagaaaccaaaggacctggaccaacagtcaaaccttcaggacatctgttctttaggaaacttggaaaccttgtgaagaacaccctagaaaaatgtcagagagaaaatggatttatwywmwwycmaaammwkmmramrrarksmmmmcwgykrrarmtcmaracawmwwayskymwmrkrrmrcykrkwsctwymkmwyywmsksckasmwkkrwkswrwgsmyaycmswaacmytskcykcrktygatctcaccaaaagacccaaggatgacagtactaaacccaaaccagaagaagaagtgaaacctgtgaaagaaccagacatcaaacctcaaaaggacactgggtgctacatctcctaaaatacaacttgcacttagaatttctctagcagtaaataagataaaccacagaatttcagttcttatttctcaaaatgatttctctgaagcttgtagaataactattattcagagggttatctgccttnaacttacdnnttcttaatttttaatacagcggagatgtttnttgaaaaaaaaaaaaaaaaaaattgcggccgnavgcttnaaagggttttangagtcgtattagaagcccnatttctgcagatatccatcacactggcg

SEQ ID NO: 79 - DNA, 1744 bp, Homo sapiens
  misc_feature 24, 29, 30, 95, 103, 353, 374, 383, 411, 426, 439, 442,449, 476, 497, 507, 530, 549, 558, 1605, 1614, 1615, 1646, 1678,1685, 1697, 1716: n = A,T,C or G
ttttaaaacccttttgccctcgcngccannacttcggcacgaggccgatatattgcccttcttcccttagaagaactgctgaaccgactctgttnatttgttntccaaccaaaatctagtactttgctattcaaaagtgtctggggaccagcagcatcagcatctcaggtcctgccccaggctattgaattagagcctgcattggacaaaaccacccacssmyakmwrmmrwmtksrgmwwatgacccattggtttcataggaacccattaaaagccacagctyctgtgtcttttaabtactatggtgtagtcactggcccttctgcttcaaaaatatgcaatgacttgaggncatccagggcacgactccttnaactgttcnctgmttttaattactatgghghagtcantggccvttctgcttnaaaaatatgcaanganttgaggncatccagggcacgactccttgaactgntcactgatttgagctgtaatncagaaatgangaagaatgcagcagattcatatntctcacttttacaaggttncataaattntttggatgaatctacccaagaaagcaagttacgatatattcaaadtttcaagtggactgatacattgcaaggacaggttccaagtataacagaagatgaagcaaaagaagttcatcgaagcctaaagattgcagctgggatttttaaacatttaaaggaaagtcatctcccaaaactcattacacctgcggaaaaaggaagagatttagagtcacgactcatagaagcatacgttattcaatgtcaggctgaagctcaagaagtaacaattgctcgagcaattgaactaaaacatgctcctggactaattgctgcactggcgtatgaaacagccaatttctatcaaaaagctgatcatactttatccagtttggagcctgcatattctgccaaatggagaaaatatcttcacttgaagatgtgtttctacacagcttatgcttactgttaccatggtgagactttattggctagtgataaatgcggtgaagcaatcaggtctctccaagaagcagaaaaattgtatgcaaaggcagaagcactgtgtaaagaatatggagaagccaaaggacctggaccaacagtcaaaccttcaggacatctgttctttaggaaacttggaaaccttgtgaagaacaccctagaaaaatgtcagagagaaaatggatttatttactttcaaaaaattccaacagaagccccacagctggaactcaaagcaaattatggtctcgtagagcctatacctttcgaatttcctcctacaagtgttcagtggacaccagaaacattggctgcatttgatctcaccaaaagacccaaggatgacagtactaaacccaaaccagaagaagaagtgaaacctgtgaaagaaccagacatcaaacctcaaaaggacactgggtgctacatctcctaaaatacaacttgcacttagaatttctctagcagtaaataagataaaccacagaatttcagttcttatttctcaaaatgatttctctgaagcttgtagaataactattattcagagggttatctgccttnaacttacdnnttcttaatttttaatacagcggagatgtttnttgaaaaaaaaaaaaaaaaaaattgcggccgnavgcttnaaagggttttangagtcgtattagaagcccnatttctgcagatatccatcacactggcg

SEQ ID NO: 80 - DNA, 30 bp, Artificial Sequence
  PCR primer
tttatggtataccaaatatgcttcaagact

SEQ ID NO: 81 - DNA, 30 bp, Artificial Sequence
  PCR primer
cagccagtcttgaagcatatttggtatacc

SEQ ID NO: 82 - DNA, 30 bp, Artificial Sequence
  PCR primer
cttgaagatgtgtttctacacagcttatgc

SEQ ID NO: 83 - DNA, 30 bp, Artificial Sequence
  PCR primer
gactgttggtccaggtcctttggtttctcc

SEQ ID NO: 84 - DNA, 28 bp, Artificial Sequence
  PCR primer
caggaaacagctatgaccatgattacgc

SEQ ID NO: 85 - DNA, 29 bp, Artificial Sequence
  PCR primer
gtaatacgactcactatagggcgaattgg

SEQ ID NO: 86 - DNA, 33 bp, Artificial Sequence
  PCR primer
aatcccatggaaattaattcaaaaacagcatcc

SEQ ID NO: 87 - DNA, 31 bp, Artificial Sequence
  PCR primer
ggtgagactttattggctagtgataaatgcg

SEQ ID NO: 88 - DNA, 31 bp, Artificial Sequence
  PCR primer
gtgcttctgcctttgcatacaatttttctgc

SEQ ID NO: 89 - DNA, 32 bp, Artificial Sequence
  PCR primer
tattcaaaatttcaagtggactgatacattgc

SEQ ID NO: 90 - DNA, 30 bp, Artificial Sequence
  PCR primer
ggatgaatctacccaagaaagcaagttacg

SEQ ID NO: 91 - DNA, 31 bp, Artificial Sequence
  PCR primer
aacttgctttcttgggtagattcatccaaag

SEQ ID NO: 92 - DNA, 32 bp, Artificial Sequence
  PCR primer
gtatcagtccacttgaaattttgaatatatcg

SEQ ID NO: 93 - DNA, 33 bp, Artificial Sequence
  PCR primer
cagaagatgaaggaaaagaagttcatcgaagcc

SEQ ID NO: 94 - DNA, 29 bp, Artificial Sequence
  PCR primer
ggattggtggcgacgactcctggagcccg

SEQ ID NO: 95 - DNA, 30 bp, Artificial Sequence
  PCR primer
agaccaactggtaatggtagcgaccggcgc

SEQ ID NO: 96 - DNA, 32 bp, Artificial Sequence
  PCR primer
gcaaaggcagaagcactgtgtaaagaatatgg

SEQ ID NO: 97 - DNA, 30 bp, Artificial Sequence
  PCR primer
ctgttctttaggaaacttggaaaccttgtg

SEQ ID NO: 98 - DNA, 31 bp, Artificial Sequence
  PCR primer
ctggaaaatttagtgttattggaatcaaagg

SEQ ID NO: 99 - DNA, 33 bp, Artificial Sequence
  PCR primer
tgaacagttcaaggagtcgtgccctggatgacc

SEQ ID NO: 100 - DNA, 30 bp, Artificial Sequence
  PCR primer
ccagggcacgactccttgaactgttcactg

SEQ ID NO: 101 - DNA, 32 bp, Artificial Sequence
  PCR primer
atcccttgagtcttattgattagtactaactc

SEQ ID NO: 102 - DNA, 33 bp, Artificial Sequence
  PCR primer
cgaatttaatacgactcactatagggaatttgg

SEQ ID NO: 103 - DNA, 30 bp, Artificial Sequence
  PCR primer
tgccaagctaaaattaaccctcactaaagg

SEQ ID NO: 104 - DNA, 29 bp, Artificial Sequence
  PCR primer
tatcaaaaagctgatcatactttatccag

SEQ ID NO: 105 - DNA, 40 bp, Artificial Sequence
  PCR primer
ttaggagatgtagcacccagtgtccttttgaggtttgatg

SEQ ID NO: 106 - DNA, 37 bp, Artificial Sequence
  PCR primer
ggagatgtagcacccagtgtccttttgaggtttgatg

SEQ ID NO: 107 - DNA, 35 bp, Artificial Sequence
  PCR primer
atgacccattggtttcataggaacccattaaaagc

SEQ ID NO: 108 - DNA, 47 bp, Artificial Sequence
  PCR primer
cagcaggatgctgtttttgaattaatttccatgggatttaatgtagc

SEQ ID NO: 109 - DNA, 27 bp, Artificial Sequence
  PCR primer
aaggacaggttccaagtataacagaag

SEQ ID NO: 110 - DNA, 31 bp, Artificial Sequence
  PCR primer
cacttgtaggaggaaattcgaaaggtatagg

SEQ ID NO: 111 - DNA, 31 bp, Artificial Sequence
  PCR primer
ttctggtttgggtttagtataaatccatttt

SEQ ID NO: 112 - DNA, 17 bp, Artificial Sequence
  PCR primer
gctggtaaatgtcctct

SEQ ID NO: 113 - DNA, 21 bp, Artificial Sequence
  PCR primer
tctggataatcacaaggtttc

SEQ ID NO: 114 - DNA, 1744 bp, Homo sapiens
  misc_feature 24, 29, 30, 95, 103, 1605, 1614, 1615, 1646, 1678, 1685,1697, 1716: n = A,T,C or G
ttttaaaacccttttgccctcgcngccannacttcggcacgaggccgatatattgcccttcttcccttagaagaactgctgaaccgactctgttnatttgttntccaaccaaaatctagtactttgctattcaaaagtgtctggggaccagcagcatcagcatctcaggtcctgccccaggctattgaattagagcctgcattggacaaaaccacccacccccagaaaacatctggagaaaatgacccattggtttcataggaacccattaaaagccacagctcctgtgtcttttaattactatggtgtagtcactggcccttctgcttcaaaaatatgcaatgacttgaggtcatccagggcacgactccttgaactgttcactgatttgagctgtaatccagaaatgatgaagaatgcagcagattcatatttctcacttttacaaggtttcataaattctttggatgaatctacccaagaaagcaagttacgatatattcaaaatttcaagtggactgatacattgcaaggacaggttccaagtgcccagcaggatgctgtttttgaattaatttccatgggatttaatgtagctttatggtataccaaatatgcttcaagactggctggaaaagaaaatataacagaagatgaagcaaaagaagttcatcgaagcctaaagattgcagctgggatttttaaacatttaaaggaaagtcatctcccaaaactcattacacctgcggaaaaaggaagagatttagagtcacgactcatagaagcatacgttattcaatgtcaggctgaagctcaagaagtaacaattgctcgagcaattgaactaaaacatgctcctggactaattgctgcactggcgtatgaaacagccaatttctatcaaaaagctgatcatactttatccagtttggagcctgcatattctgccaaatggagaaaatatcttcacttgaagatgtgtttctacacagcttatgcttactgttaccatggtgagactttattggctagtgataaatgcggtgaagcaatcaggtctctccaagaagcagaaaaattgtatgcaaaggcagaagcactgtgtaaagaatatggagaaaccaaaggacctggaccaacagtcaaaccttcaggacatctgttctttaggaaacttggaaaccttgtgaagaacaccctagaaaaatgtcagagagaaaatggatttatttactttcaaaaaattccaacagaagccccacagctggaactcaaaacaaattatggtctcgtagagcctatacctttcgaatttcctcctacaagtgttcagtggacaccagaaacattggctgcatttgatctcaccaaaagacccaaggatgacagtactaaacccaaaccagaagaagaagtgaaacctgtgaaagaaccagacatcaaacctcaaaaggacactgggtgctacatctcctaaaatacaacttgcacttagaatttctctagcagtaaataagataaaccacagaatttcagttcttatttctcaaaatgatttctctgaagcttgtagaataactattattcagagggttatctgccttnaacttacdnnttcttaatttttaatacagcggagatgtttnttgaaaaaaaaaaaaaaaaaaattgcggccgnavgcttnaaagggttttangagtcgtattagaagcccnatttctgcagatatccatcacactggcg

SEQ ID NO: 115 - DNA, 443 bp, Homo sapiens
  misc_feature 66, 139, 435, 443: n = A,T,C or G
atgacccattggtttcataggaacccattaaaagccacagctcctgtgtcttttaattactatggngtagtcactggcccttctgcttcaaaaatatgcaatgacttgaggtcatccagggcacgactccttgaactgntcactgatttgagctgtaatccagaaatgatgaagaatgcagcagattcatatttctcacttttacaaggtttcataaattctttggatgaatctacccaagaaagcaagttacgatatattcaaaatttcaagtggactgatacattgcaaggacaggttccaagtgcccagcaggatgctgtttttgaattaatttccatgggatttaatgtagctttatggtataccaaatatgcttcaagactggctggaaaagaaaatataacagaagatgaagcaaaagaagttcatcgnagcctaan

SEQ ID NO: 116 - DNA, 577 bp, Homo sapiens; identical to SEQ ID NO: 63
  misc_feature 2, 207, 387, 522, 540, 562: n = A,T,C or G

SEQ ID NO: 117 - DNA, 472 bp, Homo sapiens; identical to SEQ ID NO: 60
  misc_feature 3, 5, 12, 42, 84, 109, 161, 198, 289, 412, 414: n = A,T,C or G

SEQ ID NO: 118 - DNA, 689 bp, Homo sapiens
  misc_feature 4, 30, 35, 43, 45, 64, 79, 109, 146, 149, 431, 498, 681: n = A,T,C or G
agangttcatcgaagcctaaagattgcagntgggntttttaancntttaaaggaaagtcatctnccaaaactcatttcncctgcggaaaaagggggagatttagagtcncgactcatagaagcatacgttattcaatgtcaggctnaangctcagaagtaacaattgctcgagcaattgaactaaaacatgctcctggactaattgctgcactggcgtatgaaacagccgatttctatcaaaaagctgatcatactttatccagtttggagcctgcatattctgccaaatggagaaaatatcttcacttgaagatgtgtttctacacagcttatgcttactgttaccatggtgagactttattggctagtgataaatgcggtgaagcaatcaggtctctccaagaagcagaaaaattgtatgcaaaggcanaagcactgtgtaaagaatatggagaaaccaaaggacctggaccaacagtcaaaccttcaggacatcngttctttaggaaacttggaaaccttgtgaagaacaccctagaaaaatgtcagagagaaaatggatttatttactttcaaaaaattccaacagaagccccacagctggaactcaaagcaaattatggtctcgtagagcctatacctttcgaatttcctcctacaagtgttcagtggacaccagnggcattgg

SEQ ID NO: 119 - DNA, 471 bp, Homo sapiens
  misc_feature 4, 18, 61, 94, 118, 137, 153, 171, 180, 238, 275, 315,335, 368, 369, 380, 443, 456: n = A,T,C or G
atgnaacagccaatttcnatcaaaaagctgatcatactttatccagtttggagcctgcatnttctgccaaatggagtaaatatcttcacttgangatgtgtttctacgcagcttatgnttactgttaccatggtgangctttattggctagtnataaatgcggtgaagcantcaggtctntccaagaagcagaaaaattgtatgcaaaggcagaagcactgtgtaaagaatatggagntaccaaaggacctggaccaacagtcaaaccttcaggncatctgttctttaggaaacttgggagccttgtgaagaacnccctagagaaatgtcagagngaaaatggatttatttactttcaaaaaattccnncagaagcctcncagctggaactcaaagcaaattatggtctcgtagagcctatacctttcgaatttcctcctacnagtgttcagtggnggtcctgggaaaatg

SEQ ID NO: 120 - DNA, 431 bp, Homo sapiens
  misc_feature 24, 32, 34, 108, 113, 115, 190, 296, 324, 389, 401: n = A,T,C or G
gtggagcaatcaggtctctccaanaatcagananattgtttgcagaggcataagctctgtgtaggggatatggagaagccaaaggacctggaccaacagtcaagcctncaggncntctgttctttaggaaacttggaaaccttgtgaagaacaccctagaaaaatgtcagagagaaaatggatttatttnctttcaaaaaattccaacagaagcctcacagctggaactcaaagcaaattatggtctcgtagagcctatacctttcgaatttcctcctacaagtgttcagtggacnccagaaacattggctgcatttgatctcnccgaaagacccaaggatgacagtactaaacccaagccagaagaagaagtgaaacctgtgaaaganccagacatcaancctcaaaaggacactgggtgctacatctcc

SEQ ID NO: 121 - DNA, 779 bp, Homo sapiens
  misc_feature 2, 3, 6, 15, 68, 76: n = A,T,C or G
annacntcggcacgnggccgatatattgcccttcttcccttagaagaactgctgaaccgactctgttnatttgttntccaaccaaaatctagtactttgctattcaaaagtgtctggggaccagcagcatcagcatctcaggtcctgccccaggctattgaattagagcctgcattggacaaaaccacccacccccagaaaacatctggagaaaatgacccattggtttcataggaacccattaaaagccacagctcctgtgtcttttaattactatggtgtagtcactggcccttctgcttcaaaaatatgcaatgacttgaggtcatccagggcacgactccttgaactgttcactgatttgagctgtaatccagaaatgatgaagaatgcagcagattcatatttctcacttttacaaggtttcataaattctttggatgaatctacccaagaaagcaagttacgatatattcaaaatttcaagtggactgatacattgcaaggacaggttccaagtataacagaagatgaagcaaaagaagttcatcgaagcctaaagattgcagctgggatttttaaacatttaaaggaaagtcatctcccaaaactcattacacctgcggaaaaaggaagagatttagagtcacgactcatagaagcatacgttattcaatgtcaggctgaagctcaagaagtaacaattgctcgagcaattgaactaaaacatgctcctggactaattgctgcactggcgtatgaaacagccaatttctatc

SEQ ID NO: 122 - DNA, 769 bp, Homo sapiens
  misc_feature 73, 99, 110, 111, 126, 131, 134, 206, 241, 254, 277, 282: n = A,T,C or G
acttcggcacgaggccgatatattgcccttcttcccttagaagaactgctgaaccgactctgtttttttgttntccaaccaaaatctagtactttgctnttcaaaagtgnntggggaccagcagcntcagnatntcaggtcctgccccaggctattgaattagagcctgcattggacaaaaccacccacccccagaaaacatctgnagaaaatgacccattggtttcataggaacccattnaaagccacagctnctgtgtcttttaattactatggngtagncactggcccttctgcttcaaaaatatgcaatgacttgaggtcatccagggcacgactccttgaactgttcactgatttgagctgtaatccagaaatgatgaagaatgcagcagattcatatttctcacttttacaaggtttcataaattctttggatgaatctacccaagaaagcaagttacgatatattcaaaatttcaagtggactgatacattgcaaggacaggttccaagtataacagaagatgaagcaaaagaagttcatcgaagcctaaagattgcagctgggatttttaaacatttaaaggaaagtcatctcccaaaactcattacacctgcggaaaaaggaagagatttagagtcacgactcatagaagcatacgttattcaatgtcaggctgaagctcaagaagtaacaattgctcgagcaattgaactaaaacatgctcctgggctaattgctgcactggcgtatgaaacagccaaa

SEQ ID NO: 123 - DNA, 765 bp, Homo sapiens
  misc_feature 24, 29, 30, 88, 93, 95: n = A,T,C or G
ttttaaaacccttttgccctcgcngccannaattcggcacgaggccgatatattgcccttcttcccttagaagaactgctgaaccgtntctgntnatttgttgtccaaccaaaatctagtactttgctattcaaaagtgtctggggaccagcagcatcagcatctcaggtcctgccccaggctattgaattagagcctgcattggacaaaaccacccacccccagaaaacatctggagaaaatgacccattggtttcataggaacccattaaaagccacagctcctgtgtcttttaattactatggtgtagtcactggcccttctgcttcaaaaatatgcaatgacttgaggtcatccagggcacgactccttgaactgttcactgatttgagctgtaatccagaaatgatgaagaatgcagcagattcatatttctcacttttacaaggtttcataaattctttggatgaatctacccaagaaagcaagttacgatatattcaaaatttcaagtggactgatacattgcaaggacaggttccaagtataacagaagatgaagcaaaagaagttcatcgaagcctaaagattgcagctgggatttttaaacatttaaaggaaagtcatcttccaaaactcattacacctgcggaaaaaggaagagatttagagtcacgactcatagaagcatacgttattcaatgtcaggctgaagctcaagaagtaacaattgctcgagcaattgaactaaaacatgctcctgg

SEQ ID NO: 124 - DNA, 294 bp, Homo sapiens
  misc_feature 53, 97, 108, 115, 135, 145, 161, 164, 165, 191, 192,204, 220, 260, 262, 294: n = A,T,C or G
ggacagggattgcagctgggatttttaaacatttaaaggaaagtcatctcccnaaactcattacacctgcggaaaaaggaagagatttagagtcacnactcatagaancatacgntattcaatgtcaggctgaanctcaagaagnaacaattgctcgagcnatnnaactaaaacatgctcctggactaatnnctgcactggcgnatgaaacacccaattnctatcaaaaagctgatcatactttatccagtttggagccngnatattctgccaaaaggagaaaacatcttcacn

SEQ ID NO: 125 - DNA, 434 bp, Homo sapiens
  misc_feature 19, 32, 53, 196, 199: n = A,T,C or G
acaggttccaagtataacngaagatgaagcanaagaggttcgtcaaagcctanagattgcagctgggatttttaaacatttaaaggaaagtcatctcccaaaactcattacacctgcggaaaaaggaagagatttagagtcacgactcatagaagcatacgttattcaatgtcaggctgaagctcaagaagtaacnatngctcgagcaattgaactaaaacatgctcctggactaattgctgcactggcgtatgaaacagccaatttctatcaaaaagctgatcatactttatccagtttggagcctgcatattctgccaaatggagaaaatatcttcacttgaagatgtgtttctacacagcttatgcttactgttaccatggtgagactttattggctagtgataaatgcggtgaagcaatcaggtctctcc

SEQ ID NO: 126 - DNA, 647 bp, Homo sapiens
  misc_feature 239: n = A,T,C or G
aggtttcataaattctttggatgaatctacccaagaaagcaagttacgatatattcaaaatttcaagtggactgatacattgcaaggacaggttccaagtataacagaagatgaaggaaaagaagttcatcgaagcctaaagattgcagctgggatttttaaacatttaaaggaaagtcatctcccaaaactcattacacctgcggaaaaaggaagagatttagagtcacgactcatanaagcatacgttattcaatgtcaggctgaagctcaagaagtaacaattgctcgagcaattgaactaaaacatgctcctggactaattgctgcactggcgtatgaaacagccaatttctatcaaaaagctgatcatactttatccagtttggagcctgcatattctgccaaatggagaaaatatcttcacttgaagatgtgtttctacacagcttatgcttactgttaccatggtgagactttattggctagtgataaatgcggtgaagcaatcaggtctctccaagaagcagaaaaattgtatgcaaaggcagaagcactgtgtaaagaatatggagaaaccaaaggacctggaccaacagtcaaaccttcaggacatctgttctttaggaaacttggaaaccttgtga

SEQ ID NO: 127 - DNA, 486 bp, Homo sapiens
  misc_feature 428, 435, 436, 441, 443, 464, 473: n = A,T,C or G
tataacagaagatgaagcaaaagaagttcatcgaagcctaaagattgcagctgggatttttaaacatttaaaggaaagtcatctcccaaaactcattacacctgcggaaaaaggaagagatttagagtcacgactcatagaagcatacgttattcaatgtcaggctgaagctcaagaagtaacaattgctcgagcaattgaactaaaacatgctcctggactaattgctgcactggcgtatgaaacagccaatttctatcaaaaagctgtttggagcctgcatattctgccaaatggagaaaatatcttcacttgaagatgtgtttctacacagcttatgcttactgttaccatggtgagactttattggctagtgataaatgcggtgaagcaatcaggtctctccaagaagcagaaaaattgtatgnaaaggcnnaagcncngtgtaaagaatatggagaaancaaaggacnaggaccaacagtc

SEQ ID NO: 128 - DNA, 486 bp, Homo sapiens
  misc_feature 307: n = A,T,C or G
tataacagaacatgaagcaaaagaagttcatcgaagcctaaagattgcagctgggatttttaaacatttaaaggaaagtcatctcccaaaactcattacacctgcggaaaaaggaagagatttagagtcacgactcatagaagcatacgttattcaatgtcaggctgaagctcaagaagtaacaattgctcgagcaattgaactaaaacatgctcctggactaattgctgcactggcgtatgaaacagccaatttctatcaaaaagctgtttggagcctgcatattctgccaaatggagaaaatatnttcacttgaagatgtgtttctacacagcttatgcttactgttaccatggtgagactttattggctagtgataaatgcggtgaagcaatcaggtctctccaagaagcagaaaaattgtatgcaaaggcagaagcactgtgtaaagaatatggagaaaccaaaggacctggaccaacagtc

SEQ ID NO: 129 - DNA, 474 bp, Homo sapiens
  misc_feature 1, 429: n = A,T,C or G
natcgaagcctaaagattgcagctgggatttctaaacatttaaaggaaagtcatctcccaaaactcattacacctgcggaaaaaggaagagatttagagtcacgactcatagaagcatacgttattcaatgtcaggctgaagctcaagaagtaacaattgctcgagcaattgaactaaaacatgctcctggactaattgctgcactggcgtatgaaacagccaatttctatcaaaaagctgatcatactttatccagtttggagcctgcatattctgccaaatggagaaaatatcttcacgtgaagatgtgtttctacacagcttatgcttactgttaccatggtgagactttattggctagtgataaatgcggtgaagcaatcaggtctctccaagaagcagaaaaattgtatgcaaaggcagaagcnctgtgtaaagaatatggagaaaccaaaggacctggaccaacagtc

SEQ ID NO: 130 - DNA, 277 bp, Homo sapiens
  misc_feature 5, 64, 91, 111, 113, 134, 192, 197, 206, 208, 217, 238,244, 268: n = A,T,C or G
cttgnagatgtgttggtacacagcttatgcttactggtaccatggtgagactttattggctagngataaatgcggtgaagcaatcaggtcnctccaagaagcagaaaaatngnatgcaaaggcagaagcactgngtaaagaatatggagaaaccaaaggacctggaccaacagccaaaccttcaggacatcngttcnttaggaaantnggaaacctngtgaagaacaccctagaaaancgtcanagagaaaatggatttatttacttncaaaaactc

SEQ ID NO: 131 - DNA, 152 bp, Homo sapiens
  misc_feature 47, 101, 124, 131, 142: n = A,T,C or G
tggtgagactttattggctagtgataaatgcggtgaagcaatcaggnctctccaagaagcagaaaaattgtatgcaaaggcagaagcactgtgtaaagaanatggagaaaccaaaggacctggnccaacanccaaaccttcnggacatctgt

SEQ ID NO: 132 - DNA, 682 bp, Homo sapiens
  misc_feature 13, 30, 34, 52, 68, 72, 75, 77, 116, 171, 196, 348, 433,554, 591, 600, 601, 632, 664, 671: n = A,T,C or G
tggtgggcttttnttggctagggataaatncggngaagcaatcaggtctctncaaggagcagagaatngtangcnanggcagaagcactgtgtaaagaatatggaggaaccaaagnccggggcaacagtcaaaccttcaggacatctgttctttaggaaacttgggaaccntgtgaagaacaccctagaaaaatgncagagagaaaatggatttatttactttcaaaaaattccaacagaagccccacagctggaactcaaagcaaattatggtctcgtagagcctatacctttcgaatttcctcctacaagtgttcagtggacaccagaaacattggctgcatttgntctcaccaaaagacccaaggatgacagtactaaacccaaaccagaagaagaagtgaaacctgtgaaagaaccagacatcaaaccncaaaaggacactgggtgctacatctcctaaaatacaacttgcacttagaatttctctagcagtaaataagataaaccacagaatttcagttcttatttctcaaaatgatttctctgaagcntgtagaataactattattcagagggttatctgccttnaacttacannttcttaatttttaatacagcggagatgtttnttgaaaaaaaaaaaaaaaaaaattgcggccgnaagcttnaaagggtttta

SEQ ID NO: 133 - DNA, 628 bp, Homo sapiens
  misc_feature 2, 35, 53, 69, 78, 87, 90, 117, 128, 135, 147, 150, 209,269, 294, 457, 537, 544, 546, 547, 578, 610, 617: n = A,T,C or G
gnagcaaaaaaattgtttgcaaaggcagaagcacngggtaaagaatatggagnaaccaggggcctgggngacagtcanaccttcagnacntctgttctttaggaaacttggaaaccntgtgaagagcnccctagnaaaatgtcaganagnaaatggatttatttactttcaaaaaattccaacagaagccccacagctggaactcaaancaaattatggtctcgtagagcctatacctttcgaatttcctcctacaagtgttcagtggncaccagaaacattggctgcatttgntctcaccaaaagacccaaggatgacagtactaaacccaaaccagaagaagaagtgaaacctgtgaaagaaccagacatcaaacctcaaaaggacactgggtgctacatctcctaaaatacaacttgcacttagaatttctctagcagtaaataagataaaccncagaatttcagttcttatttctcaaaatgatttctctgaagcttgtagaataactattattcagagggttatctgccttnaacttanannttcttaatttttaatacagcggagatgtttnttgaaaaaaaaaaaaaaaaaaattgcggccgnaagcttnaaagggtttta

SEQ ID NO: 134 - DNA, 245 bp, Homo sapiens
  misc_feature 8, 25, 44, 73, 111: n = A,T,C or G
gacatttnttctttaggaaactttnaaacctttttaagaacacnctagaaaaatgtcagagaggggatggttngatttactttaaaaaaattccaacagaagccccacagntggaactcaaaacaaattatggtctcgtagagcctatacctttcgaatttcctcctacaagtgttcagtggacaccagaaacattggctgcatttgatctcaccaaaagacccaaggatgacagtactaaaccc

SEQ ID NO: 135 - DNA, 504 bp, Homo sapiens
  misc_feature 7, 12, 14, 33, 47, 76, 86, 99, 101, 110, 112, 124, 143,161, 189, 192, 197, 201, 208, 212, 216, 240, 264, 269, 347, 419,427, 429, 490: n = A,T,C or G
gaaaccntgtgnanagcaccctagaaaaatgtnagagaaaaaatggntttatttactttcaaaaaattccaacagnagccccacanctggaactcaaancnaattatggncncgtagagcctanacccttcgaatttcctccnacaagtgttcagtggacnccagaaacattggctgcatttgatctcnccnaaagncccnaggatgncagnactnaacccaaaccagaagaagaagtgnaacctgtgaaagaaccagacatcnaaccncaaaaggacactgggtgctacatctcctaaaatacaacttgcacttagaatttctctagcagtaaataagataaaccncagaatttcagttcttatttctcaaaatgatttctctgaagcttgtagaataactattattcagagggttanaagccttnancttacttgttcttaatttttaatacagcggagatgtttcttgaaaaaaaaaaaaaaaaaanttgcggccgaaggt

SEQ ID NO: 136 - DNA, 505 bp, Homo sapiens
  misc_feature 332, 393, 422, 438, 442, 446, 458, 477: n = A,T,C or G
aaaaattccaacagaagccccacagctggaactcaaagcacattatggtctcgtagagcctatacctttcgaatttcctcctacaagtgttcagtggacaccagaaacattggctgcatttgatctcaccaaaagacccaaggatgacagtactaaacccaaaccagaagaagaagtgaaacctgtgaaagaaccagacatcaaacctcaaaaggacactgggtgctacatctcctaaaatacaacttgcacttagaatttctctagcagtaaataagataaaccacagaatttcagttcttatttctcaaaatgatttctctgaagctcgnagaataactattattcagagggttatctgcactccagcctgggcaacagagggagactccncctcagaaaaaaaaaaaaaaaaaaaaaangtcctctgcgttgttncccnctgnttgccctatagngagtcgtattagaagcccnatttctgcagatatccatcacactggcg

SEQ ID NO: 137 - DNA, 87 bp, Homo sapiens
  misc_feature 17, 22, 41, 78: n = A,T,C or G
tttcctcctacaagcgntcagnggacaccagaaacattggntgcatttgatctcaccaaaagacccaaggatgacagnactaaaccc

SEQ ID NO: 138 - DNA, 734 bp, Homo sapiens
  misc_feature 19, 29, 44, 65, 95, 654, 661, 662, 667, 669, 690, 699,718: n = A,T,C or G
atgcttccggctggtatgntgtgtggaantgtgagggataccantttcacccaggaaccagctangaccatgattacgccaagctatttaggtgncactatagaatactcaagctatgcatcaagcttggtaccgagctcggatccactagtaacggccgccagtgtgctggaattcggctttttaggtataccaaatatgcttcaagactggctggaaaagaaaatataacagaagatgaagcaaaagaagttcatcgaagcctaaagattgcagctgggatttttaaacatttaaaggaaagtcatctcccaaaactcattacacctgcggaaaaaggaagagatttagagtcacgactcatagaagcatacgttattcaatgtcaggctgaagctcaagaagtaacaattgctcgagcaattgaactaaaacatgctcctggactaattgctgcactggcgtatgaaacagccaatttctatcaaaaagctgtttggagcctgcatattctgccaaatggagaaaatatcttcacttgaagatgtgtttctacacagcttatgcttactgttaccatggtgagactttattggctagtgataaatgcggtgaagcaatcaggtctctccaagaagcagaaaaattgtatgnaaaggcnnaagcncngtgtaaagaatatggagaaancaaaggacnaggaccaacagtcaagcanaattctgcagatatcc

SEQ ID NO: 139 - DNA, 646 bp, Homo sapiens
  misc_feature 1, 3, 29, 48, 49, 57, 65, 66, 68, 75, 76, 97, 104, 105,445: n = A,T,C or G
nanccgacactattttacactcaagctcngcatcaagcttggtaccgnnctcggatncactagtnncngccgccnntgtgctggaattcggcttttnaggtatnncaaatatgcttcaagactggctggaaaagaaaatataacagaacatgaagcaaaagaagttcatcgaagcctaaagattgcagctgggatttttaaacatttaaaggaaagtcatctcccaaaactcattacacctgcggaaaaaggaagagatttagagtcacgactcatagaagcatacgttattcaatgtcaggctgaagctcaagaagtaacaattgctcgagcaattgaactaaaacatgctcctggactaattgctgcactggcgtatgaaacagccaatttctatcaaaaagctgtttggagcctgcatattctgccaaatggagaaaatatnttcacttgaagatgtgtttctacacagcttatgcttactgttaccatggtgagactttattggctagtgataaatgcggtgaagcaatcaggtctctccaagaagcagaaaaattgtatgcaaaggcagaagcactgtgtaaagaatatggagaaaccaaaggacctggaccaacagtcaagccgaattctgcagatatcc

SEQ ID NO: 140 - DNA, 606 bp, Homo sapiens
  misc_feature 1, 2, 9, 14, 19, 25, 41, 52, 54, 59, 61, 70, 79, 80, 99,107, 114, 115, 120, 121, 122, 550, 601: n = A,T,C or G
nngtccggncgacnttgcncagggnatatccgcgggaattntgctttttatngnatacnanatatgcttnaagcactgnntggaaaagaaaatataacnggggatgnggcaaanntaagnnnatcgaagcctaaagattgcagctgggatttctaaacatttaaaggaaagtcatctcccaaaactcattacacctgcggaaaaaggaagagatttagagtcacgactcatagaagcatacgttattcaatgtcaggctgaagctcaagaagtaacaattgctcgagcaattgaactaaaacatgctcctggactaattgctgcactggcgtatgaaacagccaatttctatcaaaaagctgatcatactttatccagtttggagcctgcatattctgccaaatggagaaaatatcttcacgtgaagatgtgtttctacacagcttatgcttactgttaccatggtgagactttattggctagtgataaatgcggtgaagcaatcaggtctctccaagaagcagaaaaattgtatgcaaaggcagaagcnctgtgtaaagaatatggagaaaccaaaggacctggaccaacagtcaagccnaattc

SEQ ID NO: 141 - DNA, 1744 bp, Homo sapiens; identical to SEQ ID NO: 114
  misc_feature 24, 29, 30, 95, 103, 1605, 1614, 1615, 1646, 1678, 1685,1697, 1716: n = A,T,C or G

SEQ ID NO: 142 - DNA, 560 bp, Homo sapiens; identical to SEQ ID NO: 68
  misc_feature 7, 28, 29, 46, 69, 175, 199, 329, 370, 384, 434, 446,457, 469, 476, 482, 502, 522, 540, 545: n = A,T,C or G

SEQ ID NO: 143 - DNA, 436 bp, Homo sapiens
  misc_feature 197, 273, 289, 311, 364, 378, 379, 397, 404, 433: n = A,T,C or G
ttgagagggctagggataggcttaccttcaagctcgcccttatgacccattggtttcataggaacccattaaaagccacagctcctgtgtcttttaattactatggtgtagtcactggcccttctgcttcaaaaatatgcaatgacttgaggtcatccagggcacgactccttgaactgttcactgatttgagctgnaatccagaaatgatgaagaatgcagcagattcatatttctcacttttacaaggtttcataaattctttggatgaanctacccaagaaagcangttacgatatattcaaaatttnaagtggactgatacattgcaaggacaggttccaagtgcccagcaggatgctgnttttgaattaattnncatgggatttaatgtagntttatgntataccaaatatgcttcaagactggttgnaaa

SEQ ID NO: 144 - DNA, 515 bp, Homo sapiens
  misc_feature 7, 8, 32, 57, 130, 159, 188, 199, 234, 249, 254, 380,445, 457, 462, 491: n = A,T,C or G
gactccnngaactgttcactgatttgagctgnaatccagaaatgatgaagaatgcancagattcatatttctcacttttacaaggtttcataaattctttggatgaatctacccaagaaagcaagttacnatatattcaaaatttcaagtggactgatncattgcaaggacaggttccaagtgcccancaggatgctgnttttgaattaattcccatgggatttaatgtagctntatggtataccaaanatgcntcaagactggctggaaaagaaaatataacagaagatgaagcaaaagaagttcatcgaagcctaaagattgcagctgggatttttaaacatttaaaggaaagtcatctcccaaaactcattacaccngcggaaaaaggaagagatttagagtcacgactcatagaagcatacgttattcaatgtcaggctgnagctcaagaagnaacanttgctcgagcaattgaactaaaacatgcncctggactaactgctgcactggcg

SEQ ID NO: 145 - DNA, 425 bp, Homo sapiens
  misc_feature 30, 54, 56, 57, 60, 88, 106, 133, 142, 152, 154, 157,159, 165, 167, 179, 184, 192, 202, 204, 234, 246, 253, 265, 267,289, 292, 310, 319, 361, 365, 373, 374, 388, 399: n = A,T,C or G
ctcacttttacaaggtttcataaattcttnggatgaatctacccaagaaagcangnnacnacatattcaaaattccaagaggactganacattgcaaggacaggcnccaagtgcccagcaggatgctgttttngaattaatntccatgggancnaangnagctcnanggtataccaaanatgcntcaagacnggctggaaangnaaatataacagaagatgaagcaaaagaagntcatcgaagccnaaagatngcagctgggatntntaaacatttaaaggaaagacangtnccaaaacccactacaccngcggaaaanggaagagatttagagtcacgactcatagaagcatacgttatncaangtcaggcnnaagctcaagaagtnacaattgctcnagcaactgaactaaaacatgctcctg

SEQ ID NO: 146 - DNA, 525 bp, Homo sapiens
  misc_feature 3, 17, 20, 25, 28, 39, 53, 106, 208, 227, 401, 433, 455,507, 518, 520, 525: n = A,T,C or G
agntcgtcgaagcctanagnttgcngcngggatttttanacctttaaaggaangtcatctcccaaaactcattacacctgcggaaaaaggaagagatttagagtcncaactcatagaagcatacgttattcaatgtcaggctgaagctcaagaagtagcaattgctcgagcaattgaactaaaacatgctcctggactaattgctgcnctggcgtatgaaacagccnatttctaccaaaaagctgatcatactttatccagtttggagcctgcatattctgccgaatggagagaatatcttcgcttgaagatgtgtttctacacagcttatgcttactgttgccatggtgagactttattggctagtgataaatgcggtgaagcaatcaggtctctccaanaagcagaaaaattgtatgcaaaggcagaagcnctgtgtaaagaatatggagaanccaaaggacctggaccagcagtcagaccttcaggacatctgttctttagganacttggaaacngngtgan

SEQ ID NO: 147 - DNA, 452 bp, Homo sapiens
  misc_feature 12, 32, 33, 57, 144, 156, 187, 244, 307, 316, 365, 408,416, 417, 444: n = A,T,C or G
aaactcattacncctgcggaaaaaggaagagnnttagagtcacgactcatagaagcntacgttattcaatgtcaggctgaagctcaagaagtagcaattgctcgagcaattgaactaaaacatgctcctggactaattgctgcnctggcgtataanacagccaatttctatcaaaaagctggtcatnctttatccagtttggagcctgcatattctgtcaaatggagaaaatatcttcacttgnagatgtgtttctacacagcttatgcttactgttaccatggtgaggctttattggctagtgatnaatgcggtnaagcaatcaggtctctccaagaagcagaaaaattgtatgcaaaggcagnagcactgtgtaaagaatatggagaaaccaaaggacctggaccnacagtcannccttcagggcatctgttctttaggaancgtggttg

SEQ ID NO: 148 - DNA, 513 bp, Homo sapiens
  misc_feature 18, 52, 57, 198, 246, 251, 290, 326, 346, 424: n = A,T,C or G
aacatgctcctgggctanttgctgcactggcgtatgaaacagccaatttctnccaanaagctgatcatactttatccagtttggagcctgcatattctgccaaatggagaaaatatcttcacttgaagatgtgtttctacacagcttatgcttactgttaccatggtgagactttattggctagtgataaatgcggtnaagcaatcaggtctctccaagaagcagaaaaattgtatgcaaaggcanaagcnctgtgtaaagaatatggagaaaccaaaggacctggaccnacagtcaaaccttcaggacatctgttctttaggaancttggaaaccttgtgaagancaccctagaaaaatgtcagagagaaaatggatttatactaaacccaaaccagaagaagaagtgaaacctgtgaaaganccagacatcaaacctcaaaaggacactgggtgctacatctccaagggcgagcttgaaggtaagcctatccctaaccctctcctcggtcg

SEQ ID NO: 149 - DNA, 1744 bp, Homo sapiens; identical to SEQ ID NO: 114
  misc_feature 24, 29, 30, 95, 103, 1605, 1614, 1615, 1646, 1678, 1685,1697, 1716: n = A,T,C or G

SEQ ID NO: 150 - DNA, 853 bp, Homo sapiens
  misc_feature 39, 71, 90, 94, 136, 814, 816, 832, 844: n = A,T,C or G
acggatatctgcagaattcggcttatgacccattggttncataggaacccattaaaagccacagcttctgngtcttttaattactatggngtantcactggcccttctgcttcaaaaatatgcaatgacttgaggncatccagggcacgactccttgaactgttcactgatttgagctgtaatccagaaatgatgaagaatccagcagattcatatttctcacttttacaaggtttcataaattctttggatgaatctacccaagaaagcaagttacgatatattcaaaatttcaagtggactgatacattgcaaggacaggttccaagtgcccagcaggatgctgtttttgaattaatttccatgggatttaatgtagctttatggtataccaaatatgcttcaagactggctggaaaagaaaatataacagaagatgaagcaaaagaagttcatcgaagcctaaagattgcagctgggatttttaaacatttaaaggaaagtcatctcccaaaactcattacacctgcggaaaaaggaagagatttagagtcacgactcatagaagcatacgttattcaatgtcaggctgaagctcaagaagtaacaattgctcgagcaattgaactaaaacatgctcctggactaattgctgcactggcgtatgaaacagccaatttctatcaaaaagctgatcatactttatccagtttggagcctgcatattctgccaaatggagaaaatatcttcacttgaagatgtgtttctacacagcttatgcttactgttaccatggtgagactttattggctngngataaatgcggtgaancaatcaggtctnttcaagaag

SEQ ID NO: 151 - DNA, 884 bp, Homo sapiens
  misc_feature 1, 11, 86, 95, 865, 876: n = A,T,C or G
ntcttttaacnactatggtgtagtcactggcccttctgcttcaaaaatatgcaatgacttgaggtcatccagggcacgactccttnaactgttcnctgatttgagctgtaatccagaaatgatgaagaatccagcagattcatatttctcacttttacaaggtttcataaattctttggatgaatctacccaagaaagcaagttacgatatattcaaaatttcaagtggactgatacattgcaaggacaggttccaagtgcccagcaggatgctgtttttgaattaatttccatgggatttaatgtagctttatggtataccaaatatgcttcaagactggctggaaaagaaaatataacagaagatgaagcaaaagaagttcatcgaagcctaaagattgcagctgggatttttaaacatttaaaggaaagtcatctcccaaaactcattacacctgcggaaaaaggaagagatttagagtcacgactcatagaagcatacgttattcaatgtcaggctgaagctcaagaagtaacaattgctcgagcaattgaactaaaacatgctcctggactaattgctgcactggcgtatgaaacagccaatttctatcaaaaagctgatcatactttatccagtttggagcctgcatattctgccaaatggagaaaatatcttcacttgaagatgtgtttctacacagcttatgcttactgttaccatggtgagactttattggctagtgataaatgcggtgaagcaatcaggtctctccaagaagcagaaaaattgtatgcaaaggcagaagcactgtgtaaagagtatggagaagccaaaggacctggaccaacagtcaaaccttcnggacatctgtnctttagga

SEQ ID NO: 152 - DNA, 879 bp, Homo sapiens
  misc_feature 4, 786, 860: n = A,T,C or G
ggcncgccagtgtggatgggatatctgcaggaattcggcttatgacccattggtttcataggaacccattaaaagccacagcttctgtgtcttttaattactatggtgtagtcactggcccttctgcttcaaaaatatgcaatgacttgaggtcatccagggcacgactccttgaactgttcactgatttgagctgtaatccagaaatgatgaagaatgcagcagattcatatttctcacttttacaaggtttcataaattctttggatgaatctacccaagaaagcaagttacgatatattcaaaatttcaagtggacggatacattgcaaggacaggttccaagaagatgaagcaaaagaagttcatcgaagcctaaagattgcagctgggatttttaaacatttaaaggaaagtcatctcccaaaactcattacacctgcggaaaaaggaagagatttagagtcacgactcatagaagcatacgttattcaatgtcaggctgaagctcaagaagtaacaattgctcgagcaattgaactaaaacatgctcctggactaattgctgcactggcgtatgaaacagccaatttctatcaaaaagctgtttggggcctgcatattctgccaaatggagaaaatatcttcacttgaagatgtgtttctacacagcttatgcttactgttaccatggtgagactttattggctagtgataaatgcggtgaagcaatcaggtctctccaagaagcagaaaaattgtatgcaaaggcagaagcactgtgtnaagaatatggagaaaccaaaggacctggaccaacagacaaaccttcaggacatctgttctttaggaaacttggnaaccttgggaagaacaccc

SEQ ID NO: 153 - DNA, 484 bp, Homo sapiens
  misc_feature 18, 19, 37, 56, 67, 82, 95, 98, 105, 110, 132, 153, 163,186, 196, 205, 206, 214, 253, 255, 263, 275, 280, 292, 325,326, 343, 355, 357, 359, 390, 399, 401, 427, 432, 434, 442,448, 452, 455, 460, 471, 481: n = A,T,C or G
aaaggccccataggaacnnagtaaaagggacagcttntgtgacttttaattactanggcgcagtcantggccgttatgcttnaaaaatatgcaanganttgaggncatcnagggcacgactccttgaactgntcactgatttgagctgtaatncagaaatgangaagaatgcagcagattcatatntctcactttnacaaggttnnataaattntttggatgaatctacccaagaaagcaagttacgatatananaaaatttnaagtggactganacatngcaaggacaggntccaagtataacagaagatgaagcaaaagaagnncatcgaagcctaaagantgcagatgggantntnaaacatctaaaggaaagtcaactaccaaaantcattacancngcggaaaaaggaagagatttagaganacgantnatagaagnatacgntatnaantgatnggctgaagctnaagaagtaanaat

SEQ ID NO: 154 - DNA, 853 bp, Homo sapiens
  misc_feature 83, 792, 823, 835, 851: n = A,T,C or G
ttttaattactatggtgtagtcactggcccttctgcttcaaaaatatgcaatgacttgaggtcatccagggcacgactccttntactgttcactgatttgagctgtaatccagaaatgatgaagaatgcagcagattcatatttctcacttttacaaggtttcataaattctttggatgaatctacccaagaaagcaagttacgatatattcaaaatttcaagtggactgatacattgcaaggacaggttccaagtataacagaagatgaagcaaaagaagttcatcgaagcctaaagattgcagctgggatttttaaacatttaaaggaaagtcatctcccaaaactcattacacctgcggaaaaaggaagagatttagagtcacgactcatagaagcatacgttattcaatgtcaggctgaagctcaagaagtaacaattgctcgagcaattgaactaaaacatgctcctggactaattgctgcactggcgtatgaaacagccaatttctatcaaaaagctgatcatactttatccagtttggagcctgcatattctgccaaatggagaaaatatcttcacttgaagatgagtttctacacagcttatgcttactgttaccatggtgagactttattggctagtgataagtgcggtgaagcaatcaggtctctccaagaagcagaaaaattgtatgcaaaggcagaagcactgtgtaaagaatatggagaagccaaaggacctggaccaacagtcaaaccttcaggacatctgttctttaggaaactggnaaaccttgtgaagaacaccctagaaaaatgntagagagaaaanggatttatttactttnaa

SEQ ID NO: 155 - DNA, 869 bp, Homo sapiens
  misc_feature 84, 804: n = A,T,C or G
cttttaattactatggtgtagtcactggcccttctgcttcaaaaatatgcaatgacttgaggtcatccagggcacgactccttnaactgttcactgatttgagctgtaatccagaaatgatgaagaatgcagcagattcatatttctcacttttacaaggtttcataaattctttggatgaatctacccaagaaagcaagttacgatatattcaaaatttcaagtggacggatacattgcaaggacaggttccaagaagatgaagcaaaagaagttcatcgaagcctaaagattgcagctgggatttttaaacatttaaaggaaagtcatctcccaaaactcattacacctgcggaaaaaggaagagatttagagtcacgactcatagaagcatacgttattcaatgtcaggctgaagctcaagaagtaacaattgctcgagcaattgaactaaaacatgctcctggactaattgctgcactggcgtatgaaacagccaatttctatcaaaaagctgtttggggcctgcatattctgccaaatggagaaaatatcttcacttgaagatgtgtttctacacagcttatgcttactgttaccatggtgagactttattggctagtgataaatgcggtgaagcaatcaggtctctccaagaagcagaaaaattgtatgcaaaggcagaagcactgtgtaaagaatatggagaaaccaaaggacctggaccaacagacaaaccttcaggacatctgttctttaggaaacttggaaaccttgtgaagaacaccctagaaaaatgtcagngagaaaatggatttatttactttcaaaaaattccaacagaagccccacagctggaactcaaagca

SEQ ID NO: 156 - DNA, 871 bp, Homo sapiens
  misc_feature 7, 18, 43, 53, 70, 145, 170, 773, 774: n = A,T,C or G
ggatgantctacccaagnaagcaagttacgatatattcaaatnttcaagtggnctgatacattgcaaggncaggttccaagtataacagaagatgaagcaaaagaagttcatcgaagcctaaagattgcagctgggatttttaancatttaaaggaaagtcatctcccanaactcattacacctgcggaaaaaggaagagatttagagtcacgactcatagaagcatacgttattcaatgtcaggctgaagctcaagaagtaacaattgctcgagcaattgaactaaaacatgctcctggactaattgctgcactggcgtatgaaacagccaatttctatcaaaaagctgatcatactttatccagtttggagcctgcatattctgccaaatggagaaaatatcttcacttgaagatgagtttctacacagcttatgcttactgttaccatggtgagactttattggctagtgataagtgcggtgaagcaatcaggtctctccaagaagcagaaaaattgtatgcaaaggcagaagcactgtgtaaagaatatggagaagccaaaggacctggaccaacagtcaaaccttcaggacatctgttctttaggaaacttggaaaccttgtgaagaacaccctagaaaaatgttagagagaaaatggatttatttactttcaaaaaattccaacagaagccccacagctggaactcaaagcaaattatggtctcgtagagcctatacctttcgaatttcctcctacaagtgtccagnngacaccaaaaacattggctgcatttgatctcaccaaaagacccaaggatgacagtactaaacccaaaccagaagaagaagtgaaacctgtgaaagag

SEQ ID NO: 157 - DNA, 816 bp, Homo sapiens
  misc_feature 5, 36, 39, 40, 50, 74, 86, 105, 114, 161, 361, 783: n = A,T,C or G
gcaanagaagttcgtcgaagcctaaagattgcagcnggnntttttagacntttaaaggagagtcatctcccaanactcattacacntgcggaaaaaggaagaganttagagtcncgactcatagaagcatacgttattcaatgtcaggctgaagctcaagnagtaacaattgctcgagcaattgaactaaaacatgctcctggactaattgctgcactggcgtatgaaacagccaatttctatcaaaaagctgatcatactttatccagtttggagcctgcatattctgccaaatggagaaaatatcttcacttgaagatgagtttctacacagcttatgcttactgttaccatggtgagnctttattggctagtgataagtgcggtgaagcaatcaggtctctccaagaagcagaaaaattgtatgcaaaggcagaagcactgtgtaaagaatatggagaagccaaaggacctggaccaacagtcaaaccttcaggacatctgttctttaggaaacttggaaaccttgtgaagaacaccctagaaaaatgttagagagaaaatggatttatttactttcaaaaaattccaacagaagccccacagctggaactcaaagcaaattatggtctcgtagagcctatacctttcgaatttcctcctacaagtgtccagtggacaccagaaacattggctgcatttgatctcaccaaaagacccaaggatgacagtactaaacccaaaccagaagaagaagtgaaacctgtgaaagaaccagacatnaaacctcaagaggacactgggtgctacatctcc

SEQ ID NO: 158 - DNA, 747 bp, Homo sapiens
  misc_feature 11, 12, 14, 29, 80, 146, 660, 661, 662: n = A,T,C or G
ctgggattttnnancatttaaagaaaagncatctcccaaaactcattacacctgcggaaaaaggaagagatttagagtcncgactcatagaagcatacgttattcaatgtcaggctgaagctcaagaagtaacaattgctcgagcnattgaactaaaacatgctcctggactaattgctgcactggcgtatgaaacagccaatttctatcaaaaagctgatcatactttatccagtttggagcctgcatattctgccaaatggagaaaatatcttcacttgaagatgtgtttctacacagcttatgcttactgttaccatggtgagactttattggctagtgataaatgcggtgaagcaatcaggtctctccaagaagcagaaaaattgtatgcaaaggcagaagcactgtgtaaagagtatggagaagccaaaggacctggaccaacagtcaaaccttcaggacatctgttctttaggaaacttggagaccttgtgaagaacaccctagaaaaatgtcagagagaaaatggatttatttactttcaaaaaattccaacagaagccccacagctggaactcaaagcaaattatggtctcgtagagcctatacctttcgaatttcctcctacaagtgctcagtggacaccaaaaacattgnnngcatttgatctcaccaaaagacccaaggatgacagtactaaacccaaaccagaagaagaagtgaaacctgtgaaggaaccagaaa

SEQ ID NO: 159 - DNA, 654 bp, Homo sapiens
  misc_feature 24, 25, 32, 69, 79, 142, 189, 433, 461, 600, 602: n = A,T,C or G
gtcaggctgaagctcaagaagtannaattgcncgagcaattgaactaaaacatgctcctggactaattnctgcactggngtatgaaacagccaatttctatcaaaaagctgatcatactttatccagtttggagcctgcatnttctgccaaatggagaaaatatcttcacttgaagatgtgtttctacncagcttatgcttactgttaccatggtgagactttattggctagtgataaatgcggtgaagcaatcaggtctctccaagaagcagaaaaattgtatgcaaaggcagaagcactgtgtaaagagtatggagaagccaaaggacctggaccaacagtcaaaccttcaggacatctgttctttaggaaacttggagaccttgtgaagaacaccctagaaaaatgtcagagagaaaatggatttatttnctttcaaaaaattccaacagaagccccncagctggaactcaaagcaaattatggtctcgtagagcctatacctttcgaatttcctcctacaagtgctcagtggacaccagaaacattggctgcatttgatctcaccaaaagacccaaggatgacagtactaaacccnanccagaagaagaagtgaaacctgtgaaggaaccagacatcaaacctcaaaagg

SEQ ID NO: 160 - DNA, 589 bp, Homo sapiens
  misc_feature 19, 24, 29, 34, 37, 41, 171, 584: n = A,T,C or G
catgctcctggactaattnctgcnctggngtatnaancagncaatttctatcaaaaagctgtttggggcctgcatattctgccaaatggagaaaatatcttcacttgaagatgtgtttctacacagcttatgcttactgttaccatggtgagactttattggctagtgatnaatgcggtgaagcaatcaggtctctccaagaagcagaaaaattgtatgcaaaggcagaagcactgtgtaaagaatatggagaaaccaaaggacctggaccaacagacaaaccttcaggacatctgttctttaggaaacttggaaaccttgtgaagaacaccctagaaaaatgtcagagagaaaatggatttatttactttcaaaaaattccaacagaagccccacagctggaactcaaagcaaattatggtctcgtagagcctatacctttcgaatttcctcctacaagtgttcagtggacaccagaaacattggctgcatttgatctcaccaaaagacccaaggatgacagtactaaacccaaaccagaagaagaagtgaaacctgtgaaagaaccagacatcaaacctcanaagga

SEQ ID NO: 161 - DNA, 551 bp, Homo sapiens; identical to SEQ ID NO: 58
  misc_feature 14, 60, 467, 476: n = A,T,C or G

SEQ ID NO: 162 - protein, 43 aa, Homo sapiens
LQGFINSLDESTQESKLRYIQNFKWTDTLQGQVPSAQQDAVFE

SEQ ID NO: 163 - protein, 21 aa, Saccharomyces sp.
VQFAINKVDKITHESKLRYLQ

SEQ ID NO: 164 - protein, 20 aa, Mus musculus
FHWYDSLTGVPAQQRALAFE

SEQ ID NO: 165 - protein, 6 aa, Homo sapiens
AQQDAV

SEQ ID NO: 166 - protein, 5 aa, Homo sapiens
FYQKA

SEQ ID NO: 167 - protein, 5 aa, Homo sapiens
KYLHL

SEQ ID NO: 168 - protein, 308 aa, Homo sapiens
MKIGPSFKANAITDAQQDAVFELISMGFNVALWYTKYASRLAGKENITEDEAKEVHRSLKIAAGIFKHLKESHLPKLITPAEKGRDLESRLIEAYVIQCQAEAQEVTIARAIELKHAPGLIAALAYETANFYQKAAKYLHLKMCFYTAYAYCYHGETLLASDKCGEAIRSLQEAEKLYAKAEALCKEYGETKGPGPTVKPSGHLFFRKLGNLVKNTLEKCQRENGFIYFQKIPTEAPQLELKANYGLVEPIPFEFPPTSVQWTPETLAAFDLTKRPKDDSTKPKPEEEVKPVKEPDIKPQKDTGCYIS

SEQ ID NO: 169 - DNA, 850 bp, Homo sapiens
atgaaaatcggcccgtcttttaaagccaatgccattaccgatgcccagcaggatgctgtttttgaattaatttccatgggatttaatgtagctttatggtataccaaatatgcttcaagactggctggaaaagaaaatataacagaagatgaagcaaaagaagttcatcgaagcctaaagattgcagctgggatttttaaacatttaaaggaaagtcatctcccaaaactcattacacctgcggaaaaaggaagagatttagagtcacgactcatagaagcatacgttattcaatgtcaggctgaagctcaagaagtaacaattgctcgagcaattgaactaaaacatgctcctggactaattgctgcactggcgtatgaaacagccaatttctatcaaaaagctgcaaaatatcttcacttgaagatgtgtttttacacagcttatgcttactgttaccatggtgagactttattggctagtgataaatgcggtgaagcaatcaggtctctccaagaagcagaaaaattgtatgcaaaggcagaagcactgtgtaaagaatatggagaaaccaaaggacctggaccaacagtcaaaccttcaggacatctgttctttaggaaacttggaaaccttgtgaagaacaccctagaaaaatgtcagagagaaaatggatttatttactttcaaaaaattccaacagaagccccacagctggaactcaaagcaaattatggtctcgtagagcctatacctttcgaatttcctcctacaagtgttcagtggacaccagaaacattggctgcatttgatctcaccaaaagacccaaggatgacagtactaaaccca

SEQ ID NO: 170 - protein, 8 aa, Homo sapiens
ESKLRYIQ

SEQ ID NO: 171 - protein, 8 aa, Sacchromyces sp.
ESKLRYLQ

SEQ ID NO: 172 - DNA, 20 bp, Artificial Sequence
  PCR primer
accaaatatgcttcaagact

SEQ ID NO: 173 - DNA, 27 bp, Artificial Sequence
  PCR primer
cacgtcatccagcagagaatggaaact

SEQ ID NO: 174 - DNA, 28 bp, Artificial Sequence
  PCR primer
tgaccaagatgttgatgttggataagag

(19) United States
(12) Patent Application Publication
(10) Pub. No.: US 2002/0106765 A1
(43) Pub. Date: Aug. 8, 2002

(54) PAL-18 polypeptides, nucleic acids encoding the same and methods for screening for or modulating the same

(76) Inventors: Robert J. Kinders, Woodinville, WA (US); Michael J. Corey, Bellevue, WA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 09/804,682

(22) Filed: Mar. 12, 2001

Related U.S. Application Data
(60) Provisional application No. 60/188,586, filed on Mar. 10, 2000.

(51) Int. Cl. C07H 21/04; C12N 9/00; C12P 21/02; C12Q 1/68

(52) U.S. Cl. 435/183; 435/69.1; 435/325; 435/320.1; 536/23.2; 435/6

(57) ABSTRACT

Methods of screening for and/or detecting the presence or absence of cancers or treating cancers are described. In an aspect of the present invention, the screening methods are based on the detection of the PAL-18 protein, variants thereof, or a nucleic acid molecules encoding the same, found to be associated with the presence of cancer. Additional methods for imaging using antibodies for PAL-18 or modulating PAL-18 are provided. Preferred embodiments to the methods include detection based on immunological properties, physical properties, enzymatic properties and combinations thereof, or detection of a nucleic acid molecule encoding antigen (e.g., PAL-18) based on nucleic acid amplification.

### CROSS REFERENCE TO RELATED APPLICATION

The present application claims priority to U.S. Provisional Application No. 60/188,586, filed Mar. 10, 2000.

### TECHNICAL FIELD

The present invention is generally directed toward the discovery of novel nucleic acid molecules and PAL-18 polypeptides encoded thereby. More specifically, the invention is directed to these nucleic acid molecules and polypeptides encoded thereby for screening for or modulating a PAL-18 polypeptide in vitro and/or in vivo. In addition, the invention is related to detecting a PAL-18 polypeptide, or a nucleic acid molecule encoding such a protein, associated with the presence of cancer, and to modulating the presence or activity of such a protein.

### BACKGROUND OF THE INVENTION

Over the past twenty years many elements of the biochemical pathways associated with abnormal growth and dysregulation of programmed cell death (also known as apoptosis) in human cancer have been elucidated; however, our knowledge of these pathways is far from comprehensive. Gaps in our understanding include the fact that the catalog of genes and proteins affecting these pathways is incomplete. Such incompleteness is evidenced in that proteins have been found in various eukaryotes which have been shown to be involved with Mitogen Activated Protein Kinase (MAPK) associated pathways, but closely similar human proteins related to these MAPK-associated proteins, which might perform parallel functions in humans, have not yet been identified. MAPKs are related to cancer through their interactions with the so-called “RAS-initiated” pathways. In the normal growth-regulation pathway, RAS is activated directly by proteins that interact with growth factors. Activated RAS brings about a cascade of kinase activation, including activation of the MAPKs, followed by phosphorylation of many other proteins involved in growth regulation, including the well-known oncoproteins ETS, MYC, FOS, and JUN. Proteins which directly interact with MAPKs could therefore modulate these signaling pathways and profoundly influence growth regulation, and, by extension, the development of cancer. Another important mechanism whereby cancer cells escape the normal checks on growth is by dysregulation of apoptosis, and MAPKs such as p38 have also been shown to be involved in regulation of the apoptotic process. However, the proteins mentioned above that are active in modulating MAPK pathways in other eukaryotes, but for which no strongly similar human homologs are known, represent a gap in the understanding of these growth-regulation and apoptotic pathways in humans, and therefore in the ability to intervene in patients with aberrant regulation associated with cancer.

Accordingly, there is a need in the art for advancing the understanding of such proteins and for methods for detection and utilization of such genes as related to disease indications. In particular, such genes and gene products may be utilized in the treatment, detection, and/or prevention of cancer and related disorders. The present invention fulfills this need and further provides other related advantages.

### SUMMARY OF THE INVENTION

The surprising discovery and characterization of a particular human gene, PAL-18 are presented herein. This gene exhibits significant homologies to a number of proteins from other species which are implicated in MAPK signaling pathways. The present invention discloses that upregulation of the PAL-18 message is associated with all colon cancers studied and expression levels and/or certain single nucleotide polymorphisms may also be associated with familial predisposition to certain types of cancer such as prostate cancer. Briefly stated, the present invention provides a variety of methods and compositions for screening for cancer, and for treating tumor cells. The screening methods and compositions may be used on a one-time basis when cancer is suspected or on a periodic basis, e.g., to monitor an individual with an elevated risk of acquiring or reacquiring cancer.

In one aspect, the present invention provides an isolated PAL-18 polypeptide comprising an amino acid sequence that is encoded by a polynucleotide sequence selected from a sequence recited in SEQ ID NOs: 1-71, sequences that hybridize to a sequence recited in SEQ ID NOs:1-71 or 75-79 under moderately stringent conditions and complements of the polynucleotide sequences of (a) and (b).

In a related aspect an isolated polypeptide that comprises an amino acid sequence that is encoded by a polynucleotide sequence recited in SEQ ID NOs:75-79 or a complement thereof is provided as are polypeptides comprising SEQ ID NOs:72-74 and fragments thereof of at least 15 contiguous amino acid residues of SEQ ID NOs:72-74.

In another aspect, the present invention provides isolated polynucleotides encoding at least 15 amino acid residues of a PAL-18 polypeptide, or a variant thereof that differs in one or more substitutions, deletions, additions and/or insertions such that the ability of the variant to react with antigen-specific antisera is not substantially diminished, wherein the PAL-18 polypeptide comprises an amino acid sequence that is encoded by a polynucleotide comprising a sequence recited in SEQ ID NOs:1-71, 75-79 or a complement thereof. Also provided are isolated polynucleotides encoding a PAL-18 polypeptide or a variant thereof, wherein the PAL-18 polypeptide comprises an amino acid sequence that is encoded by a polynucleotide comprising a sequence recited in SEQ ID NOs: 1-71, 75-79, a complement thereof, or a sequence having at least 70% identity with SEQ ID NOs: 1-71 or 75-79. In related aspects the isolated polynucleotide comprises a sequence recited in SEQ ID NOs: 1-71 or 75-79 or complement thereof or isolated polynucleotides comprising a sequence that hybridizes to a sequence recited in SEQ ID NOs:1-71 or 75-79 under moderately stringent conditions or isolated polynucleotides comprising a sequence having at least 70% identity with SEQ ID NOs:1-71 or 75-79. Expression vectors containing any of the above polynucleotide sequences are also provided as are host cell containing these vectors.

In a further aspect the invention provides an isolated antibody, or antigen-binding fragment thereof, that specifically binds to a PAL-18 polypeptide or fragment thereof that comprises an amino acid sequence that is encoded by a polynucleotide sequence recited in SEQ ID NOs: 1-71, 75-79 or a complement thereof.

Yet other aspects of the present invention provide pharmaceutical compositions comprising a physiologically acceptable carrier and at least one PAL-18 polypeptide, a fragment of a PAL-18 polypeptide, a PAL-18 encoding polynucleotide, or an antibody reactive with a PAL-18 polypeptide.

Also provided are methods for determining the presence or absence of a cancer in a patient, comprising contacting a biological sample obtained from a patient with a binding agent that binds to a PAL-18 polypeptide, wherein the PAL-18 polypeptide comprises an amino acid sequence that is encoded by a polynucleotide sequence recited in SEQ ID NOs: 1-71, 75-79 or a complement thereof, detecting in the sample an amount of polypeptide that binds to the binding agent and comparing the amount of polypeptide to a predetermined cut-off value, and therefrom determining the presence or absence of a cancer in the patient. In related aspects the binding agent is an antibody and/or the antibody is a monoclonal antibody. In further aspects, the cancer being detected is breast, prostate or colon cancer.

In yet other related aspects the detecting step involves detection of various molecular weight isoforms of PAL-18 or expression levels of PAL-18. Other related aspects utilize a biological sample selected from a tissue sample, a urine sample, a blood sample, and a stool sample.

In yet other embodiments a method for monitoring the progression of a cancer in a patient is provided and comprises contacting a biological sample obtained from a patient at a first point in time with a binding agent that binds to a PAL-18 polypeptide or fragment thereof, wherein the PAL-18 polypeptide or fragment thereof comprises at least 15 contiguous amino acid residues encoded by a polynucleotide sequence recited in SEQ ID NOs:1-71, 75-79 or a complement thereof, detecting in the sample an amount of polypeptide that binds to the binding agent, repeating the previous steps using a biological sample obtained from the patient at a subsequent point in time and comparing the amount of polypeptide detected in step (c) to the amount detected in step (b) and therefrom monitoring the progression of the cancer in the patient. In certain embodiments the binding agent is an antibody and in yet other embodiments the antibody is a monoclonal antibody. In yet other related embodiments, the cancer being detected is breast, colon, or prostate cancer.

Additional related aspects of the methods comprise a detecting step that involves detection of various molecular weight isoforms of PAL-18 or that involves detection of expression levels of PAL-18.

In still yet other aspects, the present invention provides methods for determining the presence or absence of a cancer in a patient, comprising contacting a biological sample obtained from a patient with an oligonucleotide that hybridizes to a polynucleotide that encodes a PAL-18 polypeptide or fragment thereof, wherein the PAL-18 polypeptide comprises at least 15 contiguous amino acid residues encoded by a polynucleotide sequence recited in SEQ ID NOs:1-71, 75-79 or a complement thereof and wherein the at least 15 contiguous amino acid residues retain the ability to react with an a PAL-18-specific antisera, detecting in the sample an amount of a polynucleotide that hybridizes to the oligonucleotide and comparing the amount of polynucleotide that hybridizes to the oligonucleotide to a predetermined cut-off value, and therefrom determining the presence or absence of a cancer in the patient. Related aspects include wherein the amount of polynucleotide that hybridizes to the oligonucleotide is determined using a polymerase chain reaction, wherein the polymerase chain reaction is RT-PCR, orwherein the amount of polynucleotide that hybridizes to the oligonucleotide is determined using a hybridization assay.

Also provided by the present invention are methods for monitoring the progression of a cancer in a patient, comprising contacting a biological sample obtained from a patient with an oligonucleotide that hybridizes to a polynucleotide that encodes a PAL-18 polypeptide or fragment thereof, wherein the PAL-18 polypeptide comprises at least 15 contiguous amino acid residues encoded by a polynucleotide sequence recited in SEQ ID NOs:1-71, 75-79 or a complement thereof and wherein the at least 15 contiguous amino acid residues retain the ability to react with an a PAL-18-specific antisera, detecting in the sample an amount of a polynucleotide that hybridizes to the oligonucleotide, repeating the above steps using a biological sample obtained from the patient at a subsequent point in time and comparing the amount of polynucleotide detected in step (c) to the amount detected in step (b) and therefrom monitoring the progression of the cancer in the patient.

Further provided are diagnostic kits comprising one or more antibodies according to claim 13 and a detection reagent comprising a reporter group. In related embodiments the antibodies are immobilized on a solid support and/or the detection reagent comprises an anti-immunoglobulin, protein G, protein A or lectin. In yet additional related aspects the kit may contain a reporter group such as radioisotopes, fluorescent groups, luminescent groups, enzymes, biotin and dye particles.

Aspects of the invention are also related to an oligonucleotide comprising 10 to 40 contiguous nucleotides that hybridize under highly stringent conditions to a polynucleotide that encodes a PAL-18 polypeptide or fragment thereof, wherein the PAL-18 polypeptide or fragment thereof comprises an amino acid sequence that is encoded by a polynucleotide sequence recited in any one of SEQ ID NOs:1-71, 75-79 or a complement thereof.

Further aspects of the invention provide diagnostic kits comprising an oligonucleotide as described above and a diagnostic reagent for use in a polymerase chain reaction or hybridization assay.

Another aspect of the present invention provides a method of treating a tumor cell comprising the step of modulating a tumor-associated PAL-18 polypeptide or a nucleic acid molecule encoding said polypeptide, said nucleic acid molecule characterized by the ability of said nucleic acid molecule to hybridize under moderate stringency with any one of SEQ ID NOs:1-71, 75-79, or a complement thereof. Also provided are related aspects comprising the step of modulating the antigen and wherein the tumor cell is a colorectal or prostate tumor cell.

Other aspects of the present invention include an agent that modulates a tumor-associated PAL-18 polypeptide or a nucleic acid molecule encoding said polypeptide, said nucleic acid molecule characterized by the ability of said nucleic acid molecule to hybridize under moderate stringency with any one of SEQ ID NOs:1-71, 75-79, or a complement thereof as well as compositions comprising an agent that modulates a PAL-18 polypeptide or a nucleic acid molecule encoding said polypeptide, said nucleic acid molecule characterized by the ability of said nucleic acid molecule to hybridize under moderate stringency with any one of SEQ ID NOs:1-71, 75-79, or a complement thereof, in combination with a pharmaceutically acceptable carrier or diluent.

Still yet other aspects provided are methods for diagnosing abnormal PAL-18, comprising isolating PAL-18 encoding nucleic acid molecules from a sample and subsequently screening for single nucleotide polymorphisms, whereby detection of a single nucleotide polymorphism indicates an abnormal PAL-18.

Further provided is a method for facilitating in vivo imaging, comprising administering to an animal an binding agent that specifically binds a PAL-18 polypeptide, said binding agent having an imaging agent attached thereto.

These and other aspects of the present invention will become evident upon reference to the following detailed description and attached drawings.

### BRIEF DESCRIPTION OF THE DRAWINGS

FIG. 1 depicts a scanned image of agarose gel electrophoresis of culture PCR screening of TA cloned amplicons from five libraries. Colony 13 demonstrates a positive clone.

FIG. 2 depicts local homologies of PAL-18 (SEQ ID NOS: 162 & 164) with yeast calnexin (SEQ ID NO:163) and mouse rhophilin (SEQ ID NO:165).

FIG. 3 represents a scanned image of agarose gel electrophoresis of PAL-18 message detected in matched pairs of cancer (left lane of each sample) and normal (right lane of each sample) tissue from six prostate-cancer patients. Left most lane represents molecular weight markers (PCR Rulers® Biorad Corp.).

FIG. 4 represents a scanned image of agarose gel electrophoresis of the PAL-18 message presence in matched pairs of cancer (C) and normal (N) tissue from six colon cancer patients. The molecular weight markers (M) are the PCR ruler from Biorad Corp. and the Mic represents a control of microglobulin.

FIG. 5 is the sequence of contiguous sequence 1 (SEQ ID NO:75) compiled from gene walking experiments, including alignments with various other sequences (SEQ ID NOS: 114-120).

FIG. 6 is the sequence of contiguous sequence 2 (SEQ ID NO:76) compiled from gene walking experiments, including alignments with various other sequences (SEQ ID NOS: 121-137).

FIG. 7 is the sequence of contiguous sequence 3 (SEQ ID NO:77) compiled from gene walking experiments, including alignments with various other sequences (SEQ ID NOS: 138-140).

FIG. 8 is the sequence of contiguous sequence 4 (SEQ ID NO:78) compiled from gene walking experiments, including alignments with various other sequences (SEQ ID NOS: 141-148).

FIG. 9 is the sequence of contiguous sequence 5 (SEQ ID NO:79) compiled from gene walking experiments, including alignments with various other sequences (SEQ ID NOS: 149-161).

### DETAILED DESCRIPTION OF THE INVENTION

The invention consists of a set of compositions of matter of PAL-18 and related genes, with encoded proteins, and the uses of these genes and proteins in diagnosing, characterizing, and treating disease, and in determining disease susceptibility. In addition the invention provides methods of analysis to detect and/or quantify alternatively expressed forms of PAL-18 or genes related to PAL-18. The invention also describes methods of finding PAL-18-related sequences by low-fidelity RT-PCR and identification of qualifying sequences by database searching with specific criteria for inclusion and exclusion of candidate related sequences.

Prior to setting forth details of the invention, it may be helpful to an understanding thereof to set forth definitions of certain terms that will be used hereinafter.

A “PAL-18 polypeptide”, as used herein, refers to a polypeptide having at least about 70% amino acid identity with any one of SEQ ID NOS:72-74 or the Sequence set forth in Example 1 or the polypeptides encoded by SEQ ID NOS:1-71, 75-79, or the polypeptide encoded by the cDNA set forth in Example 1. The PAL-18 polypeptides of the subject invention also include variants (including alleles) of the native protein sequence. Briefly, such variants may result from natural polymorphisms or may be synthesized by recombinant methodology, and differ from wild-type protein by one or more amino acid substitutions, insertions, deletions, or the like. Variants generally have at least about 70% nucleotide identity to native sequence, preferably at least 80%-85%, and most preferably at least 90% nucleotide identity.

Typically, when engineered, amino acid substitutions will be conservative, i.e., substitution of amino acids within groups of polar, non-polar, aromatic, charged, etc. amino acids. In the region of homology to the native sequence, variants should preferably have at least 50% amino acid sequence identity, and within certain embodiments, greater than 75%, 80%, 90%, 92%, 95%, or 97% identity. More typically, the amino acid sequence identity will be at least about any percentage from (and including) 50% to 100%, such as at least about 55%, 60%, 65%, 70%, 75%, 80%, 85%, 90% or 95% identity. Variants that nearly identical to a PAL-18 polypeptide have at least about 85% or 90% identity. Accordingly, one method for determining amino acid sequence “identity” is determined by the alignment of amino acid sequences and establishment of identical amino acid residues using the program GeneJockey II (1993) for Macintosh (Philip L. Taylor, published by Biosoft, Cambridge, UK). The program is run in the amino acid homology mode, using program default parameters. In the comparison of two sequences aligned by the program, the percent identity is calculated only for those positions where there is an amino acid residue present in both of the two sequences. Such amino acid sequence identity may be determined by other standard methodologies, including use of the National Center for Biotechnology Information BLAST search methodology available at www.ncbi.nlm.nih.gov using the BLAST 2.0 algorithm and default parameters (also available from CGC, Madison, Wis.). The identity methodologies most preferred are those described in U.S. Pat. No. 5,691,179 (specifically, the GAP algorithm) and Altschul et al., Nucleic Acids Res. 25:3389-3402, 1997, both of which are incorporated herein by reference. Typically, when such identity algorithms are used they are carried out with default parameters.

Within the context of the present invention, polypeptides may comprise at least an immunogenic portion of a PAL-18 polypeptide or a variant thereof, as described herein. As noted above, a “PAL-18 antigen” is a protein that is expressed by a variety of cell types and may be variably expressed at differing levels and/or expressed in various isoforms in cancer cells. Polypeptides as described herein may be of any length. Additional sequences derived from the native protein and/or heterologous sequences may be present, and such sequences may (but need not) possess further immunogenic or antigenic properties.

An “immunogenic portion,” as used herein is a portion of a protein that is recognized (i.e., specifically bound) by a B-cell and/or T-cell surface antigen receptor. Such immunogenic portions generally comprise at least 5 amino acid residues, more preferably at least 10, and still more preferably at least 20 amino acid residues of an PAL-18 polypeptide or a variant thereof. Certain immunogenic portions may include peptides in which an N-terminal leader sequence have been deleted. Other preferred immunogenic portions may contain a small N- and/or C-terminal deletion (e.g., 1-30 amino acids, preferably 5-15 amino acids), relative to the mature protein.

Immunogenic portions may generally be identified using well known techniques, such as those summarized in Paul, Fundamental Immunology, 3rd ed., 243-247 (Raven Press, 1993) and references cited therein. Such techniques include screening polypeptides for the ability to react with antigen-specific antibodies, antisera and/or T-cell lines or clones. As used herein, antisera and antibodies are “antigen-specific” if they specifically bind to an antigen (i.e., they react with the protein in an ELISA or other immunoassay, and do not react detectably with unrelated proteins). Such antisera and antibodies may be prepared as described herein, and using well known techniques. An immunogenic portion of a PAL-18 polypeptide is a portion that reacts with such antisera and/or T-cells at a level that is not substantially less than the reactivity of the full length polypeptide (e.g., in an ELISA and/or T-cell reactivity assay). Such immunogenic portions may react within such assays at a level that is similar to or greater than the reactivity of the full length polypeptide. Such screens may generally be performed using methods well known to those of ordinary skill in the art, such as those described in Harlow and Lane, Antibodies: A Laboratory Manual, Cold Spring Harbor Laboratory, 1988. For example, a polypeptide may be immobilized on a solid support and contacted with patient sera to allow binding of antibodies within the sera to the immobilized polypeptide. Unbound sera may then be removed and bound antibodies detected using, for example, ¹²⁵I-labeled Protein A.

In certain embodiments a PAL-18 polypeptide variant differs from a native PAL-18 polypeptide sequence in one or more substitutions, deletions, additions and/or insertions, such that the immunogenicity of the polypeptide is not substantially diminished. In other words, the ability of a variant to react with antigen-specific antisera may be enhanced or unchanged, relative to the native protein, or may be diminished by less than 50%, and preferably less than 20%, relative to the native protein. Such variants may generally be identified by modifying one of the above polypeptide sequences and evaluating the reactivity of the modified polypeptide with antigen-specific antibodies or antisera as described herein. Preferred variants include those in which one or more portions, such as an N-terminal leader sequence, have been removed. Other preferred variants include variants in which a small portion (e.g., 1-30 amino acids, preferably 5-15 amino acids) has been removed from the N- and/or C-terminal of the mature protein.

As will be appreciated by those skilled in the art, a nucleotide sequence encoding a PAL-18 polypeptide or variant thereof may differ from known native sequence, due to codon degeneracies, nucleotide polymorphisms, or amino acid differences. In certain embodiments, variants will preferably hybridize to the native nucleotide sequence at conditions of normal (moderate) stringency, which is approximately 25-30° C. below Tm of the native duplex (e.g., 5×SSPE, 0.5% SDS, 5×Denhardt's solution, 50% formamide, at 42° C. or equivalent conditions; see generally, Sambrook et al. Molecular Cloning: A Laboratory Manual, 2nd ed., Cold Spring Harbor Press, 1989; Ausubel et al., Current Protocols in Molecular Biology, Greene Publishing, 1995). By way of comparison, low stringency hybridizations utilize conditions approximately 40° C. below Tm, and high stringency hybridizations utilize conditions approximately 10° C. below Tm. Accordingly, within certain embodiments, moderate stringency hybridization and PCR amplification conditions are defined as those performed at the calculated melting temperature (Tm) of the probe/primer with the target. The recommended formula for calculating Tm, and its limitations, are well known in the art (i.e., are found in Sambrook, J., Fritsch, E. F. and T. Maniatis, Molecular Cloning, 2d Edition, Cold Spring Harbor Laboratory press, pp. 9.51-9.52, 1989). Highest stringency conditions are defined within this application as hybridization/amplification performed at least 4° C. above the calculated Tm. Further illustrative conditions for suitable moderately stringent conditions include, for example, prewashing in a solution of 5×SSC, 0.5% SDS, 1.0 mM EDTA (pH 8.0); hybridizing at 50° C.-70° C., 5×SSC for 1-16 hours; followed by washing once or twice at 22-65° C. for 20-40 minutes with one or more each of 2×, 0.5× and 0.2×SSC containing 0.05-0.1% SDS. For additional stringency, conditions may include a wash in 0.1×SSC and 0.1% SDS at 50-60° C. for 15 minutes.

As known to those having ordinary skill in the art, variations in stringency of hybridization conditions may be achieved by altering the time, temperature and/or concentration of the solutions used for prehybridization, hybridization and wash steps, and suitable conditions may also depend in part on the particular nucleotide sequences of the probe used, and of the blotted, PAL-18 nucleic acid sample. Accordingly, it will be appreciated that suitably stringent conditions can be readily selected without undue experimentation where a desired selectivity of the probe is identified, based on its ability to hybridize to one or more certain sequences while not hybridizing to certain other sequences.

It will also be appreciated by those of ordinary skill in the art that, as a result of the degeneracy of the genetic code, there are many nucleotide sequences that encode a polypeptide as described herein. Some of these polynucleotide s bear minimal homology to the nucleotide sequence of any native gene. Nonetheless, polynucleotides that vary due to differences in codon usage are specifically contemplated by the present invention.

An “isolated nucleic acid molecule” refers to a polynucleotide molecule in the form of a separate fragment or as a component of a larger nucleic acid construct, that has been separated from its source cell (including the chromosome it normally resides in) at least once, and preferably in a substantially pure form. Nucleic acid molecules may be comprised of a wide variety of nucleotides, including DNA, RNA, nucleotide analogues, or combination thereof and may include variants as noted above.

The term “in vitro” refers to systems outside the body including organ culture.

The term “in vivo” refers to whole cell systems, which include, for example, primary and secondary cell culture, whole organs culture, whole organisms, and similar systems as known to those of ordinary skill in the art.

The term “predetermined cut-off value”, as used herein, refers to a background value or a value normally present in the tissue type or biological sample.

A. PAL-18 Nucleic Acid Molecules and Encoded Products Thereof

1. PAL-18 Nucleic Acid Molecules

The present invention unexpectedly provides PAL-18 nucleic acid molecules which, in certain embodiments, are derived from human or animal libraries. PAL-18 nucleic acid molecules used in the subject invention may be isolated from either genomic DNA or preferably cDNA sources. Isolation of PAL-18 nucleic acid molecules from genomic DNA or cDNA typically can proceed by, first, generating an appropriate DNA library through techniques for constructing libraries that are known in the art (see Sambrook et al., Molecular Cloning: A Laboratory Manual, Cold Spring Harbor Press, 1989) or purchased from commercial sources (e.g., Clontech, Palo Alto, Calif.). Briefly, cDNA libraries can be constructed in bacteriophage vectors (e.g., λZAPII), plasmids, or others, which are suitable for screening, while genomic DNA libraries can be constructed in chromosomal vectors, such as YACs (yeast artificial chromosomes), bacteriophage vectors, such as λEMBL3, λgt10, cosmids, or plasmids.

In one embodiment, PAL-18 sequences set forth herein may be utilized to design an oligonucleotide hybridization probe suitable for screening genomic or cDNA libraries. Preferably, such oligonucleotide probes are 20-35 bases in length. To facilitate hybridization detection, the oligonucleotide may be conveniently labeled, generally at the 5′ end, with a reporter molecule, such as a radionuclide, (e.g., ³²P), enzymatic label, protein label, fluorescent label, or biotin. Such libraries are then generally plated as phage or colonies, depending upon the vector used. Subsequently, a nitrocellulose or nylon membrane, to which the colonies or phage have been transferred, is probed to identify candidate clones which contain the gene. Such candidates may be verified as containing the desired DNA by any of various means including, for example, DNA sequence analysis or hybridization with a second, non-overlapping probe.

Once a library is identified as containing a PAL-18 nucleic acid molecule, the molecule can be isolated by amplification. Primers for amplification are preferably derived from sequences in the 5′ and 3′ untranslated region in order to isolate a full-length cDNA. The primers preferably have a GC content of about 50% and contain restriction sites to facilitate cloning and do not have self-complementary sequences nor do they contain complementary sequences at their 3′ end (to prevent primer-dimer formation). The primers are annealed to cDNA or genomic DNA and sufficient amplification cycles are performed to yield a product readily visualized by gel electrophoresis and staining. The amplified fragment is purified and inserted into a vector, such as λgt10 or pBS(M13+), and propagated. Confirmation of the nature of the fragment may be obtained by DNA sequence analysis, or indirectly through amino acid sequencing of the encoded protein.

Other methods may also be used to obtain a PAL-18 encoding nucleic acid molecule. For example, a nucleic acid molecule encoding a PAL-18 polypeptide may be obtained from an expression library by screening with an antibody or antibodies reactive to such a PAL-18 polypeptide or fragment thereof (see, Sambrook, et al. Molecular Cloning: A Laboratory Manual, 2nd Ed., Cold Spring Harbor Laboratory Press, NY, 1989; Ausubel, et al. Current Protocols in Molecular Biology, Greene Publishing Associates and Wiley-Interscience, NY, 1995).

PAL-18 nucleic acid molecules from a variety of species may be isolated using the compositions provided herein. For closely related species, the human sequence or portion thereof may be utilized as a probe on a genomic or cDNA library. For example, a fragment of nucleic acid that encodes a portion of the PAL-18 polypeptide may be labeled and used as a probe on a library constructed from mouse, primate, rat, dog, or other vertebrate, warm-blooded or mammalian species. An initial hybridization at normal stringency may yield candidate clones or fragments. If no hybridization is initially observed, varying degrees of stringency may be used (see Sambrook et al., supra, and other well-known sources for stringency conditions). While such probes may also be used to probe libraries from evolutionarily diverse species, such as Drosophila, hybridization conditions will likely be less stringent.

While relaxed hybridization conditions using probes designed from human sequences may identify PAL-18 nucleic acid molecules of evolutionarily diverse species, it may be more beneficial to attempt to directly isolate these molecules from a library using methods which do not require the human sequence per se. These methods include, but are not limited to, amplification using primers derived from conserved areas, amplification using degenerate primers from various regions, antibody probing of expression libraries, and the like. For example, random-primed amplification (e.g., polymerase chain reaction) may be employed (see, e.g., Methods Enzymol. 254:275, 1995; Trends Genet. 11:242, 1995; Liang and Pardee, Science 257:967, 1992; Welsh et al., Nucl. Acids Res. 20:4965, 1992). In addition, variations of random-primed PCR may also be used, especially when a particular gene or gene family is desired. In such a method, one of the amplification primers is an “anchored oligo(dT) (oligo(dT)dN)” and the other primer is a degenerate primer based upon amino acid or nucleotide sequence of a related gene. A gene sequence is identified as a PAL-18 molecule by amino acid similarity and/or nucleic acid similarity. Generally, amino acid similarity is preferred.

Variants of PAL-18 nucleic acid molecules provided herein may be engineered from natural variants (e.g., polymorphisms, splice variants, mutants), synthesized or constructed. Many methods have been developed for generating mutants (see, generally, Sambrook et al, supra; Ausubel, et al., supra, and the discussion above). Briefly, preferred methods for generating nucleotide substitutions utilize an oligonucleotide that spans the base or bases to be mutated and contains the mutated base or bases. The oligonucleotide is hybridized to complementary single stranded nucleic acid and second strand synthesis is primed from the oligonucleotide. The double-stranded nucleic acid is prepared for transformation into host cells, typically E. coli, but alternatively, other prokaryotes, yeast or other eukaryotes. Standard screening and vector growth protocols are used to identify mutant sequences and obtain high yields.

Similarly, deletions and/or insertions of a PAL-18 encoding nucleic acid molecule may be constructed by any of a variety of known methods as discussed supra. For example, the nucleic acid molecule can be digested with restriction enzymes and religated, thereby deleting or religating a sequence with additional sequences, such that an insertion or large substitution is made. Other means of generating variant sequences may be employed using methods known in the art, for example those described in Sambrook et al., supra; Ausubel et al., supra. Verification of variant sequences is typically accomplished by restriction enzyme mapping, sequence analysis, or probe hybridization.

As one of skill in the art can readily appreciate, once DNA sequences for a gene have been identified the production of antisense nucleic acid molecules is trivial. Such complementary antisense polynucleotides may include substitutions, additions, deletions, or transpositions, as long as specific hybridization to the relevant target sequence in any one of SEQ ID NOs:1-71 or 75-79 (or the sequences set forth in Example I) is retained as a functional property of the polynucleotide. Antisense polynucleotides that prevent transcription and/or translation of mRNA corresponding to PAL-18 may enhance or inhibit apoptosis or a MAP kinase signaling pathway and/or inhibit cancer progression. Antisense polynucleotides of various lengths may be produced and used, however, the sequence length is typically at least 20 consecutive nucleotides that are substantially or wholly identical to a sequence contained within any one of SEQ ID NOs:1-71 or 75-79. (see U.S. Pat. No. 5,691,179 and Antisense RNA and DNA, D. A. Melton, Ed., Cold Spring Harbor Laboratory, Cold Spring Harbor, N.Y. 1988, each of which is incorporated herein by reference).

2. PAL-18Polypeptides and Fragments Thereof

PAL-18 polypeptides vary in exon lengths as indicated by SEQ ID NOs:72-74 and nucleic acid sequences SEQ ID NOs:75-79 which represent composites of various clones. PAL-18 polypeptides of the present invention have calculated amino acid lengths of 379 and 411 amino acids (SEQ ID NOs:72-74) which exhibit homologies with known genes. In addition, as is clear form the various sequences, PAL-18 polypeptides of other lengths are encoded by splice variant forms. For diagnostic purposes all fragments even those disclosed in the art are applicable with this newly identified use. This includes all sequences identified in Example 1.

The polypeptides appear to be a human homolog to the Bro1 family of proteins, which interact with MAP kinase signaling pathway proteins (see, e.g., Nickas and Yaffe, Mol. Cell. Biol. 16(6):2585-2593, 1996). When using, the 411 amino acid sequence for searching databases using the BLAST 2.0 Algorithm, the protein homologies are: BRO1 25% identity and 41% conservation over 229 residues; C. elegans YNK1 23% identity, 40% conservation over 286 residues; PTP-TD14 (a rodent protein-tyrosine phosphatase involved in ras-dependent pathways; Cao et al., J. Biol. Chem. 273(33):21077-21083, 1998) 21% identity, 42% conservation over 213 residues; C. elegans B0507.2 39% identity, 58% conservation over the full 411 residues of SEQ ID NO:79/74. Further, searching with the full 411 residue PAL-18 polypeptide using the BLASTP 2.1.2 Algorithm (Altschul et al., Nuc. Acid Res. 25:3389-3402, 1997) reveals 89% identity, 93% conservation over 101 residues with putative mouse protein AK002489; 25% identity, 41% conservation over 247 residues of palA (Z83333, accession); 21% identity, 39% conservation over 258 residues of Yor275cp; and 27% identity, 43% conservation over 159 residues of programmed cell death 6-interacting protein and ALG-2 interacting protein 1 (NP037506.1 and AF151793, accession numbers); and 22% identity, 34% conservation over 333 residues of Alix (mus musculus, programmed cell death 6 interacting protein, AJ005073). Accordingly, the PAL-18 polypeptide is likely involved in the MAP kinase signaling pathway and/or in related apoptotic pathways as is predicated for BRO1, see Vito et al., J. Biol. Chem. 274(3):1533-1540, 1999; Che et al., J. Biol. Chem. 274(9):5522-5531, 1999; Missotten et al., Cell Death Differ 6(2):124-129, 1999. As such, modulation of PAL-18 may be critical in controlling cell-death and/or signaling pathways and thus related indications such as cancer. In this regard the crossover of the MAP kinase pathways and apoptotic pathways as well as the relation of the same to cancer progression has been the discussion of several recent journal articles, see e.g., Miki et al., Anticancer Res. 19(6B):5283-5291, 1999; Yin et al., Anticancer Res. 19(5B):4297-4303, 1999; Marushige et al., Anticancer Res. 19(5B):3865-3871, 1999. Accordingly, PAL-18 polypeptides are of interest as not only as modulators of cancer growth and progression, but also as a potentially drug target for MAP kinase signaling pathway and apoptotic pathway related indications such as autoimmune diseases etc.

In other embodiments, a PAL-18 polypeptide is fused to other polypeptide sequences to aid in expression and/or purification. For example, fusions with His6, T7, and Flag tags may aid in purification and/or immunoidentification.

PAL-18 polypeptides of the subject invention may be constructed from the described PAL-18 sequences by a variety of methods known in the art. A preferred method is amplification (e.g., polymerase chain reaction (PCR)) to selectively amplify the individual regions and place these in cloning vectors such as pUC. Moreover, such PCR reactions can be performed in a variety of ways such that the primers used for amplification contain specific restriction endonuclease sites to facilitate insertion into a vector.

Further, a variety of other methodologies besides PCR may be used to attain the desired construct. For example, one skilled in the art may employ isothermal methods to amplify the nucleotide sequence of interest, using existing restriction endonuclease sites present in the nucleotide sequence to excise and insert sequences, or by the introduction of distinct restriction endonuclease sites by site-directed mutagenesis followed by excision and insertion. These and other methods are described in Sambrook et al., supra; Ausubel, et al., supra. Briefly, one methodology is to generate single-stranded cDNA encoding PAL-18, followed by annealing a primer, which is complementary except for the desired alteration (e.g., a small insertion, deletion, or mutation such that a unique restriction site is created between the domains). Bacterial cells are transformed and screened for those cells which contain the desired construct. This construct is then digested to liberate the desired sequences, which can then be purified and religated into the appropriate orientation.

In addition, the invention provides fragments of SEQ ID NOs:72-74 and sequences with at least about 70% identity therewith. A fragment of SEQ ID NOs:72-74 may be defined both structurally and functionally in that it has the same contiguous sequence as a portion of SEQ ID NOs:72-74 and those sequences with at least about 70% identity therewith and at least one activity characteristic of PAL-18. Such activities can be binding and/or activating or deactivating MAP kinase pathway proteins and/or apoptotic pathway proteins, or acting as an antigen or as a tag for imaging. More specifically the PAL-18 polypeptide or fragment thereof as defined above, may enhance or inhibit phosphorylation or apoptosis, or may bind or induce the production of an anti-PAL18 antibody. A fragment of SEQ ID NOs:72-74 and those sequences with at least about 70% identity therewith comprise at least 8 contiguous residues. In other embodiments of the invention, a fragment comprises an amino acid sequence of at least 10 or 12 contiguous residues. In further embodiments of the invention, a fragment comprises an amino acid sequence of at least 15 or 20 contiguous residues. In yet other embodiments of the invention, a fragment comprises an amino acid sequence of at least 25 or 30 contiguous residues. In another embodiment of the invention, a fragment comprises an amino acid sequence of at least 50 contiguous residues. However, an amino acid sequence that consists of the identical amino acid sequence encoded by ESTs having GenBank accession numbers AA386306, AI243754, AA295847, AA994364, AI911339, AA884496, AI243754 or any contiguous portion thereof, is not a functional fragment of SEQ ID NOs:72-74 and those sequences with at least about 70% identity therewith encompassed within the invention.

It is understood that fragments of a PAL-18 polypeptide include fragments with substitutions of conserved and non-essential amino acids of portions of SEQ ID NOs:72-74 and, therefore, include, for example, fragments of eukaryotic homologs of SEQ ID NO:72-74 and sequences having at least about 70% identity therewith.

B. Vectors, Host Cells and Methods of Expressing and Producing Protein

PAL-18 may be expressed in a variety of host organisms. In certain embodiments, PAL-18 is produced in bacteria, such as E. coli, or mammalian cells (e.g., CHO and COS-7), for which many expression vectors have been developed and are available. Other suitable host organisms include other bacterial species, and eukaryotes, such as yeast (e.g., Saccharomyces cerevisiae), and insect cells (e.g., Sf9).

In one embodiment, a DNA sequence encoding a PAL-18 polypeptide is introduced into an expression vector appropriate for the host cell. In certain embodiments, nucleic acid sequences encoding PAL-18 are inserted into a vector such that a fusion protein is produced. The PAL-18 sequence is derived as described herein. As discussed above, the sequence may contain alternative codons for each amino acid with multiple codons. The alternative codons can be chosen as “optimal” for the host species. Restriction sites are typically incorporated into the primer sequences and are chosen with regard to the cloning site of the vector. If necessary, translational initiation and termination codons can be engineered into the primer sequences.

At a minimum, the vector will contain a promoter sequence. As used herein, a “promoter” refers to a nucleotide sequence that contains elements that direct the transcription of a linked gene. At a minimum, a promoter contains an RNA polymerase binding site. More typically, in eukaryotes, promoter sequences contain binding sites for other transcriptional factors that control the rate and timing of gene expression. Such sites include TATA box, CAAT box, POU box, AP1 binding site, and the like. Promoter regions may also contain enhancer elements. When a promoter is linked to a gene so as to enable transcription of the gene, it is “operatively linked”.

Other regulatory sequences may be included. Such sequences include a transcription termination sequence, secretion signal sequence, origin of replication, selectable marker, and the like. The regulatory sequences are operationally associated with one another to allow transcription or translation.

The expression vectors used herein include a promoter designed for expression of the proteins in a host cell (e.g., bacterial). Suitable promoters are widely available and are well known in the art. Inducible or constitutive promoters are preferred. Such promoters for expression in bacteria include promoters from the T7 phage and other phages, such as T3, T5, and SP6, and the trp, lpp, and lac operons. Hybrid promoters (see, U.S. Pat. No. 4,551,433), such as tac and trc, may also be used. Promoters for expression in eukaryotic cells include the P10 or polyhedron gene promoter of baculovirus/insect cell expression systems (see, e.g., U.S. Pat. Nos. 5,243,041, 5,242,687, 5,266,317, 4,745,051, and 5,169,784), MMTV LTR, CMV IE promoter, RSV LTR, SV40, metallothionein promoter (see, e.g., U.S. Pat. No. 4,870,009), ecdysone response element system, tetracycline-reversible silencing system (tet-on, tet-off), and the like.

The promoter controlling transcription of PAL-18 may itself be controlled by a repressor. In some systems, the promoter can be derepressed by altering the physiological conditions of the cell, for example, by the addition of a molecule that competitively binds the repressor, or by altering the temperature of the growth media. Preferred repressor proteins include, but are not limited to the E. coli lacI repressor responsive to IPTG induction, the temperature sensitive λcI857 repressor, and the like.

In other optional embodiments, the vector also includes a transcription termination sequence. A “transcription terminator region” has either a sequence that provides a signal that terminates transcription by the polymerase that recognizes the selected promoter and/or a signal sequence for polyadenylation.

In one aspect, the vector is capable of replication in the host cells. Thus, when the host cell is a bacterium, the vector preferably contains a bacterial origin of replication. Bacterial origins of replication include the fl-ori and col E1 origins of replication, especially the ori derived from pUC plasmids. In yeast, ARS or CEN sequences can be used to assure replication. A well-used system in mammalian cells is SV40 ori.

The plasmids also preferably include at least one selectable marker that is functional in the host. A selectable marker gene includes any gene that confers a phenotype on the host that allows transformed cells to be identified and selectively grown. Suitable selectable marker genes for bacterial hosts include the ampicillin resistance gene (Amp^{r}), tetracycline resistance gene (Tc^{r}) and the kanamycin resistance gene (Kan^{r}). The kanamycin resistance gene is presently preferred. Suitable markers for eukaryotes usually require a complementary deficiency in the host (e.g., thymidine kinase (tk) in tk−hosts). However, drug markers are also available (e.g., G418 resistance and hygromycin resistance).

The sequence of nucleotides encoding PAL-18 may also include a secretion signal, whereby the resulting peptide is a precursor protein processed and secreted. The resulting processed protein may be recovered from the periplasmic space or the fermentation medium. Secretion signals suitable for use are widely available and are well known in the art (von Heijne, J. Mol. Biol. 184:99-105, 1985). Prokaryotic and eukaryotic secretion signals that are functional in E. coli (or other host) may be employed. The presently preferred secretion signals include, but are not limited to, those encoded by the following E. coli genes: pe1B (Lei et al., J. Bacteriol. 169:4379, 1987), phoA, ompA, ompT, ompF, ompC, beta-lactamase, and alkaline phosphatase.

One skilled in the art will appreciate that there are a wide variety of suitable vectors for expression in bacterial cells and which are readily obtainable. Vectors such as the pET series (Novagen, Madison, Wis.), the tac and trc series (Pharmacia, Uppsala, Sweden), pTTQ18 (Amersham International plc, England), pACYC 177, pGEX series, and the like are suitable for expression of a PAL-18 polypeptide. Baculovirus vectors, such as pBlueBac (see, e.g., U.S. Pat. Nos. 5,278,050, 5,244,805, 5,243,041, 5,242,687, 5,266,317, 4,745,051, and 5,169,784; available from Invitrogen, San Diego) may be used for expression in insect cells, such as Spodoptera frugiperda sf9 cells (see, U.S. Pat. No. 4,745,051). The choice of a bacterial host for the expression of a PAL-18 polypeptide is dictated in part by the vector. Commercially available vectors are paired with suitable hosts.

A wide variety of suitable vectors for expression in eukaryotic cells are also available. Such vectors include pCMVLacI, pXT1 (Stratagene Cloning Systems, La Jolla, Calif.); pCDNA series, pREP series, pEBVHis (Invitrogen, Carlsbad, Calif.). In certain embodiments, the PAL-18 nucleic acid molecule is cloned into a gene targeting vector, such as pMC1neo, a pOG series vector (Stratagene Cloning Systems).

PAL-18 polypeptides may be isolated by standard methods, such as affinity chromatography, size exclusion chromatography, metal ion chromatography, ionic exchange chromatography, HPLC, and other known protein isolation methods. (see generally Ausubel et al. supra; Sambrook et al. supra). An isolated purified protein gives a single band on SDS-PAGE when stained with Coomassie blue. PAL-18 polypeptides may be expressed using tags to facilitate isolation such as a hexa-his (His6) fusion protein and isolated by metal-containing chromatography, such as nickel-coupled beads. Briefly, a sequence encoding His6 is linked to a DNA sequence encoding a PAL-18 polypeptide. Although the His6 sequence can be positioned anywhere in the molecule, preferably it is linked at the 5′ end or at the 3′ end immediately preceding the termination codon. The fusion may be constructed by any of a variety of methods. A convenient method is amplification of the PAL-18 nucleic acid molecule using a downstream primer that contains the codons for His6. In a similar manner T7, Flag, and a variety of other fusions are possible.

In further embodiments, these proteins may also be crystallized and subjected to X-ray analysis to determine the 3-dimensional structure for rational drug design or utilized to generate antibodies.

C. Uses of PAL-18 Nucleic Acid Molecules and Encoded Products Thereof

As noted above, the present invention in one aspect is directed toward methods of screening for (detecting or monitoring) or treating an abnormality (such as cancer or autoimmune disorders) in which a PAL-18 polypeptide is associated. As disclosed in the present invention, PAL-18 polypeptides and/or nucleic acid molecules (including splice variants and single nucleotide polymorphisms) may be found to be associated with the presence of tumor cells and found to survive in detectable concentrations in specimens from patients with tumors. In this regard, varying levels of all or certain PAL-18 polypeptides or mRNA molecules may be present in a particular cancer, tissue type or biological sample. Furthermore, varying PAL-18 levels or varying levels of splice variants or SNPs may be found associated with a variety of cancers, including colorectal, renal, bladder and other cancers, and may be detected in a variety of ways, including by detecting PAL-18 itself or a nucleic acid molecule encoding PAL-18. Methods for detecting the presence (i.e., qualitative or quantitative) of PAL-18 include those based on physical properties, immunological properties, biochemical properties and combinations thereof (e.g., physical size of the molecule, nucleic acid sequence, amino acid sequence, binding by monoclonal or polyclonal antibodies, ligand binding, enzymatic properties, and combinations thereof). For example, relative levels of expression of PAL-18 or PAL-18 isoforms/variants may be relevant to cancer diagnosis. In one example, PAL-18 appears to be upregulated in colorectal cancers.

Purified antigen (PAL-18 or fragments thereof), partially purified antigen or biological samples containing antigen may be used to produce antibodies that specifically bind to the antigen. Antibodies that specifically bind are those with an affinity of about 10⁶ liters/mol or greater. Either polyclonal antibodies or monoclonal antibodies may be generated. Polyclonal antibodies may be produced by immunization of an animal and subsequent collection of its sera. It is generally preferred to follow the initial immunization with one or more booster immunizations prior to sera collection. Monoclonal antibodies are generally produced by the method of Kohler and Milstein (Nature 256:495-497, 1975; Eur. J. Immunol. 6:511-519, 1976). Briefly, the lymph nodes and/or spleens of an animal injected with antigen in pure or impure form are fused with myeloma cells to form hybrid cell lines (“hybridomas” or “clones”). Each hybridoma secretes a single type of immunoglobulin specific for the antigen and, like the myeloma cells, has the potential for indefinite cell division.

Antigen in pure or impure form (“immunogen”) is used for the immunization. Preferably, the animals are immunized with at least 100 ng each of the immunogen, most preferably greater than 500 ng each. For immunization, the immunogen may be adsorbed to a solid phase matrix, preferably to nitrocellulose paper. The paper is then introduced into the animal. Techniques for introduction of the adsorbed antigen preparation include implantation (U.S. Pat. No. 4,689,220) or solubilization of the solid phase and injection of the solubilized material (Knudsen, Anal. Biochem. 147:285-288, 1985). The solid phase matrix may be solubilized in an appropriate organic solvent (e.g., DMSO) and either mixed with adjuvant or saline, or injected directly.

Alternatively, the immunogen may be injected in the absence of a solid matrix and/or adjuvant. Injection or implantation may be intraperitoneal, intra-foot pad, subcutaneous, intramuscular or intravenous, but preferably intraperitoneal. The animals may also be injected with antigen complexed with adjuvant, such as Freund's adjuvant. Single or multiple booster immunizations are used. Between one and seven days prior to the fusion date, preferably on days one through four, intravenous injections of the immunogen may be given daily.

Between one and seven days, preferably four days, after the administration of the final booster immunization, spleens or portions thereof are harvested from the immunized animals. At this time, the lymph nodes may also be harvested and included in the cell preparation. The harvested organs are minced using techniques which disrupt the structure of the organ, but which are not detrimental to the lymphocytes. The organs are preferably minced with scissors, passed through a mesh screen and mixed with growth medium to enrich the preparation for lymphocytes. The minced and strained tissue is harvested by centrifugation, then mixed with growth medium to form a cell suspension. The red blood cells may be lysed by adding a hypotonic or hypertonic solution to the cell suspension. A preferred method for cell lysis is to add distilled water to the suspensions and quickly return the suspensions to an isotonic state with a hypertonic sodium chloride solution. Any remaining tissue may be removed by filtration through gauze.

The harvested cell suspension is then mixed with a myeloma cell line, preferably one which is syngeneic with the immunized animal. Myeloma cell lines from various species are widely available through, for example, American Type Culture Collection (ATCC), Rockville, Md. Myeloma cell lines commonly used include P3X63Ag8 (ATCC TIB 9), SP2/0-Ag14 (ATCC CRL 1581), FO (ATCC CRL 1646) and 210-RCY-Ag1 (Galfre et al., Nature 277:131, 1979).

The myeloma cells are cultured in an appropriate mammalian cell growth medium, a variety of which are generally known in the art and available from commercial sources. Mammalian cell lines are routinely grown between 36° C. and 40° C. under conditions which maintain an optimal pH between 6.0 and 8.0, preferably about pH 7.2. pH may be maintained through the use of a variety of buffer systems known in the art. A preferred buffer system involves growing the cells in a bicarbonate buffer in a humidified incubator containing CO₂, preferably about 7% CO₂.

The fusion between the lymphocytes from the immunized animal and the myeloma cells may be carried out by a variety of methods described in the literature. These methods include the use of polyethylene glycol (PEG) (Brown et al., J. Biol. Chem. 255:4980-4983, 1980) and electrofusion (Zimmerman and Vienken, J. Membrane Biol. 67:165-182, 1982). An electrofusion generator is commercially available from Biotechnologies and Experimental Research, Inc., San Diego, Calif.

Following the fusion, the cells are plated into multi-well culture plates, preferably 96-well plates. A reagent which selectively allows for the growth of the fused myeloma cells over the unfused cells is added to the culture medium. A preferred selection technique uses HAT (hypoxanthine, aminopterin, thymidine) selection. Other selection techniques may also be used depending on the myeloma cell line chosen.

Alternative methods of producing monoclonal antibodies utilize in vitro immunization techniques. Lymphocytes may be harvested from lymphoid organs, such as spleen or lymph nodes, or from whole blood as peripheral blood lymphocytes. The lymphocytes are put into culture in the presence of the appropriate immunogen. Often immunostimulatory polypeptides will be added to the culture medium concurrently. At various times following the culturing of the lymphocytes in vitro, the lymphocytes are harvested and fused with a myeloma cell line as described above.

Other techniques for producing and maintaining antibody secreting lymphocyte cell lines in culture include viral transfection of the lymphocyte to produce a transformed cell line which will continue to grow in culture. Epstein-Barr virus (EBV) has been used for this technique. EBV transformed cells do not require fusion with a myeloma cell to allow continued growth in culture.

Thymocytes may be used as a feeder layer to condition the medium for the fused cells. Alternatively, peritoneal macrophages or non-immune spleen cells may be used as a feeder layer. Another alternative is to use conditioned medium from thymocytes or macrophages. Thymocytes may be prepared from juvenile mice less than 8 weeks old. The thymus glands are harvested and minced using techniques which disrupt the thymus gland but are not detrimental to the thymocytes. This procedure is preferably carried out using scissors to mince the tissue, followed by passage of the tissue through a mesh screen. The minced and strained cell material is then harvested by centrifugation. Cell suspensions are made using growth medium. Any remaining connective tissue may be removed by filtration through gauze.

At an appropriate time following the day the cells are fused, the fused cells (hybridomas) are then analyzed for the production of antibody against the antigen. This “screening” can be done by a wide variety of techniques, including Western blot, ELISA, immunoprecipitation, effect on biological activity assays and immunocytochemical staining. These techniques and others are well described in the literature. (See, for example, J. G. R. Hurrell (ed.), Monoclonal Hybridoma Antibodies: Techniques and Applications, CRC Press Inc., Boca Raton, Fla., 1982.) Introduction of a screening procedure permits further definition of antibodies of useful reactivity. For example, antigen purified from a biological sample of a patient with a bladder cancer may be used in any of the above-named techniques to define antibodies which react, for example, to determinants which are common to patients with the disease.

Hybridomas which secrete antibodies of interest are maintained in culture. The cells are expanded in culture and at the same time may be cloned in such a manner as to obtain colonies originating from single cells. This provides for the monoclonal nature of the antibodies obtained from the hybridomas. A wide variety of techniques exist for cloning cells, including limiting dilution, soft agar cloning and fluorescence-activated cell sorting.

Once clones of cells are obtained, they are re-assayed for the production of the antibody of interest. These cells are then expanded in culture to allow for the production of larger amounts of the antibody. Methods for expansion of the cells include maintaining the cells in culture, placement of the cells in a bioreactor or other type of large-scale cell culture environment, or culturing the cells using various agar or gelatin carrier matrices. Antibodies are then isolated from the cell culture media.

Antibodies may be purified from conditioned media or ascites fluid by a variety of methods known in the art. These methods include ammonium sulfate precipitation, ion exchange chromatography (see Hurrell, ibid.) and high pressure liquid chromatography using a hydroxylapatite support (Stanker et al., J. Immunol. Methods 76:157, 1985). A preferred method for purifying antibodies from conditioned media or ascites fluid utilizes a commercially available Protein A-Sepharose® CL-4B column or Protein G Sepharose® (Pharmacia, Piscataway, N.J.; Sigma, St. Louis, Mo.) or ABX mixed ion exchange resin (J T Baker, Phillipsburg, N.J.). Antibodies may be purified with these columns using conditions suggested by the manufacturer.

As can be readily appreciated by those of ordinary skill in the art the presence of antigen may be detected by immunological means using an antibody produced as detailed above. The means for detecting the presence of antigen may be in a direct or indirect test format. In a direct test format, that which is observed or measured is proportional to (i.e., directly reflective of) antigen present in a sample. Conversely, in an indirect test format, that which is observed or measured is inversely proportional to (i.e., indirectly reflective of) antigen present in a sample. Indirect formats include competitive and inhibition assay formats. As used herein, the term “antibody” includes both polyclonal and monoclonal antibodies as well as single chain antibodies; and may be an intact molecule, a fragment thereof, or a functional equivalent thereof; and may be genetically engineered. Examples of antibody fragments include F(ab′)₂, Fab′, Fab and Fv. Detection may be, for example, by Western blot analysis utilizing antigen immobilized on nitrocellulose or Immobilon or similar matrix, in conjunction with specific antibodies to the antigen. Detection can also be achieved by immunoassay. In one embodiment, antigen is isolated from a sample and contacted with an appropriate detection antibody. Antigen may be isolated by capture on a solid support or with a “capture” antibody prior to or simultaneous with a “detection” antibody. In another embodiment, immunocomplexes are formed between an antibody and antigen, without prior purification of the antigen. Incubation of a sample with an antibody is under conditions and for a time sufficient to allow immunocomplexes to form. Detection of antigen by immunological means is also amenable to quantification where it is desired to determine the amount of antigen.

Detection of one or more immunocomplexes formed between antigen and an antibody specific for the antigen may be accomplished by a variety of known techniques, including radioimmunoassays (RIA) and enzyme linked immunosorbent assays (ELISA).

The immunoassays known in the art include the double monoclonal antibody sandwich immunoassay technique of David et al. (U.S. Pat. No. 4,376,110); monoclonal-polyclonal antibody sandwich assays (Wide et al., in Kirkham and Hunter (eds.), Radioimmunoassay Methods, E. and S. Livingstone, Edinburgh, 1970); the “western blot” method of Gordon et al. (U.S. Pat. No. 4,452,901); immunoprecipitation of labeled ligand (Brown et al., J. Biol. Chem. 255:4980-4983, 1980); enzyme-linked immunosorbant assays as described by, for example, Raines and Ross (J. Biol. Chem. 257:5154-5160, 1982); immunocytochemical techniques, including the use of fluorochromes (Brooks et al., Clin. Exp. Immunol. 39: 477, 1980); and neutralization of activity (Bowen-Pope et al., Proc. Natl. Acad. Sci. USA 81:2396-2400, 1984). In addition to the immunoassays described above, a number of other immunoassays are available, including those described in U.S. Pat. Nos.: 3,817,827; 3,850,752; 3,901,654; 3,935,074; 3,984,533; 3,996,345; 4,034,074; and 4,098,876.

For detection purposes, the antibodies may either be labeled or unlabeled. When unlabeled, the antibodies find use in agglutination assays. In addition, unlabeled antibodies can be used in combination with other labeled antibodies (second antibodies) that are reactive with the antibody, such as antibodies specific for immunoglobulin. Alternatively, the antibodies can be directly labeled. Where they are labeled, the reporter group can include radioisotopes, fluorophores, enzymes, luminescers, or visible particles (e.g., colloidal gold and dye particles). These and other labels are well known in the art and are described, for example, in the following U.S. Pat. Nos.: 3,766,162; 3,791,932; 3,817,837; 3,996,345; and 4,233,402.

Typically in an ELISA assay the target antigen (for a competitive or inhibition assay format) or immobilized capture antibody is adsorbed to the surface of a microtiter well. Residual protein-binding sites on the surface are then blocked with an appropriate agent, such as bovine serum albumin (BSA), heat-inactivated normal goat serum (NGS), or BLOTTO (buffered solution of nonfat dry milk which also contains a preservative, salts, and an antifoaming agent). The well is then incubated with a sample suspected of containing antigen. The sample can be applied neat, or, more often, it can be diluted, usually in a buffered solution which contains a small amount (0.1%-5.0% by weight) of protein, such as BSA, NGS, or BLOTTO. After incubating for a sufficient length of time to allow specific binding to occur, the well is washed to remove unbound protein and then incubated with a detection antibody labeled with a reporter group, or an anti-immunoglobulin antibody labeled with a reporter group. The reporter group can be chosen from a variety of enzymes, including horseradish peroxidase, beta-galactosidase, alkaline phosphatase, and glucose oxidase. Sufficient time is allowed for specific binding to occur, the well is again washed to remove unbound conjugate, and the substrate for the enzyme is added. Color is allowed to develop and the optical density of the contents of the well is determined visually or instrumentally.

In one preferred embodiment of the present invention, a reporter group is bound to the detection antibody. The step of detecting an immunocomplex involves removing substantially any unbound antibody and then detecting the presence or absence of the reporter group.

In another preferred embodiment, a reporter group is bound to a second antibody capable of binding to the antibody specific for antigen. The step of detecting an immunocomplex involves (a) removing substantially any unbound antibody, (b) adding the second antibody, (c) removing substantially any unbound second antibody and then (d) detecting the presence or absence of the reporter group. Where the antibody specific for the fragment is derived from a mouse, the second antibody is an anti-murine antibody.

In a third preferred embodiment for detecting an immunocomplex, a reporter group is bound to a molecule capable of binding to the immunocomplex. The step of detecting involves (a) adding the molecule, (b)removing substantially any unbound molecule, and then (c) detecting the presence or absence of the reporter group. An example of a molecule capable of binding to the immunocomplex is protein A.

It will be evident to one skilled in the art that a variety of methods for detecting the immunocomplex may be employed within the present invention. Reporter groups suitable for use in any of the methods include radioisotopes, fluorophores, enzymes, luminescers, and visible particles (e.g., colloidal gold and dye particles).

Alternatively, rather than detecting PAL-18 itself, a nucleic acid molecule encoding PAL-18, PAL-18 variants, and/or fragments of either can be detected. Such a nucleic acid molecule may be a deoxyribonucleic acid (DNA) or a ribonucleic acid (RNA). Generally, a nucleic acid molecule encoding for PAL-18 is detected by amplification of the nucleic acid. A variety of methods may be utilized in order to amplify a selected sequence, including, for example, RNA amplification (see Lizardi et al., Bio/Technology 6:1197-1202, 1988; Kramer et al., Nature 339:401-402, 1989; Lomeli et al., Clinical Chem. 35(9):1826-1831, 1989; U.S. Pat. No. 4,786,600), and DNA amplification utilizing ligase chain reaction (“LCR”) or polymerase chain reaction (“PCR”) (see U.S. Pat. Nos. 4,683,195, 4,683,202, and 4,800,159) (see also U.S. Pat. Nos. 4,876,187 and 5,011,769, which describe an alternative detection/amplification system comprising the use of scissile linkages), or other nucleic acid amplification procedures that are well within the level of ordinary skill in the art. With respect to PCR, for example, the method may be modified as known in the art. Transcriptional enhancement of PCR may be accomplished by incorporation of bacteriophage T7 RNA polymerase promoter sequences in one of the primary oligonucleotides, and immunoenzymatic detection of the products from the enhanced emitter may be effected using anti-RNA:DNA antibodies (Blais, Appl. Environ. Microbiol. 60:348-352, 1994). PCR may also be used in combination with reverse dot-blot hybridization (Iida et al., FEMS Microbiol. Lett. 114:167-172, 1993). PCR products may be quantitatively analyzed by incorporation of dUTP (Duplàa et al., Anal. Biochem. 212:229-236, 1993), and samples may be filter sampled for PCR-gene probe detection (Bej et al., Appl. Environ. Microbiol. 57:3529-3534, 1991).

Primers for the amplification of a selected sequence should be selected from sequences that are highly specific to PAL-18 encoding nucleic acid sequences and form stable duplexes with the target sequence. The primers should also be non-complementary, especially at the 3′ end, should not form dimers with themselves or other primers, and should not form secondary structures or duplexes with other regions of DNA. In general, primers (such as those described in greater detail below) of about 20 to 35 nucleotides are preferred, and can be easily synthesized using techniques well known in the art. PCR products, and other nucleic acid amplification products, may be quantitated using techniques known in the art (Duplàa et al., Anal. Biochem. 212:229-236, 1993; Higuchi et al., Bio/Technology 11:1026-1030).

A preferred embodiment involves assaying for the presence of specific messenger RNA (mRNA) encoding a PAL-18 polypeptide. More specifically, for example, as described herein, a cell sample may be lysed and the mRNA isolated, amplified and examined for the presence of mRNA specific for PAL-18. A variety of procedures may be used to detect the presence of antigen-specific mRNA. A particularly preferred method includes RT-PCR (Reverse Transcriptase based Polymerase Chain Reaction) amplification of mRNA.

Detecting the presence of PAL-18 in a sample (for example, a cell or tissue, a fecal specimen, a voided urine sample, a biopsy sample or material extracted from a cervical swab) has a variety of uses. See, e.g., Davidson et al., Carcinogenesis 19(2):252-257, 1998; Loktionov et al., Clin. Cancer Res. 4:337-342, 1998; Machiels et al., BioTechniques 28:286-290, 2000. For example, the present invention may be used for diagnostic purposes to screen warm-blooded animals, such as humans, for cancers such as colorectal cancer, renal cancer, bladder cancer, prostate cancer or related cancers (depending upon the source of the particular sample). Preferred sample sources for a particular cancer would be evident to one of ordinary skill in the art. For example, using a voided urine sample, one may screen for renal or bladder cancer. In a similar manner, the present invention may be used to monitor warm-blooded animals. In particular, a preferred use is to follow patients who have been previously diagnosed and treated for colorectal cancer, renal cancer, bladder cancer, prostate, or cervical cancer. Patients who are in remission (or may in fact be cured) can be monitored for the reappearance of colorectal, prostate, renal, bladder or cervical cancer. It may be desirable to use the present invention in conjunction with one or more other tests for colorectal, prostate, renal, bladder or cervical cancer to confirm positive or negative results obtained from use of the present invention.

Irrespective of the exact function(s) of PAL-18 in tumor cells, the present invention provides for the modulation of PAL-18 as a means of treating cancers. As used herein, the term “treating” cancer refers to one or more of a variety of beneficial effects, including, for example killing tumor cells, arresting the growth of a tumor, or prolonging the survival time of a tumor host. It will be evident to those of ordinary skill in the art that PAL-18 may be modulated in a variety of ways. For example, PAL-18 may be modulated by interrupting the production of PAL-18 by tumor cells or “inactivating” PAL-18 (e.g., blocking PAL-18, or its effect) following production by tumor cells. A preferred method of interrupting the production of the antigen is by use of DNA, or PNA (peptide nucleic acid), constructs with base sequence complementary to the antigen's mRNA. Such an approach is generically termed antisense technology. Typically, the PAL-18 antisense DNA is inserted into an appropriate vector (virus) which delivers it to the tumor cells. Once inside the target cells, the antisense construct specifically binds to mRNA coding for the PAL-18, thereby preventing its translation. Primary among other methods which may be used to interrupt production of the antigen is the use of specific molecules which block the transcription of the specific gene or genes coding for the PAL-18. Chemicals designed to block the ability of the tumor cell to produce antigen are preferably delivered in the vicinity of the tumor, rather than systemically. An alternative to use of antisense is to utilize ribozyme technology to effectuate the same (see, e.g., Lieber and Struass, Mol. Cell Biol. 15(1):540-551, 1995).

Another approach to antigen modulation is to use reagents to inhibit the activity of, or interfere with the binding sites on, PAL-18. One family of such reagents includes monoclonal antibodies, or fragments thereof (e.g., antigen binding fragments). With such reagents, as with those described above, delivery is preferably administered to the tumor site, rather than systemically. For the antibodies described above, reagent affinities should be at least about 10⁶ liters/mole and doses should be within the range of about 0.01 μg/kg body weight to 10 mg/kg body weight. In addition, the preferred type of tumor to be treated in this manner would be distinctly separate from the circulatory system. An antibody may be replaced, or supplemented, with a small organic molecule or amino acid based molecule (such as a peptide) with similar functional properties to the antibody. Thus, PAL-18 may be modulated such that the killing of cancer cells by the complement system is promoted.

The present invention also provides for the modulation of PAL-18 as a means for treating MAP kinase pathway or apoptotic pathway related disorders.

The above-described molecules (antibodies, peptides, organic compounds and antisense nucleic acids or peptide nucleic acids) are representative examples of agents that may modulate a PAL-18 polypeptide or a nucleic acid molecule encoding said polypeptide, for use in the treatment of a tumor cell. Such agents may be combined with a pharmaceutically acceptable carrier or diluent to form a composition. Additional components, such as traditional chemotherapeutic compounds, may be included with such an agent or a composition thereof.

Additionally, PAL-18 polypeptides may be used as targets for cancer or various tissue imaging. In this regard a binding agent, such as an antibody of the invention reactive with a PAL-18 polypeptide or fragment thereof is also useful for in vivo diagnostic applications for the detection of human carcinomas. One such approach involves the detection of tumors in vivo by tumor imaging techniques. According to this approach, for example, the antibody is labeled with an appropriate imaging reagent that produces a detectable signal. Examples of imaging reagents that can be used include, but are not limited to, radiolabels such as ¹³¹I, ¹¹¹In, ¹²³I, ⁹⁹mTc, ³²P, ¹²⁵I, ³H, and ¹⁴C, fluorescent labels such as fluorescein and rhodamine, and chemiluminescers such as luciferin. The antibody can be labeled with such reagents using techniques known in the art. For example, see Wensel and Meares, Radioimmunoimaging and Radioimmunotherapy, Elsevier, N.Y. (1983) for techniques relating to the radiolabeling of antibodies (see also Colcher et al., “Use Of Monoclonal Antibodies As Radiopharmaceuticals For The Localization Of Human Carcinoma Xenografts In Athymic Mice”, Meth. Enzymol., 121:802-16 (1986)).

In the case of radiolabeled antibody, the antibody is administered to the patient, localizes to the tumor bearing the antigen with which the antibody reacts, and is detected or “imaged” in vivo using known techniques such as radionuclear scanning using, e.g., a gamma camera or emission tomography (see, e.g., Bradwell et al., “Developments In Antibody Imaging”, in Monoclonal Antibodies For Cancer Detection And Therapy, Baldwin et al. (eds.), pp. 65-85 (Academic Press 1985)). The antibody is administered to the patient in a pharmaceutically acceptable carrier such as water, saline, Ringer's solution, Hank's solution or nonaqueous carriers such as fixed oils. The carrier may also contain substances that enhance isotonicity and chemical stability of the antibody such as buffers or preservatives. The antibody formulation is administered, for example, intravenously, at a dosage sufficient to provide enough gamma emission to allow visualization of the tumor target site. Sufficient time should be allowed between administration of the antibody and detection to allow for localization to the tumor target. For a general discussion of tumor imaging, see Allum et al., “Monoclonal Antibodies In The Diagnosis And Treatment of Malignant Conditions”, Surg. Ann. 18:41-64 (1986).

The compositions and modulating agents of the present invention may also be formulated with pharmaceutical excipients. Administration of such compositions will generally follow established protocols and exact amounts will be determined by clinical trials. The compositions of the present invention may be administered either alone, or as a pharmaceutical composition. Briefly, pharmaceutical compositions of the present invention may comprise one or more of compositions or modulating agents as described herein, in combination with one or more pharmaceutically or physiologically acceptable carriers, diluents or excipients. Such compositions may comprise buffers such as neutral buffered saline, phosphate buffered saline and the like, carbohydrates such as glucose, mannose, sucrose or dextrans, mannitol, proteins, polypeptides or amino acids such as glycine, antioxidants, chelating agents such as EDTA or glutathione, adjuvants (e.g., aluminum hydroxide) and preservatives. In addition, pharmaceutical compositions of the present invention may also contain one or more additional active ingredients.

Compositions of the present invention may be formulated for the manner of administration indicated, including for example, for oral, nasal, venous, intracranial, intraperitoneal, subcutaneous, or intramuscular administration. Within other embodiments of the invention, the compositions described herein may be administered as part of a sustained release implant. Within yet other embodiments, compositions of the present invention may be formulized as a lyophilizate, utilizing appropriate excipients which provide stability as a lyophilizate, and subsequent to rehydration. One skilled in the art may further formulate the enhancers or inhibitors of this invention in an appropriate manner, and in accordance with accepted practices, such as those disclosed in Remington's Pharmaceutical Sciences, Gennaro, Ed., Mack Publishing Co., Easton, Pa. 1990.

Materials and reagents utilized in various aspects of the present invention may be assembled in a kit. A kit for carrying out any of the detection or screening methods set forth herein may comprise an appropriate container, binding agents or hybridizing probes, and instructions for their use. In some instances, a kit may comprise of more than one binding agent and/or hybridizing probe.

Components of the kit may be provided in a dried or lyophilized form or in one or more liquid solutions. When the components are provided in liquid solution, the liquid solution is preferably a sterile, aqueous solution. When components are provided in a dried form, the dried form may be capable of reconstitution upon addition of a suitable solvent. Kits will generally be packaged in an outer container suitable for commercial sale and distribution. Kits of the invention may also comprise or be packaged with instructions for use and instruments for assisting in use.

All of the references including patents, patent applications, journal articles, etc., cited herein are hereby incorporated by reference in their entirety.

The following examples are offered by way of illustration and not by way of limitation.

### EXAMPLES

### Example I

### Cloning of PAL-18

Materials—Cloning vectors were purchased from Invitrogen (Carlsbad, Calif.) Other molecular biology materials were purchased from Clontech (Palo Alto, Calif.), Promega (Madison, Wis.), Stratagene (La Jolla, Calif.), or BioRad (Hercules, Calif.) as specified. primers were purchased from Midland Certified Reagents (Midland, Tex.) and presented in below Common reagents were purchased from Sigma Chemical Company (St. Louis Mo.).

TABLE 1

| Primer | Primer | | |
|---|---|---|---|
| SEQ ID | name | Sequence | Notes |
| 80 | MCPLF01 | TTTATGGTATACCAAATATGCTTCAAGACT | Improved PAL-175 |
| 81 | MCPLR01 | CAGCCAGTCTTGAAGCATATTTGGTATACC | PAL-175 reverse |
| 82 | MCPLF02 | CTTGAAGATGTGTTTCTACACAGCTTATGC | Forward from 541* |
| 83 | MCPLR02 | GACTGTTGGTCCAGGTCCTTTGGTTTCTCC | Backward from 715* |
| 84 | MCXLF01 | CAGGAAACAGCTATGACCATGATTACGC | TOPO-XL vector |
| | | | forward primer |
| 85 | MCXLR01 | GTAATACGACTCACTATAGGGCGAATTGG | TOPO-XL vector |
| | | | reverse pnmer |
| 86 | MCPLR03 | AATCCCATGGAAATTAATTCAAAAACAGC | Backward from 156* |
| | | ATCC | |
| 87 | MCPLF03 | GGTGAGACTTTATTGGCTAGTGATAAATGC | Forward from 584* |
| | | G | |
| 88 | MCPLR04 | GTGCTTCTGCCTTTGCATACAATTTTTCTGC | Backward from 671* |
| 89 | MCPLF04 | TATTCAAAATTTCAAGTGGACTGATACATT | Forward from 67* |
| | | GC | |
| 90 | MCPLF05 | GGATGAATCTACCCAAGAAAGCAAGTTAC | Forward from 34* |
| | | G | |
| 91 | MCPLR05 | AACTTGCTTTCTTGGGTAGATTCATCCAAA | Backward from 60* |
| | | G | |
| 92 | MCPLR06 | GTATCAGTCCACTTGAAATTTTGAATATAT | Backward from 93* |
| | | CG | |
| 93 | MCPLF06 | CAGAAGATGAAGGAAAAGAAGTTCATCGA | Nested vs. MCPLF01 |
| | | AGCC | |
| 94 | MCGTF01 | GGATTGGTGGCGACGACTCCTGGAGCCCG | Vector primer for HFL |
| | | | library |
| 95 | MCGTR01 | AGACCAACTGGTAATGGTAGCGACCGGCG | Vector primer for HFL |
| | | C | library |
| 96 | MCPLF07 | GCAAAGGCAGAAGCACTGTGTAAAGAATA | Forward from 656* |
| | | TGG | |
| 97 | MCPLF08 | CTGTTCTTTAGGAAACTTGGAAACCTTGTG | Forward from 731* |
| 98 | MCPLF09 | CTGGAAAATTTAGTGTTATTGGAATCAAA | Forward from 702 of |
| | | GG | 64.2.5.F03 |
| 99 | MCPLR07 | TGAACAGTTCAAGGAGTCGTGCCCTGGAT | Forward from 474 of |
| | | GACC | 64.2.5.F01 inverted |
| 100 | MCPLF10 | CCAGGGCACGACTCCTTGAACTGTTCACTG | Backward from 500 of |
| | | | 64.2.5.F01 inverted |
| 101 | MCPLR08 | ATCCCTTGAGTCTTATTGATTAGTACTAAC | Backward from 196 of |
| | | TC | 64.2.5.F04 |
| 102 | MCPTF01 | CGAATTTAATACGACTCACTATAGGGAATT | Image Consortium |
| | | TGG | vector forward primer |
| 103 | MCPTR01 | TGCCAAGCTAAAATTAACCCTCACTAAAG | Image Consortium |
| | | G | vector reverse primer |
| 104 | MCPLF11 | TATCAAAAAGCTGATCATACTTTATCCAG | 16-base exon-specific |
| | | | primer |
| 105 | MCPLR09 | TTAGGAGATGTAGCACCCAGTGTCCTTTTG | Expression primer |
| | | AGGTTTGATG | with stop codon |
| 106 | MCPLR10 | GGAGATGTAGCACCCAGTGTCCTTTTGAG | Expression primer |
| | | GTTTGATG | without stop codon |
| 107 | MCPLF12 | ATGACCCATTGGTTTCATAGGAACCCATTA | Forward from |
| | | AAAGC | beginning of ORF |
| 108 | MCPLF13 | CAGCAGGATGCTGTTTTTGAATTAATTTCC | 96-base exon-specific |
| | | ATGGGATTTAATGTAGC | primer |
| 109 | MCPLF14 | AAGGACAGGTTCCAAGTATAACAGAAG | Skips 96-base exon |
| 110 | MCPLR11 | CACTTGTAGGAGGAAATTCGAAAGGTATA | 160-base exon-specific |
| | | GG | primer |
| 111 | MCPLR12 | TTCTGGTTTGGGTTTAGTATAAATCCATTT | Skips 160-base exon |
| | | T | |
| 112 | 42M | GCTGGTAAATGTCCTCT | Upstream Factor H |
| | | | primer |
| 113 | 1040RT | TCTGGATAATCACAAGGTTTC | Downstream Factor H |
| | | | primer |
| | | | *Numbering according |
| | | | to original 778-base |
| | | | amplicon |

DNA Sequencing

All DNA sequencing was performed in the laboratory of Dr. Leroy Hood, using an ABI Prisms 373 DNA Analyzer from PE Applied Biosystems (Foster City, Calif.). Sequencing reactions made use of PE Applied Biosystems big-dye terminator kits. Gels were analyzed with ABI Prism Sequencing Analysis Version 3.3 (PE Applied Biosystems).

Agarose Gel Electrophoresis (AGE)

0.75 g refined agarose and 0.6 uL of 10 mg/mL ethidium bromide were added to 50 mL of Tris-acetate-EDTA buffer (Sigma; 0.04 M Tris acetate, 0.01 M EDTA, pH 8.3) and heated in a microwave at full power for 100 seconds with occasional mixing and poured with a ten-well comb, a 14-well comb, or both. The gels were allowed to cool for at least 20 minutes before use.

After cooling, the gels were covered with Tris-acetate-EDTA buffer and loaded with 6 uL sample or molecular weight standard (BioRad Amplisize 50-2000 bp standards or BioRad PCR ruler) mixed with 1.2 uL 6×loading buffer (0.25% bromophenol blue, 40% sucrose).

TA Cloning

A cocktail was made containing enough volume for at least 10% more than the planned reactions. This cocktail contained (per reaction):

0.3 uL ligation buffer (60 mM Tris-HCl, pH 7.5; 60 mM MgCl₂; 50 mM NaCl; 1 mg/mL bovine serum albumin; 70 mM B-mercaptoethanol; 1 mM ATP; 20 mM dithiothreitol; 10 mM spermidine)

0.6 uL TA vector (pCR II) (25 ng/uL in 10 mM Tris-HCl, 1 mM EDTA, pH 7.5)

0.3 uL T4 ligase (4.0 Weiss units/uL)

1.05 uL H₂O

After 2.25 uL of the cocktail was aliquoted into each tube on ice, 0.75 uL of the PCR product to be cloned was added and the mixture was transferred to an air incubator or thermocycler maintaining 14° C. for 4-30 hours.

It was later determined that although this method is workable, a lower background of self-ligated plasmids was obtained if the ligase was withheld from the cocktail and added after the PCR product into the individual reaction vessels.

Plasmid Growth and Preparation

2-mL cultures of Luria-Bertani medium (1.0% tryptone, 0.5% yeast extract, 1.0% NaCl, pH 7.0) with 60 ug/mL ampicillin or carbenicillin were inoculated with well-isolated colonies of E. coli potentially containing the plasmid of interest. Cultures which proved to be positive by screening were used to prepare plasmid. 1.5 mL of the resuspended culture were transferred to a 1.5-mL microcentifuge tube and centrifuged at maximum speed in a microfuge for 30 sec. Unless otherwise specified, plasmid was prepared from the pellet according to the BioRad QuantumPrep protocol.

Initial Cloning of PAL-18 Sequences

Reverse Transcription

20-uL Reverse Transcription reactions contained 4 uL of 25 mM MgCl₂, 2 uL of 10×PCR buffer (Promega catalog #M190A), 2 uL each of 10 mM dATP, dCTP, dGTP, and dTTP, 0.5 uL of RNAse inhibitor (Promega catalog #N251A), 1 uL of MuLV reverse transcriptase (Perkin Elmer), and 1 uL 50 uM random hexamers (Perkin Elmer). 4 ug of RNA (prepared by RNAzol B extraction, Tel-Test, Inc.) were added in 4 uL for a final volume of 20.5 uL and the reactions were incubated at 20° C. for 15 min., 42° C. for 90 min., and finally 99° C. for 5 min. The reactions were then stored at −20° C.

PCR

The PCR's contained 6 uL 25 mM MgCl₂, 8 uL 10×PCR buffer (Promega, Madison, Wis.; catalog #M190A), 0.5 uL 100 uM primer 42M, 0.5 uL 100 uM primer 1040RT, 0.5 uL Taq polymerase (Promega catalog #M186A), 64.5 uL H₂O, and 20 uL of the corresponding reverse-transcription reaction. The reactions were amplified as follows: 95° C.×1 min.; 30 cycles: 94° C.×20 sec., 50° C.×1 min., 70° C.×2 min.; 10 cycles: 94° C.×20 sec., 50° C.×1 min., 70° C.×2.5 min.; 1 cycle: 70° C.×7 min., 4° C.×∞.

The above primers were utilized to achieve discovery and amplification of the PAL-18 message in a quite unexpected manner. In this regard, it was the goal of the original experiments to identify Factor H messages in various cell types, including three different human cancer-derived cell lines: HeLaS3 (cervical), HTB9 (bladder), and LS174T (colon) using low fidelity PCR. Instead however, the PAL-18 message was identified thereby and it was later determined that only the 1040RT primer has homology to both 5′ and 3′ ends of the message, thus the 42M apparently played no role in the amplification. Contiguous sequences were then created following gene walking and expansion.

The PCR products were isolated by cutting the individual bands from a 2% agarose gel. Bands of approximately 750 bp were cut from the lane representing each of the three cell lines, and the BioRad Prep-A-Gene® DNA Purification Kit was used to purify the DNA. All three bands of ˜750 bp were visible on AGE after the purification process. The products were polished with Stratagene (La Jolla, Calif.) catalog #200409 and blunt-end cloned using Invitrogen catalog #K2700-20. The clones were screened by the same PCR method and submitted for sequencing.

Extension of Known Sequence: Genome Walking

The Genome Walker® kit (Clontech, Palo Alto, Calif.), including five human genomic libraries predigested with specified restriction endonucleases and ligated with defined adaptors, as well as primers for specific amplification of the adaptor sequences, was used. The libraries were amplified by PCR (7 cycles: 94°×2 sec., 72°×3 min.; 32 cycles: 94°×2 sec., 67°×3 min.; 1 cycle: 67°×4 min., 4°×∞), in 20 uL reactions of the composition indicated in the GenomeWalker® User Manual (Clontech PT1116-1), with 200 nM primer MCPLR01. The AGE result showed that the process yielded bands with libraries 3 and 4, of approximately 500 and 400 base pairs, respectively. Aliquots of the indicated reactions were therefore subjected to a secondary, nested PCR, using the composition recommended in the Clontech manual, with 200 nM primer MCPLR03 (a primer which is nested relative to MCPLR01), and amplification of 5 cycles: 94°×2 sec., 72°×3 min.; 20 cycles: 94°×2 sec., 67°×3 min.; 1 cycle: 67°×4 min., 4°×∞. Parallel negative-control experiments were performed in which the nested primer was absent. Faint bands are seen in the negative-control lanes, probably because small amounts of the original primer carried over from the primary PCR are sufficient to cause detectable amplification.

The secondary PCR products, designated 39.1 and 39.2, were cloned into the Invitrogen (Carlsbad, Calif.) pCR®II TA vector (see TA cloning). The clones were screened by culture PCR using the vector-specific primers MCXLF01 and MCXLR01 with amplification: 1 cycle: 94°×5 min.; 30 cycles: 94°×10 sec., 60°×30 sec., 72°×1 min.; 1 cycle: 72°×7 min., 4°×∞. In this case all colonies screened were positive for the bands of interest (the negative control was negative). Plasmid was prepared from colonies 44.1.1, 44.1.2, 44.2.2, and 44.2.3 and sequenced.

Reaction 38.10, which initially did not show a band, was amplified for a further five cycles (5×94°×2 sec., 67°×3 min.; 1×4°×∞). This was analyzed by AGE, showing that a band has appeared at >1000 bp. This reamplified 38.10 was subjected to secondary PCR as above and analyzed by AGE. The product was cloned as transformation 53.2, which was screened by culture PCR. Positive clones 53.2.3 and 53.2.4 were grown for plasmid and sequenced.

Further 5′ Extension by Genome Walking

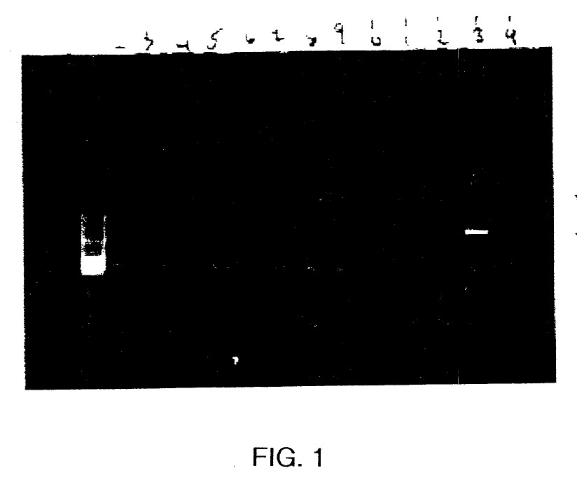

A primer at the extreme 5′ end of the known region (MCPLR05) was used for further genome walking. This primer was used with the five Clontech libraries in GenomeWalker® PCR experiments as above, except that 37 cycles with 67° annealing were initially performed instead of 32. The reactions were analyzed by AGE. Libraries 2 and 4 both yielded bands of ˜2000 bp. These amplicons were TA cloned and screened as above (see FIG. 1). Positive clones 64.2.5 and 64.2.24 were grown for plasmid and sequenced.

Sequence Extension at 3′ End by Rapid Amplification of cDNA Ends (RACE)

Clontech SMART RACE® kit #K-1811-1 was purchased to enable further extension of the known sequence. 1 uL of RNA prepared from HeLa (9-15-97) was subjected to reverse transcription as described in the Clontech technical sheet for this product. Superscript II® (Life Technologies, Gaithersburg, Md.) was used as the reverse transcriptase. 2.5 uL of the reverse transcription reaction was then used in a 50-uL PCR with universal primer mix as described in the Clontech documentation for SMART RACE®. Amplification was 5 cycles: 94×5 sec., 72°×3 min.; 5 cycles: 94°×70°×10 sec., 72°×3 min.; 27 cycles: 94°×5 sec., 68°×10 sec., 72°×3 min., 1 cycle: 4°×∞. Gene-specific primers MCPLF01 and MCPLF02 were each used at 200 nM in separate reactions. Both primer MCPLF01 and primer MCPLF02 yielded bands of >2000 bp. The reactions were subjected to secondary amplification using the nested primer MCPLF03 and the nested universal primer mix supplied by Clontech. Fewer cycles were used than recommended for secondary PCR in the Clontech manual because undiluted primary PCR was used. The amplification was 1 cycle: 94°×5 min.; 12 cycles: 94°×5 sec., 68°×10 sec., 72°×3 min.; 1 cycle: 72°×7 min., 4°×∞. AGE analysis showed that the secondary PCR of the MCPLF02 primary reaction had produced a more distinct product than either the negative-control reaction or either of the reactions with the MCPLF01 primary reaction. This amplification product, as well as the original primary PCR product of the MCPLF02 reaction, were used for TA cloning. Colonies were screened as above. While no full-length clones of the expected amplicon size were seen, some clones (e.g., 58.1.1, 58.1.2, 58.1.6, 58.2.7) were shown to have PAL-18 specific sequences by subsequent PCR using an internal (PAL-18) primer and a vector-specific primer. These clones were grown for plasmid and sequenced.

Confirmation of Known PAL-18 Regions by Cloning and Sequencing of Fetal Liver cDNA Library

A human fetal liver cDNA library from Clontech (Product #HL5003b) was subjected to a PCR as follows: 2 uL Advantage Taq 2 buffer (Clontech), either 0.4 uL primer MCPLF01 and 0.4 uL primer MCPLR02 or 0.4 uL primer MCPLF04 and 0.4 uL primer MCPLR04, 0.4 uL 10 mM (total) dNTP's, 0.4 uL Advantage Taq 2, and 16 uL H₂O were mixed with 0.4 uL fetal liver library. Amplification was 1 cycle: 94°×5 min.; 25 cycles: 94°×10 sec., 72°×1 min.; 1 cycle: 72°×7 min., 4°×∞. Both reactions yielded bands of expected size on AGE analysis, while negative controls were negative. These PCR products were TA cloned and cultures grown from the colonies were screened with vector-specific primers. Positive cultures 72.1.1, 72.1.4, and 72.2.3 were grown for plasmid and sequenced.

Sequencing of Image Clones

Strong homologies were observed between the sequences obtained by 3′ RACE and ESTs reported in the BLAST DBest database using default settings and the algorithm described by Altschul et al., Nuc. Acids Res. 25:3389-3402, 1997. The longest clones with strong homologies were Accession #'s AA994364 and AI243754. These clones were purchased from the Image Consortium. Vector-specific primers MCPTF01 and MCPTR01 were designed to sequence into the cloning vector used. The clones were grown in 2-mL cultures and checked for inserts using these primers. Plasmids were then prepared and submitted for sequencing.

Discovery of Human Genes Related to PAL-18 by Low-Fidelity RT-PCR

The same method used in the initial discovery of PAL-18 transcripts can be modified to use in discovering genes and gene products related to PAL-18. The method involved use of 1) primers with intermediate annealing temperatures (37-52° C.), to facilitate association with genes that are related to but not identical with the known PAL-18 constructs; 2) 35-40 PCR cycles to permit appearance of minor bands; and possibly 3) synthesis and use of degenerate primers, i.e., primers synthesized with small admixtures of bases other than the homologous PAL-18 bases at each or selected positions, to permit enhanced homology with related but non-identical genes or gene products. According to our previous experience, “doping” percentages so as to yield an average of 1-3 mismatches per molecule produce primers that are readily used in low-fidelity PCR. For example, a degenerate primer of 18 bases in length containing 10 A/G bases and 8 C/T bases, designed to anneal at 51° C. (using the simple formula Tm=([AG]*4°)+([CT]*2)−5°) would be synthesized with a 4% admixture of each non-homologous base, yielding an average of 12% mutagenesis at each base position, or an expectation of 2.16 mismatches per primer molecule. These mismatches would reduce the average expected annealing temperature of the degenerate primer to ˜38-46°, and initial low-fidelity PCR experiments would be carried out with annealing temperatures in this range.

Identification of Chromosomal Location 1Q41 FOR PAL-18

Release of the sequence data from the human genome project allowed for subsequent identification of the chromosomal site for the PAL-18 gene (1q41). The significance of this particular chromosomal location is that this location is known to be associated with a variety of maladies and thus, supports the herein noted utilities for probing for progression and identification of certain cancer types, but also implicates a potential role for PAL-18 in other disease/dysfunctional states.

For example, homeobox gene in 1q41-42 homologous with Xenopus laevis Mix.1; another homeobox gene HLXB9 linked to dominant inherited sacral agenesis reported to map to 1q41-42.1.

Amplification of 1q41 in 39% of breast cancer cell lines; several other papers about amplification in breast cancer. This site is not associated with any established oncogene. Rearrangements and breakpoints are also seen in breast cancer involving 1q41.

Linkage of USH2A locus at 1q41 to User Syndrome Type II (congenital sensorineural hearing loss and retinitis pigmentosa).

MAP kinase phosphatase MKP-5 (genes DUSP10) localized to 1q41, especially in African-Americans.

Human systemic lupus erythematosus genetically linked to D1S229 at 1q41.

Rippling muscle disease linked to 1q41-42.

Poly(ADP-ribose) polymerase is located at 1q41-44; PARP overexpression is associated with low genetic instability in breast cancer specimens; another paper reports 1q41-44 amplified in breast cancer.

53BP2: p53-binding protein 53BP2 is mapped to 1q41-42.

Human potassium channel gene TREK-1 (KCNK2) maps to 1q41.

UDP-N-acetylgalactosamine: polypeptide N-acetylgalactosaminyltransferase GALNT2 maps to 1q41-42.

STK6 encoding a mitotic centrosomal protein kinase Aik and/or an STK6 pseudogene maps to 1q41-42.

Toll/Interleukin-1 receptor-like gene 3 involved in cytokine-mediated IL-1 receptor/I-kappaB/NF-kappaB activation cascade maps to 1q41-42.

Histone H3F3A gene maps to 1q41.

Nuclear VCP-like protein NVL of the AAA family of ATPases maps to 1q41-42.2.

Serine/threonine protein kinase PK428 is 65% homologous in the kinase domain to the myotonic dystrophy protein kinase DM-PK and 33% homologous to cyclic AMP-dependent protein kinase; PK428 maps to 1q41-42.

A human senescence gene and a gene associate with mental retardation are reported to be in 1q41-42. I will try to get ahold of this information this week.

Human protein phosphatase 2A regulatory subunit gene b56alpha maps to 1q41.

Enolase processed pseudogene ENO1P maps to 1q41-42.

A renal transplant infected with Epstein-Barr virus had a chromosomal translocation 46, XY, inv (1)(p35; q41) involving the TGFB2 gene in 1q41.

1q41 is reported as a heritable fragile site leading to spontaneous abortions.

Clones expressed in retinal pigment epithelium but not in any other tissue tested include two cDNAs from 1q41-42.

Homeobox HLX1 involved in hemopoietic development maps to 1q41-42.

There is a dinucleotide repeat polymorphism for D1S70 (CRI-L461) on 1q41.

Van der Woude syndrome involving clefting of the palate, hypodontia or lower lip pits maps to candidate region 1q32-1q41.

Human homeobox genes HB24 and HB9 map to 1q41-42.

Human inositol 1,4,5-trisphosphate 3-kinase B (ITPKB) maps to 1q41-43.

A partial duplication of 1q41 was observed in a case of congenital glaucoma.

A terminal deletion at 1q41 was observed. in a fetus with symptoms compatible with distal 1q deletion syndrome.

Human nuclear NAD+ADP-ribosyltransferase maps to 1q41-42.

DAPI is a compound which specifically causes breakage at AT sites in DNA; the 1q41-42 interface is one of three sites in the human genome susceptible to cleavage.

Two siblings were observed to have partial trisomy for 1q24-1q41.

As is evident from the above, listing this chromosomal location has been associated with a variety of indications. As such, the discovery of PAL-18 may lead to more effective therapies or to more readily performed diagnostics to determine the presence, absence, or progression of a disease causing gene abnormality.

Identification of the location on 1q41 was performed by the BLAST algorithm as previously defined, which identified a protein that was 93% identical and 93% conserved over 312 amino acids. The results indicated a nearly perfect match with PAL-18 containing the 96 residue exon, with the exception of the 5′ and at least one exon insertion. The portion beginning AQQDAV (SEQ ID NO:168) is a match for the 411 amino acid PAL-18 sequence, except for an A between FYQKA (SEQ ID NO:169) and KYLHL (SEQ ID NO:170).

Protein sequence of A.ctg12788-000005.2.0 as predicted from chromosome location 1q41 bases 256410710-256465015. Underlining demonstrates the overlap

MKIGPSFKANAITDAQQDAVFELISMGFNVALWYTKYASRLAGKEN ITEDEAKEVHRSLKIAAGIFKHLKESHLPKLITPAEKGRDLESRLIEAYVIQCQ AEAQEVTIARAIELKHAPGLIAALAYETANFYQKAAKYLHLKMCFYTAYA YCYHGETLLASDKCGEAIRSLQEAEKLYAKAEALCKEYGETKGPGPTVKP SGHLFFRKLGNLVKNTLEKCQRENGFIYFQKIPTEAPQIELKANYGLVEP IPFEFPPTSVQWTPETLAAFDLTKRPKDDSTKPKPEEEVKPVKEPDIKPQ KDTGCYIS (SEQ ID NO:166)

This sequence is predicted from the following cDNA:

| | |
|---|---|
| ATGAAAATCGGCCCGTCTTTTAAAGCCAATGCCATTACCGATGCCCAGCA | (SEQ ID NO:167) |
| GGATGCTGTTTTTGAATTAATTTCCATGGGATTTAATGTAGCTTTATGGT | |
| ATACCAAATATGCTTCAAGACTGGCTGGAAAAGAAAATATAACAGAAGAT | |
| GAAGCAAAAGAAGTTCATCGAAGCCTAAAGATTGCAGCTGGGATTTTTAA | |
| ACATTTAAAGGAAAGTCATCTCCCAAAACTCATTACACCTGCGGAAAAAG | |
| GAAGAGATTTAGAGTCACGACTCATAGAAGCATACGTTATTCAATGTCAG | |
| GCTGAAGCTCAAGAAGTAACAATTGCTCGAGCAATTGAACTAAAACATGC | |
| TCCTGGACTAATTGCTGCACTGGCGTATGAAACAGCCAATTTCTATCAAA | |
| AAGCTGCAAAATATCTTCACTTGAAGATGTGTTTTTACACAGCTTATGCT | |
| TACTGTTACCATGGTGAGACTTTATTGGCTAGTGATAAATGCGGTGAAGC | |
| AATCAGGTCTCTCCAAGAAGCAGAAAAATTGTATGCAAAGGCAGAAGCAC | |
| TGTGTAAAGAATATGGAGAAACCAAAGGACCTGGACCAACAGTCAAACCT | |
| TCAGGACATCTGTTCTTTAGGAAACTTGGAAACCTTGTGAAGAACACCCT | |
| AGAAAAATGTCAGAGAGAAAATGGATTTATTTACTTTCAAAAAATTCCAA | |
| CAGAAGCCCCACAGCTGGAACTCAAAGCAAATTATGGTCTCGTAGAGCCT | |
| ATACCTTTCGAATTTCCTCCTACAAGTGTTCAGTGGACACCAGAAACATT | |
| GGCTGCATTTGATCTCACCAAAAGACCCAAGGATGACAGTACTAAACCCA | |
| AACCAGAAGAAGAAGTGAAACCTGTGAAAGAACCAGACATCAAACCTCAA | |
| AAGGACACTGGGTGCTACATCTCCTAA | |

### Example II

### Homology Searches Using PAL-18 Sequences

A predicted protein fragment was constructed by translating the nucleotide sequence obtained by sequencing the Factor H 1040RT primer amplicon. This protein fragment was used in a homology search of the nonredundant GenBank CDS database. This search identified B0507.2 gene of C. elegans, BRO1 of yeast, and ptp-td14 of rat, and YNK of yeast as having some degree of homology these full sequences where then compared by the BLAST algorithm available from the Genetics Computer Group of Madison Wisconsin or from the National Center for Biotechnology Information using the BLAST two sequences program, which uses the Smith-Waterman algorithm described above by Altschul et al.

A sequence of 46 nucleotides from the same amplicon was used in a search of the DBest EST GenBank database.

The entire sequence obtained with clone 64.2.5 using primer MCXLF01 was used in searches of the nonredundant nucleotide EST GenBank+EMBL+DDBJ+PDB sequence databases and DBest both of which identified small 43 and 26 base matches.

The entire sequence obtained with clone 64.2.5 using primer MCXLF03 was used in a search of DBest.

The sequence obtained with clone 58.2.7 with primer MCPLF07 was used in a search of DBest.

The translated sequence of the calculated 411-residue protein was used in searches of pdb and Swissprot.

Analysis of the homology with calnexin and calnexin-like proteins revealed a short, homologous region, including a match of seven out of eight consecutive residues (PAL-18=ESKLRYIQ vs. calnexin=ESKLRYLQ). A further search was done with the calnexin sequence as the query sequence, using a higher expectation value of 1000 in order to identify additional potentially important local homologies.

More extensive analysis was performed of the homologies with various proteins, as reflected in Table 1.

TABLE 1
Examples of Protein-Level Homologies of the PAL-18
Predicted Protein (SEQ ID NO: 76).

| Related | Comparison | % | % | PAL-18 |
|---|---|---|---|---|
| Protein | Method | Identity | Conserved | Region |
| Ptp-td14 | 2-sequence BLAST | 21 | 42 | 190-397 |
| Bro 1 | ″ | 25 | 41 | 54-271 |
| YNK1 | ″ | 20 | 36 | 2-361 |
| C. elegans | | | | |
| B0507.2 | ″ | 39 | 58 | 1-405 |

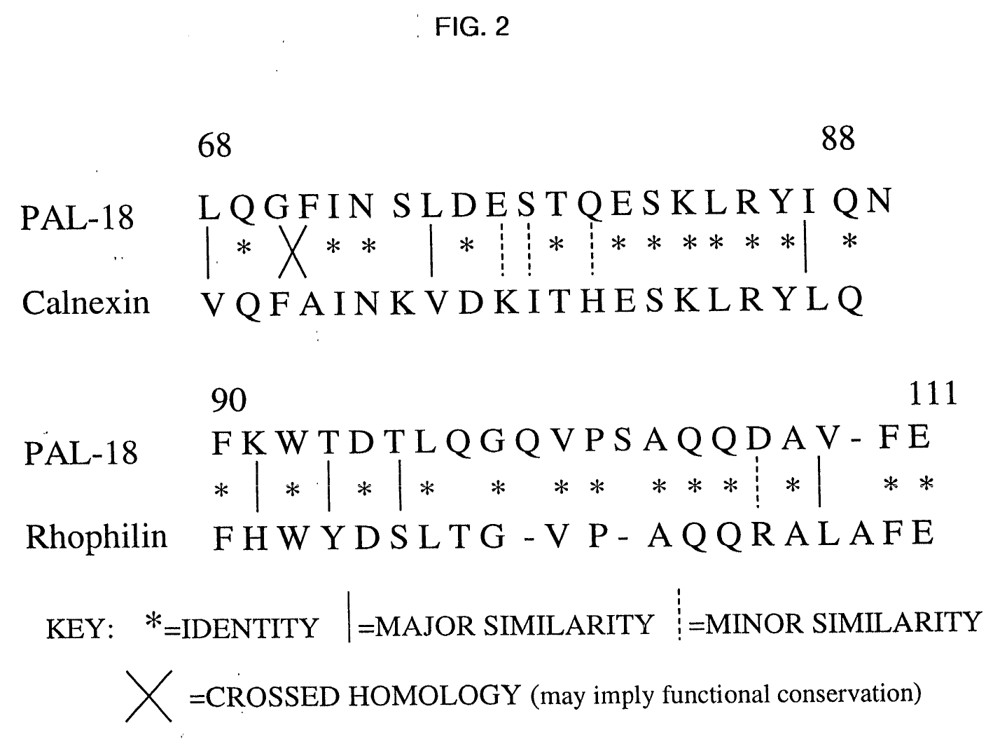

A further interesting and potentially significant feature of the PAL-18 protein is that there are adjacent strong local homologies to two known proteins: yeast calnexin and mus rhophilin. These adjacent homologies are depicted in FIG. 2.

The general criterion for significance of a homology “match” is considered to be p<0.05 of a single match of the indicated quality across the entire database under search. In other words, if the expectation of a match in the whole database is less than one in twenty, then the observed match is presumed to represent a true relationship between the two similar sequences, and not merely an adventitious similarity that is due to the very great size of the database. Examples of such adventitious similarities may be found in Search F, where, for example, a number of matches of apparent statistical significance are found for a sequence beginning at base 506 of the query sequence (PAL-18). While the a priori significance of the match is sufficient to render it of potential interest, the fact that this very sequence appears so many times in the database implies that is has been specifically amplified at some time, and therefore that its appearance within a given gene does not necessarily imply a true relationship to other genes that have the sequence. Another indication that the match is adventitious is the fact that the relationship is not “extensible”; i.e., regions outside of the similarity in the two genes exhibit no similarity. On the other hand, a good test of a true match is that the similarity often extends beyond the regions initially identified. For example, the relationship between PAL-18 and the B0507.2 protein from C. elegans was initially identified by searching with only half of the known sequence of the hypothetical PAL-18 protein as the query sequence. When the match was identified, a search with the rest of the known sequence strongly confirmed the relationship. Thus the matches which are of greatest interest (Table 1) mostly represent homologies over large stretches of the PAL-18 sequence. All of these matches also pass the statistical criterion described above.

The adjacent matches with calnexin and rhophilin are also of interest (see FIG. 2). These matches are likely to be of significance because, first, they are locally quite strong, and, secondly, because they are almost precisely adjacent—i.e., they do not overlap, yet there is only a single amino-acid residue intervening. There is therefore a good chance that they represent adjacent functional domains in the protein. Overlapping matches of equal individual significance would be less likely to imply coordinated functions.

The strong local match between the calnexin-like sequence and the tssk-1 and tssk-2 kinase substrate proteins may be significant, in spite of the fact that it fails the p<0.05 test, because sequences that modulate kinase specificity are often short domains of larger proteins; the homology may therefore indicate that this region of PAL-18 interacts with kinases or shares a similar function with tssk-1 and tssk-2 substrates. However, unlike the homologies noted above, this type of highly localized, strong homology which is not extensible is more likely to represent functional similarity (which often appears by convergent evolution) than descent from a common ancestral gene.

### Example III

### Detection of PAL-18 Message

Any pair of primers capable of forming an amplicon within the PAL-18 message, including pairs of several of the primers listed herein, would in principle be usable in detecting the message or gene. The methods used for detection are similar to those used originally used for cloning.

Method 1

Detection of PAL-18 message in cancer cell lines by 42M and 1040RT primers.

Reverse Transcription

Reverse Transcription was performed as described under Example 1 above.

PCR

PCR was performed as described under Example 1 above.

Method 2

Detection of PAL-18 message by PCR using primers specifically designed for PAL-18.

The PCR methods used for this process are identical to that described above under CONFIRMATION OF KNOWN PAL-18 REGIONS BY CLONING AND SEQUENCING OF FETAL LIVER cDNA LIBRARY. Accordingly, any pair of primers capable of generating an amplicon can be used for this purpose.

### Example IV

### Message Variants and Detection Thereof

The following variants of the PAL-18 message are described herein:

Full-length (main variant), represented by contig ID 1 (SEQ ID NO:75)

Full minus 96-bp exon, represented by contig ID 2 (SEQ ID NO:76)

Full minus 16-bp exon, represented by contig ID 3, sequences 72.1.1.XLR01.broad.extract and 72.1.1.XLF01.broad.extract (SEQ ID NO:77)

Full minus 160-bp exon, represented by contig ID 4, sequence 95.3.6.BDR01.rev.comp. (SEQ ID NO:78)

Full minus 104-bp and 16-bp exons, represented by contig ID 5, sequences 89.7.10.XLR01 and 89.7.10.PLF12 (SEQ ID NO:79)

The 104-bp exon consists of the 96-bp exon followed by a contiguous exon of 8bp.

The term “exon” refers to a region of the message which has been observed to be absent in certain clones. However, it is also possible that these “exons” represent transcription from different genes. The transcription products of these different genes, if they exist, may undergo splicing which is distinct from the splicing experience by the products of the primary PAL-18 gene, or the splicing may be identical. These hypothetical additional genes may have deletions and/or insertions in them which may combine with the splicing effects to give the observed effects. One tentative indication that alternative genes may be involved is the fact that the canonical “AG” sequence is observed for only a portion of the putative splice sites. However, it should be recognized that splicing at other sites is possible, such as for type II introns and the like.

Given what is known about the nature of RNA splicing, exons may be combined in various ways by multiple splicing events. For example, there may be a message in which both the 96-bp and 160-bp exons are absent, although it has not yet been observed. There may also be messages in which one or more of a set of exons which have so far been observed to be present or absent as a group may be present without some or all the other members of the group being present. An example of this is the putative 8-bp exon, which has been observed to be absent only when the 96-bp exon is absent; however, the 8-bp exon may be present without the 96-bp exon in other messages which have not been observed. The same is true of the 8-bp exon and the 16bp exon. Moreover, it is clear to one of skill in the art that these variant messages may be translated by any means of the well-known genetic code to yield further predicted proteins in addition to those specified by sequence ID 72-74.

Detection of Message Variants

Special primers were designed for detection of the presence or absence of specific exons. The presence or absence of the 96-bp exon in a given cDNA preparation or clone may be detected by mobility difference in AGE, using PCR as under CONFIRMATION OF KNOWN PAL-18 REGIONS BY CLONING AND SEQUENCING OF FETAL LIVER cDNA LIBRARY above. However, the variant missing the exon is generally present in small amounts (but see exception, below), which makes specific detection desirable. Specific detection of messages which contain the exon is accomplished by the use of a primer which anneals within the exon. Specific detection of messages which do not contain the exon of interest is accomplished by designing a primer which anneals under stringent conditions across the exon-exon boundary formed by the missing exon, but anneals only very poorly to the structure including the exon. Both of these principles are illustrated a PCR experiment. Primer 13 anneals within the 96-bp exon. A 1:10,000 dilution of a plasmid containing this exon is easily detected, while neither a 1:100 nor a 1:10,000 dilution of a plasmid missing this exon is detected. Conversely, the plasmid missing the exon is detected at 1:100 dilution (and very slightly at 1:10,000 dilution), while the plasmid containing the exon is not detected at either concentration. The same effects may be observed in cDNA from human tissues. The mid-sized variants (contig Ids 2 (SEQ ID NO:76) and 5 (SEQ ID NO:78) above) appear to be abundantly present in human prostate tissue, but nearly absent in peripheral blood leukocytes. This is conveniently confirmed by use of the variant-specific primers which detect only mid-sized variants lacking the 96-bp exon. These variants are easily detected in a 1:100 PCR of prostate cDNA, but only a very slight signal is seen with PBL cDNA.

Use of the Variants in Detection, Diagnosis, and Prognosis of Prostate and Colon Cancer

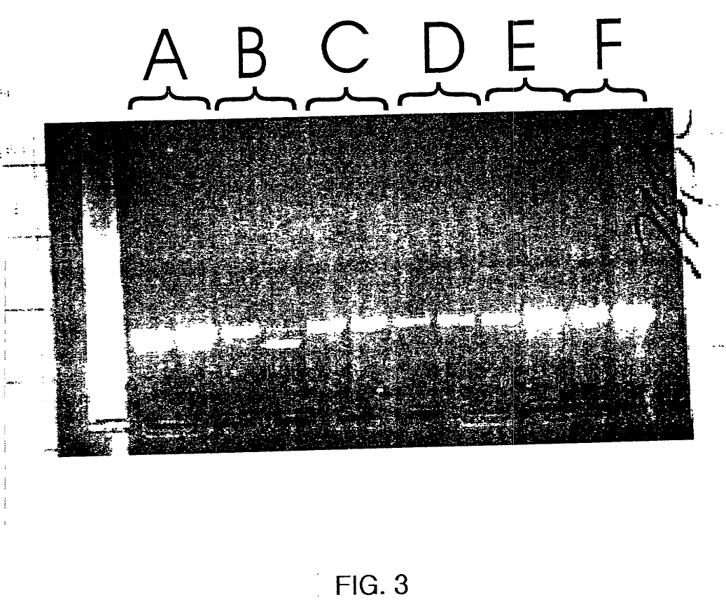

The ratio of expression of the PAL-18 variants in the prostate varies with the individual, and changing or aberrant ratios may be symptomatic of particular disease states, disease-susceptibility states, or genetic predisposition to disease. For example, cDNA made from RNA extracted from the prostate tissue of an individual (“patient B”) with prostate cancer was observed to have a noticeably higher level of lower molecular weight variants and a lower level of the normal, full-length message than tissue from other patients (FIG. 3). This patient had a strong family history of prostate cancer, with four male relatives suffering from the disease. FIG. 3 demonstrates the levels of PAL-18 in six prostate cancer positive individual using the MCPLF12 and MCPLR10 primers.

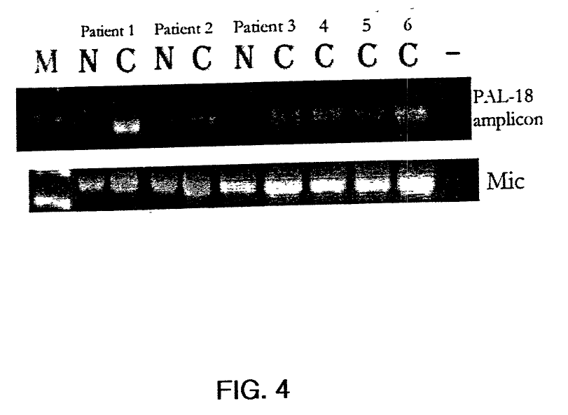

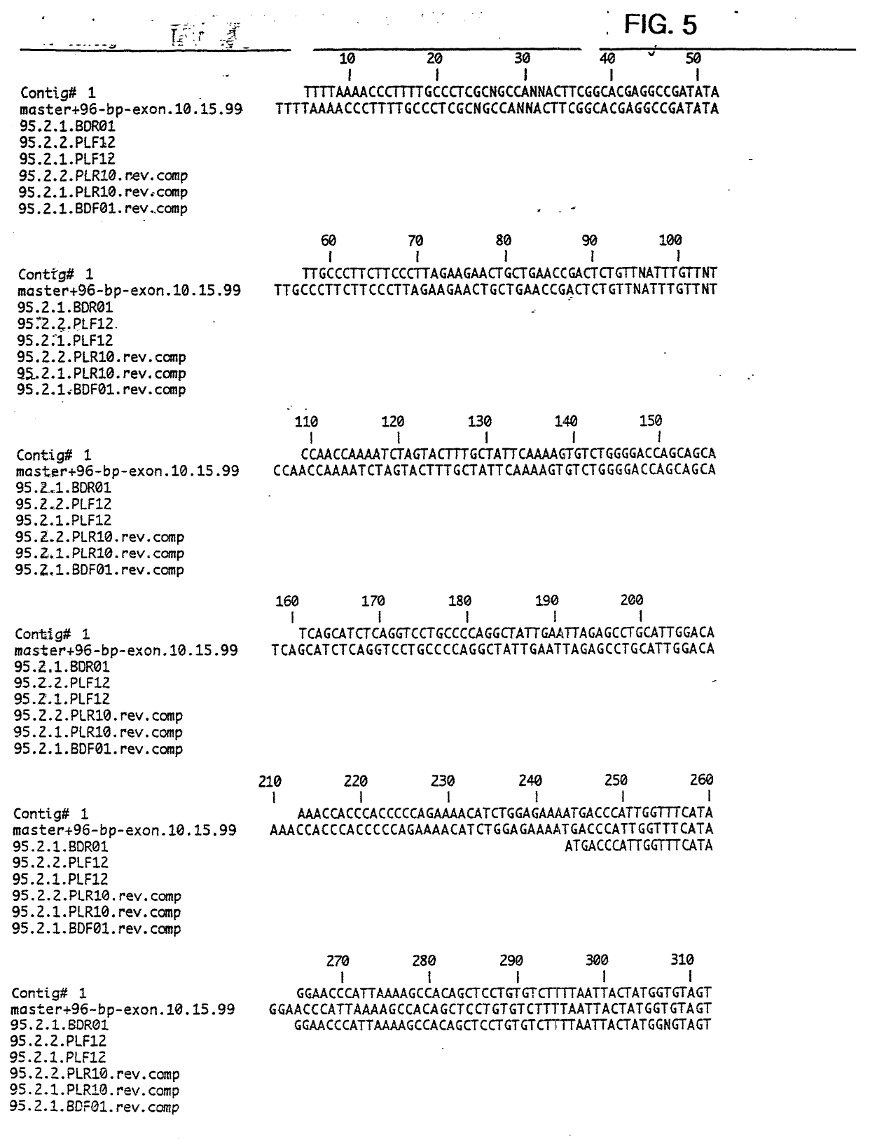

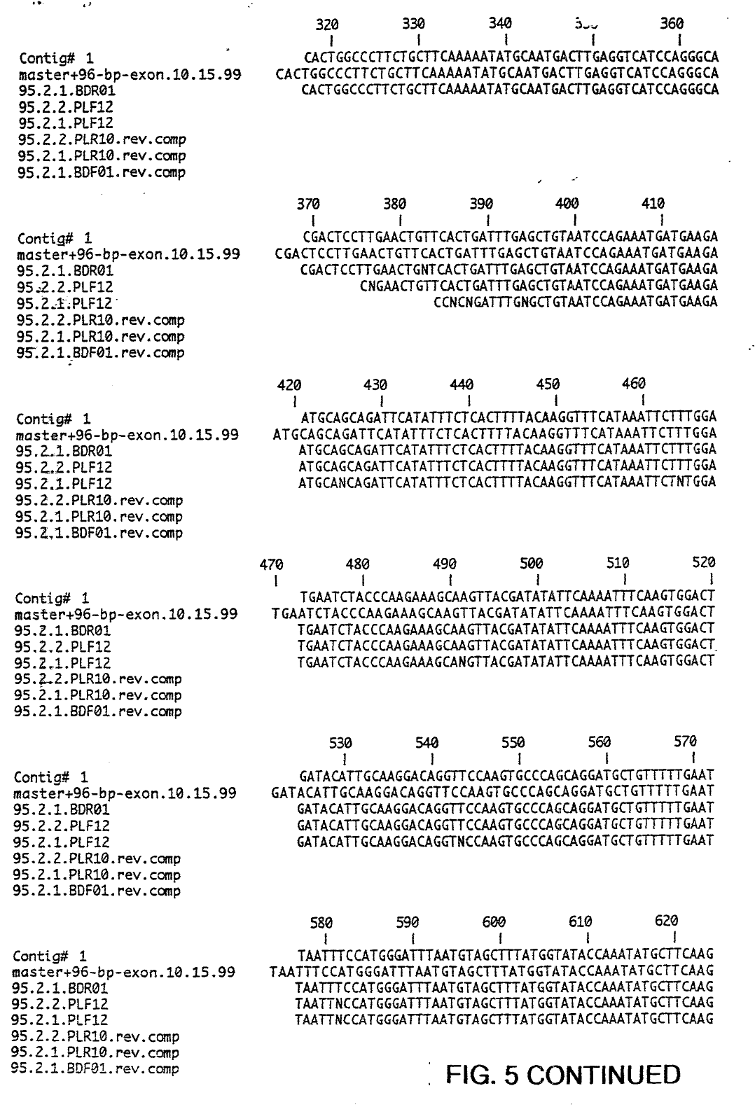

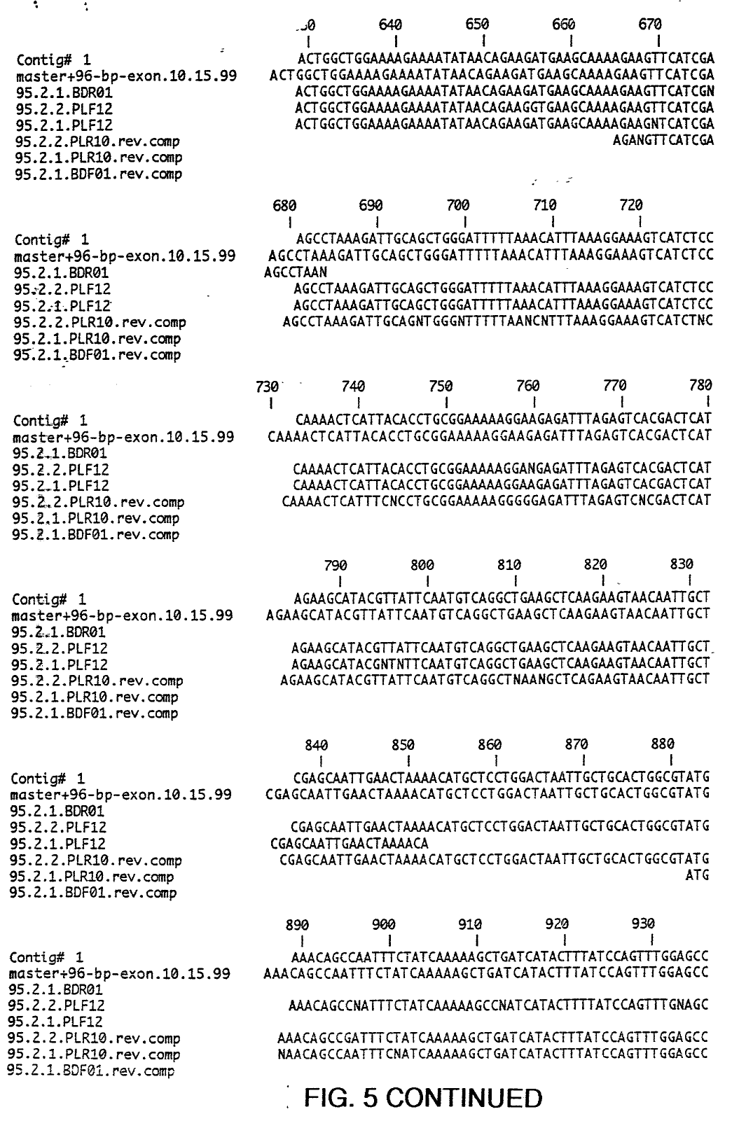

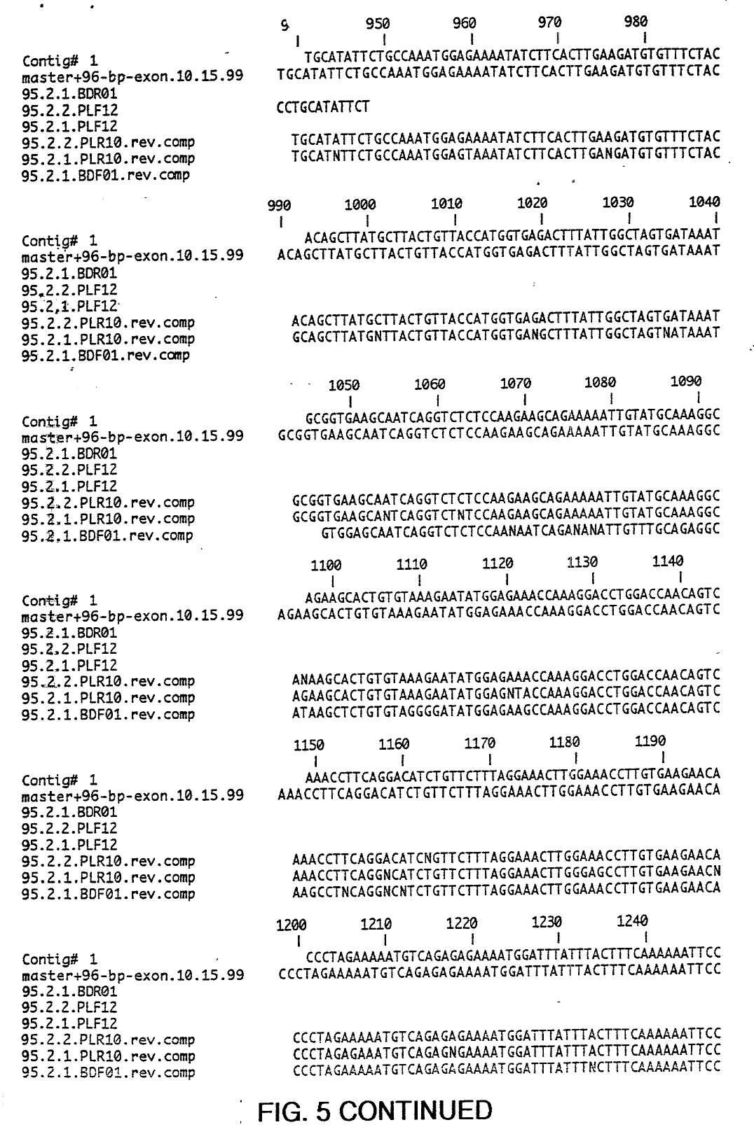

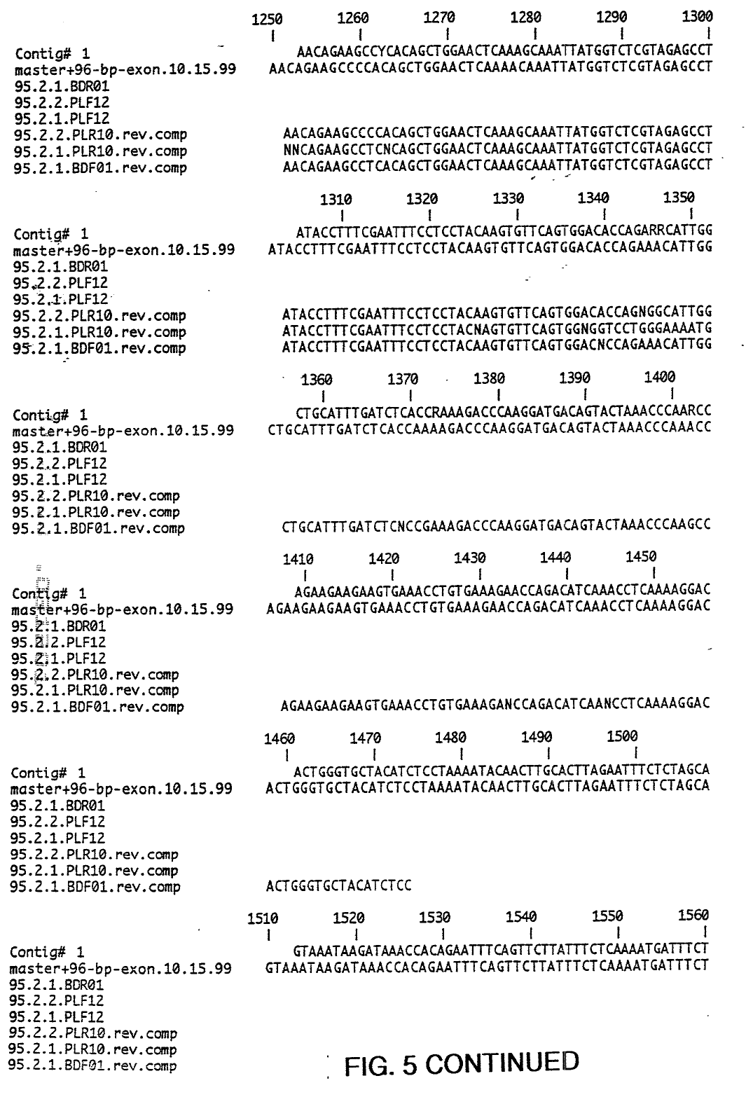

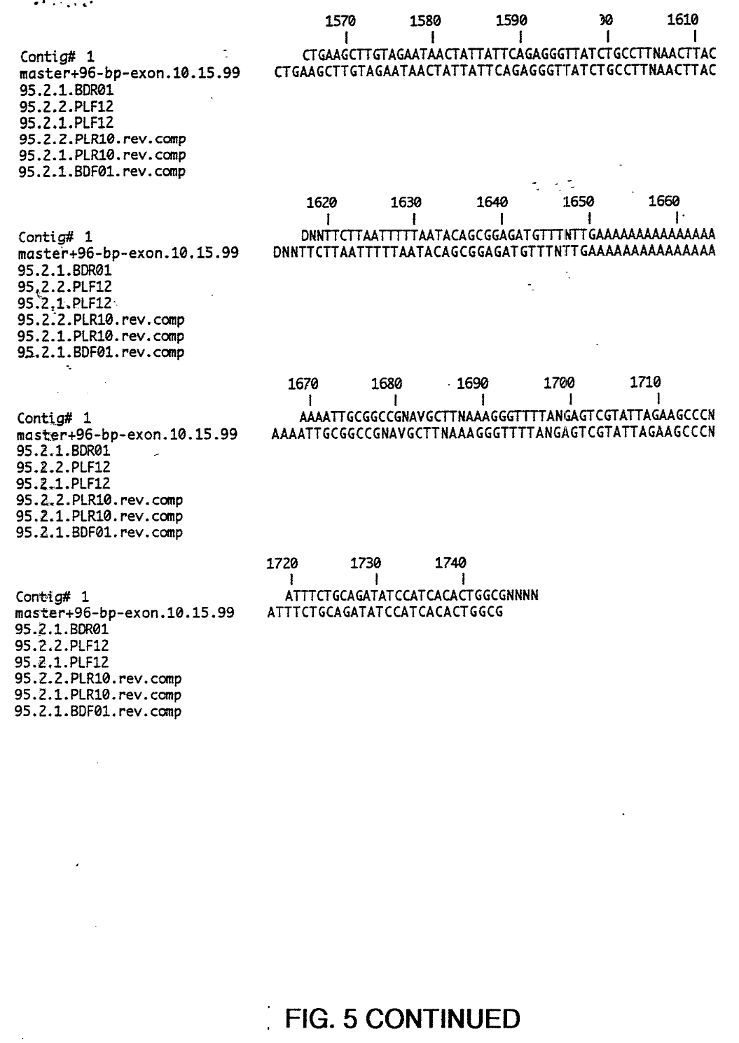

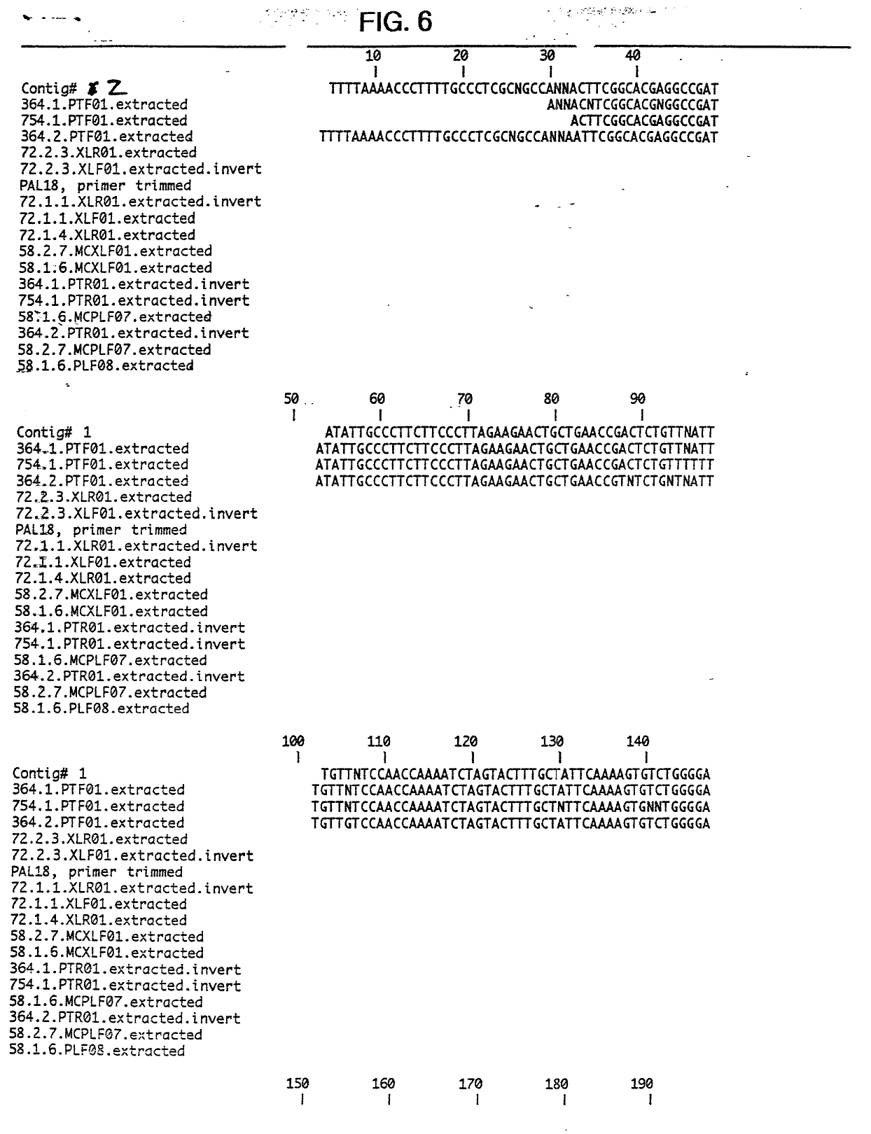

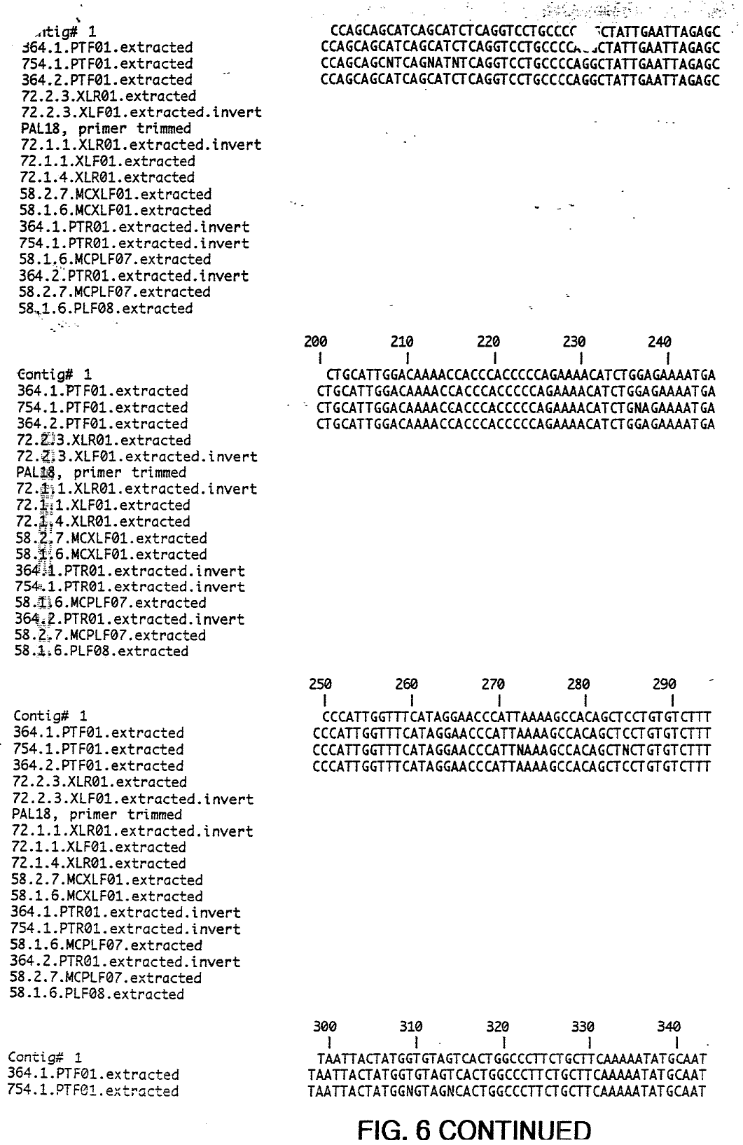

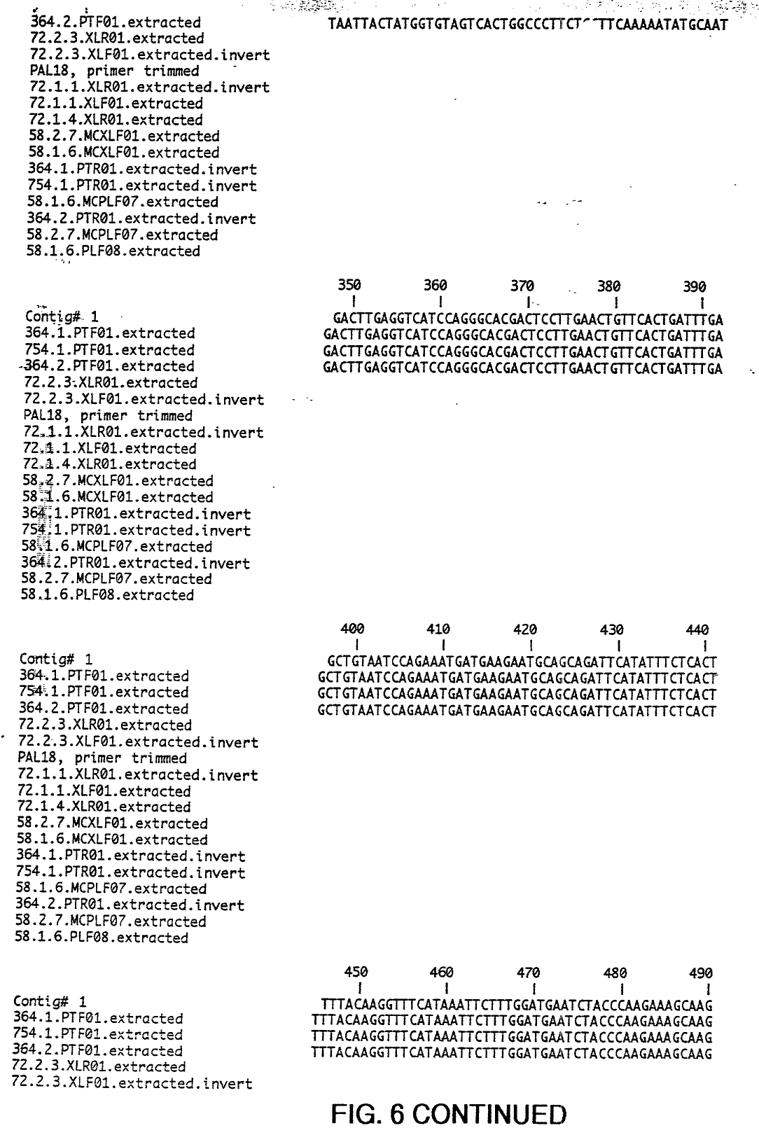

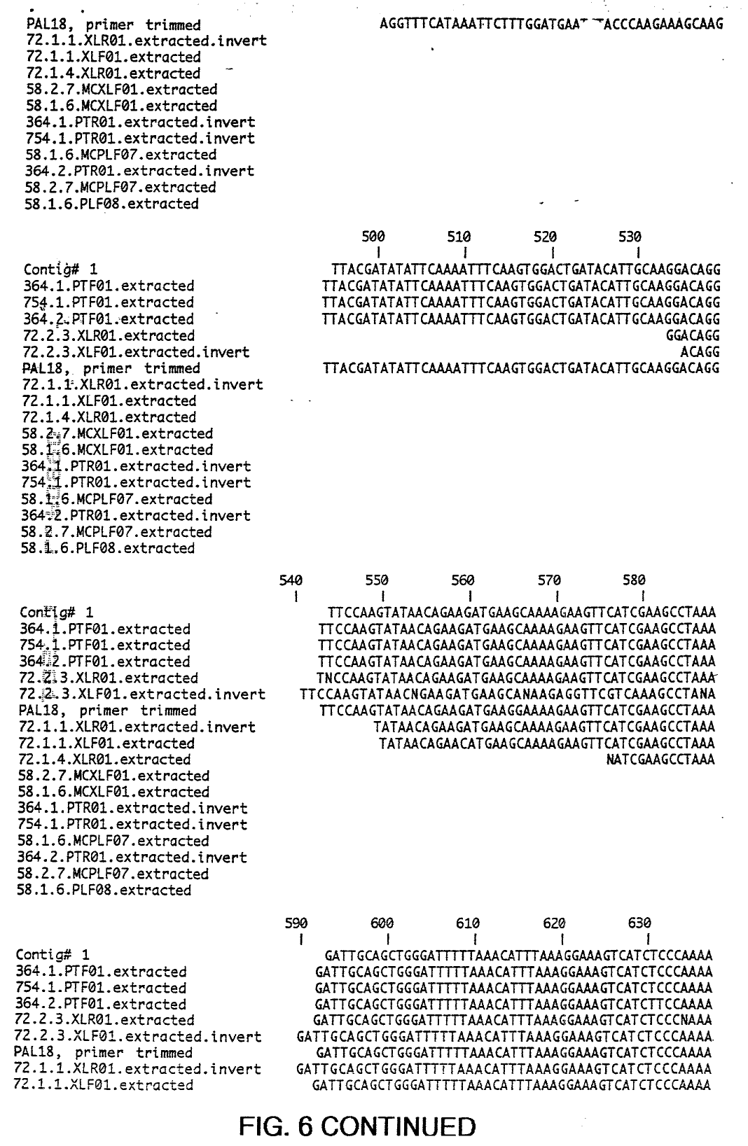

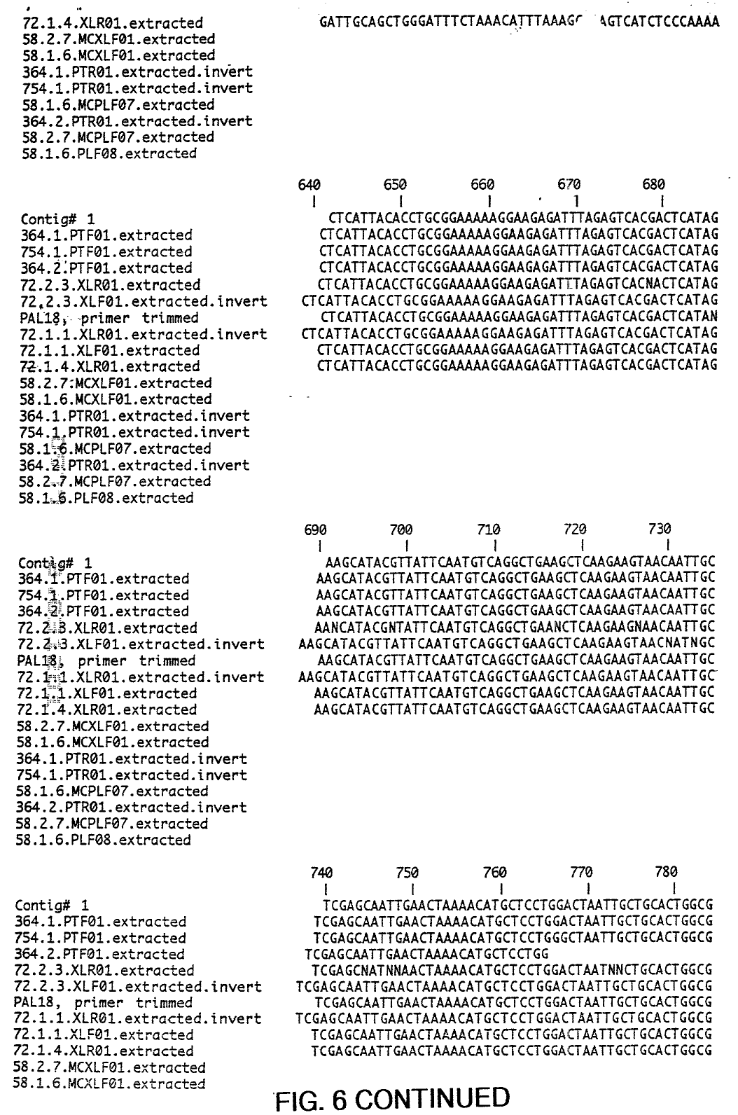

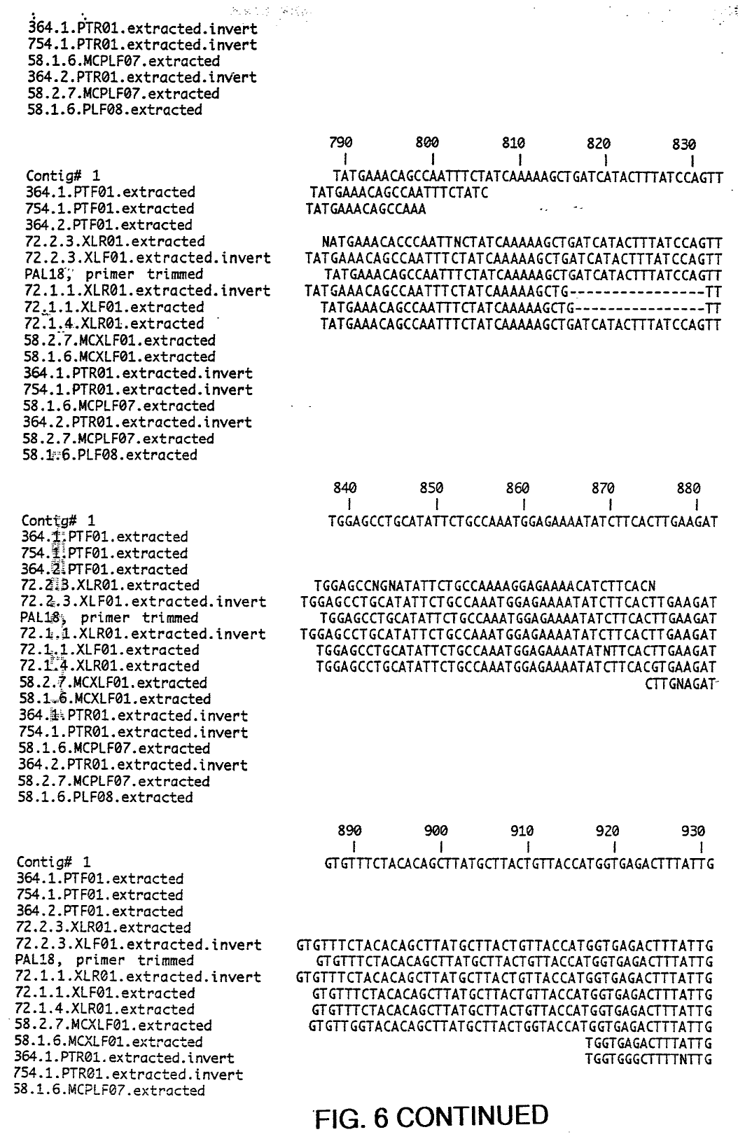

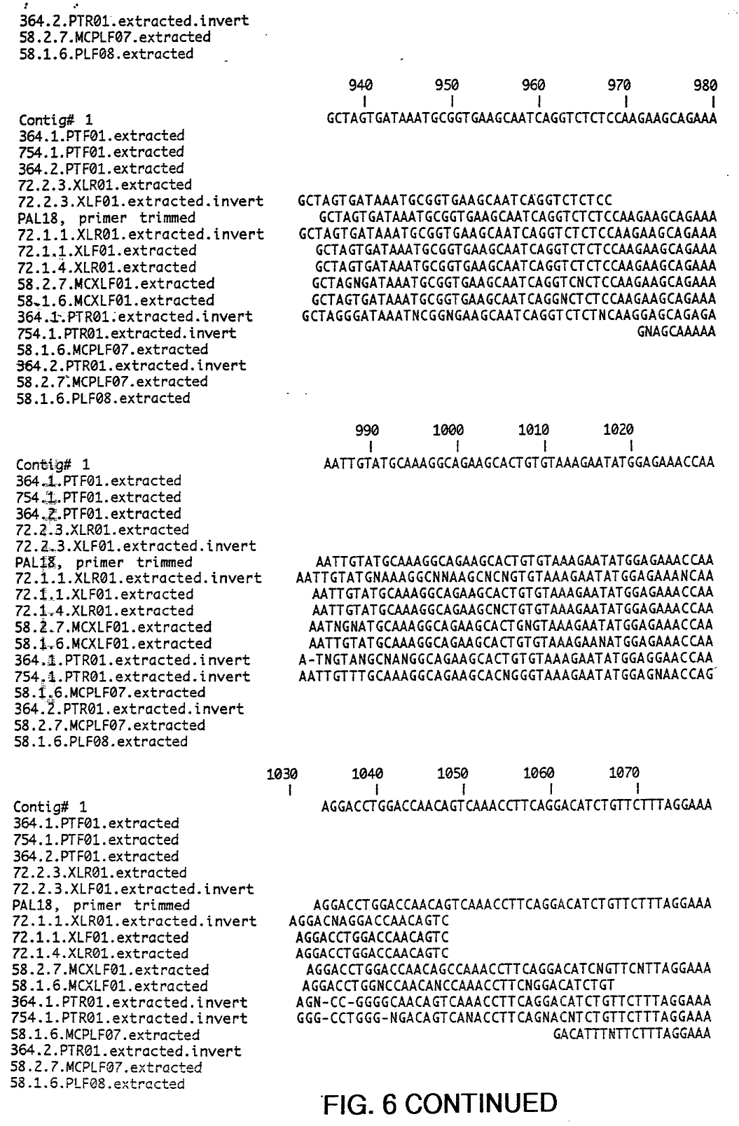

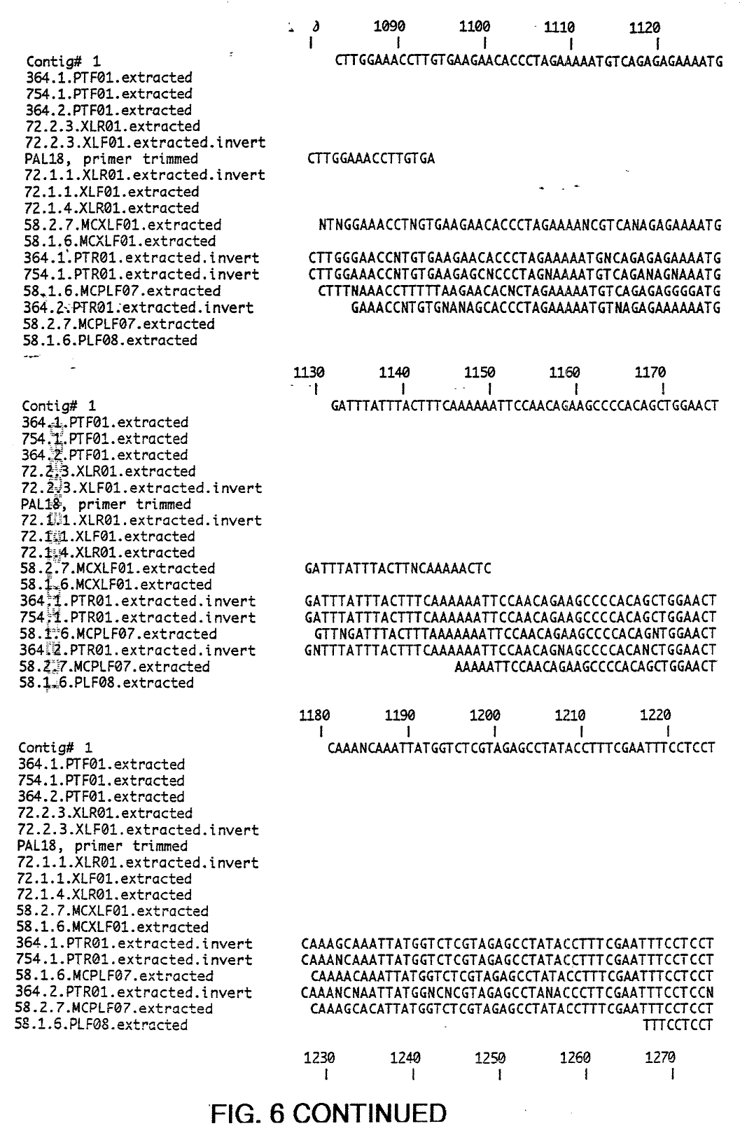

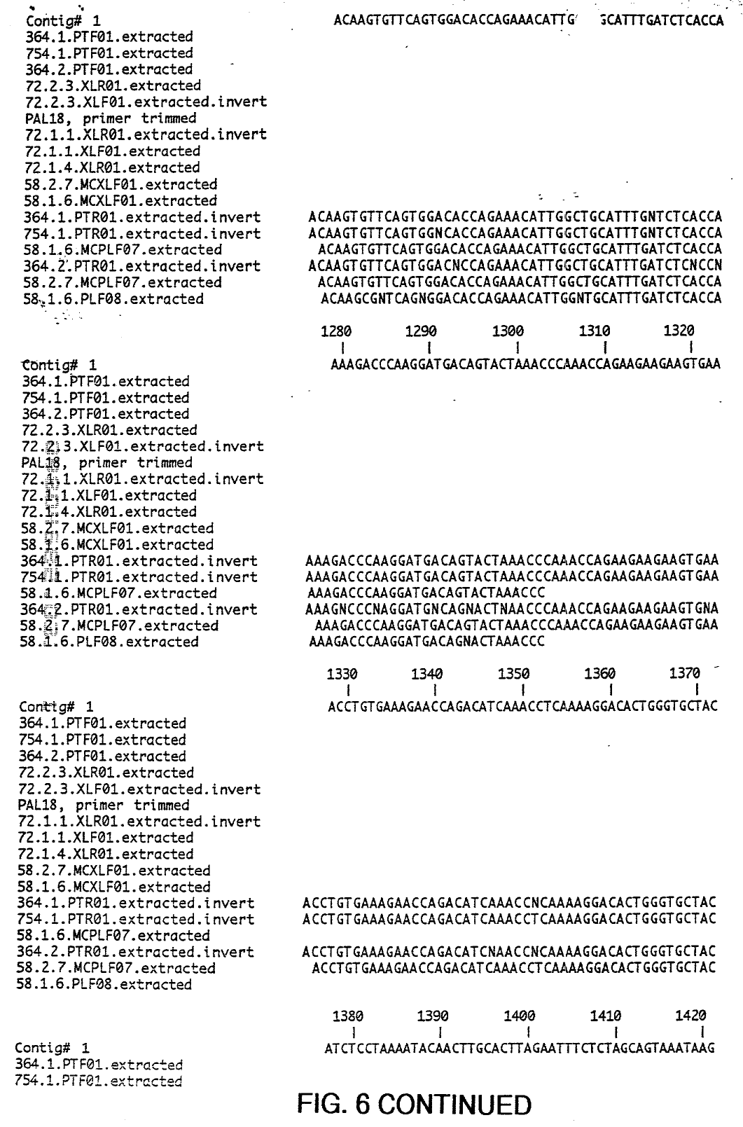

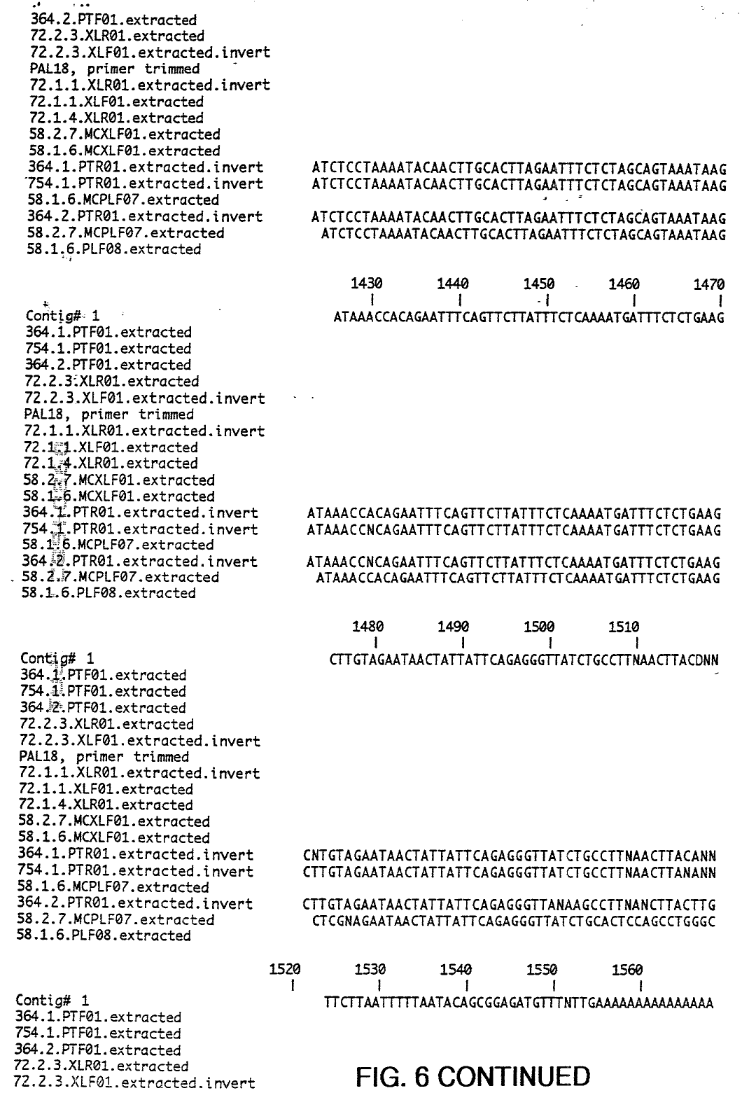

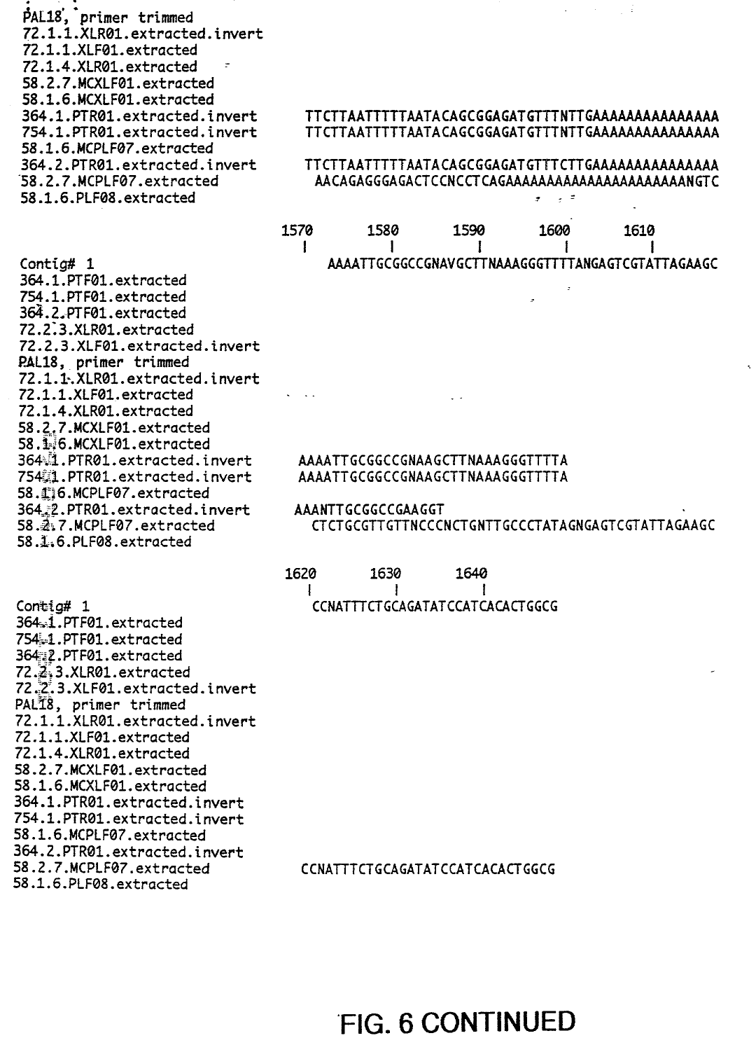

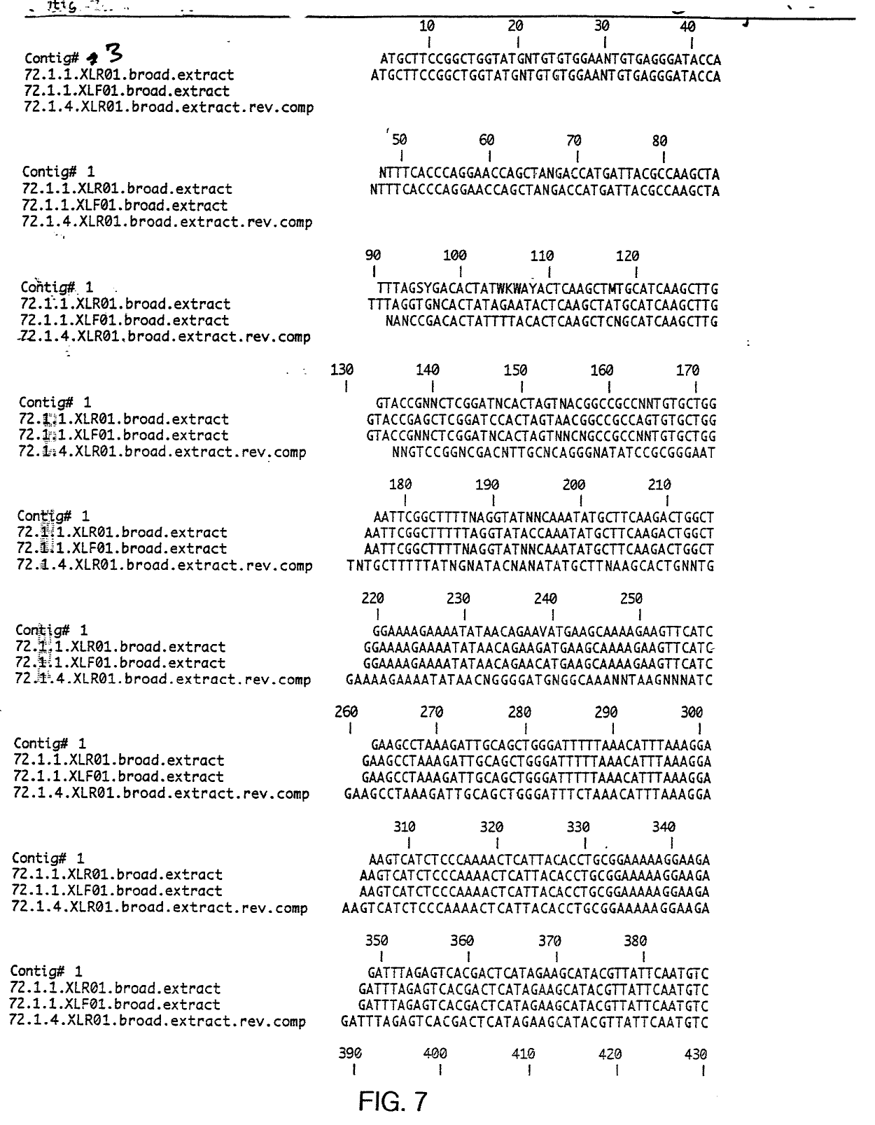

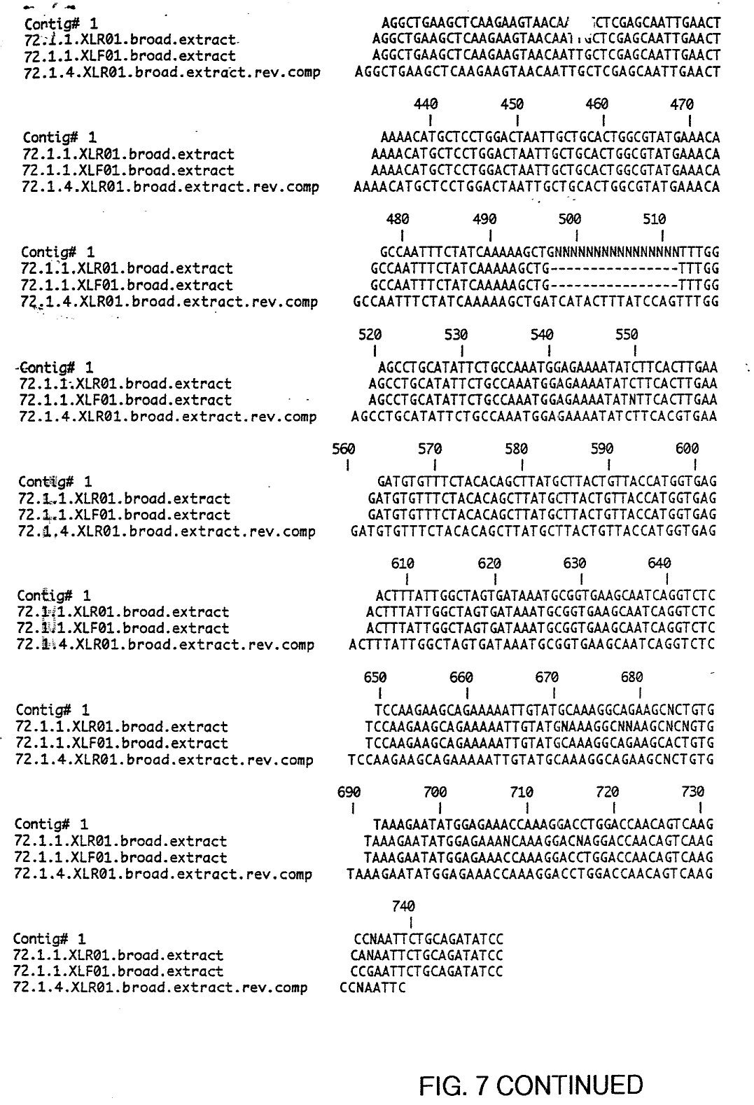

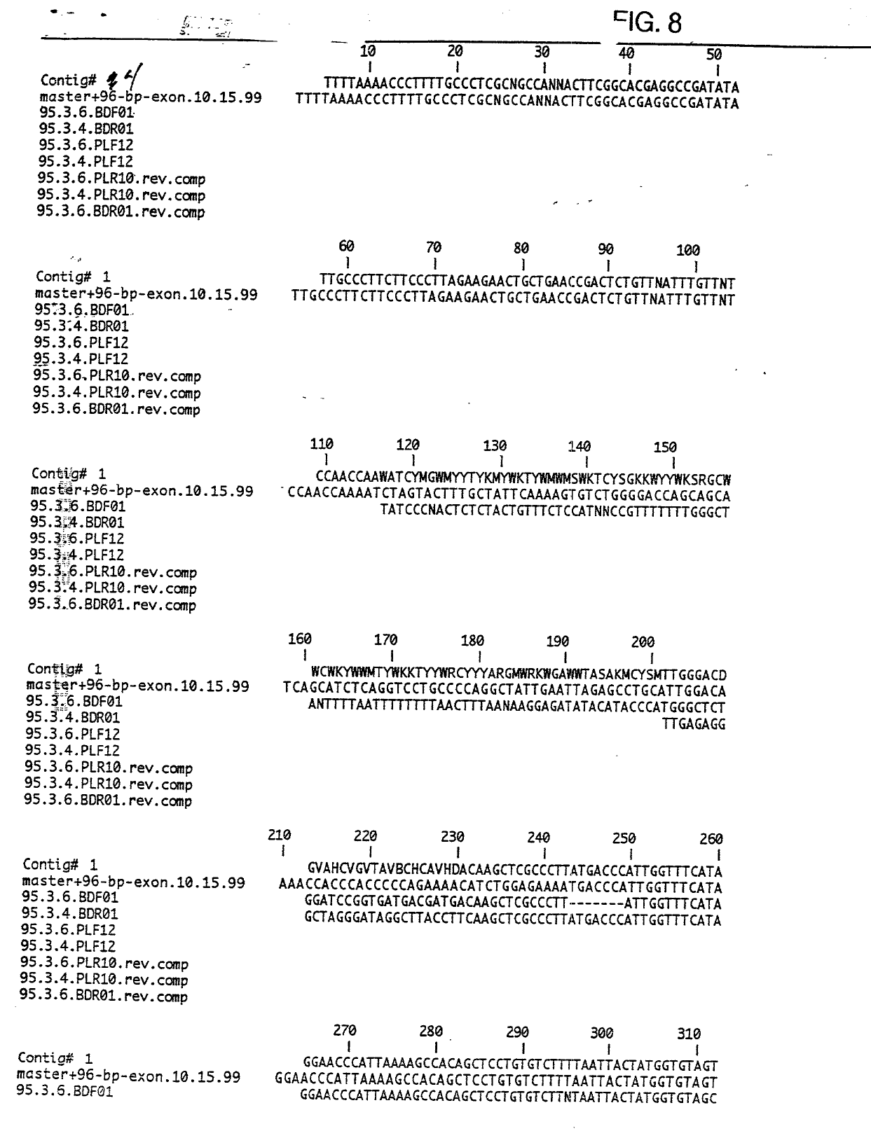

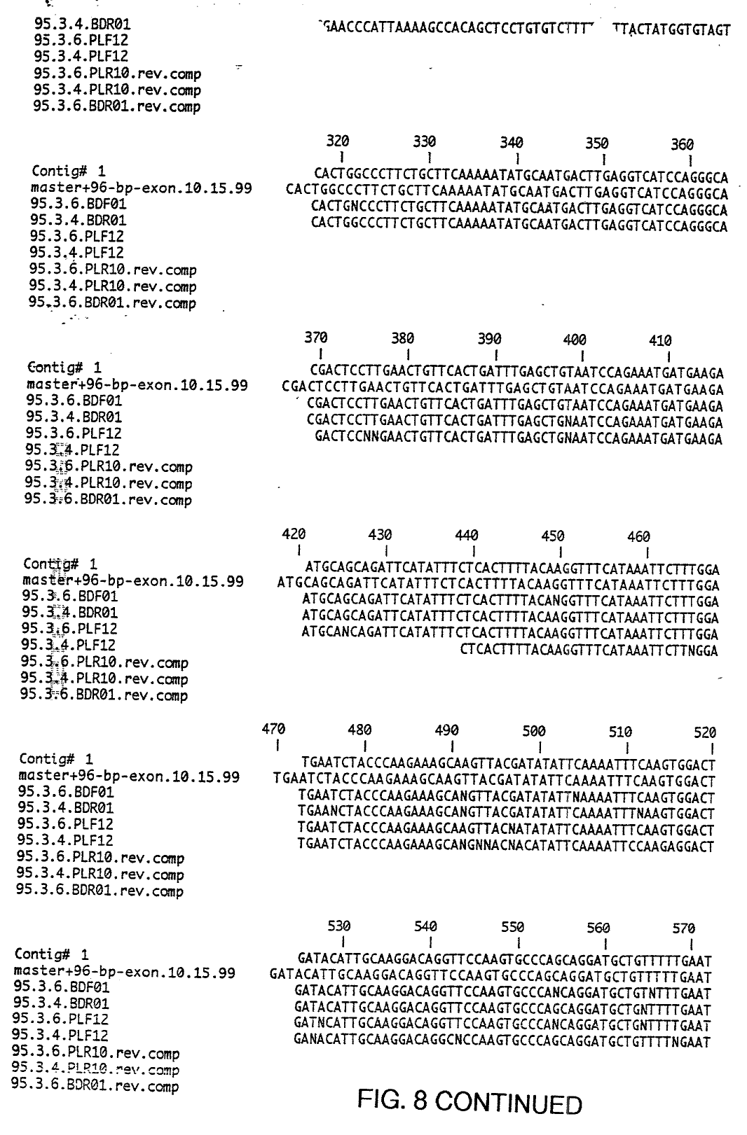

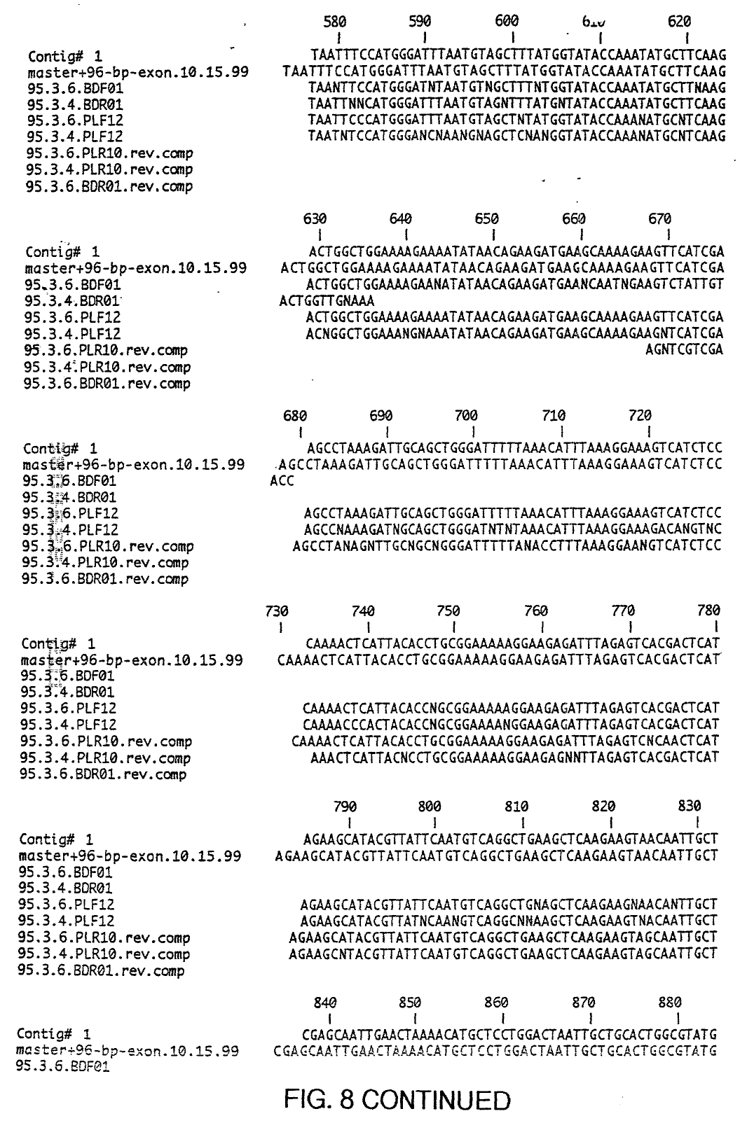

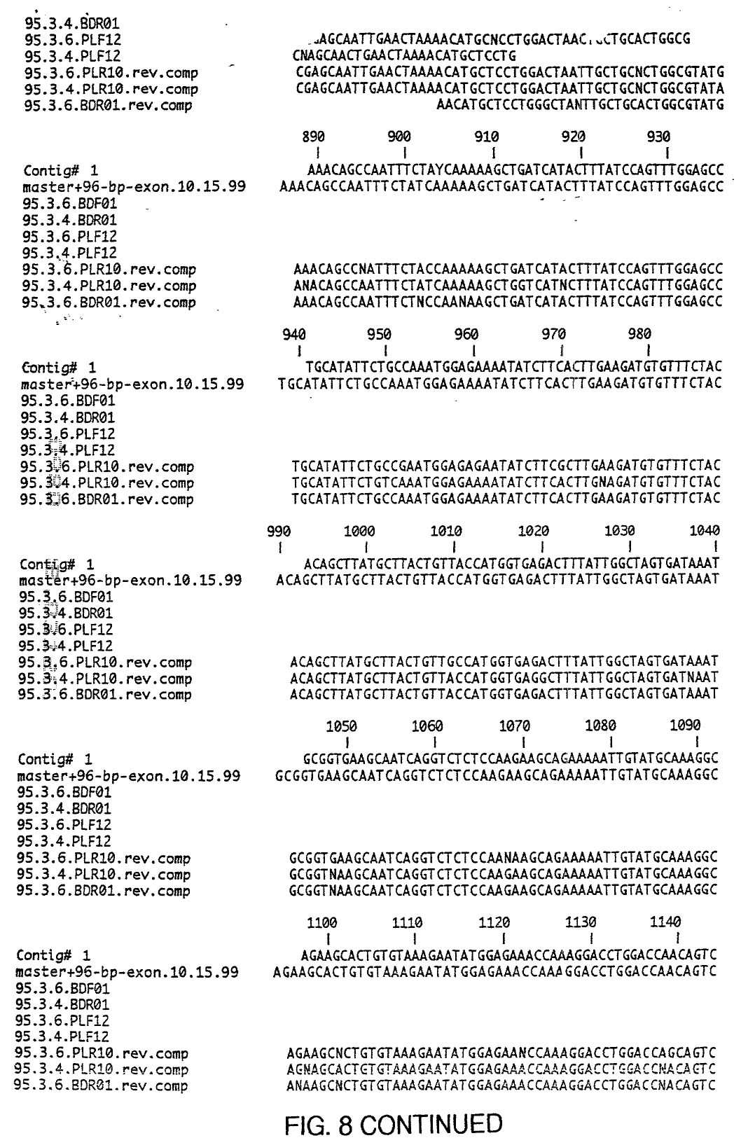

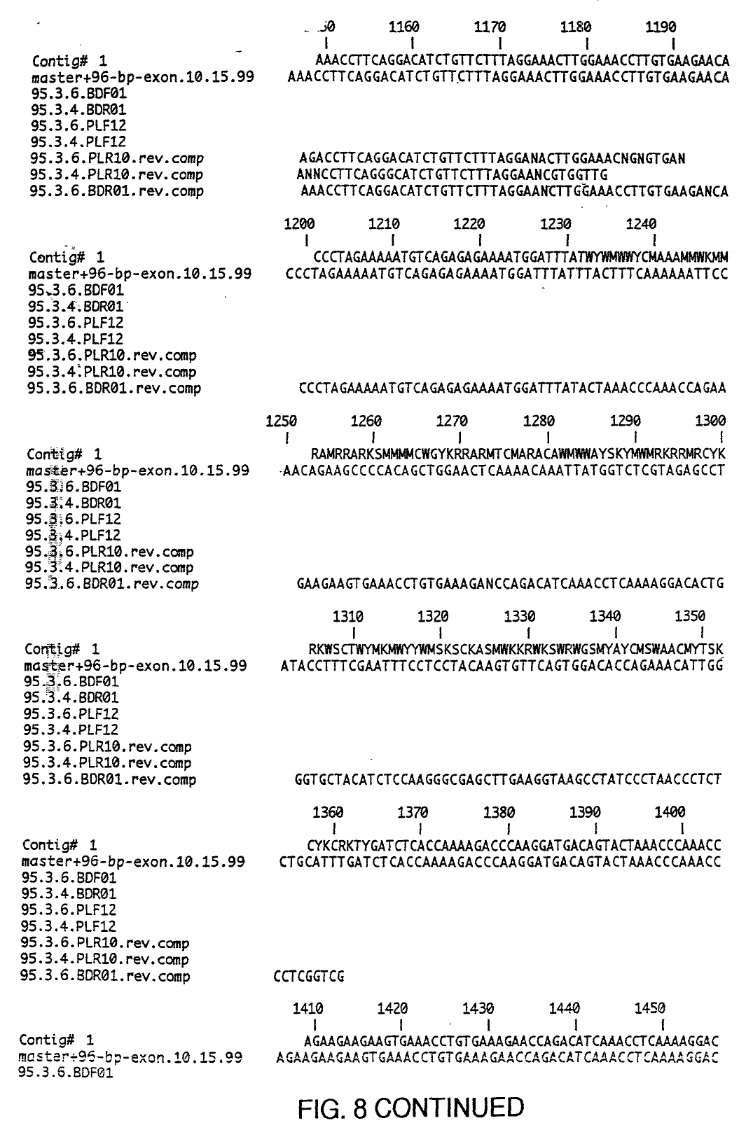

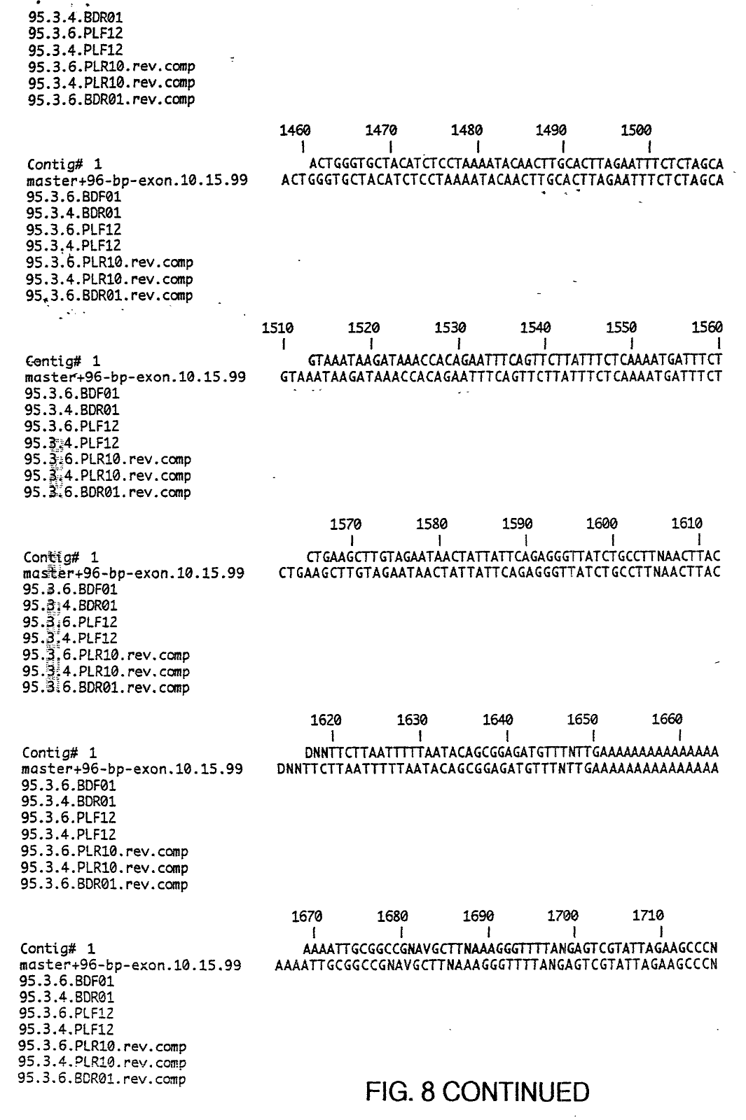

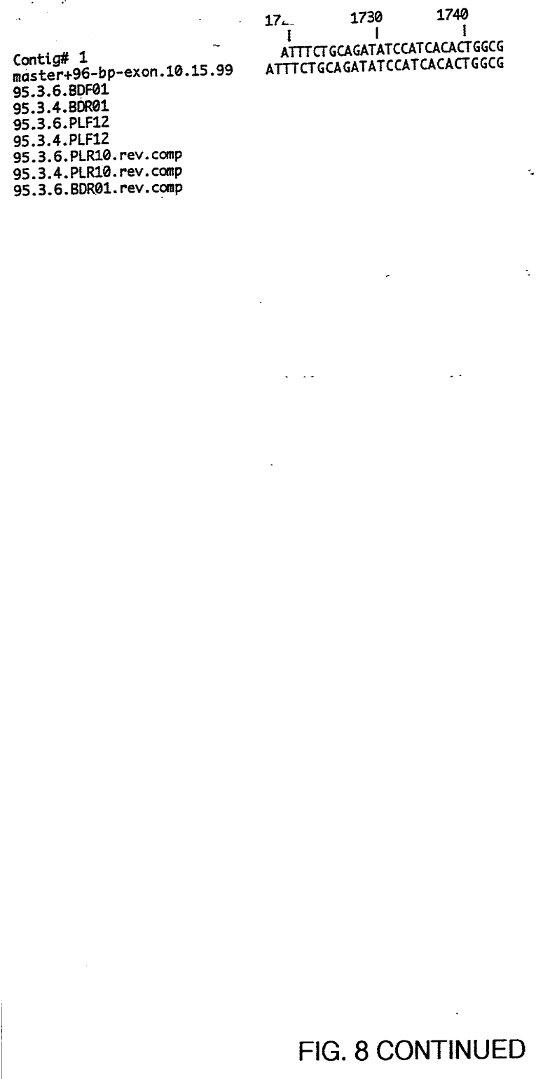

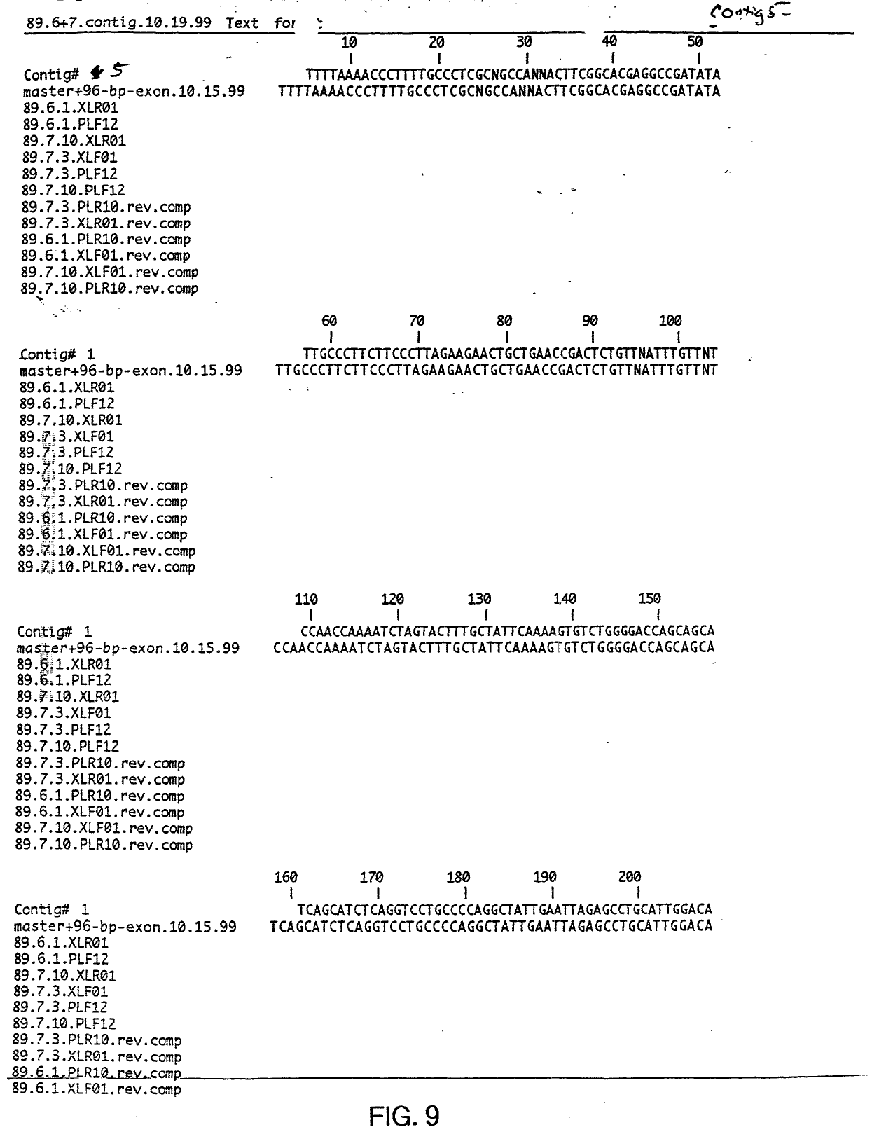

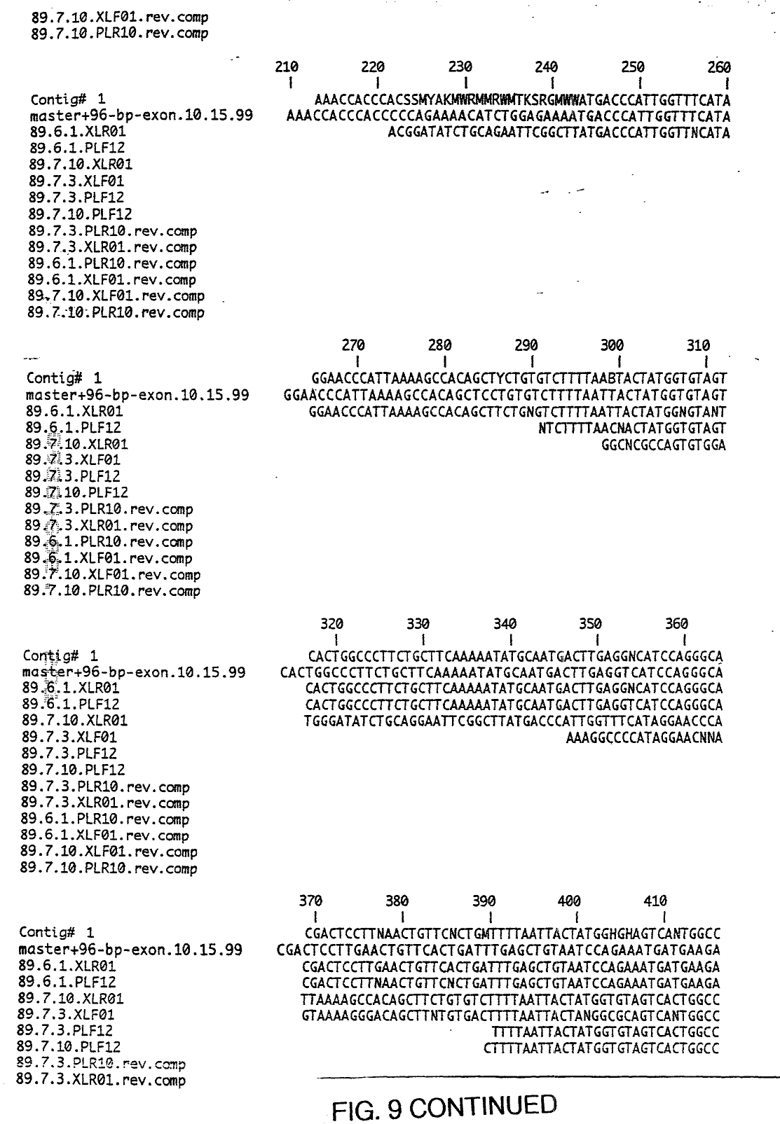

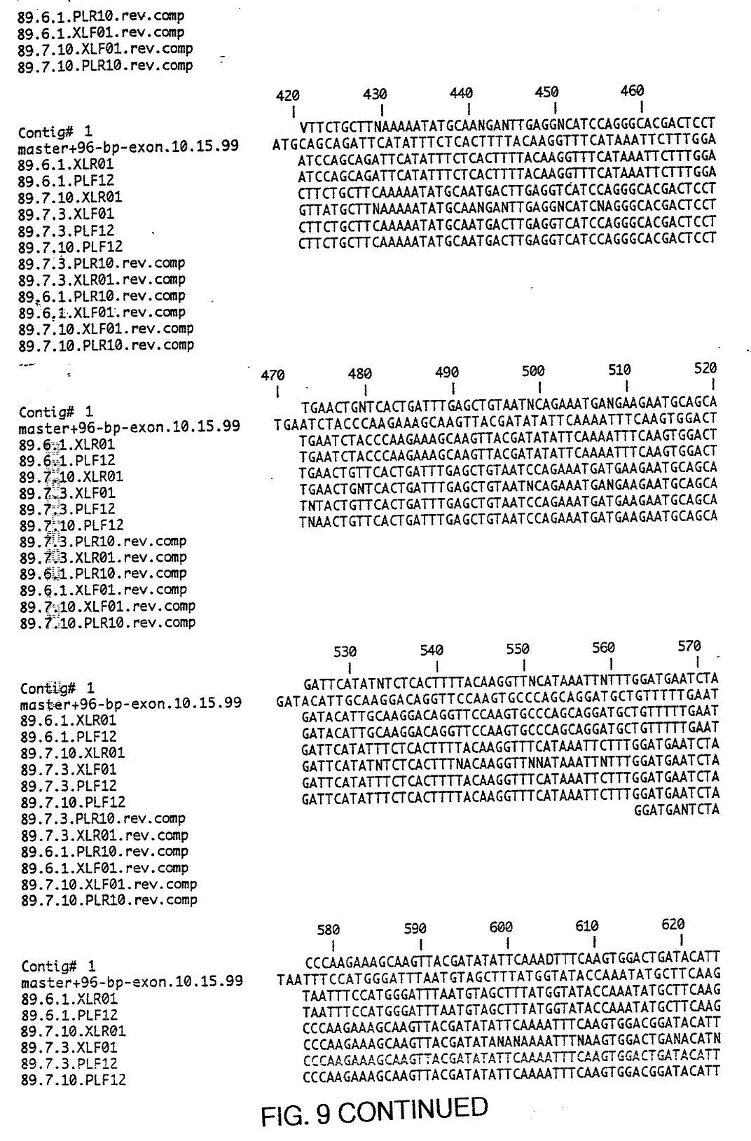

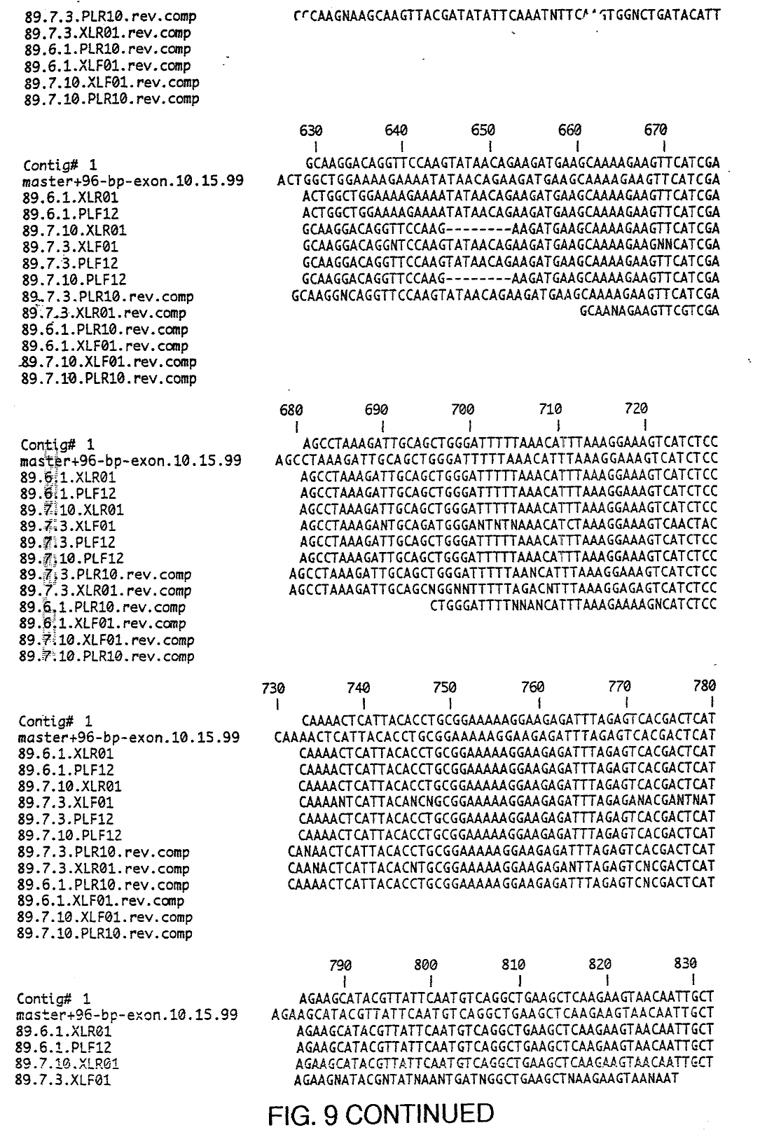

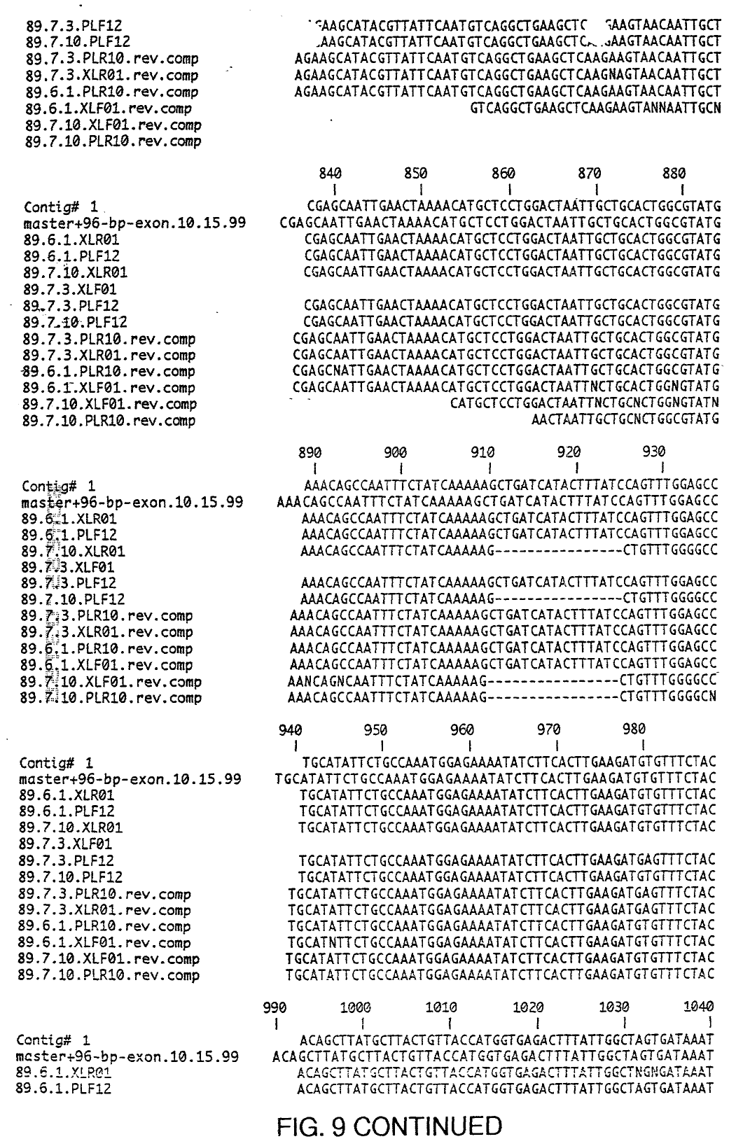

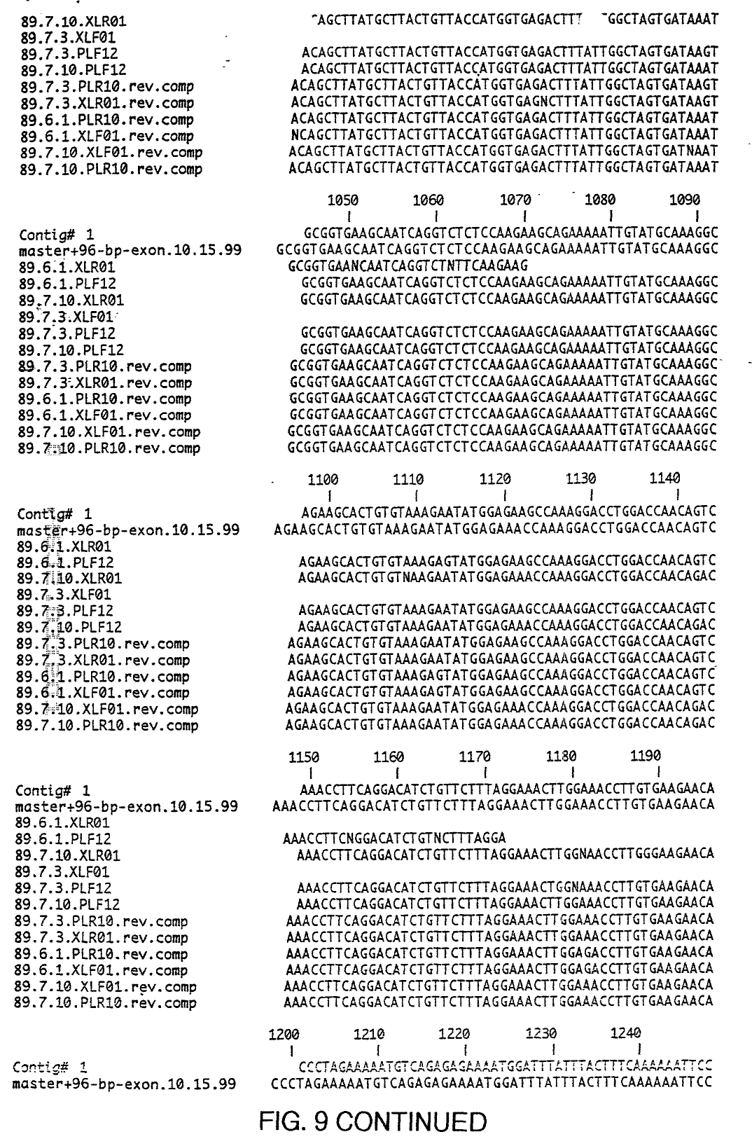

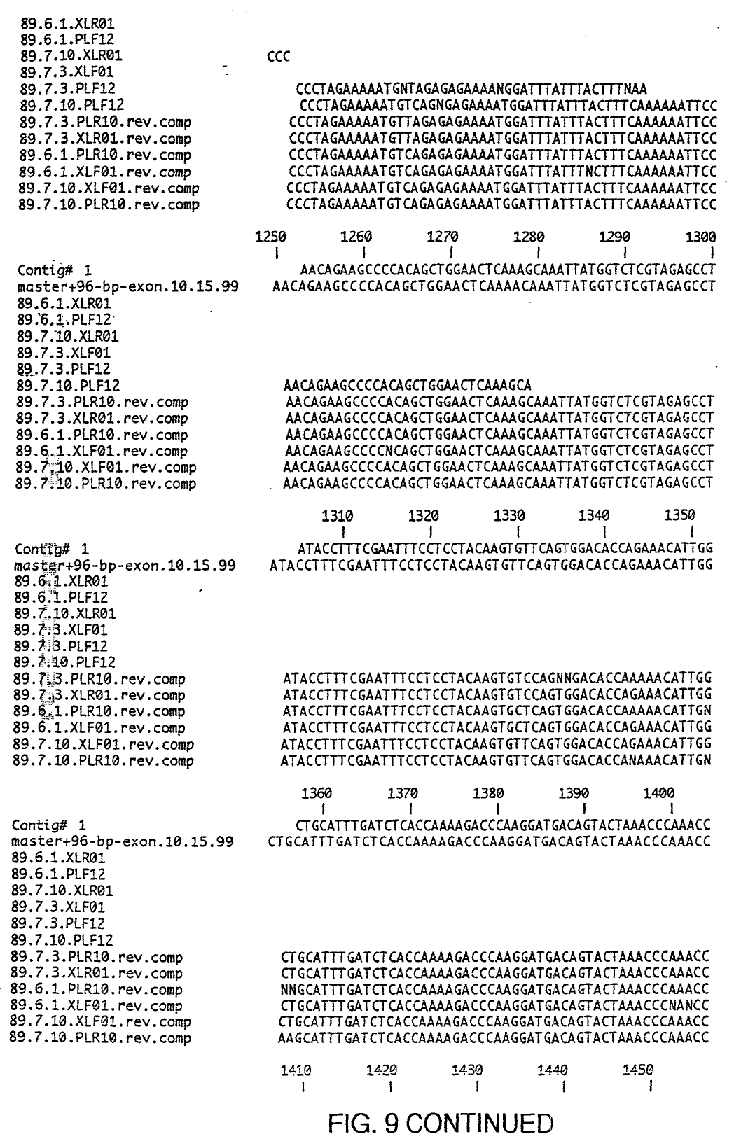

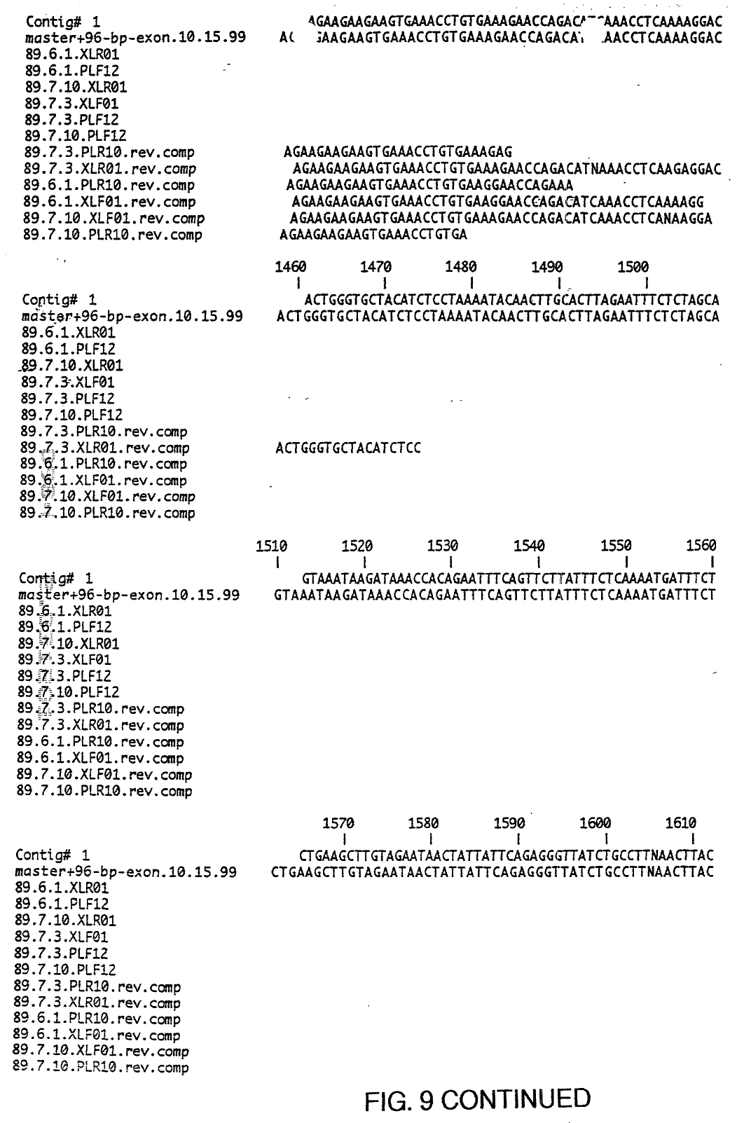

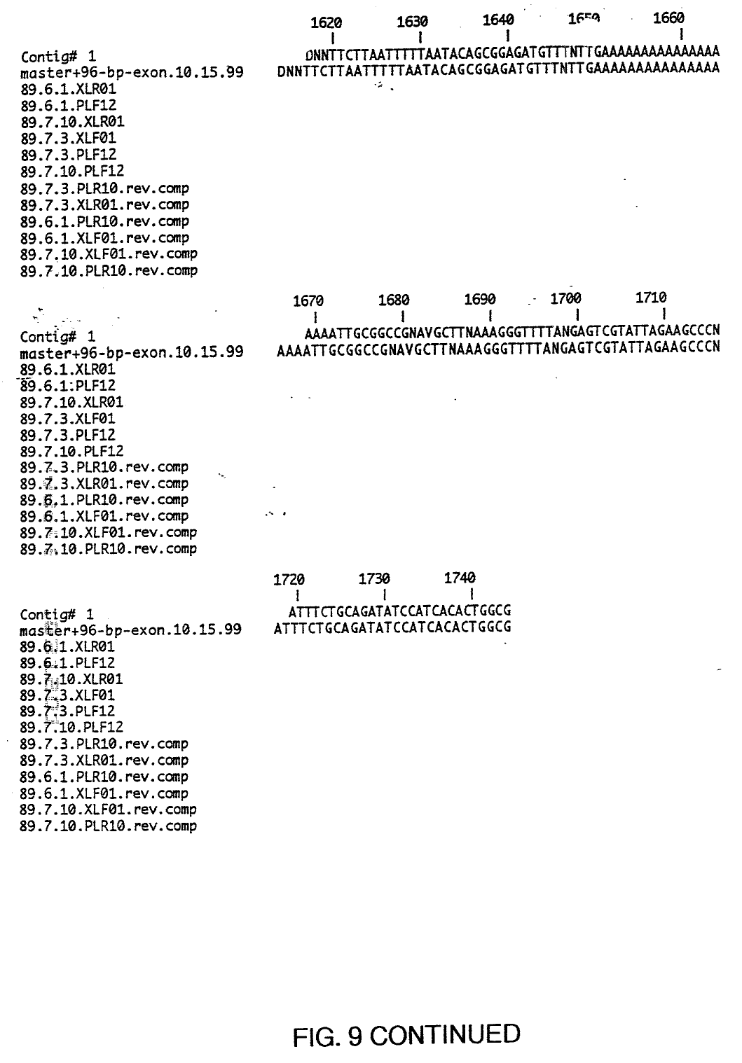

Similarly, cDNA made from RNA extracted colon cells was analyzed as below (FIG. 4 M=molecular weight marker; C=colon cancer; N=normal colon tissue from the same patients; Mic=microglobulin control for RNA uantity and quality. The three C's without matching N's are samples from cancers for which no matching normal control tissue was available).

Patients 1 & 2

PAL-18: PAL-175/1040RT primers, 95o×1′ initially, 30 cycles (94°×20″, 54°×1′, 70°×2′), 10 cycles (94°×20″, 54°×1′, 70°×2.5′), 70°×7′, 4° indefinitely.

Sequence of PAL-175: ACCAAATATGCTTCAAGACT (SEQ ID NO:173)

Microglobulin: Mic148 and Mic2533 primers. Same PCR conditions as for PAL-18 except annealing step was 70°×1′ instead of 54°. Primer sequences:

| | |
|---|---|
| Mic148: CACGTCATCCAGCAGAGAATGGAAACT | (SEQ ID NO:174) |
| Mic2533: TGACCAAGATGTTGATGTTGGATAAGAG | (SEQ ID NO:175) |

Patients 3-6

PAL-18: Same primers and conditions as above except 95°×2′ initially.

Microglobulin: Same primers and conditions as for microglobulin above except 95°×2′ initially.

### Example V

### Single Nucleotide Polymorphisms

Single Nucleotide Polymorphisms (SNPs) are individual variable positions in a gene sequence. SNPs can be useful for functional analysis of a gene product, “genetic fingerprinting,” and studies of disease susceptibility, as well as the possibility of “tailoring” a disease treatment for a patient. Possible SNPs in the known regions of the PAL-18 message are as follows:

Sequences based on SEQ ID NO:76, containing all except 96-bp exon

Pos.

359: G in clone 89.2.6, A elsewhere

520: G in clone 89.7.10, T elsewhere

565: G in clone 89.3.10, A elsewhere

620: A in 95.1.4, G elsewhere

704: C in clone 89.2.9, T elsewhere

776: A in clone 89.3.6, G elsewhere

800: C in clone 89.3.6, T elsewhere

821: ambiguity-clone 95.1.5 reads C in one sequence, G in the other; elsewhere it's A

830: T in clone 89.7.10, A elsewhere

837: G in clone 89.3.6 and clone 89.7.10, A elsewhere

884: A in clone 89.7.3, T elsewhere

893: Apparently G in clones 95.1.4 and 95.1.5, A elsewhere

943: G in clone 89.7.3, A elsewhere

946: G in clone 89.3.6, C elsewhere

987: G in clone 95.1.4, maybe T in Image clone 754, A elsewhere

1015: G in clone 89.6.1, A elsewhere

1025: G in clones 89.6.1 and 89.7.3, A elsewhere

1039: G in Image clones, A in others

1047: C in 3′ RACE clones, A in clone 89.7.10, T elsewhere

1098: A in prostate clones, 3′ RACE clones and one Image clone sequence, G in another sequence of the same clone and another Image clone, G and A both found in SW480 clones (95.1.4, 95.1.5)

1115: T in clone 89.7.3, C elsewhere

1161: T in 95.1.4, C elsewhere

1163: T in 95.2.1, C elsewhere

1170: A in clone 89.2.6, T elsewhere

1181: A or G variably; possibly some sequencing artifacts at this position

1234-1235: CT in 89.6.1, TC in 89.7.3, TT elsewhere

(Sequence based on contig ID 1 (SEQ ID NO:75), bases 549-645) base 43 of 96-bp exon: G in clone 89.3.10, A elsewhere

Use of SNPS in Diagnosis, Prognosis, Susceptibility Determination, and Determination of Optimum Treatment in Cancer and Other Diseases

Using the methods and principles discussed above, it would be possible to characterize an individual patient such as prostate-cancer patient B above according to the exact genotype of his PAL-18 by determining all SNPs, or only those found to be relevant to disease or susceptibility status. This may constitute part or all of a “genetic profile” which may be useful in diagnosis, prognosis, determining susceptibility, and/or specifying effective treatments in prostate cancer and other diseases.

The well-known genetic code can be used to translate nucleotide sequences incorporating these SNPs to yield hypothetical proteins in addition to those specified by sequence ID 72-74. It is also clear to one of skill in the art that variation in these SNPs may be combined with splice variation to produce additional species. However, if some or all of the putative “splice variants” discussed above are in fact the products of multiple genes, then some or all of the SNPs observed may in future be seen to occur with only certain of the “splice variants” (or products of multiple genes), and the SNPs may therefore be additionally useful as an alternative means of quantifying relative or absolute expression of these gene products (i.e., in addition to RT-PCR). Similarly, the relative or absolute abundance of a given “splice variant” or product associated with one of multiple genes could be used to infer the presence or amount of one or more SNPs associated with that particular gene, and therefore also with any functional variation associated with the SNP or SNPs.

### Example VI

### Assays for the Antigen

Given the characteristics of the antigen as described above and given the disclosure herein for generating and selecting antibodies and the development of certain assays described herein to detect the antigen, a number of additional assay formats beyond those described herein for this antigen may be readily developed by those of ordinary skill in the art. Suitable assay formats include competitive formats, sandwich formats (Examples VI.A., VI.B. and VI.C), assays based on the biological or chemical properties of the antigen (Example VI.D. and VI.E.), assays based on the simultaneous binding of the antigen to a specific macromolecule (e.g., PAL-18) and to a monoclonal antibody (Example VI.D.), assays based on the appearance of a band of appropriate size in partially-purified specimens (Example VI.F.), and RT-PCR (Example VI.G.). A preferred format involves sandwich immunoassays and the most preferred employs a monoclonal antibody immobilized on a solid surface and a second monoclonal antibody, which recognizes an epitope distinct from that of the first, conjugated to a detection agent. That detection agent could be an enzyme (Example VI.B.), colloidal gold (Example VI.C.), or any of a number of other such agents known to those of ordinary skill in the art. These include fluorescent molecules, radioisotopes, and biotin (which would subsequently bind to avidin or streptavidin-labeled detecting agent).

A. Identifying Potential Antibody Pairs

Definitions for the Section

| | |
|---|---|
| Indirect Assay Format: | Antigen coated on plate; reaction with MAb; |
| | signal generation by Goat Anti-mouse |
| | conjugated to alkaline phosphatase (GAM-AP). |
| Direct Assay Format: | Antigen coated on plate; reaction with and |
| | signal generation by specific MAb-AP. |
| Sandwich Assay Format: | As usual |

Initial screening of the cell culture supernatants is carried out using an ELISA in an indirect format. The assay consisted of the following in order: (1) diluted samples are adsorbed on a microtiter plate; (2) following washing, the microtiter plate wells is incubated with supernatants of cell cultures of the clones of interest; (3) following another wash, the plates are incubated with alkaline phosphatase-conjugated goat anti-mouse IgG; (4) following a final wash, the plates are incubated with p-nitrophenyl phosphate substrate (pNPP); and, finally, (5) the reactions are stopped by addition of concentrated EDTA to each well and the color measured at a wavelength of 410 mn on a microplate reader.

Initial testing of antibodies conjugated to alkaline phosphatase (AP), as described in Example VI.B.2., is carried out utilizing an assay in the direct format as follows: (1) diluted samples were adsorbed on a microtiter plate; (2) following washing, the plates were incubated with AP-conjugated antibodies from specific clones; (3) following a final wash, the plates were incubated with pNPP; and, finally, (4) the reactions are stopped and measured as above.

The sandwich ELISA format can be used as follows: (1) individual capture antibodies were adsorbed on microtiter plates; (2) following washing, diluted samples were added to the wells and incubated to allow binding of the antigen to the antibody; (3) following another wash, single conjugates (as described in B. above) are added to individual wells and incubated to allow binding to the antibody-bound antigen, if present; (4) following a final wash, the plates are incubated with pNPP; and, finally, (5) the reactions are stopped and measured as above.

B. Sandwich ELISA

The sandwich ELISA, utilizing the most preferred pair as selected above, is further optimized with respect to the following items: (1) coating level of capture antibody; (2) concentration of conjugate; (3) enzyme-to-antibody ratio in the conjugate; (4) reaction kinetics/incubation times; (4) composition of assay and wash buffers and of conjugate and specimen diluents; and (5) formulation of standards and controls. The assay as optimized is performed as follows:

1. Preparation of Coated Plates

The plates are coated with 150 μl per well of monoclonal antibody at a concentration of 5 μg/mL in carbonate buffer at pH 9.6. The plates are then blocked with 2% bovine serum albumin in phosphate-buffered saline at pH 7.4, followed by blocking with 4% sucrose. The sucrose solution is decanted, and the plates are dried overnight at room temperature.

2. Preparation of MAb-Alkaline Phosphatase Conjugates

Antibodies are purified by chromatography on immobilized Protein G or Protein A by standard techniques. Although antibody-enzyme conjugates could be prepared using a variety of coupling techniques (for review see Scouten, W. H., Methods in Enzymology 135:30-65, 1987), a minor variation of a method described by S. Hashida and E. Ishikawa (Anal. Lett. 18, B9:1143-1155, 1985) is preferred. Briefly, purified monoclonal antibodies are treated with excess N-acetylhomocysteine thiolactone (AHTL) at neutral pH to introduce reactive thiol groups, and then desalted to remove excess AHTL. Separately, alkaline phosphatase (AP) is treated with excess sulfosuccinimidyl 4-(N-maleimido-methyl) cyclohexane-l-carboxylate to introduce maleimido groups, and excess reagent is removed by desalting. The conjugates are prepared by mixing antibody and enzyme derivatives, which become covalently coupled via thioether bonds. Any excess maleimido groups are then capped by reaction with cysteamine.

3. Assay Format

A volume of 175 μl of assay buffer is pipetted into each well to be utilized in carrying out the assay. The buffer is followed by 25 μl of samples, standards, or controls, thus yielding a 1/8 dilution in the well. Incubation of the covered plate is performed at 37° C. for 60 minutes. Following washing, 200 μl of working dilution of conjugate is added to the aspirated well. The covered plate is again incubated for 60 minutes at 37° C. Following a final wash, 200 μl of pNPP substrate is pipetted into each well, and the covered plate is incubated at 37° C. for 30 minutes. After pipetting, 50 μl of stop solution into each well, the reaction mixtures in each well is measured at 410 nm.

C. PAL-18-MAb ELISA

1. Method

Immulon 4 (Dynatech, Chantilly, Va.) microtiter strip wells are coated with 50 μl per well of 5 μg/ml PAL-18 in 50 mM carbonate buffer, pH 9.6, either overnight at 4° C. or for two hours at 37° C. A control plate is coated with 50 μl per well of 2% BSA in PBS for two hours at 37° C. After a single wash with Tris-buffered saline (TBS) containing 0.1% Tween −20 (wash buffer), the plates are blocked with 100 μl per well of a 2% BSA solution in PBS for two hours at 37° C. and washed four times. Antigen, diluted in assay diluent (1% BSA in TBS with 0.15M MgCl₂, 0.15M ZnCl₂), is added at 50 μl per well and incubated for one hour at 37° C. The plates are washed four times and then the detection antibody (MAb-alkaline phosphatase) is applied at 0.25 μg/ml, 50 μl per well, and incubated at 37° C. for 30 minutes. After four washes, 50 μl per well of p-nitrophenyl phosphate (Sigma, St. Louis, Mo.) at 1 mg/ml in 1M diethanolamine (DEA) is added and the plate incubated for 30 minutes at 37° C. The reaction is stopped with 25 μl per well of stop solution (0.1 M EDTA, pH 9.8) and the plate read at 405 nm on a Dynatech MR7000 reader.

D. RT-PCR Assay

1. Cell Lines

Several cell lines, particularly cell lines HTB-9, which is derived from Transitional Cell Carcinoma (TCC) of the bladder and HeLaS3, which is derived from adenocarcinoma of the cervix and LS174T, derived from colon carcinoma (all from American Type Culture Collection, Rockville, Md.), are tested to determine whether they produce mRNA coding for the antigen. Although the method selected for cell line analysis is RT-PCR (Reverse Transcriptase based Polymerase Chain Reaction amplification of messenger RNA, mRNA), a variety of procedures used to detect the presence of specific RNA can be used. Controls are performed using PCR target materials provided with commercial PCR kits.

2. Preparation of mRNA

Preparation of mRNA is facilitated by the use of a Lysis Buffer containing: 7.5 M Guanidine HCl, 25 mM TES, 10 mM EDTA, 0.05% Taurodeoxycholate, 1 mM 2-mercaptoethanol, pH 7.5 (all reagents Molecular Biology grade from Sigma, St. Louis, Mo.). This buffer eliminates the necessity for grinding or icing samples and resulted in a stable preparation of DNA and RNA.

Cells are lysed in 1 mL lysis buffer per 10⁸ cells/mL cell culture media (IMDM, Irvine Scientific; Irvine, Calif.) supplemented with 15% FBS (Hyclone; Logan, Utah). The lysate is extracted with equal volumes of phenol and chloroform/isoamyl alcohol. The aqueous phase was aspirated and re-extracted with an equal volume of chloroform/isoamyl alcohol. The aqueous phase is precipitated with 7/13 volumes 10M LiCl (all reagents Molecular Biology Grade from Sigma Chemicals, St. Louis, Mo.). The mRNA is prepared from the total RNA produced in the previous steps using a PolyATtract kit (Promega, Madison, Wis.).

3. RT-PCR Amplification

RT-PCR amplification of antigen sequences is performed on a Perkin-Elmer 2400 Thermal Cycler using a GeneAmp PCR kit (Perkin-Elmer/Roche Molecular Systems, Branchburg, N.J.). Amplification is performed with 3 μL purified mRNA, 35 cycles, for the first amplification step of each reaction. The RT primer may be any of those described above or derived from the PAL-18 sequences. For the nested PCR, 20 μL of product from the first PCR reaction is re-amplified for 35 cycles using at least one additional prime. All primer concentrations are set at 0.2 μM, and the annealing temperature is set at 48° C.

The invention is in no way limited except as set forth by the attached claims.

1. An isolated PAL-18 polypeptide comprising an amino acid sequence that is encoded by a polynucleotide sequence selected from the group consisting of:
(a) a sequence recited in SEQ ID NOs:1-71;
(b) sequences that hybridize to a sequence recited in SEQ ID NOs:1-71 or 75-79 under moderately stringent conditions; and
(c) complements of the polynucleotide sequences of (a) and (b).

2. An isolated polypeptide according to claim 1, wherein the polypeptide comprises an amino acid sequence that is encoded by a polynucleotide sequence recited in SEQ ID NOs:75-79 or a complement thereof.

3. An isolated PAL-18 polypeptide comprising SEQ ID NOs:72-74.

4. A fragment of a PAL-18 polypeptide comprising at least 15 contiguous amino acid residues of SEQ ID NOs:72-74.

5. An isolated polynucleotide encoding at least 15 amino acid residues of a PAL-18 polypeptide, or a variant thereof that differs in one or more substitutions, deletions, additions and/or insertions such that the ability of the variant to react with antigen-specific antisera is not substantially diminished, wherein the PAL-18 polypeptide comprises an amino acid sequence that is encoded by a polynucleotide comprising a sequence recited in SEQ ID NOs:1-71, 75-79 or a complement thereof.

6. An isolated polynucleotide encoding a PAL-18 polypeptide or a variant thereof, wherein the PAL-18 polypeptide comprises an amino acid sequence that is encoded by a polynucleotide comprising a sequence recited in SEQ ID NOs: 1-71, 75-79, a complement thereof, or a sequence having at least 70% identity with SEQ ID NOs: 1-71 or 75-79.

7. An isolated polynucleotide, comprising a sequence recited in SEQ ID NOs: 1-71 or 75-79.

8. An isolated polynucleotide, comprising a sequence that hybridizes to a sequence recited in SEQ ID NOs:1-71 or 75-79 under moderately stringent conditions.

9. An isolated polynucleotide, comprising a sequence having at least 70% identity with SEQ ID NOs:1-71 or 75-79.

10. An isolated polynucleotide complementary to a polynucleotide according to any one of claims 5-9.

11. An expression vector, comprising a polynucleotide according to any one of claims 5-9.

12. A host cell transformed or transfected with an expression vector according to claim 11.

13. An isolated antibody, or antigen-binding fragment thereof, that specifically binds to a PAL-18 polypeptide or fragment thereof that comprises an amino acid sequence that is encoded by a polynucleotide sequence recited in SEQ ID NOs: 1-71, 75-79 or a complement thereof.

14. A pharmaceutical composition, comprising a physiologically acceptable carrier and at least one component selected from the group consisting of:
(a) a polypeptide according to claim 1;
(b) a fragment according to claim 4;
(c) a polynucleotide according to claim 5; and
(d) an antibody according to claim 13.

15. A method for determining the presence or absence of a cancer in a patient, comprising the steps of:
(a) contacting a biological sample obtained from a patient with a binding agent that binds to a PAL-18 polypeptide, wherein the PAL-18 polypeptide comprises an amino acid sequence that is encoded by a polynucleotide sequence recited in SEQ ID NOs: 1-71, 75-79 or a complement thereof;
(b) detecting in the sample an amount of polypeptide that binds to the binding agent; and
(c) comparing the amount of polypeptide to a predetermined cut-off value, and therefrom determining the presence or absence of a cancer in the patient.

16. The method according to claim 15, wherein the binding agent is an antibody.

17. The method according to claim 16, wherein the antibody is a monoclonal antibody.

18. The method according to claim 15, wherein the cancer is prostate or colon cancer.

19. The method according to claim 15, wherein the cancer is breast cancer.

20. The method according to claim 15, wherein the detecting step involves detection of various molecular weight isoforms of PAL-18.

21. The method according to claim 15, wherein the detecting step involves detection of expression levels of PAL-18.

22. The method according to claim 15, wherein the biological sample is selected from the group consisting of a tissue sample, a urine sample, a blood sample, and a stool sample.

23. The method according to claim 22, wherein the biological sample is a stool sample.

24. A method for monitoring the progression of a cancer in a patient, comprising the steps of:
(a) contacting a biological sample obtained from a patient at a first point in time with a binding agent that binds to a PAL-18 polypeptide or fragment thereof, wherein the PAL-18 polypeptide or fragment thereof comprises at least 15 contiguous amino acid residues encoded by a polynucleotide sequence recited in SEQ ID NOs:1-71, 75-79 or a complement thereof;
(b) detecting in the sample an amount of polypeptide that binds to the binding agent;
(c) repeating steps (a) and (b) using a biological sample obtained from the patient at a subsequent point in time; and
(d) comparing the amount of polypeptide detected in step (c) to the amount detected in step (b) and therefrom monitoring the progression of the cancer in the patient.

25. The method according to claim 24, wherein the binding agent is an antibody.

26. The method according to claim 25, wherein the antibody is a monoclonal antibody.

27. The method according to claim 24, wherein the cancer is colon cancer or prostate cancer.

28. The method according to claim 24, wherein the cancer is breast cancer.

29. The method according to claim 24, wherein the detecting step involves detection of various molecular weight isoforms of PAL-18.

30. The method according to claim 24, wherein the detection step involves detection of expression levels of PAL-18.

31. The method according to claim 24, wherein the biological sample is selected from the group consisting of a tissue sample, a urine sample, a blood sample, and a stool sample.

32. The method according to claim 31, wherein the biological sample is a stool sample.

33. A method for determining the presence or absence of a cancer in a patient, comprising the steps of:
(a) contacting a biological sample obtained from a patient with an oligonucleotide that hybridizes to a polynucleotide that encodes a PAL-18 polypeptide or fragment thereof, wherein the PAL-18 polypeptide comprises at least 15 contiguous amino acid residues encoded by a polynucleotide sequence recited in SEQ ID NOs:1-71, 75-79 or a complement thereof and wherein the at least 15 contiguous amino acid residues retain the ability to react with an a PAL18-specific antisera;
(b) detecting in the sample an amount of a polynucleotide that hybridizes to the oligonucleotide; and
(c) comparing the amount of polynucleotide that hybridizes to the oligonucleotide to a predetermined cut-off value, and therefrom determining the presence or absence of a cancer in the patient.

34. The method according to claim 33, wherein the amount of polynucleotide that hybridizes to the oligonucleotide is determined using a polymerase chain reaction.

35. The method according to claim 34, wherein the polymerase chain reaction is RT-PCR.

36. The method according to claim 34, wherein the amount of polynucleotide that hybridizes to the oligonucleotide is determined using a hybridization assay.

37. A method for monitoring the progression of a cancer in a patient, comprising the steps of:
(a) contacting a biological sample obtained from a patient with an oligonucleotide that hybridizes to a polynucleotide that encodes a PAL-18 polypeptide or fragment thereof, wherein the PAL-18 polypeptide comprises at least 15 contiguous amino acid residues encoded by a polynucleotide sequence recited in SEQ ID NOs:1-71, 75-79 or a complement thereof and wherein the at least 15 contiguous amino acid residues retain the ability to react with an a PAL-18-specific antisera;
(b) detecting in the sample an amount of a polynucleotide that hybridizes to the oligonucleotide;
(c) repeating steps (a) and (b) using a biological sample obtained from the patient at a subsequent point in time; and
(d) comparing the amount of polynucleotide detected in step (c) to the amount detected in step (b) and therefrom monitoring the progression of the cancer in the patient.

38. The method according to claim 37, wherein the amount of polynucleotide that hybridizes to the oligonucleotide is determined using a polymerase chain reaction.

39. The method according to claim 38, wherein the polymerase chain reaction is RT-PCR.

40. The method according to claim 38, wherein the amount of polynucleotide that hybridizes to the oligonucleotide is determined using a hybridization assay.

41. A diagnostic kit, comprising:
(a) one or more antibodies according to claim 13; and
(b) a detection reagent comprising a reporter group.

42. The kit according to claim 41, wherein the antibodies are immobilized on a solid support.

43. The kit according to claim 41, wherein the detection reagent comprises an anti-immunoglobulin, protein G, protein A or lectin.

44. The kit according to claim 41, wherein the reporter group is selected from the group consisting of radioisotopes, fluorescent groups, luminescent groups, enzymes, biotin and dye particles.

45. An oligonucleotide comprising 10 to 40 contiguous nucleotides that hybridize under highly stringent conditions to a polynucleotide that encodes a PAL-18 polypeptide or fragment thereof, wherein the PAL-18 polypeptide or fragment thereof comprises an amino acid sequence that is encoded by a polynucleotide sequence recited in any one of SEQ ID NOs:1-71, 75-79 or a complement thereof.

46. A oligonucleotide according to claim 45, wherein the oligonucleotide comprises 10-40 contiguous nucleotides recited in any one of SEQ ID NOs:75-79.

47. A diagnostic kit, comprising:
(a) an oligonucleotide according to claim 45 or claim 46; and
(b) a diagnostic reagent for use in a polymerase chain reaction or hybridization assay.

48. A method of treating a tumor cell comprising the step of modulating a tumor-associated PAL-18 polypeptide or a nucleic acid molecule encoding said polypeptide, said nucleic acid molecule characterized by the ability of said nucleic acid molecule to hybridize under moderate stringency with any one of SEQ ID NOs:1-71, 75-79, or a complement thereof.

49. The method according to claim 48, wherein the method comprises the step of modulating the antigen.

50. The method according to claim 48 or 49, wherein the tumor cell is a colorectal or prostate tumor cell.

51. An agent that modulates a tumor-associated PAL-18 polypeptide or a nucleic acid molecule encoding said polypeptide, said nucleic acid molecule characterized by the ability of said nucleic acid molecule to hybridize under moderate stringency with any one of SEQ ID NOs:1-71, 75-79, or a complement thereof.

52. A composition comprising an agent that modulates a PAL-18 polypeptide or a nucleic acid molecule encoding said polypeptide, said nucleic acid molecule characterized by the ability of said nucleic acid molecule to hybridize under moderate stringency with any one of SEQ ID NOs:1-71, 75-79, or a complement thereof, in combination with a pharmaceutically acceptable carrier or diluent.

53. A method for diagnosing abnormal PAL-18, comprising isolating PAL-18 encoding nucleic acid molecules from a sample and subsequently screening for single nucleotide polymorphisms, whereby detection of a single nucleotide polymorphism indicates an abnormal PAL-18.

54. A method for in vivo imaging, comprising administering to an animal an binding agent that specifically binds a PAL-18 polypeptide, said binding agent having an imaging agent attached thereto.

55. The method according to claim 54, wherein the binding agent is an antibody.
